SEQUENCE LISTING (755 sequences)

SEQ ID NO: 1 - DNA, 533 bp, Homo sapiens
  source 1..533: mol_type = genomic DNA
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 2 - DNA, 533 bp, Homo sapiens
  source 1..533: mol_type = genomic DNA
catagcagcttcttgtccagctttattgggaggccagcatgcctggaggggggccaggcatgaggtggggtaggagagcactgagaatactgtcccttttaagcaacctacaggggcagccctggagattgcaggacccaaggagctcgcaggatggataggcaggtggacttggggtattgaggtctcaggcagccacggctgaagttggtctgacctcagggtccaaatcccagaactcagagaacttgtccttaacggtgctccagtagtctttcagggaactgaagccatcggtcacccagcccctggcctgctgggccacctgggactcctgcacgctgctcagtgcatccttggcggtcttggtggcgtgcttcatgtaaccctgcatgaagctgagaagggaggcatcctcggcctctgaagctcgggcagaggccaggagcgccaggagggcaacaacaaggagtacccggggctgcatggcacctctgttcctggagcagctgcctctagggatgaactgagca

SEQ ID NO: 3 - DNA, 567 bp, Macaca fascicularis
  source 1..567: mol_type = genomic DNA
aatataaaacaggtcagaaccctcctgcctgcctgctctgttcatccctagaggcagctgctccaggaacagaggcgccatgcagccccgggtactccttgttgctgccctgctgtcactcctggcctctgccagagcttcagaggccgaggacacctcccttcttggcttcatgcagggctacatgcagcatgccaccaagaccgccaaggatgcactgaccagcgtccaggagtcccaggtggcccagcaggccagaggctgggtgaccgatggcttcagttccctgaaagactactggagcaccgttaaggacaagttatctgggttctgggatttgaaccctgaggccaaacccactctggctgaggctgcctgagacctcaataccccaagtccacctgcctgtccatcctgccagctccttgggtcctgcagcctccagggctgcccctgtaggttgcttaaaagggacagtattctcagtgccctcctaccgcacctcatgcctggcccccctccaggcagggtgtcctcccaataaagctggacaagaagctgctatga

SEQ ID NO: 4 - DNA, 567 bp, Macaca fascicularis
  source 1..567: mol_type = genomic DNA
tcatagcagcttcttgtccagctttattgggaggacaccctgcctggaggggggccaggcatgaggtgcggtaggagggcactgagaatactgtcccttttaagcaacctacaggggcagccctggaggctgcaggacccaaggagctggcaggatggacaggcaggtggacttggggtattgaggtctcaggcagcctcagccagagtgggtttggcctcagggttcaaatcccagaacccagataacttgtccttaacggtgctccagtagtctttcagggaactgaagccatcggtcacccagcctctggcctgctgggccacctgggactcctggacgctggtcagtgcatccttggcggtcttggtggcatgctgcatgtagccctgcatgaagccaagaagggaggtgtcctcggcctctgaagctctggcagaggccaggagtgacagcagggcagcaacaaggagtacccggggctgcatggcgcctctgttcctggagcagctgcctctagggatgaacagagcaggcaggcaggagggttctgacctgttttatatt

SEQ ID NO: 5 - DNA, 585 bp, Macaca mulatta
  source 1..585: mol_type = genomic DNA
gagacaatataaaacaggtcagaaccctcctgcctgcctgctctgttcatccctagaggcagctgctccaggaacagaggcgccatgcagccccgggtactccttgttgctgccctgctgtcactcctggcctctgccagagcttcagaggccgaggacacctcccttcttggcttcatgcaggactacatgcagcatgccaccaagaccgccaaggatgcactgaccagcgtccaggagtcccaggtggcccagcaggccagaggctgggtgaccgatggcttcagttccctgaaagactactggagcaccgttaaggacaagttatctgggttctgggatttgaaccctgaggccaaacccactctggctgaggctgcctgagacctcaataccccaagtccacctgcctgtccatcctgccagctccttgggtcctgcagcctccagggctgcccctgtaggttgcttaaaagggacagtattctcagtgccctcctaccgcacctcatgcctggcccccctccaggcagggtatcctcccaataaagctggacaagaagctgctatgagtgggccgtcaca

SEQ ID NO: 6 - DNA, 585 bp, Macaca mulatta
  source 1..585: mol_type = genomic DNA
tgtgacggcccactcatagcagcttcttgtccagctttattgggaggataccctgcctggaggggggccaggcatgaggtgcggtaggagggcactgagaatactgtcccttttaagcaacctacaggggcagccctggaggctgcaggacccaaggagctggcaggatggacaggcaggtggacttggggtattgaggtctcaggcagcctcagccagagtgggtttggcctcagggttcaaatcccagaacccagataacttgtccttaacggtgctccagtagtctttcagggaactgaagccatcggtcacccagcctctggcctgctgggccacctgggactcctggacgctggtcagtgcatccttggcggtcttggtggcatgctgcatgtagtcctgcatgaagccaagaagggaggtgtcctcggcctctgaagctctggcagaggccaggagtgacagcagggcagcaacaaggagtacccggggctgcatggcgcctctgttcctggagcagctgcctctagggatgaacagagcaggcaggcaggagggttctgacctgttttatattgtctc

SEQ ID NO: 7 - DNA, 527 bp, Mus musculus
  source 1..527: mol_type = genomic DNA
gcctgctcagttttatccctagaagcagctagctactccaggtacgtaggtgccatgcagccccggacgctcctcactgtggccctcttggctctcctggcatctgcccgagctgaagaggtagagggatccttgctgctgggctctgtacagggctacatggaacaagcctccaagacggtccaggatgcgctaagtagcgtgcaggagtccgatatagctgtggtggccaggggctggatggacaatcacttcagatccctgaaaggctactggagcaagtttactgacaagttcaccggcttctgggattctaaccctgaggaccaaccaactccagctattgagtcgtgagacttctgtgttgcagatgtgcctgttcctccatcctgctgcccccctccaggcctgccaggtggcccctgaaggttgctttaaggggaaagtatgttctcatgtcttcacccctccctagatctcacctaaacatgctgtccctaataaagctggataagaagctgctgtta

SEQ ID NO: 8 - DNA, 527 bp, Mus musculus
  source 1..527: mol_type = genomic DNA
taacagcagcttcttatccagctttattagggacagcatgtttaggtgagatctagggaggggtgaagacatgagaacatactttccccttaaagcaaccttcaggggccacctggcaggcctggaggggggcagcaggatggaggaacaggcacatctgcaacacagaagtctcacgactcaatagctggagttggttggtcctcagggttagaatcccagaagccggtgaacttgtcagtaaacttgctccagtagcctttcagggatctgaagtgattgtccatccagcccctggccaccacagctatatcggactcctgcacgctacttagcgcatcctggaccgtcttggaggcttgttccatgtagccctgtacagagcccagcagcaaggatccctctacctcttcagctcgggcagatgccaggagagccaagagggccacagtgaggagcgtccggggctgcatggcacctacgtacctggagtagctagctgcttctagggataaaactgagcaggc

SEQ ID NO: 9 - DNA, 536 bp, Rattus norvegicus
  source 1..536: mol_type = genomic DNA
aatataaaacagatcagaggcctcccggcttgcctgcccagttttatccctagaagcagctagctactccaggtgcacaggtgccatgcagccccgaatgctcctcatcgtggccctcgtggctctcctggcctctgcccgagctgatgagggagagggatccttgctgctgggctctatgcagggctacatggaacaagcctccaagacggtccaggatgcactaagcagcatgcaggagtctgatatagctgtggtggccaggggctggatggacaatcgcttcaaatccctgaaaggctactggagcaagttcactgataagttcactggcctctgggagtctggccctgaggaccaactaacaacaccaactcttgagccgtgagacctccatgttccagatgtgtctggccatctatcctgctgcctccgaaggttgctctaaggggaaagtatattctcatgcctttatccctccccagacctcacctaaacatgctgtccctaataaagctggacacgaagctgccatg

SEQ ID NO: 10 - DNA, 536 bp, Rattus norvegicus
  source 1..536: mol_type = genomic DNA
catggcagcttcgtgtccagctttattagggacagcatgtttaggtgaggtctggggagggataaaggcatgagaatatactttccccttagagcaaccttcggaggcagcaggatagatggccagacacatctggaacatggaggtctcacggctcaagagttggtgttgttagttggtcctcagggccagactcccagaggccagtgaacttatcagtgaacttgctccagtagcctttcagggatttgaagcgattgtccatccagcccctggccaccacagctatatcagactcctgcatgctgcttagtgcatcctggaccgtcttggaggcttgttccatgtagccctgcatagagcccagcagcaaggatccctctccctcatcagctcgggcagaggccaggagagccacgagggccacgatgaggagcattcggggctgcatggcacctgtgcacctggagtagctagctgcttctagggataaaactgggcaggcaagccgggaggcctctgatctgttttatatt

SEQ ID NO: 11 - DNA, 610 bp, Oryctolagus cuniculus
  source 1..610: mol_type = genomic DNA
tgtcctggggcagtataaaaacaggccggcagcctcttgctgggcttctactcagttcatccccagaagcagtggctccaggaacagaggtgccgccatgcgtccctggctgctcctggttgtcaccctcctggcgctcctggcctctgcccgagcaatggaggccgaggaccccaaggacgcctcccttctcagcgtcatgcagggctatgtgcaacacgccaccaagacggcccatgacgcgctgagcagcatgcaggagtcccaaatggcccagcaggccaggggctgggtggacgctggcatcagctccctgaaaggctacttgagcacgtttgcggacaagttctctgggttctgggacctgagccctgaggccagtccgacccaggcgtctgaggctgtctgagacctcagcacccgcagtctgcctgcccatccgtcccgccgttgccctggctcccgtgggctccagagctgtccccacgcgtggcttgaagggacagtaacctcagtgccccctcgccccccccccccgacctggctcacttccaagcatactgcctcccaataaagctggacgagaagccgcggagagcgggatgtccca

SEQ ID NO: 12 - DNA, 610 bp, Oryctolagus cuniculus
  source 1..610: mol_type = genomic DNA
tgggacatcccgctctccgcggcttctcgtccagctttattgggaggcagtatgcttggaagtgagccaggtcggggggggggggcgagggggcactgaggttactgtcccttcaagccacgcgtggggacagctctggagcccacgggagccagggcaacggcgggacggatgggcaggcagactgcgggtgctgaggtctcagacagcctcagacgcctgggtcggactggcctcagggctcaggtcccagaacccagagaacttgtccgcaaacgtgctcaagtagcctttcagggagctgatgccagcgtccacccagcccctggcctgctgggccatttgggactcctgcatgctgctcagcgcgtcatgggccgtcttggtggcgtgttgcacatagccctgcatgacgctgagaagggaggcgtccttggggtcctcggcctccattgctcgggcagaggccaggagcgccaggagggtgacaaccaggagcagccagggacgcatggcggcacctctgttcctggagccactgcttctggggatgaactgagtagaagcccagcaagaggctgccggcctgtttttatactgccccaggaca

SEQ ID NO: 13 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 14 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 15 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 16 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 17 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 18 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 19 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 20 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 21 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 22 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 23 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 24 - protein, 16 aa, unidentified
  REGION 1..16: note = /note="Description of Unknown: RFGF peptide"
AAVALLPAVLLALLAP

SEQ ID NO: 25 - protein, 11 aa, unidentified
  REGION 1..11: note = /note="Description of Unknown: RFGF analogue peptide"
AALLPVLLAAP

SEQ ID NO: 26 - protein, 13 aa, Human immunodeficiency virus
GRKKRRQRRRPPQ

SEQ ID NO: 27 - protein, 16 aa, Drosophila sp.
RQIKIWFQNRRMKWKK

SEQ ID NO: 28 - DNA, 21 bp, synthetic construct
  misc_feature 1..21: note = /note="Description of Combined DNA/RNA Molecule: Synthetic oligonucleotide"
  modified_base 2..3: mod_base = OTHER; note = uracil
  modified_base 8: mod_base = OTHER; note = uracil
  modified_base 12: mod_base = OTHER; note = uracil
  modified_base 15..16: mod_base = OTHER; note = uracil
  source 1..21: mol_type = other DNA
cttacgctgagtacttcgatt

SEQ ID NO: 29 - DNA, 21 bp, synthetic construct
  misc_feature 1..21: note = /note="Description of Combined DNA/RNA Molecule: Synthetic oligonucleotide"
  modified_base 1: mod_base = OTHER; note = uracil
  modified_base 7: mod_base = OTHER; note = uracil
  modified_base 10: mod_base = OTHER; note = uracil
  modified_base 16: mod_base = OTHER; note = uracil
  source 1..21: mol_type = other DNA
tcgaagtactcagcgtaagtt

SEQ ID NO: 30 - RNA, 21 nt, synthetic construct
  misc_feature 1..21: note = /note="Description of Artificial Sequence: Synthetic oligonucleotide"
  source 1..21: mol_type = other RNA
accaagaccgccaaggatgca

SEQ ID NO: 31 - RNA, 21 nt, synthetic construct
  misc_feature 1..21: note = /note="Description of Artificial Sequence: Synthetic oligonucleotide"
  source 1..21: mol_type = other RNA
accaagaccgccaaggatgca

SEQ ID NO: 32 - RNA, 21 nt, synthetic construct
  misc_feature 1..21: note = /note="Description of Artificial Sequence: Synthetic oligonucleotide"
  source 1..21: mol_type = other RNA
caagaccgccaaggatgcact

SEQ ID NO: 33 - RNA, 21 nt, synthetic construct
  misc_feature 1..21: note = /note="Description of Artificial Sequence: Synthetic oligonucleotide"
  source 1..21: mol_type = other RNA
caagaccgccaaggatgcact

SEQ ID NO: 34 - RNA, 21 nt, synthetic construct
  misc_feature 1..21: note = /note="Description of Artificial Sequence: Synthetic oligonucleotide"
  source 1..21: mol_type = other RNA
ccgatggcttcagttccctga

SEQ ID NO: 35 - RNA, 21 nt, synthetic construct
  misc_feature 1..21: note = /note="Description of Artificial Sequence: Synthetic oligonucleotide"
  source 1..21: mol_type = other RNA
ccgatggcttcagttccctga

SEQ ID NO: 36 - RNA, 21 nt, synthetic construct
  misc_feature 1..21: note = /note="Description of Artificial Sequence: Synthetic oligonucleotide"
  source 1..21: mol_type = other RNA
cgatggcttcagttccctgaa

SEQ ID NO: 37 - RNA, 21 nt, synthetic construct
  misc_feature 1..21: note = /note="Description of Artificial Sequence: Synthetic oligonucleotide"
  source 1..21: mol_type = other RNA
cgatggcttcagttccctgaa

SEQ ID NO: 38 - RNA, 21 nt, synthetic construct
  misc_feature 1..21: note = /note="Description of Artificial Sequence: Synthetic oligonucleotide"
  source 1..21: mol_type = other RNA
agactactggagcaccgttaa

SEQ ID NO: 39 - RNA, 21 nt, synthetic construct
  misc_feature 1..21: note = /note="Description of Artificial Sequence: Synthetic oligonucleotide"
  source 1..21: mol_type = other RNA
agactactggagcaccgttaa

SEQ ID NO: 40 - RNA, 21 nt, synthetic construct
  misc_feature 1..21: note = /note="Description of Artificial Sequence: Synthetic oligonucleotide"
  source 1..21: mol_type = other RNA
ctactggagcaccgttaagga

SEQ ID NO: 41 - RNA, 21 nt, synthetic construct
  misc_feature 1..21: note = /note="Description of Artificial Sequence: Synthetic oligonucleotide"
  source 1..21: mol_type = other RNA
ctactggagcaccgttaagga

SEQ ID NO: 42 - RNA, 21 nt, synthetic construct
  misc_feature 1..21: note = /note="Description of Artificial Sequence: Synthetic oligonucleotide"
  source 1..21: mol_type = other RNA
actggagcaccgttaaggaca

SEQ ID NO: 43 - RNA, 21 nt, synthetic construct
  misc_feature 1..21: note = /note="Description of Artificial Sequence: Synthetic oligonucleotide"
  source 1..21: mol_type = other RNA
actggagcaccgttaaggaca

SEQ ID NO: 44 - RNA, 21 nt, synthetic construct
  misc_feature 1..21: note = /note="Description of Artificial Sequence: Synthetic oligonucleotide"
  source 1..21: mol_type = other RNA
ctggagcaccgttaaggacaa

SEQ ID NO: 45 - RNA, 21 nt, synthetic construct
  misc_feature 1..21: note = /note="Description of Artificial Sequence: Synthetic oligonucleotide"
  source 1..21: mol_type = other RNA
ctggagcaccgttaaggacaa

SEQ ID NO: 46 - RNA, 21 nt, synthetic construct
  misc_feature 1..21: note = /note="Description of Artificial Sequence: Synthetic oligonucleotide"
  source 1..21: mol_type = other RNA
ggagcaccgttaaggacaagt

SEQ ID NO: 47 - RNA, 21 nt, synthetic construct
  misc_feature 1..21: note = /note="Description of Artificial Sequence: Synthetic oligonucleotide"
  source 1..21: mol_type = other RNA
ggagcaccgttaaggacaagt

SEQ ID NO: 48 - RNA, 21 nt, synthetic construct
  misc_feature 1..21: note = /note="Description of Artificial Sequence: Synthetic oligonucleotide"
  source 1..21: mol_type = other RNA
gagcaccgttaaggacaagtt

SEQ ID NO: 49 - RNA, 21 nt, synthetic construct
  misc_feature 1..21: note = /note="Description of Artificial Sequence: Synthetic oligonucleotide"
  source 1..21: mol_type = other RNA
gagcaccgttaaggacaagtt

SEQ ID NO: 50 - RNA, 21 nt, synthetic construct
  misc_feature 1..21: note = /note="Description of Artificial Sequence: Synthetic oligonucleotide"
  source 1..21: mol_type = other RNA
gtggctgcctgagacctcaat

SEQ ID NO: 51 - RNA, 21 nt, synthetic construct
  misc_feature 1..21: note = /note="Description of Artificial Sequence: Synthetic oligonucleotide"
  source 1..21: mol_type = other RNA
gtggctgcctgagacctcaat

SEQ ID NO: 52 - RNA, 21 nt, synthetic construct
  misc_feature 1..21: note = /note="Description of Artificial Sequence: Synthetic oligonucleotide"
  source 1..21: mol_type = other RNA
gcctgagacctcaatacccca

SEQ ID NO: 53 - RNA, 21 nt, synthetic construct
  misc_feature 1..21: note = /note="Description of Artificial Sequence: Synthetic oligonucleotide"
  source 1..21: mol_type = other RNA
gcctgagacctcaatacccca

SEQ ID NO: 54 - RNA, 21 nt, synthetic construct
  misc_feature 1..21: note = /note="Description of Artificial Sequence: Synthetic oligonucleotide"
  source 1..21: mol_type = other RNA
ggctgcctgagacctcaatac

SEQ ID NO: 55 - RNA, 21 nt, synthetic construct
  misc_feature 1..21: note = /note="Description of Artificial Sequence: Synthetic oligonucleotide"
  source 1..21: mol_type = other RNA
cctgagacctcaataccccaa

SEQ ID NO: 56 - RNA, 21 nt, synthetic construct
  misc_feature 1..21: note = /note="Description of Artificial Sequence: Synthetic oligonucleotide"
  source 1..21: mol_type = other RNA
cctgagacctcaataccccaa

SEQ ID NO: 57 - RNA, 21 nt, synthetic construct
  misc_feature 1..21: note = /note="Description of Artificial Sequence: Synthetic oligonucleotide"
  source 1..21: mol_type = other RNA
gctgcctgagacctcaatacc

SEQ ID NO: 58 - RNA, 21 nt, synthetic construct
  misc_feature 1..21: note = /note="Description of Artificial Sequence: Synthetic oligonucleotide"
  source 1..21: mol_type = other RNA
ctgcctgagacctcaataccc

SEQ ID NO: 59 - RNA, 21 nt, synthetic construct
  misc_feature 1..21: note = /note="Description of Artificial Sequence: Synthetic oligonucleotide"
  source 1..21: mol_type = other RNA
gacctcaataccccaagtcca

SEQ ID NO: 60 - RNA, 21 nt, synthetic construct
  misc_feature 1..21: note = /note="Description of Artificial Sequence: Synthetic oligonucleotide"
  source 1..21: mol_type = other RNA
gacctcaataccccaagtcca

SEQ ID NO: 61 - RNA, 21 nt, synthetic construct
  misc_feature 1..21: note = /note="Description of Artificial Sequence: Synthetic oligonucleotide"
  source 1..21: mol_type = other RNA
tgagacctcaataccccaagt

SEQ ID NO: 62 - RNA, 21 nt, synthetic construct
  misc_feature 1..21: note = /note="Description of Artificial Sequence: Synthetic oligonucleotide"
  source 1..21: mol_type = other RNA
atctccagggctgcccctgta

SEQ ID NO: 63 - RNA, 21 nt, synthetic construct
  misc_feature 1..21: note = /note="Description of Artificial Sequence: Synthetic oligonucleotide"
  source 1..21: mol_type = other RNA
gctgcccctgtaggttgctta

SEQ ID NO: 64 - RNA, 21 nt, synthetic construct
  misc_feature 1..21: note = /note="Description of Artificial Sequence: Synthetic oligonucleotide"
  source 1..21: mol_type = other RNA
gctgcccctgtaggttgctta

SEQ ID NO: 65 - RNA, 21 nt, synthetic construct
  misc_feature 1..21: note = /note="Description of Artificial Sequence: Synthetic oligonucleotide"
  source 1..21: mol_type = other RNA
tgcccctgtaggttgcttaaa

SEQ ID NO: 66 - RNA, 21 nt, synthetic construct
  misc_feature 1..21: note = /note="Description of Artificial Sequence: Synthetic oligonucleotide"
  source 1..21: mol_type = other RNA
tgcccctgtaggttgcttaaa

SEQ ID NO: 67 - RNA, 21 nt, synthetic construct
  misc_feature 1..21: note = /note="Description of Artificial Sequence: Synthetic oligonucleotide"
  source 1..21: mol_type = other RNA
gcccctgtaggttgcttaaaa

SEQ ID NO: 68 - RNA, 21 nt, synthetic construct
  misc_feature 1..21: note = /note="Description of Artificial Sequence: Synthetic oligonucleotide"
  source 1..21: mol_type = other RNA
gcccctgtaggttgcttaaaa

SEQ ID NO: 69 - RNA, 21 nt, synthetic construct
  misc_feature 1..21: note = /note="Description of Artificial Sequence: Synthetic oligonucleotide"
  source 1..21: mol_type = other RNA
ctgcccctgtaggttgcttaa

SEQ ID NO: 70 - RNA, 21 nt, synthetic construct
  misc_feature 1..21: note = /note="Description of Artificial Sequence: Synthetic oligonucleotide"
  source 1..21: mol_type = other RNA
ctgtaggttgcttaaaaggga

SEQ ID NO: 71 - RNA, 21 nt, synthetic construct
  misc_feature 1..21: note = /note="Description of Artificial Sequence: Synthetic oligonucleotide"
  source 1..21: mol_type = other RNA
ctgtaggttgcttaaaaggga

SEQ ID NO: 72 - RNA, 21 nt, synthetic construct
  misc_feature 1..21: note = /note="Description of Artificial Sequence: Synthetic oligonucleotide"
  source 1..21: mol_type = other RNA
cccctgtaggttgcttaaaag

SEQ ID NO: 73 - RNA, 21 nt, synthetic construct
  misc_feature 1..21: note = /note="Description of Artificial Sequence: Synthetic oligonucleotide"
  source 1..21: mol_type = other RNA
gtaggttgcttaaaagggaca

SEQ ID NO: 74 - RNA, 21 nt, synthetic construct
  misc_feature 1..21: note = /note="Description of Artificial Sequence: Synthetic oligonucleotide"
  source 1..21: mol_type = other RNA
gtaggttgcttaaaagggaca

SEQ ID NO: 75 - RNA, 21 nt, synthetic construct
  misc_feature 1..21: note = /note="Description of Artificial Sequence: Synthetic oligonucleotide"
  source 1..21: mol_type = other RNA
ccctgtaggttgcttaaaagg

SEQ ID NO: 76 - RNA, 21 nt, synthetic construct
  misc_feature 1..21: note = /note="Description of Artificial Sequence: Synthetic oligonucleotide"
  source 1..21: mol_type = other RNA
gttgcttaaaagggacagtat

SEQ ID NO: 77 - RNA, 21 nt, synthetic construct
  misc_feature 1..21: note = /note="Description of Artificial Sequence: Synthetic oligonucleotide"
  source 1..21: mol_type = other RNA
gttgcttaaaagggacagtat

SEQ ID NO: 78 - RNA, 21 nt, synthetic construct
  misc_feature 1..21: note = /note="Description of Artificial Sequence: Synthetic oligonucleotide"
  source 1..21: mol_type = other RNA
ttgcttaaaagggacagtatt

SEQ ID NO: 79 - RNA, 21 nt, synthetic construct
  misc_feature 1..21: note = /note="Description of Artificial Sequence: Synthetic oligonucleotide"
  source 1..21: mol_type = other RNA
ttgcttaaaagggacagtatt

SEQ ID NO: 80 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 81 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 82 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 83 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 84 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 85 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 86 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 87 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 88 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 89 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 90 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 91 - RNA, 21 nt, synthetic construct
  misc_feature 1..21: note = /note="Description of Artificial Sequence: Synthetic oligonucleotide"
  source 1..21: mol_type = other RNA
gcttaaaatggacagtattct

SEQ ID NO: 92 - RNA, 21 nt, synthetic construct
  misc_feature 1..21: note = /note="Description of Artificial Sequence: Synthetic oligonucleotide"
  source 1..21: mol_type = other RNA
gcttaaaagggacagtcttct

SEQ ID NO: 93 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 94 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 95 - RNA, 21 nt, synthetic construct
  misc_feature 1..21: note = /note="Description of Artificial Sequence: Synthetic oligonucleotide"
  source 1..21: mol_type = other RNA
gcttaaaagggacagttttct

SEQ ID NO: 96 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 97 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 98 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 99 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 100 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 101 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 102 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 103 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 104 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 105 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 106 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 107 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 108 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 109 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 110 - RNA, 21 nt, synthetic construct
  misc_feature 1..21: note = /note="Description of Artificial Sequence: Synthetic oligonucleotide"
  source 1..21: mol_type = other RNA
gcttaaaagggacagtgttct

SEQ ID NO: 111 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 112 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 113 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 114 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 115 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 116 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 19: mod_base = OTHER; note = uracil
  modified_base 21: mod_base = OTHER; note = uracil
  source 1..21: mol_type = other DNA
gcttaaaagggacagtattct

SEQ ID NO: 117 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 118 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 119 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 120 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 121 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 122 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 123 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 124 - RNA, 21 nt, synthetic construct
  misc_feature 1..21: note = /note="Description of Artificial Sequence: Synthetic oligonucleotide"
  source 1..21: mol_type = other RNA
cttaaaagggacagtattctc

SEQ ID NO: 125 - RNA, 21 nt, synthetic construct
  misc_feature 1..21: note = /note="Description of Artificial Sequence: Synthetic oligonucleotide"
  source 1..21: mol_type = other RNA
aagggacagtattctcagtgc

SEQ ID NO: 126 - RNA, 21 nt, synthetic construct
  misc_feature 1..21: note = /note="Description of Artificial Sequence: Synthetic oligonucleotide"
  source 1..21: mol_type = other RNA
ggcctcccaataaagctggac

SEQ ID NO: 127 - RNA, 21 nt, synthetic construct
  misc_feature 1..21: note = /note="Description of Artificial Sequence: Synthetic oligonucleotide"
  source 1..21: mol_type = other RNA
gcctcccaataaagctggaca

SEQ ID NO: 128 - RNA, 21 nt, synthetic construct
  misc_feature 1..21: note = /note="Description of Artificial Sequence: Synthetic oligonucleotide"
  source 1..21: mol_type = other RNA
cctcccaataaagctggacaa

SEQ ID NO: 129 - RNA, 21 nt, synthetic construct
  misc_feature 1..21: note = /note="Description of Artificial Sequence: Synthetic oligonucleotide"
  source 1..21: mol_type = other RNA
ctcccaataaagctggacaag

SEQ ID NO: 130 - RNA, 21 nt, synthetic construct
  misc_feature 1..21: note = /note="Description of Artificial Sequence: Synthetic oligonucleotide"
  source 1..21: mol_type = other RNA
tcccaataaagctggacaaga

SEQ ID NO: 131 - RNA, 21 nt, synthetic construct
  misc_feature 1..21: note = /note="Description of Artificial Sequence: Synthetic oligonucleotide"
  source 1..21: mol_type = other RNA
cccaataaagctggacaagaa

SEQ ID NO: 132 - RNA, 21 nt, synthetic construct
  misc_feature 1..21: note = /note="Description of Artificial Sequence: Synthetic oligonucleotide"
  source 1..21: mol_type = other RNA
gctggacaagaagctgctatg

SEQ ID NO: 133 - RNA, 23 nt, synthetic construct
  misc_feature 1..23: note = /note="Description of Artificial Sequence: Synthetic oligonucleotide"
  source 1..23: mol_type = other RNA
tgcatccttggcggtcttggtgg

SEQ ID NO: 134 - RNA, 23 nt, synthetic construct
  misc_feature 1..23: note = /note="Description of Artificial Sequence: Synthetic oligonucleotide"
  source 1..23: mol_type = other RNA
tgcatccttggcggtcttggtgg

SEQ ID NO: 135 - RNA, 23 nt, synthetic construct
  misc_feature 1..23: note = /note="Description of Artificial Sequence: Synthetic oligonucleotide"
  source 1..23: mol_type = other RNA
agtgcatccttggcggtcttggt

SEQ ID NO: 136 - RNA, 23 nt, synthetic construct
  misc_feature 1..23: note = /note="Description of Artificial Sequence: Synthetic oligonucleotide"
  source 1..23: mol_type = other RNA
agtgcatccttggcggtcttggt

SEQ ID NO: 137 - RNA, 23 nt, synthetic construct
  misc_feature 1..23: note = /note="Description of Artificial Sequence: Synthetic oligonucleotide"
  source 1..23: mol_type = other RNA
tcagggaactgaagccatcggtc

SEQ ID NO: 138 - RNA, 23 nt, synthetic construct
  misc_feature 1..23: note = /note="Description of Artificial Sequence: Synthetic oligonucleotide"
  source 1..23: mol_type = other RNA
tcagggaactgaagccatcggtc

SEQ ID NO: 139 - RNA, 23 nt, synthetic construct
  misc_feature 1..23: note = /note="Description of Artificial Sequence: Synthetic oligonucleotide"
  source 1..23: mol_type = other RNA
ttcagggaactgaagccatcggt

SEQ ID NO: 140 - RNA, 23 nt, synthetic construct
  misc_feature 1..23: note = /note="Description of Artificial Sequence: Synthetic oligonucleotide"
  source 1..23: mol_type = other RNA
ttcagggaactgaagccatcggt

SEQ ID NO: 141 - RNA, 23 nt, synthetic construct
  misc_feature 1..23: note = /note="Description of Artificial Sequence: Synthetic oligonucleotide"
  source 1..23: mol_type = other RNA
ttaacggtgctccagtagtcttt

SEQ ID NO: 142 - RNA, 23 nt, synthetic construct
  misc_feature 1..23: note = /note="Description of Artificial Sequence: Synthetic oligonucleotide"
  source 1..23: mol_type = other RNA
ttaacggtgctccagtagtcttt

SEQ ID NO: 143 - RNA, 23 nt, synthetic construct
  misc_feature 1..23: note = /note="Description of Artificial Sequence: Synthetic oligonucleotide"
  source 1..23: mol_type = other RNA
tccttaacggtgctccagtagtc

SEQ ID NO: 144 - RNA, 23 nt, synthetic construct
  misc_feature 1..23: note = /note="Description of Artificial Sequence: Synthetic oligonucleotide"
  source 1..23: mol_type = other RNA
tccttaacggtgctccagtagtc

SEQ ID NO: 145 - RNA, 23 nt, synthetic construct
  misc_feature 1..23: note = /note="Description of Artificial Sequence: Synthetic oligonucleotide"
  source 1..23: mol_type = other RNA
tgtccttaacggtgctccagtag

SEQ ID NO: 146 - RNA, 23 nt, synthetic construct
  misc_feature 1..23: note = /note="Description of Artificial Sequence: Synthetic oligonucleotide"
  source 1..23: mol_type = other RNA
tgtccttaacggtgctccagtag

SEQ ID NO: 147 - RNA, 23 nt, synthetic construct
  misc_feature 1..23: note = /note="Description of Artificial Sequence: Synthetic oligonucleotide"
  source 1..23: mol_type = other RNA
ttgtccttaacggtgctccagta

SEQ ID NO: 148 - RNA, 23 nt, synthetic construct
  misc_feature 1..23: note = /note="Description of Artificial Sequence: Synthetic oligonucleotide"
  source 1..23: mol_type = other RNA
ttgtccttaacggtgctccagta

SEQ ID NO: 149 - RNA, 23 nt, synthetic construct
  misc_feature 1..23: note = /note="Description of Artificial Sequence: Synthetic oligonucleotide"
  source 1..23: mol_type = other RNA
acttgtccttaacggtgctccag

SEQ ID NO: 150 - RNA, 23 nt, synthetic construct
  misc_feature 1..23: note = /note="Description of Artificial Sequence: Synthetic oligonucleotide"
  source 1..23: mol_type = other RNA
acttgtccttaacggtgctccag

SEQ ID NO: 151 - RNA, 23 nt, synthetic construct
  misc_feature 1..23: note = /note="Description of Artificial Sequence: Synthetic oligonucleotide"
  source 1..23: mol_type = other RNA
aacttgtccttaacggtgctcca

SEQ ID NO: 152 - RNA, 23 nt, synthetic construct
  misc_feature 1..23: note = /note="Description of Artificial Sequence: Synthetic oligonucleotide"
  source 1..23: mol_type = other RNA
aacttgtccttaacggtgctcca

SEQ ID NO: 153 - RNA, 23 nt, synthetic construct
  misc_feature 1..23: note = /note="Description of Artificial Sequence: Synthetic oligonucleotide"
  source 1..23: mol_type = other RNA
attgaggtctcaggcagccacgg

SEQ ID NO: 154 - RNA, 23 nt, synthetic construct
  misc_feature 1..23: note = /note="Description of Artificial Sequence: Synthetic oligonucleotide"
  source 1..23: mol_type = other RNA
attgaggtctcaggcagccacgg

SEQ ID NO: 155 - RNA, 23 nt, synthetic construct
  misc_feature 1..23: note = /note="Description of Artificial Sequence: Synthetic oligonucleotide"
  source 1..23: mol_type = other RNA
tggggtattgaggtctcaggcag

SEQ ID NO: 156 - RNA, 23 nt, synthetic construct
  misc_feature 1..23: note = /note="Description of Artificial Sequence: Synthetic oligonucleotide"
  source 1..23: mol_type = other RNA
tggggtattgaggtctcaggcag

SEQ ID NO: 157 - RNA, 23 nt, synthetic construct
  misc_feature 1..23: note = /note="Description of Artificial Sequence: Synthetic oligonucleotide"
  source 1..23: mol_type = other RNA
gtattgaggtctcaggcagccac

SEQ ID NO: 158 - RNA, 23 nt, synthetic construct
  misc_feature 1..23: note = /note="Description of Artificial Sequence: Synthetic oligonucleotide"
  source 1..23: mol_type = other RNA
ttggggtattgaggtctcaggca

SEQ ID NO: 159 - RNA, 23 nt, synthetic construct
  misc_feature 1..23: note = /note="Description of Artificial Sequence: Synthetic oligonucleotide"
  source 1..23: mol_type = other RNA
ttggggtattgaggtctcaggca

SEQ ID NO: 160 - RNA, 23 nt, synthetic construct
  misc_feature 1..23: note = /note="Description of Artificial Sequence: Synthetic oligonucleotide"
  source 1..23: mol_type = other RNA
ggtattgaggtctcaggcagcca

SEQ ID NO: 161 - RNA, 23 nt, synthetic construct
  misc_feature 1..23: note = /note="Description of Artificial Sequence: Synthetic oligonucleotide"
  source 1..23: mol_type = other RNA
gggtattgaggtctcaggcagcc

SEQ ID NO: 162 - RNA, 23 nt, synthetic construct
  misc_feature 1..23: note = /note="Description of Artificial Sequence: Synthetic oligonucleotide"
  source 1..23: mol_type = other RNA
tggacttggggtattgaggtctc

SEQ ID NO: 163 - RNA, 23 nt, synthetic construct
  misc_feature 1..23: note = /note="Description of Artificial Sequence: Synthetic oligonucleotide"
  source 1..23: mol_type = other RNA
tggacttggggtattgaggtctc

SEQ ID NO: 164 - RNA, 23 nt, synthetic construct
  misc_feature 1..23: note = /note="Description of Artificial Sequence: Synthetic oligonucleotide"
  source 1..23: mol_type = other RNA
acttggggtattgaggtctcagg

SEQ ID NO: 165 - RNA, 23 nt, synthetic construct
  misc_feature 1..23: note = /note="Description of Artificial Sequence: Synthetic oligonucleotide"
  source 1..23: mol_type = other RNA
tacaggggcagccctggagattg

SEQ ID NO: 166 - RNA, 23 nt, synthetic construct
  misc_feature 1..23: note = /note="Description of Artificial Sequence: Synthetic oligonucleotide"
  source 1..23: mol_type = other RNA
taagcaacctacaggggcagccc

SEQ ID NO: 167 - RNA, 23 nt, synthetic construct
  misc_feature 1..23: note = /note="Description of Artificial Sequence: Synthetic oligonucleotide"
  source 1..23: mol_type = other RNA
taagcaacctacaggggcagccc

SEQ ID NO: 168 - RNA, 23 nt, synthetic construct
  misc_feature 1..23: note = /note="Description of Artificial Sequence: Synthetic oligonucleotide"
  source 1..23: mol_type = other RNA
tttaagcaacctacaggggcagc

SEQ ID NO: 169 - RNA, 23 nt, synthetic construct
  misc_feature 1..23: note = /note="Description of Artificial Sequence: Synthetic oligonucleotide"
  source 1..23: mol_type = other RNA
tttaagcaacctacaggggcagc

SEQ ID NO: 170 - RNA, 23 nt, synthetic construct
  misc_feature 1..23: note = /note="Description of Artificial Sequence: Synthetic oligonucleotide"
  source 1..23: mol_type = other RNA
ttttaagcaacctacaggggcag

SEQ ID NO: 171 - RNA, 23 nt, synthetic construct
  misc_feature 1..23: note = /note="Description of Artificial Sequence: Synthetic oligonucleotide"
  source 1..23: mol_type = other RNA
ttttaagcaacctacaggggcag

SEQ ID NO: 172 - RNA, 23 nt, synthetic construct
  misc_feature 1..23: note = /note="Description of Artificial Sequence: Synthetic oligonucleotide"
  source 1..23: mol_type = other RNA
ttaagcaacctacaggggcagcc

SEQ ID NO: 173 - RNA, 23 nt, synthetic construct
  misc_feature 1..23: note = /note="Description of Artificial Sequence: Synthetic oligonucleotide"
  source 1..23: mol_type = other RNA
tcccttttaagcaacctacaggg

SEQ ID NO: 174 - RNA, 23 nt, synthetic construct
  misc_feature 1..23: note = /note="Description of Artificial Sequence: Synthetic oligonucleotide"
  source 1..23: mol_type = other RNA
tcccttttaagcaacctacaggg

SEQ ID NO: 175 - RNA, 23 nt, synthetic construct
  misc_feature 1..23: note = /note="Description of Artificial Sequence: Synthetic oligonucleotide"
  source 1..23: mol_type = other RNA
cttttaagcaacctacaggggca

SEQ ID NO: 176 - RNA, 23 nt, synthetic construct
  misc_feature 1..23: note = /note="Description of Artificial Sequence: Synthetic oligonucleotide"
  source 1..23: mol_type = other RNA
tgtcccttttaagcaacctacag

SEQ ID NO: 177 - RNA, 23 nt, synthetic construct
  misc_feature 1..23: note = /note="Description of Artificial Sequence: Synthetic oligonucleotide"
  source 1..23: mol_type = other RNA
tgtcccttttaagcaacctacag

SEQ ID NO: 178 - RNA, 23 nt, synthetic construct
  misc_feature 1..23: note = /note="Description of Artificial Sequence: Synthetic oligonucleotide"
  source 1..23: mol_type = other RNA
ccttttaagcaacctacaggggc

SEQ ID NO: 179 - RNA, 23 nt, synthetic construct
  misc_feature 1..23: note = /note="Description of Artificial Sequence: Synthetic oligonucleotide"
  source 1..23: mol_type = other RNA
atactgtcccttttaagcaacct

SEQ ID NO: 180 - RNA, 23 nt, synthetic construct
  misc_feature 1..23: note = /note="Description of Artificial Sequence: Synthetic oligonucleotide"
  source 1..23: mol_type = other RNA
atactgtcccttttaagcaacct

SEQ ID NO: 181 - RNA, 23 nt, synthetic construct
  misc_feature 1..23: note = /note="Description of Artificial Sequence: Synthetic oligonucleotide"
  source 1..23: mol_type = other RNA
aatactgtcccttttaagcaacc

SEQ ID NO: 182 - RNA, 23 nt, synthetic construct
  misc_feature 1..23: note = /note="Description of Artificial Sequence: Synthetic oligonucleotide"
  source 1..23: mol_type = other RNA
aatactgtcccttttaagcaacc

SEQ ID NO: 183 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 184 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 185 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 186 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 187 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 188 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 189 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 190 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 191 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 192 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 193 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 194 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 195 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 196 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 197 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 198 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 199 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 200 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 201 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 202 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 203 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 204 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 14..17: mod_base = OTHER; note = uracil
  source 1..23: mol_type = other DNA
agaatactgtcccttttaagcaa

SEQ ID NO: 205 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 206 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 207 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 208 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 209 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 210 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 211 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 212 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 213 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 214 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 215 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 216 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 217 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 14..17: mod_base = OTHER; note = uracil
  source 1..23: mol_type = other DNA
agaatactgtcccttttaagcaa

SEQ ID NO: 218 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15: mod_base = OTHER; note = uracil
  modified_base 17: mod_base = OTHER; note = uracil
  source 1..23: mol_type = other DNA
agaatactgtcccttttaagcaa

SEQ ID NO: 219 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 220 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 221 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 222 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 223 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 224 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 225 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 226 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 227 - RNA, 23 nt, synthetic construct
  misc_feature 1..23: note = /note="Description of Artificial Sequence: Synthetic oligonucleotide"
  source 1..23: mol_type = other RNA
gagaatactgtcccttttaagca

SEQ ID NO: 228 - RNA, 23 nt, synthetic construct
  misc_feature 1..23: note = /note="Description of Artificial Sequence: Synthetic oligonucleotide"
  source 1..23: mol_type = other RNA
gcactgagaatactgtccctttt

SEQ ID NO: 229 - RNA, 23 nt, synthetic construct
  misc_feature 1..23: note = /note="Description of Artificial Sequence: Synthetic oligonucleotide"
  source 1..23: mol_type = other RNA
gtccagctttattgggaggccag

SEQ ID NO: 230 - RNA, 23 nt, synthetic construct
  misc_feature 1..23: note = /note="Description of Artificial Sequence: Synthetic oligonucleotide"
  source 1..23: mol_type = other RNA
tgtccagctttattgggaggcca

SEQ ID NO: 231 - RNA, 23 nt, synthetic construct
  misc_feature 1..23: note = /note="Description of Artificial Sequence: Synthetic oligonucleotide"
  source 1..23: mol_type = other RNA
ttgtccagctttattgggaggcc

SEQ ID NO: 232 - RNA, 23 nt, synthetic construct
  misc_feature 1..23: note = /note="Description of Artificial Sequence: Synthetic oligonucleotide"
  source 1..23: mol_type = other RNA
cttgtccagctttattgggaggc

SEQ ID NO: 233 - RNA, 23 nt, synthetic construct
  misc_feature 1..23: note = /note="Description of Artificial Sequence: Synthetic oligonucleotide"
  source 1..23: mol_type = other RNA
tcttgtccagctttattgggagg

SEQ ID NO: 234 - RNA, 23 nt, synthetic construct
  misc_feature 1..23: note = /note="Description of Artificial Sequence: Synthetic oligonucleotide"
  source 1..23: mol_type = other RNA
ttcttgtccagctttattgggag

SEQ ID NO: 235 - RNA, 23 nt, synthetic construct
  misc_feature 1..23: note = /note="Description of Artificial Sequence: Synthetic oligonucleotide"
  source 1..23: mol_type = other RNA
catagcagcttcttgtccagctt

SEQ ID NO: 236 - RNA, 21 nt, synthetic construct
  misc_feature 1..21: note = /note="Description of Artificial Sequence: Synthetic oligonucleotide"
  source 1..21: mol_type = other RNA
ggatccttgctgctgggctct

SEQ ID NO: 237 - RNA, 21 nt, synthetic construct
  misc_feature 1..21: note = /note="Description of Artificial Sequence: Synthetic oligonucleotide"
  source 1..21: mol_type = other RNA
ggatccttgctgctgggctct

SEQ ID NO: 238 - RNA, 21 nt, synthetic construct
  misc_feature 1..21: note = /note="Description of Artificial Sequence: Synthetic oligonucleotide"
  source 1..21: mol_type = other RNA
gctacatggaacaagcctcca

SEQ ID NO: 239 - RNA, 21 nt, synthetic construct
  misc_feature 1..21: note = /note="Description of Artificial Sequence: Synthetic oligonucleotide"
  source 1..21: mol_type = other RNA
gctacatggaacaagcctcca

SEQ ID NO: 240 - RNA, 21 nt, synthetic construct
  misc_feature 1..21: note = /note="Description of Artificial Sequence: Synthetic oligonucleotide"
  source 1..21: mol_type = other RNA
ctacatggaacaagcctccaa

SEQ ID NO: 241 - RNA, 21 nt, synthetic construct
  misc_feature 1..21: note = /note="Description of Artificial Sequence: Synthetic oligonucleotide"
  source 1..21: mol_type = other RNA
ctacatggaacaagcctccaa

SEQ ID NO: 242 - RNA, 21 nt, synthetic construct
  misc_feature 1..21: note = /note="Description of Artificial Sequence: Synthetic oligonucleotide"
  source 1..21: mol_type = other RNA
acatggaacaagcctccaaga

SEQ ID NO: 243 - RNA, 21 nt, synthetic construct
  misc_feature 1..21: note = /note="Description of Artificial Sequence: Synthetic oligonucleotide"
  source 1..21: mol_type = other RNA
acatggaacaagcctccaaga

SEQ ID NO: 244 - RNA, 21 nt, synthetic construct
  misc_feature 1..21: note = /note="Description of Artificial Sequence: Synthetic oligonucleotide"
  source 1..21: mol_type = other RNA
ggaacaagcctccaagacggt

SEQ ID NO: 245 - RNA, 21 nt, synthetic construct
  misc_feature 1..21: note = /note="Description of Artificial Sequence: Synthetic oligonucleotide"
  source 1..21: mol_type = other RNA
ggaacaagcctccaagacggt

SEQ ID NO: 246 - RNA, 21 nt, synthetic construct
  misc_feature 1..21: note = /note="Description of Artificial Sequence: Synthetic oligonucleotide"
  source 1..21: mol_type = other RNA
acaagcctccaagacggtcca

SEQ ID NO: 247 - RNA, 21 nt, synthetic construct
  misc_feature 1..21: note = /note="Description of Artificial Sequence: Synthetic oligonucleotide"
  source 1..21: mol_type = other RNA
acaagcctccaagacggtcca

SEQ ID NO: 248 - RNA, 21 nt, synthetic construct
  misc_feature 1..21: note = /note="Description of Artificial Sequence: Synthetic oligonucleotide"
  source 1..21: mol_type = other RNA
gcctccaagacggtccaggat

SEQ ID NO: 249 - RNA, 21 nt, synthetic construct
  misc_feature 1..21: note = /note="Description of Artificial Sequence: Synthetic oligonucleotide"
  source 1..21: mol_type = other RNA
gcctccaagacggtccaggat

SEQ ID NO: 250 - RNA, 21 nt, synthetic construct
  misc_feature 1..21: note = /note="Description of Artificial Sequence: Synthetic oligonucleotide"
  source 1..21: mol_type = other RNA
ccctgaaaggctactggagca

SEQ ID NO: 251 - RNA, 21 nt, synthetic construct
  misc_feature 1..21: note = /note="Description of Artificial Sequence: Synthetic oligonucleotide"
  source 1..21: mol_type = other RNA
ccctgaaaggctactggagca

SEQ ID NO: 252 - RNA, 21 nt, synthetic construct
  misc_feature 1..21: note = /note="Description of Artificial Sequence: Synthetic oligonucleotide"
  source 1..21: mol_type = other RNA
cctgaaaggctactggagcaa

SEQ ID NO: 253 - RNA, 21 nt, synthetic construct
  misc_feature 1..21: note = /note="Description of Artificial Sequence: Synthetic oligonucleotide"
  source 1..21: mol_type = other RNA
cctgaaaggctactggagcaa

SEQ ID NO: 254 - RNA, 21 nt, synthetic construct
  misc_feature 1..21: note = /note="Description of Artificial Sequence: Synthetic oligonucleotide"
  source 1..21: mol_type = other RNA
tgaaaggctactggagcaagt

SEQ ID NO: 255 - RNA, 21 nt, synthetic construct
  misc_feature 1..21: note = /note="Description of Artificial Sequence: Synthetic oligonucleotide"
  source 1..21: mol_type = other RNA
tgaaaggctactggagcaagt

SEQ ID NO: 256 - RNA, 21 nt, synthetic construct
  misc_feature 1..21: note = /note="Description of Artificial Sequence: Synthetic oligonucleotide"
  source 1..21: mol_type = other RNA
gaaaggctactggagcaagtt

SEQ ID NO: 257 - RNA, 21 nt, synthetic construct
  misc_feature 1..21: note = /note="Description of Artificial Sequence: Synthetic oligonucleotide"
  source 1..21: mol_type = other RNA
gaaaggctactggagcaagtt

SEQ ID NO: 258 - RNA, 21 nt, synthetic construct
  misc_feature 1..21: note = /note="Description of Artificial Sequence: Synthetic oligonucleotide"
  source 1..21: mol_type = other RNA
aacatgctgtccctaataaag

SEQ ID NO: 259 - RNA, 21 nt, synthetic construct
  misc_feature 1..21: note = /note="Description of Artificial Sequence: Synthetic oligonucleotide"
  source 1..21: mol_type = other RNA
taaggggaaagtatgttctca

SEQ ID NO: 260 - RNA, 21 nt, synthetic construct
  misc_feature 1..21: note = /note="Description of Artificial Sequence: Synthetic oligonucleotide"
  source 1..21: mol_type = other RNA
gcagatgtgcctgttcctcca

SEQ ID NO: 261 - RNA, 21 nt, synthetic construct
  misc_feature 1..21: note = /note="Description of Artificial Sequence: Synthetic oligonucleotide"
  source 1..21: mol_type = other RNA
aagtatgttctcatgtcttca

SEQ ID NO: 262 - RNA, 21 nt, synthetic construct
  misc_feature 1..21: note = /note="Description of Artificial Sequence: Synthetic oligonucleotide"
  source 1..21: mol_type = other RNA
tcacctaaacatgctgtccct

SEQ ID NO: 263 - RNA, 21 nt, synthetic construct
  misc_feature 1..21: note = /note="Description of Artificial Sequence: Synthetic oligonucleotide"
  source 1..21: mol_type = other RNA
ttaaggggaaagtatgttctc

SEQ ID NO: 264 - RNA, 21 nt, synthetic construct
  misc_feature 1..21: note = /note="Description of Artificial Sequence: Synthetic oligonucleotide"
  source 1..21: mol_type = other RNA
ccctagatctcacctaaacat

SEQ ID NO: 265 - RNA, 21 nt, synthetic construct
  misc_feature 1..21: note = /note="Description of Artificial Sequence: Synthetic oligonucleotide"
  source 1..21: mol_type = other RNA
tccctaataaagctggataag

SEQ ID NO: 266 - RNA, 21 nt, synthetic construct
  misc_feature 1..21: note = /note="Description of Artificial Sequence: Synthetic oligonucleotide"
  source 1..21: mol_type = other RNA
aaacatgctgtccctaataaa

SEQ ID NO: 267 - RNA, 21 nt, synthetic construct
  misc_feature 1..21: note = /note="Description of Artificial Sequence: Synthetic oligonucleotide"
  source 1..21: mol_type = other RNA
ccctaataaagctggataaga

SEQ ID NO: 268 - RNA, 21 nt, synthetic construct
  misc_feature 1..21: note = /note="Description of Artificial Sequence: Synthetic oligonucleotide"
  source 1..21: mol_type = other RNA
cctaataaagctggataagaa

SEQ ID NO: 269 - RNA, 21 nt, synthetic construct
  misc_feature 1..21: note = /note="Description of Artificial Sequence: Synthetic oligonucleotide"
  source 1..21: mol_type = other RNA
ctgaaggttgctttaagggga

SEQ ID NO: 270 - RNA, 21 nt, synthetic construct
  misc_feature 1..21: note = /note="Description of Artificial Sequence: Synthetic oligonucleotide"
  source 1..21: mol_type = other RNA
aaggggaaagtatgttctcat

SEQ ID NO: 271 - RNA, 21 nt, synthetic construct
  misc_feature 1..21: note = /note="Description of Artificial Sequence: Synthetic oligonucleotide"
  source 1..21: mol_type = other RNA
agctggataagaagctgctgt

SEQ ID NO: 272 - RNA, 21 nt, synthetic construct
  misc_feature 1..21: note = /note="Description of Artificial Sequence: Synthetic oligonucleotide"
  source 1..21: mol_type = other RNA
acctaaacatgctgtccctaa

SEQ ID NO: 273 - RNA, 21 nt, synthetic construct
  misc_feature 1..21: note = /note="Description of Artificial Sequence: Synthetic oligonucleotide"
  source 1..21: mol_type = other RNA
tttaaggggaaagtatgttct

SEQ ID NO: 274 - RNA, 21 nt, synthetic construct
  misc_feature 1..21: note = /note="Description of Artificial Sequence: Synthetic oligonucleotide"
  source 1..21: mol_type = other RNA
tcgtgagacttctgtgttgca

SEQ ID NO: 275 - RNA, 21 nt, synthetic construct
  misc_feature 1..21: note = /note="Description of Artificial Sequence: Synthetic oligonucleotide"
  source 1..21: mol_type = other RNA
attgagtcgtgagacttctgt

SEQ ID NO: 276 - RNA, 21 nt, synthetic construct
  misc_feature 1..21: note = /note="Description of Artificial Sequence: Synthetic oligonucleotide"
  source 1..21: mol_type = other RNA
gtccctaataaagctggataa

SEQ ID NO: 277 - RNA, 21 nt, synthetic construct
  misc_feature 1..21: note = /note="Description of Artificial Sequence: Synthetic oligonucleotide"
  source 1..21: mol_type = other RNA
ttctgtgttgcagatgtgcct

SEQ ID NO: 278 - RNA, 21 nt, synthetic construct
  misc_feature 1..21: note = /note="Description of Artificial Sequence: Synthetic oligonucleotide"
  source 1..21: mol_type = other RNA
tggcccctgaaggttgcttta

SEQ ID NO: 279 - RNA, 21 nt, synthetic construct
  misc_feature 1..21: note = /note="Description of Artificial Sequence: Synthetic oligonucleotide"
  source 1..21: mol_type = other RNA
gttgctttaaggggaaagtat

SEQ ID NO: 280 - RNA, 21 nt, synthetic construct
  misc_feature 1..21: note = /note="Description of Artificial Sequence: Synthetic oligonucleotide"
  source 1..21: mol_type = other RNA
tgagacttctgtgttgcagat

SEQ ID NO: 281 - RNA, 21 nt, synthetic construct
  misc_feature 1..21: note = /note="Description of Artificial Sequence: Synthetic oligonucleotide"
  source 1..21: mol_type = other RNA
gctggataagaagctgctgtt

SEQ ID NO: 282 - RNA, 21 nt, synthetic construct
  misc_feature 1..21: note = /note="Description of Artificial Sequence: Synthetic oligonucleotide"
  source 1..21: mol_type = other RNA
ctccctagatctcacctaaac

SEQ ID NO: 283 - RNA, 21 nt, synthetic construct
  misc_feature 1..21: note = /note="Description of Artificial Sequence: Synthetic oligonucleotide"
  source 1..21: mol_type = other RNA
cctaaacatgctgtccctaat

SEQ ID NO: 284 - RNA, 21 nt, synthetic construct
  misc_feature 1..21: note = /note="Description of Artificial Sequence: Synthetic oligonucleotide"
  source 1..21: mol_type = other RNA
gaaagtatgttctcatgtctt

SEQ ID NO: 285 - RNA, 21 nt, synthetic construct
  misc_feature 1..21: note = /note="Description of Artificial Sequence: Synthetic oligonucleotide"
  source 1..21: mol_type = other RNA
gcccctgaaggttgctttaag

SEQ ID NO: 286 - RNA, 21 nt, synthetic construct
  misc_feature 1..21: note = /note="Description of Artificial Sequence: Synthetic oligonucleotide"
  source 1..21: mol_type = other RNA
ccctccctagatctcacctaa

SEQ ID NO: 287 - RNA, 21 nt, synthetic construct
  misc_feature 1..21: note = /note="Description of Artificial Sequence: Synthetic oligonucleotide"
  source 1..21: mol_type = other RNA
ctgtccctaataaagctggat

SEQ ID NO: 288 - RNA, 23 nt, synthetic construct
  misc_feature 1..23: note = /note="Description of Artificial Sequence: Synthetic oligonucleotide"
  source 1..23: mol_type = other RNA
agagcccagcagcaaggatccct

SEQ ID NO: 289 - RNA, 23 nt, synthetic construct
  misc_feature 1..23: note = /note="Description of Artificial Sequence: Synthetic oligonucleotide"
  source 1..23: mol_type = other RNA
agagcccagcagcaaggatccct

SEQ ID NO: 290 - RNA, 23 nt, synthetic construct
  misc_feature 1..23: note = /note="Description of Artificial Sequence: Synthetic oligonucleotide"
  source 1..23: mol_type = other RNA
tggaggcttgttccatgtagccc

SEQ ID NO: 291 - RNA, 23 nt, synthetic construct
  misc_feature 1..23: note = /note="Description of Artificial Sequence: Synthetic oligonucleotide"
  source 1..23: mol_type = other RNA
tggaggcttgttccatgtagccc

SEQ ID NO: 292 - RNA, 23 nt, synthetic construct
  misc_feature 1..23: note = /note="Description of Artificial Sequence: Synthetic oligonucleotide"
  source 1..23: mol_type = other RNA
ttggaggcttgttccatgtagcc

SEQ ID NO: 293 - RNA, 23 nt, synthetic construct
  misc_feature 1..23: note = /note="Description of Artificial Sequence: Synthetic oligonucleotide"
  source 1..23: mol_type = other RNA
ttggaggcttgttccatgtagcc

SEQ ID NO: 294 - RNA, 23 nt, synthetic construct
  misc_feature 1..23: note = /note="Description of Artificial Sequence: Synthetic oligonucleotide"
  source 1..23: mol_type = other RNA
tcttggaggcttgttccatgtag

SEQ ID NO: 295 - RNA, 23 nt, synthetic construct
  misc_feature 1..23: note = /note="Description of Artificial Sequence: Synthetic oligonucleotide"
  source 1..23: mol_type = other RNA
tcttggaggcttgttccatgtag

SEQ ID NO: 296 - RNA, 23 nt, synthetic construct
  misc_feature 1..23: note = /note="Description of Artificial Sequence: Synthetic oligonucleotide"
  source 1..23: mol_type = other RNA
accgtcttggaggcttgttccat

SEQ ID NO: 297 - RNA, 23 nt, synthetic construct
  misc_feature 1..23: note = /note="Description of Artificial Sequence: Synthetic oligonucleotide"
  source 1..23: mol_type = other RNA
accgtcttggaggcttgttccat

SEQ ID NO: 298 - RNA, 23 nt, synthetic construct
  misc_feature 1..23: note = /note="Description of Artificial Sequence: Synthetic oligonucleotide"
  source 1..23: mol_type = other RNA
tggaccgtcttggaggcttgttc

SEQ ID NO: 299 - RNA, 23 nt, synthetic construct
  misc_feature 1..23: note = /note="Description of Artificial Sequence: Synthetic oligonucleotide"
  source 1..23: mol_type = other RNA
tggaccgtcttggaggcttgttc

SEQ ID NO: 300 - RNA, 23 nt, synthetic construct
  misc_feature 1..23: note = /note="Description of Artificial Sequence: Synthetic oligonucleotide"
  source 1..23: mol_type = other RNA
atcctggaccgtcttggaggctt

SEQ ID NO: 301 - RNA, 23 nt, synthetic construct
  misc_feature 1..23: note = /note="Description of Artificial Sequence: Synthetic oligonucleotide"
  source 1..23: mol_type = other RNA
atcctggaccgtcttggaggctt

SEQ ID NO: 302 - RNA, 23 nt, synthetic construct
  misc_feature 1..23: note = /note="Description of Artificial Sequence: Synthetic oligonucleotide"
  source 1..23: mol_type = other RNA
tgctccagtagcctttcagggat

SEQ ID NO: 303 - RNA, 23 nt, synthetic construct
  misc_feature 1..23: note = /note="Description of Artificial Sequence: Synthetic oligonucleotide"
  source 1..23: mol_type = other RNA
tgctccagtagcctttcagggat

SEQ ID NO: 304 - RNA, 23 nt, synthetic construct
  misc_feature 1..23: note = /note="Description of Artificial Sequence: Synthetic oligonucleotide"
  source 1..23: mol_type = other RNA
ttgctccagtagcctttcaggga

SEQ ID NO: 305 - RNA, 23 nt, synthetic construct
  misc_feature 1..23: note = /note="Description of Artificial Sequence: Synthetic oligonucleotide"
  source 1..23: mol_type = other RNA
ttgctccagtagcctttcaggga

SEQ ID NO: 306 - RNA, 23 nt, synthetic construct
  misc_feature 1..23: note = /note="Description of Artificial Sequence: Synthetic oligonucleotide"
  source 1..23: mol_type = other RNA
acttgctccagtagcctttcagg

SEQ ID NO: 307 - RNA, 23 nt, synthetic construct
  misc_feature 1..23: note = /note="Description of Artificial Sequence: Synthetic oligonucleotide"
  source 1..23: mol_type = other RNA
acttgctccagtagcctttcagg

SEQ ID NO: 308 - RNA, 23 nt, synthetic construct
  misc_feature 1..23: note = /note="Description of Artificial Sequence: Synthetic oligonucleotide"
  source 1..23: mol_type = other RNA
aacttgctccagtagcctttcag

SEQ ID NO: 309 - RNA, 23 nt, synthetic construct
  misc_feature 1..23: note = /note="Description of Artificial Sequence: Synthetic oligonucleotide"
  source 1..23: mol_type = other RNA
aacttgctccagtagcctttcag

SEQ ID NO: 310 - RNA, 23 nt, synthetic construct
  misc_feature 1..23: note = /note="Description of Artificial Sequence: Synthetic oligonucleotide"
  source 1..23: mol_type = other RNA
ctttattagggacagcatgttta

SEQ ID NO: 311 - RNA, 23 nt, synthetic construct
  misc_feature 1..23: note = /note="Description of Artificial Sequence: Synthetic oligonucleotide"
  source 1..23: mol_type = other RNA
tgagaacatactttccccttaaa

SEQ ID NO: 312 - RNA, 23 nt, synthetic construct
  misc_feature 1..23: note = /note="Description of Artificial Sequence: Synthetic oligonucleotide"
  source 1..23: mol_type = other RNA
tggaggaacaggcacatctgcaa

SEQ ID NO: 313 - RNA, 23 nt, synthetic construct
  misc_feature 1..23: note = /note="Description of Artificial Sequence: Synthetic oligonucleotide"
  source 1..23: mol_type = other RNA
tgaagacatgagaacatactttc

SEQ ID NO: 314 - RNA, 23 nt, synthetic construct
  misc_feature 1..23: note = /note="Description of Artificial Sequence: Synthetic oligonucleotide"
  source 1..23: mol_type = other RNA
agggacagcatgtttaggtgaga

SEQ ID NO: 315 - RNA, 23 nt, synthetic construct
  misc_feature 1..23: note = /note="Description of Artificial Sequence: Synthetic oligonucleotide"
  source 1..23: mol_type = other RNA
gagaacatactttccccttaaag

SEQ ID NO: 316 - RNA, 23 nt, synthetic construct
  misc_feature 1..23: note = /note="Description of Artificial Sequence: Synthetic oligonucleotide"
  source 1..23: mol_type = other RNA
atgtttaggtgagatctagggag

SEQ ID NO: 317 - RNA, 23 nt, synthetic construct
  misc_feature 1..23: note = /note="Description of Artificial Sequence: Synthetic oligonucleotide"
  source 1..23: mol_type = other RNA
cttatccagctttattagggaca

SEQ ID NO: 318 - RNA, 23 nt, synthetic construct
  misc_feature 1..23: note = /note="Description of Artificial Sequence: Synthetic oligonucleotide"
  source 1..23: mol_type = other RNA
tttattagggacagcatgtttag

SEQ ID NO: 319 - RNA, 23 nt, synthetic construct
  misc_feature 1..23: note = /note="Description of Artificial Sequence: Synthetic oligonucleotide"
  source 1..23: mol_type = other RNA
tcttatccagctttattagggac

SEQ ID NO: 320 - RNA, 23 nt, synthetic construct
  misc_feature 1..23: note = /note="Description of Artificial Sequence: Synthetic oligonucleotide"
  source 1..23: mol_type = other RNA
ttcttatccagctttattaggga

SEQ ID NO: 321 - RNA, 23 nt, synthetic construct
  misc_feature 1..23: note = /note="Description of Artificial Sequence: Synthetic oligonucleotide"
  source 1..23: mol_type = other RNA
tccccttaaagcaaccttcaggg

SEQ ID NO: 322 - RNA, 23 nt, synthetic construct
  misc_feature 1..23: note = /note="Description of Artificial Sequence: Synthetic oligonucleotide"
  source 1..23: mol_type = other RNA
atgagaacatactttccccttaa

SEQ ID NO: 323 - RNA, 23 nt, synthetic construct
  misc_feature 1..23: note = /note="Description of Artificial Sequence: Synthetic oligonucleotide"
  source 1..23: mol_type = other RNA
acagcagcttcttatccagcttt

SEQ ID NO: 324 - RNA, 23 nt, synthetic construct
  misc_feature 1..23: note = /note="Description of Artificial Sequence: Synthetic oligonucleotide"
  source 1..23: mol_type = other RNA
ttagggacagcatgtttaggtga

SEQ ID NO: 325 - RNA, 23 nt, synthetic construct
  misc_feature 1..23: note = /note="Description of Artificial Sequence: Synthetic oligonucleotide"
  source 1..23: mol_type = other RNA
agaacatactttccccttaaagc

SEQ ID NO: 326 - RNA, 23 nt, synthetic construct
  misc_feature 1..23: note = /note="Description of Artificial Sequence: Synthetic oligonucleotide"
  source 1..23: mol_type = other RNA
tgcaacacagaagtctcacgact

SEQ ID NO: 327 - RNA, 23 nt, synthetic construct
  misc_feature 1..23: note = /note="Description of Artificial Sequence: Synthetic oligonucleotide"
  source 1..23: mol_type = other RNA
acagaagtctcacgactcaatag

SEQ ID NO: 328 - RNA, 23 nt, synthetic construct
  misc_feature 1..23: note = /note="Description of Artificial Sequence: Synthetic oligonucleotide"
  source 1..23: mol_type = other RNA
ttatccagctttattagggacag

SEQ ID NO: 329 - RNA, 23 nt, synthetic construct
  misc_feature 1..23: note = /note="Description of Artificial Sequence: Synthetic oligonucleotide"
  source 1..23: mol_type = other RNA
aggcacatctgcaacacagaagt

SEQ ID NO: 330 - RNA, 23 nt, synthetic construct
  misc_feature 1..23: note = /note="Description of Artificial Sequence: Synthetic oligonucleotide"
  source 1..23: mol_type = other RNA
taaagcaaccttcaggggccacc

SEQ ID NO: 331 - RNA, 23 nt, synthetic construct
  misc_feature 1..23: note = /note="Description of Artificial Sequence: Synthetic oligonucleotide"
  source 1..23: mol_type = other RNA
atactttccccttaaagcaacct

SEQ ID NO: 332 - RNA, 23 nt, synthetic construct
  misc_feature 1..23: note = /note="Description of Artificial Sequence: Synthetic oligonucleotide"
  source 1..23: mol_type = other RNA
atctgcaacacagaagtctcacg

SEQ ID NO: 333 - RNA, 23 nt, synthetic construct
  misc_feature 1..23: note = /note="Description of Artificial Sequence: Synthetic oligonucleotide"
  source 1..23: mol_type = other RNA
aacagcagcttcttatccagctt

SEQ ID NO: 334 - RNA, 23 nt, synthetic construct
  misc_feature 1..23: note = /note="Description of Artificial Sequence: Synthetic oligonucleotide"
  source 1..23: mol_type = other RNA
gtttaggtgagatctagggaggg

SEQ ID NO: 335 - RNA, 23 nt, synthetic construct
  misc_feature 1..23: note = /note="Description of Artificial Sequence: Synthetic oligonucleotide"
  source 1..23: mol_type = other RNA
attagggacagcatgtttaggtg

SEQ ID NO: 336 - RNA, 23 nt, synthetic construct
  misc_feature 1..23: note = /note="Description of Artificial Sequence: Synthetic oligonucleotide"
  source 1..23: mol_type = other RNA
aagacatgagaacatactttccc

SEQ ID NO: 337 - RNA, 23 nt, synthetic construct
  misc_feature 1..23: note = /note="Description of Artificial Sequence: Synthetic oligonucleotide"
  source 1..23: mol_type = other RNA
cttaaagcaaccttcaggggcca

SEQ ID NO: 338 - RNA, 23 nt, synthetic construct
  misc_feature 1..23: note = /note="Description of Artificial Sequence: Synthetic oligonucleotide"
  source 1..23: mol_type = other RNA
ttaggtgagatctagggaggggt

SEQ ID NO: 339 - RNA, 23 nt, synthetic construct
  misc_feature 1..23: note = /note="Description of Artificial Sequence: Synthetic oligonucleotide"
  source 1..23: mol_type = other RNA
atccagctttattagggacagca

SEQ ID NO: 340 - RNA, 21 nt, synthetic construct
  misc_feature 1..21: note = /note="Description of Artificial Sequence: Synthetic oligonucleotide"
  source 1..21: mol_type = other RNA
accaagaccgccaaggatgca

SEQ ID NO: 341 - RNA, 21 nt, synthetic construct
  misc_feature 1..21: note = /note="Description of Artificial Sequence: Synthetic oligonucleotide"
  source 1..21: mol_type = other RNA
accaagaccgccaaggatgca

SEQ ID NO: 342 - RNA, 21 nt, synthetic construct
  misc_feature 1..21: note = /note="Description of Artificial Sequence: Synthetic oligonucleotide"
  source 1..21: mol_type = other RNA
caagaccgccaaggatgcact

SEQ ID NO: 343 - RNA, 21 nt, synthetic construct
  misc_feature 1..21: note = /note="Description of Artificial Sequence: Synthetic oligonucleotide"
  source 1..21: mol_type = other RNA
caagaccgccaaggatgcact

SEQ ID NO: 344 - RNA, 21 nt, synthetic construct
  misc_feature 1..21: note = /note="Description of Artificial Sequence: Synthetic oligonucleotide"
  source 1..21: mol_type = other RNA
ccgatggcttcagttccctga

SEQ ID NO: 345 - RNA, 21 nt, synthetic construct
  misc_feature 1..21: note = /note="Description of Artificial Sequence: Synthetic oligonucleotide"
  source 1..21: mol_type = other RNA
ccgatggcttcagttccctga

SEQ ID NO: 346 - RNA, 21 nt, synthetic construct
  misc_feature 1..21: note = /note="Description of Artificial Sequence: Synthetic oligonucleotide"
  source 1..21: mol_type = other RNA
cgatggcttcagttccctgaa

SEQ ID NO: 347 - RNA, 21 nt, synthetic construct
  misc_feature 1..21: note = /note="Description of Artificial Sequence: Synthetic oligonucleotide"
  source 1..21: mol_type = other RNA
cgatggcttcagttccctgaa

SEQ ID NO: 348 - RNA, 21 nt, synthetic construct
  misc_feature 1..21: note = /note="Description of Artificial Sequence: Synthetic oligonucleotide"
  source 1..21: mol_type = other RNA
agactactggagcaccgttaa

SEQ ID NO: 349 - RNA, 21 nt, synthetic construct
  misc_feature 1..21: note = /note="Description of Artificial Sequence: Synthetic oligonucleotide"
  source 1..21: mol_type = other RNA
agactactggagcaccgttaa

SEQ ID NO: 350 - RNA, 21 nt, synthetic construct
  misc_feature 1..21: note = /note="Description of Artificial Sequence: Synthetic oligonucleotide"
  source 1..21: mol_type = other RNA
ctactggagcaccgttaagga

SEQ ID NO: 351 - RNA, 21 nt, synthetic construct
  misc_feature 1..21: note = /note="Description of Artificial Sequence: Synthetic oligonucleotide"
  source 1..21: mol_type = other RNA
ctactggagcaccgttaagga

SEQ ID NO: 352 - RNA, 21 nt, synthetic construct
  misc_feature 1..21: note = /note="Description of Artificial Sequence: Synthetic oligonucleotide"
  source 1..21: mol_type = other RNA
actggagcaccgttaaggaca

SEQ ID NO: 353 - RNA, 21 nt, synthetic construct
  misc_feature 1..21: note = /note="Description of Artificial Sequence: Synthetic oligonucleotide"
  source 1..21: mol_type = other RNA
actggagcaccgttaaggaca

SEQ ID NO: 354 - RNA, 21 nt, synthetic construct
  misc_feature 1..21: note = /note="Description of Artificial Sequence: Synthetic oligonucleotide"
  source 1..21: mol_type = other RNA
ctggagcaccgttaaggacaa

SEQ ID NO: 355 - RNA, 21 nt, synthetic construct
  misc_feature 1..21: note = /note="Description of Artificial Sequence: Synthetic oligonucleotide"
  source 1..21: mol_type = other RNA
ctggagcaccgttaaggacaa

SEQ ID NO: 356 - RNA, 21 nt, synthetic construct
  misc_feature 1..21: note = /note="Description of Artificial Sequence: Synthetic oligonucleotide"
  source 1..21: mol_type = other RNA
ggagcaccgttaaggacaagt

SEQ ID NO: 357 - RNA, 21 nt, synthetic construct
  misc_feature 1..21: note = /note="Description of Artificial Sequence: Synthetic oligonucleotide"
  source 1..21: mol_type = other RNA
ggagcaccgttaaggacaagt

SEQ ID NO: 358 - RNA, 21 nt, synthetic construct
  misc_feature 1..21: note = /note="Description of Artificial Sequence: Synthetic oligonucleotide"
  source 1..21: mol_type = other RNA
gagcaccgttaaggacaagtt

SEQ ID NO: 359 - RNA, 21 nt, synthetic construct
  misc_feature 1..21: note = /note="Description of Artificial Sequence: Synthetic oligonucleotide"
  source 1..21: mol_type = other RNA
gagcaccgttaaggacaagtt

SEQ ID NO: 360 - RNA, 21 nt, synthetic construct
  misc_feature 1..21: note = /note="Description of Artificial Sequence: Synthetic oligonucleotide"
  source 1..21: mol_type = other RNA
gtggctgcctgagacctcaat

SEQ ID NO: 361 - RNA, 21 nt, synthetic construct
  misc_feature 1..21: note = /note="Description of Artificial Sequence: Synthetic oligonucleotide"
  source 1..21: mol_type = other RNA
gtggctgcctgagacctcaat

SEQ ID NO: 362 - RNA, 21 nt, synthetic construct
  misc_feature 1..21: note = /note="Description of Artificial Sequence: Synthetic oligonucleotide"
  source 1..21: mol_type = other RNA
gcctgagacctcaatacccca

SEQ ID NO: 363 - RNA, 21 nt, synthetic construct
  misc_feature 1..21: note = /note="Description of Artificial Sequence: Synthetic oligonucleotide"
  source 1..21: mol_type = other RNA
gcctgagacctcaatacccca

SEQ ID NO: 364 - RNA, 21 nt, synthetic construct
  misc_feature 1..21: note = /note="Description of Artificial Sequence: Synthetic oligonucleotide"
  source 1..21: mol_type = other RNA
ggctgcctgagacctcaatac

SEQ ID NO: 365 - RNA, 21 nt, synthetic construct
  misc_feature 1..21: note = /note="Description of Artificial Sequence: Synthetic oligonucleotide"
  source 1..21: mol_type = other RNA
cctgagacctcaataccccaa

SEQ ID NO: 366 - RNA, 21 nt, synthetic construct
  misc_feature 1..21: note = /note="Description of Artificial Sequence: Synthetic oligonucleotide"
  source 1..21: mol_type = other RNA
cctgagacctcaataccccaa

SEQ ID NO: 367 - RNA, 21 nt, synthetic construct
  misc_feature 1..21: note = /note="Description of Artificial Sequence: Synthetic oligonucleotide"
  source 1..21: mol_type = other RNA
gctgcctgagacctcaatacc

SEQ ID NO: 368 - RNA, 21 nt, synthetic construct
  misc_feature 1..21: note = /note="Description of Artificial Sequence: Synthetic oligonucleotide"
  source 1..21: mol_type = other RNA
ctgcctgagacctcaataccc

SEQ ID NO: 369 - RNA, 21 nt, synthetic construct
  misc_feature 1..21: note = /note="Description of Artificial Sequence: Synthetic oligonucleotide"
  source 1..21: mol_type = other RNA
gacctcaataccccaagtcca

SEQ ID NO: 370 - RNA, 21 nt, synthetic construct
  misc_feature 1..21: note = /note="Description of Artificial Sequence: Synthetic oligonucleotide"
  source 1..21: mol_type = other RNA
gacctcaataccccaagtcca

SEQ ID NO: 371 - RNA, 21 nt, synthetic construct
  misc_feature 1..21: note = /note="Description of Artificial Sequence: Synthetic oligonucleotide"
  source 1..21: mol_type = other RNA
tgagacctcaataccccaagt

SEQ ID NO: 372 - RNA, 21 nt, synthetic construct
  misc_feature 1..21: note = /note="Description of Artificial Sequence: Synthetic oligonucleotide"
  source 1..21: mol_type = other RNA
atctccagggctgcccctgta

SEQ ID NO: 373 - RNA, 21 nt, synthetic construct
  misc_feature 1..21: note = /note="Description of Artificial Sequence: Synthetic oligonucleotide"
  source 1..21: mol_type = other RNA
gctgcccctgtaggttgctta

SEQ ID NO: 374 - RNA, 21 nt, synthetic construct
  misc_feature 1..21: note = /note="Description of Artificial Sequence: Synthetic oligonucleotide"
  source 1..21: mol_type = other RNA
gctgcccctgtaggttgctta

SEQ ID NO: 375 - RNA, 21 nt, synthetic construct
  misc_feature 1..21: note = /note="Description of Artificial Sequence: Synthetic oligonucleotide"
  source 1..21: mol_type = other RNA
tgcccctgtaggttgcttaaa

SEQ ID NO: 376 - RNA, 21 nt, synthetic construct
  misc_feature 1..21: note = /note="Description of Artificial Sequence: Synthetic oligonucleotide"
  source 1..21: mol_type = other RNA
tgcccctgtaggttgcttaaa

SEQ ID NO: 377 - RNA, 21 nt, synthetic construct
  misc_feature 1..21: note = /note="Description of Artificial Sequence: Synthetic oligonucleotide"
  source 1..21: mol_type = other RNA
gcccctgtaggttgcttaaaa

SEQ ID NO: 378 - RNA, 21 nt, synthetic construct
  misc_feature 1..21: note = /note="Description of Artificial Sequence: Synthetic oligonucleotide"
  source 1..21: mol_type = other RNA
gcccctgtaggttgcttaaaa

SEQ ID NO: 379 - RNA, 21 nt, synthetic construct
  misc_feature 1..21: note = /note="Description of Artificial Sequence: Synthetic oligonucleotide"
  source 1..21: mol_type = other RNA
ctgcccctgtaggttgcttaa

SEQ ID NO: 380 - RNA, 21 nt, synthetic construct
  misc_feature 1..21: note = /note="Description of Artificial Sequence: Synthetic oligonucleotide"
  source 1..21: mol_type = other RNA
ctgtaggttgcttaaaaggga

SEQ ID NO: 381 - RNA, 21 nt, synthetic construct
  misc_feature 1..21: note = /note="Description of Artificial Sequence: Synthetic oligonucleotide"
  source 1..21: mol_type = other RNA
ctgtaggttgcttaaaaggga

SEQ ID NO: 382 - RNA, 21 nt, synthetic construct
  misc_feature 1..21: note = /note="Description of Artificial Sequence: Synthetic oligonucleotide"
  source 1..21: mol_type = other RNA
cccctgtaggttgcttaaaag

SEQ ID NO: 383 - RNA, 21 nt, synthetic construct
  misc_feature 1..21: note = /note="Description of Artificial Sequence: Synthetic oligonucleotide"
  source 1..21: mol_type = other RNA
gtaggttgcttaaaagggaca

SEQ ID NO: 384 - RNA, 21 nt, synthetic construct
  misc_feature 1..21: note = /note="Description of Artificial Sequence: Synthetic oligonucleotide"
  source 1..21: mol_type = other RNA
gtaggttgcttaaaagggaca

SEQ ID NO: 385 - RNA, 21 nt, synthetic construct
  misc_feature 1..21: note = /note="Description of Artificial Sequence: Synthetic oligonucleotide"
  source 1..21: mol_type = other RNA
ccctgtaggttgcttaaaagg

SEQ ID NO: 386 - RNA, 21 nt, synthetic construct
  misc_feature 1..21: note = /note="Description of Artificial Sequence: Synthetic oligonucleotide"
  source 1..21: mol_type = other RNA
gttgcttaaaagggacagtat

SEQ ID NO: 387 - RNA, 21 nt, synthetic construct
  misc_feature 1..21: note = /note="Description of Artificial Sequence: Synthetic oligonucleotide"
  source 1..21: mol_type = other RNA
gttgcttaaaagggacagtat

SEQ ID NO: 388 - RNA, 21 nt, synthetic construct
  misc_feature 1..21: note = /note="Description of Artificial Sequence: Synthetic oligonucleotide"
  source 1..21: mol_type = other RNA
ttgcttaaaagggacagtatt

SEQ ID NO: 389 - RNA, 21 nt, synthetic construct
  misc_feature 1..21: note = /note="Description of Artificial Sequence: Synthetic oligonucleotide"
  source 1..21: mol_type = other RNA
ttgcttaaaagggacagtatt

SEQ ID NO: 390 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 391 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 392 - RNA, 22 nt, synthetic construct
  misc_feature 1..22: note = /note="Description of Artificial Sequence: Synthetic oligonucleotide"
  modified_base 1: mod_base = OTHER; note = 2-hydroxymethyl-tetrahydrofurane-5-phosphate abasic nucleotide
  modified_base 1: mod_base = OTHER; note = n is a, c, g, or u
  source 1..22: mol_type = other RNA
ngcttaaaagggacagtattct

SEQ ID NO: 393 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 394 - RNA, 22 nt, synthetic construct
  misc_feature 1..22: note = /note="Description of Artificial Sequence: Synthetic oligonucleotide"
  modified_base 1: mod_base = OTHER; note = 2-hydroxymethyl-tetrahydrofurane-5-phosphate abasic nucleotide
  modified_base 1: mod_base = OTHER; note = n is a, c, g, or u
  source 1..22: mol_type = other RNA
ngcttaaaagggacagtattct

SEQ ID NO: 395 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 396 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 397 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 398 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 399 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 400 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 401 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 9: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaatggacagtattct

SEQ ID NO: 402 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtcttct

SEQ ID NO: 403 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 404 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 405 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16..19: mod_base = OTHER; note = uracil
  modified_base 21: mod_base = OTHER; note = uracil
  source 1..21: mol_type = other DNA
gcttaaaagggacagttttct

SEQ ID NO: 406 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 407 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 408 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 409 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 410 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 411 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 412 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 413 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 414 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 415 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 416 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 417 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 418 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 419 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 420 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtgttct

SEQ ID NO: 421 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 422 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagttttct

SEQ ID NO: 423 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 424 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 425 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 426 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 19: mod_base = OTHER; note = uracil
  modified_base 21: mod_base = OTHER; note = uracil
  source 1..21: mol_type = other DNA
gcttaaaagggacagtattct

SEQ ID NO: 427 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 428 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 429 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 430 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 431 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 18..19: mod_base = OTHER; note = uracil
  modified_base 21: mod_base = OTHER; note = uracil
  source 1..21: mol_type = other DNA
gcttaaaagggacagtattct

SEQ ID NO: 432 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 433 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 434 - RNA, 21 nt, synthetic construct
  misc_feature 1..21: note = /note="Description of Artificial Sequence: Synthetic oligonucleotide"
  source 1..21: mol_type = other RNA
cttaaaagggacagtattctc

SEQ ID NO: 435 - RNA, 21 nt, synthetic construct
  misc_feature 1..21: note = /note="Description of Artificial Sequence: Synthetic oligonucleotide"
  source 1..21: mol_type = other RNA
aagggacagtattctcagtgc

SEQ ID NO: 436 - RNA, 21 nt, synthetic construct
  misc_feature 1..21: note = /note="Description of Artificial Sequence: Synthetic oligonucleotide"
  source 1..21: mol_type = other RNA
ggcctcccaataaagctggac

SEQ ID NO: 437 - RNA, 21 nt, synthetic construct
  misc_feature 1..21: note = /note="Description of Artificial Sequence: Synthetic oligonucleotide"
  source 1..21: mol_type = other RNA
gcctcccaataaagctggaca

SEQ ID NO: 438 - RNA, 21 nt, synthetic construct
  misc_feature 1..21: note = /note="Description of Artificial Sequence: Synthetic oligonucleotide"
  source 1..21: mol_type = other RNA
cctcccaataaagctggacaa

SEQ ID NO: 439 - RNA, 21 nt, synthetic construct
  misc_feature 1..21: note = /note="Description of Artificial Sequence: Synthetic oligonucleotide"
  source 1..21: mol_type = other RNA
ctcccaataaagctggacaag

SEQ ID NO: 440 - RNA, 21 nt, synthetic construct
  misc_feature 1..21: note = /note="Description of Artificial Sequence: Synthetic oligonucleotide"
  source 1..21: mol_type = other RNA
tcccaataaagctggacaaga

SEQ ID NO: 441 - RNA, 21 nt, synthetic construct
  misc_feature 1..21: note = /note="Description of Artificial Sequence: Synthetic oligonucleotide"
  source 1..21: mol_type = other RNA
cccaataaagctggacaagaa

SEQ ID NO: 442 - RNA, 21 nt, synthetic construct
  misc_feature 1..21: note = /note="Description of Artificial Sequence: Synthetic oligonucleotide"
  source 1..21: mol_type = other RNA
gctggacaagaagctgctatg

SEQ ID NO: 443 - RNA, 21 nt, synthetic construct
  misc_feature 1..21: note = /note="Description of Artificial Sequence: Synthetic oligonucleotide"
  source 1..21: mol_type = other RNA
ggatccttgctgctgggctct

SEQ ID NO: 444 - RNA, 21 nt, synthetic construct
  misc_feature 1..21: note = /note="Description of Artificial Sequence: Synthetic oligonucleotide"
  source 1..21: mol_type = other RNA
ggatccttgctgctgggctct

SEQ ID NO: 445 - RNA, 21 nt, synthetic construct
  misc_feature 1..21: note = /note="Description of Artificial Sequence: Synthetic oligonucleotide"
  source 1..21: mol_type = other RNA
gctacatggaacaagcctcca

SEQ ID NO: 446 - RNA, 21 nt, synthetic construct
  misc_feature 1..21: note = /note="Description of Artificial Sequence: Synthetic oligonucleotide"
  source 1..21: mol_type = other RNA
gctacatggaacaagcctcca

SEQ ID NO: 447 - RNA, 21 nt, synthetic construct
  misc_feature 1..21: note = /note="Description of Artificial Sequence: Synthetic oligonucleotide"
  source 1..21: mol_type = other RNA
ctacatggaacaagcctccaa

SEQ ID NO: 448 - RNA, 21 nt, synthetic construct
  misc_feature 1..21: note = /note="Description of Artificial Sequence: Synthetic oligonucleotide"
  source 1..21: mol_type = other RNA
ctacatggaacaagcctccaa

SEQ ID NO: 449 - RNA, 21 nt, synthetic construct
  misc_feature 1..21: note = /note="Description of Artificial Sequence: Synthetic oligonucleotide"
  source 1..21: mol_type = other RNA
acatggaacaagcctccaaga

SEQ ID NO: 450 - RNA, 21 nt, synthetic construct
  misc_feature 1..21: note = /note="Description of Artificial Sequence: Synthetic oligonucleotide"
  source 1..21: mol_type = other RNA
acatggaacaagcctccaaga

SEQ ID NO: 451 - RNA, 21 nt, synthetic construct
  misc_feature 1..21: note = /note="Description of Artificial Sequence: Synthetic oligonucleotide"
  source 1..21: mol_type = other RNA
ggaacaagcctccaagacggt

SEQ ID NO: 452 - RNA, 21 nt, synthetic construct
  misc_feature 1..21: note = /note="Description of Artificial Sequence: Synthetic oligonucleotide"
  source 1..21: mol_type = other RNA
ggaacaagcctccaagacggt

SEQ ID NO: 453 - RNA, 21 nt, synthetic construct
  misc_feature 1..21: note = /note="Description of Artificial Sequence: Synthetic oligonucleotide"
  source 1..21: mol_type = other RNA
acaagcctccaagacggtcca

SEQ ID NO: 454 - RNA, 21 nt, synthetic construct
  misc_feature 1..21: note = /note="Description of Artificial Sequence: Synthetic oligonucleotide"
  source 1..21: mol_type = other RNA
acaagcctccaagacggtcca

SEQ ID NO: 455 - RNA, 21 nt, synthetic construct
  misc_feature 1..21: note = /note="Description of Artificial Sequence: Synthetic oligonucleotide"
  source 1..21: mol_type = other RNA
gcctccaagacggtccaggat

SEQ ID NO: 456 - RNA, 21 nt, synthetic construct
  misc_feature 1..21: note = /note="Description of Artificial Sequence: Synthetic oligonucleotide"
  source 1..21: mol_type = other RNA
gcctccaagacggtccaggat

SEQ ID NO: 457 - RNA, 21 nt, synthetic construct
  misc_feature 1..21: note = /note="Description of Artificial Sequence: Synthetic oligonucleotide"
  source 1..21: mol_type = other RNA
ccctgaaaggctactggagca

SEQ ID NO: 458 - RNA, 21 nt, synthetic construct
  misc_feature 1..21: note = /note="Description of Artificial Sequence: Synthetic oligonucleotide"
  source 1..21: mol_type = other RNA
ccctgaaaggctactggagca

SEQ ID NO: 459 - RNA, 21 nt, synthetic construct
  misc_feature 1..21: note = /note="Description of Artificial Sequence: Synthetic oligonucleotide"
  source 1..21: mol_type = other RNA
cctgaaaggctactggagcaa

SEQ ID NO: 460 - RNA, 21 nt, synthetic construct
  misc_feature 1..21: note = /note="Description of Artificial Sequence: Synthetic oligonucleotide"
  source 1..21: mol_type = other RNA
cctgaaaggctactggagcaa

SEQ ID NO: 461 - RNA, 21 nt, synthetic construct
  misc_feature 1..21: note = /note="Description of Artificial Sequence: Synthetic oligonucleotide"
  source 1..21: mol_type = other RNA
tgaaaggctactggagcaagt

SEQ ID NO: 462 - RNA, 21 nt, synthetic construct
  misc_feature 1..21: note = /note="Description of Artificial Sequence: Synthetic oligonucleotide"
  source 1..21: mol_type = other RNA
tgaaaggctactggagcaagt

SEQ ID NO: 463 - RNA, 21 nt, synthetic construct
  misc_feature 1..21: note = /note="Description of Artificial Sequence: Synthetic oligonucleotide"
  source 1..21: mol_type = other RNA
gaaaggctactggagcaagtt

SEQ ID NO: 464 - RNA, 21 nt, synthetic construct
  misc_feature 1..21: note = /note="Description of Artificial Sequence: Synthetic oligonucleotide"
  source 1..21: mol_type = other RNA
gaaaggctactggagcaagtt

SEQ ID NO: 465 - RNA, 21 nt, synthetic construct
  misc_feature 1..21: note = /note="Description of Artificial Sequence: Synthetic oligonucleotide"
  source 1..21: mol_type = other RNA
aacatgctgtccctaataaag

SEQ ID NO: 466 - RNA, 21 nt, synthetic construct
  misc_feature 1..21: note = /note="Description of Artificial Sequence: Synthetic oligonucleotide"
  source 1..21: mol_type = other RNA
taaggggaaagtatgttctca

SEQ ID NO: 467 - RNA, 21 nt, synthetic construct
  misc_feature 1..21: note = /note="Description of Artificial Sequence: Synthetic oligonucleotide"
  source 1..21: mol_type = other RNA
gcagatgtgcctgttcctcca

SEQ ID NO: 468 - RNA, 21 nt, synthetic construct
  misc_feature 1..21: note = /note="Description of Artificial Sequence: Synthetic oligonucleotide"
  source 1..21: mol_type = other RNA
aagtatgttctcatgtcttca

SEQ ID NO: 469 - RNA, 21 nt, synthetic construct
  misc_feature 1..21: note = /note="Description of Artificial Sequence: Synthetic oligonucleotide"
  source 1..21: mol_type = other RNA
tcacctaaacatgctgtccct

SEQ ID NO: 470 - RNA, 21 nt, synthetic construct
  misc_feature 1..21: note = /note="Description of Artificial Sequence: Synthetic oligonucleotide"
  source 1..21: mol_type = other RNA
ttaaggggaaagtatgttctc

SEQ ID NO: 471 - RNA, 21 nt, synthetic construct
  misc_feature 1..21: note = /note="Description of Artificial Sequence: Synthetic oligonucleotide"
  source 1..21: mol_type = other RNA
ccctagatctcacctaaacat

SEQ ID NO: 472 - RNA, 21 nt, synthetic construct
  misc_feature 1..21: note = /note="Description of Artificial Sequence: Synthetic oligonucleotide"
  source 1..21: mol_type = other RNA
tccctaataaagctggataag

SEQ ID NO: 473 - RNA, 21 nt, synthetic construct
  misc_feature 1..21: note = /note="Description of Artificial Sequence: Synthetic oligonucleotide"
  source 1..21: mol_type = other RNA
aaacatgctgtccctaataaa

SEQ ID NO: 474 - RNA, 21 nt, synthetic construct
  misc_feature 1..21: note = /note="Description of Artificial Sequence: Synthetic oligonucleotide"
  source 1..21: mol_type = other RNA
ccctaataaagctggataaga

SEQ ID NO: 475 - RNA, 21 nt, synthetic construct
  misc_feature 1..21: note = /note="Description of Artificial Sequence: Synthetic oligonucleotide"
  source 1..21: mol_type = other RNA
cctaataaagctggataagaa

SEQ ID NO: 476 - RNA, 21 nt, synthetic construct
  misc_feature 1..21: note = /note="Description of Artificial Sequence: Synthetic oligonucleotide"
  source 1..21: mol_type = other RNA
ctgaaggttgctttaagggga

SEQ ID NO: 477 - RNA, 21 nt, synthetic construct
  misc_feature 1..21: note = /note="Description of Artificial Sequence: Synthetic oligonucleotide"
  source 1..21: mol_type = other RNA
aaggggaaagtatgttctcat

SEQ ID NO: 478 - RNA, 21 nt, synthetic construct
  misc_feature 1..21: note = /note="Description of Artificial Sequence: Synthetic oligonucleotide"
  source 1..21: mol_type = other RNA
agctggataagaagctgctgt

SEQ ID NO: 479 - RNA, 21 nt, synthetic construct
  misc_feature 1..21: note = /note="Description of Artificial Sequence: Synthetic oligonucleotide"
  source 1..21: mol_type = other RNA
acctaaacatgctgtccctaa

SEQ ID NO: 480 - RNA, 21 nt, synthetic construct
  misc_feature 1..21: note = /note="Description of Artificial Sequence: Synthetic oligonucleotide"
  source 1..21: mol_type = other RNA
tttaaggggaaagtatgttct

SEQ ID NO: 481 - RNA, 21 nt, synthetic construct
  misc_feature 1..21: note = /note="Description of Artificial Sequence: Synthetic oligonucleotide"
  source 1..21: mol_type = other RNA
tcgtgagacttctgtgttgca

SEQ ID NO: 482 - RNA, 21 nt, synthetic construct
  misc_feature 1..21: note = /note="Description of Artificial Sequence: Synthetic oligonucleotide"
  source 1..21: mol_type = other RNA
attgagtcgtgagacttctgt

SEQ ID NO: 483 - RNA, 21 nt, synthetic construct
  misc_feature 1..21: note = /note="Description of Artificial Sequence: Synthetic oligonucleotide"
  source 1..21: mol_type = other RNA
gtccctaataaagctggataa

SEQ ID NO: 484 - RNA, 21 nt, synthetic construct
  misc_feature 1..21: note = /note="Description of Artificial Sequence: Synthetic oligonucleotide"
  source 1..21: mol_type = other RNA
ttctgtgttgcagatgtgcct

SEQ ID NO: 485 - RNA, 21 nt, synthetic construct
  misc_feature 1..21: note = /note="Description of Artificial Sequence: Synthetic oligonucleotide"
  source 1..21: mol_type = other RNA
tggcccctgaaggttgcttta

SEQ ID NO: 486 - RNA, 21 nt, synthetic construct
  misc_feature 1..21: note = /note="Description of Artificial Sequence: Synthetic oligonucleotide"
  source 1..21: mol_type = other RNA
gttgctttaaggggaaagtat

SEQ ID NO: 487 - RNA, 21 nt, synthetic construct
  misc_feature 1..21: note = /note="Description of Artificial Sequence: Synthetic oligonucleotide"
  source 1..21: mol_type = other RNA
tgagacttctgtgttgcagat

SEQ ID NO: 488 - RNA, 21 nt, synthetic construct
  misc_feature 1..21: note = /note="Description of Artificial Sequence: Synthetic oligonucleotide"
  source 1..21: mol_type = other RNA
gctggataagaagctgctgtt

SEQ ID NO: 489 - RNA, 21 nt, synthetic construct
  misc_feature 1..21: note = /note="Description of Artificial Sequence: Synthetic oligonucleotide"
  source 1..21: mol_type = other RNA
ctccctagatctcacctaaac

SEQ ID NO: 490 - RNA, 21 nt, synthetic construct
  misc_feature 1..21: note = /note="Description of Artificial Sequence: Synthetic oligonucleotide"
  source 1..21: mol_type = other RNA
cctaaacatgctgtccctaat

SEQ ID NO: 491 - RNA, 21 nt, synthetic construct
  misc_feature 1..21: note = /note="Description of Artificial Sequence: Synthetic oligonucleotide"
  source 1..21: mol_type = other RNA
gaaagtatgttctcatgtctt

SEQ ID NO: 492 - RNA, 21 nt, synthetic construct
  misc_feature 1..21: note = /note="Description of Artificial Sequence: Synthetic oligonucleotide"
  source 1..21: mol_type = other RNA
gcccctgaaggttgctttaag

SEQ ID NO: 493 - RNA, 21 nt, synthetic construct
  misc_feature 1..21: note = /note="Description of Artificial Sequence: Synthetic oligonucleotide"
  source 1..21: mol_type = other RNA
ccctccctagatctcacctaa

SEQ ID NO: 494 - RNA, 21 nt, synthetic construct
  misc_feature 1..21: note = /note="Description of Artificial Sequence: Synthetic oligonucleotide"
  source 1..21: mol_type = other RNA
ctgtccctaataaagctggat

SEQ ID NO: 495 - RNA, 23 nt, synthetic construct
  misc_feature 1..23: note = /note="Description of Artificial Sequence: Synthetic oligonucleotide"
  source 1..23: mol_type = other RNA
tgcatccttggcggtcttggtgg

SEQ ID NO: 496 - RNA, 23 nt, synthetic construct
  misc_feature 1..23: note = /note="Description of Artificial Sequence: Synthetic oligonucleotide"
  source 1..23: mol_type = other RNA
tgcatccttggcggtcttggtgg

SEQ ID NO: 497 - RNA, 23 nt, synthetic construct
  misc_feature 1..23: note = /note="Description of Artificial Sequence: Synthetic oligonucleotide"
  source 1..23: mol_type = other RNA
agtgcatccttggcggtcttggt

SEQ ID NO: 498 - RNA, 23 nt, synthetic construct
  misc_feature 1..23: note = /note="Description of Artificial Sequence: Synthetic oligonucleotide"
  source 1..23: mol_type = other RNA
agtgcatccttggcggtcttggt

SEQ ID NO: 499 - RNA, 23 nt, synthetic construct
  misc_feature 1..23: note = /note="Description of Artificial Sequence: Synthetic oligonucleotide"
  source 1..23: mol_type = other RNA
tcagggaactgaagccatcggtc

SEQ ID NO: 500 - RNA, 23 nt, synthetic construct
  misc_feature 1..23: note = /note="Description of Artificial Sequence: Synthetic oligonucleotide"
  source 1..23: mol_type = other RNA
tcagggaactgaagccatcggtc

SEQ ID NO: 501 - RNA, 23 nt, synthetic construct
  misc_feature 1..23: note = /note="Description of Artificial Sequence: Synthetic oligonucleotide"
  source 1..23: mol_type = other RNA
ttcagggaactgaagccatcggt

SEQ ID NO: 502 - RNA, 23 nt, synthetic construct
  misc_feature 1..23: note = /note="Description of Artificial Sequence: Synthetic oligonucleotide"
  source 1..23: mol_type = other RNA
ttcagggaactgaagccatcggt

SEQ ID NO: 503 - RNA, 23 nt, synthetic construct
  misc_feature 1..23: note = /note="Description of Artificial Sequence: Synthetic oligonucleotide"
  source 1..23: mol_type = other RNA
ttaacggtgctccagtagtcttt

SEQ ID NO: 504 - RNA, 23 nt, synthetic construct
  misc_feature 1..23: note = /note="Description of Artificial Sequence: Synthetic oligonucleotide"
  source 1..23: mol_type = other RNA
ttaacggtgctccagtagtcttt

SEQ ID NO: 505 - RNA, 23 nt, synthetic construct
  misc_feature 1..23: note = /note="Description of Artificial Sequence: Synthetic oligonucleotide"
  source 1..23: mol_type = other RNA
tccttaacggtgctccagtagtc

SEQ ID NO: 506 - RNA, 23 nt, synthetic construct
  misc_feature 1..23: note = /note="Description of Artificial Sequence: Synthetic oligonucleotide"
  source 1..23: mol_type = other RNA
tccttaacggtgctccagtagtc

SEQ ID NO: 507 - RNA, 23 nt, synthetic construct
  misc_feature 1..23: note = /note="Description of Artificial Sequence: Synthetic oligonucleotide"
  source 1..23: mol_type = other RNA
tgtccttaacggtgctccagtag

SEQ ID NO: 508 - RNA, 23 nt, synthetic construct
  misc_feature 1..23: note = /note="Description of Artificial Sequence: Synthetic oligonucleotide"
  source 1..23: mol_type = other RNA
tgtccttaacggtgctccagtag

SEQ ID NO: 509 - RNA, 23 nt, synthetic construct
  misc_feature 1..23: note = /note="Description of Artificial Sequence: Synthetic oligonucleotide"
  source 1..23: mol_type = other RNA
ttgtccttaacggtgctccagta

SEQ ID NO: 510 - RNA, 23 nt, synthetic construct
  misc_feature 1..23: note = /note="Description of Artificial Sequence: Synthetic oligonucleotide"
  source 1..23: mol_type = other RNA
ttgtccttaacggtgctccagta

SEQ ID NO: 511 - RNA, 23 nt, synthetic construct
  misc_feature 1..23: note = /note="Description of Artificial Sequence: Synthetic oligonucleotide"
  source 1..23: mol_type = other RNA
acttgtccttaacggtgctccag

SEQ ID NO: 512 - RNA, 23 nt, synthetic construct
  misc_feature 1..23: note = /note="Description of Artificial Sequence: Synthetic oligonucleotide"
  source 1..23: mol_type = other RNA
acttgtccttaacggtgctccag

SEQ ID NO: 513 - RNA, 23 nt, synthetic construct
  misc_feature 1..23: note = /note="Description of Artificial Sequence: Synthetic oligonucleotide"
  source 1..23: mol_type = other RNA
aacttgtccttaacggtgctcca

SEQ ID NO: 514 - RNA, 23 nt, synthetic construct
  misc_feature 1..23: note = /note="Description of Artificial Sequence: Synthetic oligonucleotide"
  source 1..23: mol_type = other RNA
aacttgtccttaacggtgctcca

SEQ ID NO: 515 - RNA, 23 nt, synthetic construct
  misc_feature 1..23: note = /note="Description of Artificial Sequence: Synthetic oligonucleotide"
  source 1..23: mol_type = other RNA
attgaggtctcaggcagccacgg

SEQ ID NO: 516 - RNA, 23 nt, synthetic construct
  misc_feature 1..23: note = /note="Description of Artificial Sequence: Synthetic oligonucleotide"
  source 1..23: mol_type = other RNA
attgaggtctcaggcagccacgg

SEQ ID NO: 517 - RNA, 23 nt, synthetic construct
  misc_feature 1..23: note = /note="Description of Artificial Sequence: Synthetic oligonucleotide"
  source 1..23: mol_type = other RNA
tggggtattgaggtctcaggcag

SEQ ID NO: 518 - RNA, 23 nt, synthetic construct
  misc_feature 1..23: note = /note="Description of Artificial Sequence: Synthetic oligonucleotide"
  source 1..23: mol_type = other RNA
tggggtattgaggtctcaggcag

SEQ ID NO: 519 - RNA, 23 nt, synthetic construct
  misc_feature 1..23: note = /note="Description of Artificial Sequence: Synthetic oligonucleotide"
  source 1..23: mol_type = other RNA
gtattgaggtctcaggcagccac

SEQ ID NO: 520 - RNA, 23 nt, synthetic construct
  misc_feature 1..23: note = /note="Description of Artificial Sequence: Synthetic oligonucleotide"
  source 1..23: mol_type = other RNA
ttggggtattgaggtctcaggca

SEQ ID NO: 521 - RNA, 23 nt, synthetic construct
  misc_feature 1..23: note = /note="Description of Artificial Sequence: Synthetic oligonucleotide"
  source 1..23: mol_type = other RNA
ttggggtattgaggtctcaggca

SEQ ID NO: 522 - RNA, 23 nt, synthetic construct
  misc_feature 1..23: note = /note="Description of Artificial Sequence: Synthetic oligonucleotide"
  source 1..23: mol_type = other RNA
ggtattgaggtctcaggcagcca

SEQ ID NO: 523 - RNA, 23 nt, synthetic construct
  misc_feature 1..23: note = /note="Description of Artificial Sequence: Synthetic oligonucleotide"
  source 1..23: mol_type = other RNA
gggtattgaggtctcaggcagcc

SEQ ID NO: 524 - RNA, 23 nt, synthetic construct
  misc_feature 1..23: note = /note="Description of Artificial Sequence: Synthetic oligonucleotide"
  source 1..23: mol_type = other RNA
tggacttggggtattgaggtctc

SEQ ID NO: 525 - RNA, 23 nt, synthetic construct
  misc_feature 1..23: note = /note="Description of Artificial Sequence: Synthetic oligonucleotide"
  source 1..23: mol_type = other RNA
tggacttggggtattgaggtctc

SEQ ID NO: 526 - RNA, 23 nt, synthetic construct
  misc_feature 1..23: note = /note="Description of Artificial Sequence: Synthetic oligonucleotide"
  source 1..23: mol_type = other RNA
acttggggtattgaggtctcagg

SEQ ID NO: 527 - RNA, 23 nt, synthetic construct
  misc_feature 1..23: note = /note="Description of Artificial Sequence: Synthetic oligonucleotide"
  source 1..23: mol_type = other RNA
tacaggggcagccctggagattg

SEQ ID NO: 528 - RNA, 23 nt, synthetic construct
  misc_feature 1..23: note = /note="Description of Artificial Sequence: Synthetic oligonucleotide"
  source 1..23: mol_type = other RNA
taagcaacctacaggggcagccc

SEQ ID NO: 529 - RNA, 23 nt, synthetic construct
  misc_feature 1..23: note = /note="Description of Artificial Sequence: Synthetic oligonucleotide"
  source 1..23: mol_type = other RNA
taagcaacctacaggggcagccc

SEQ ID NO: 530 - RNA, 23 nt, synthetic construct
  misc_feature 1..23: note = /note="Description of Artificial Sequence: Synthetic oligonucleotide"
  source 1..23: mol_type = other RNA
tttaagcaacctacaggggcagc

SEQ ID NO: 531 - RNA, 23 nt, synthetic construct
  misc_feature 1..23: note = /note="Description of Artificial Sequence: Synthetic oligonucleotide"
  source 1..23: mol_type = other RNA
tttaagcaacctacaggggcagc

SEQ ID NO: 532 - RNA, 23 nt, synthetic construct
  misc_feature 1..23: note = /note="Description of Artificial Sequence: Synthetic oligonucleotide"
  source 1..23: mol_type = other RNA
ttttaagcaacctacaggggcag

SEQ ID NO: 533 - RNA, 23 nt, synthetic construct
  misc_feature 1..23: note = /note="Description of Artificial Sequence: Synthetic oligonucleotide"
  source 1..23: mol_type = other RNA
ttttaagcaacctacaggggcag

SEQ ID NO: 534 - RNA, 23 nt, synthetic construct
  misc_feature 1..23: note = /note="Description of Artificial Sequence: Synthetic oligonucleotide"
  source 1..23: mol_type = other RNA
ttaagcaacctacaggggcagcc

SEQ ID NO: 535 - RNA, 23 nt, synthetic construct
  misc_feature 1..23: note = /note="Description of Artificial Sequence: Synthetic oligonucleotide"
  source 1..23: mol_type = other RNA
tcccttttaagcaacctacaggg

SEQ ID NO: 536 - RNA, 23 nt, synthetic construct
  misc_feature 1..23: note = /note="Description of Artificial Sequence: Synthetic oligonucleotide"
  source 1..23: mol_type = other RNA
tcccttttaagcaacctacaggg

SEQ ID NO: 537 - RNA, 23 nt, synthetic construct
  misc_feature 1..23: note = /note="Description of Artificial Sequence: Synthetic oligonucleotide"
  source 1..23: mol_type = other RNA
cttttaagcaacctacaggggca

SEQ ID NO: 538 - RNA, 23 nt, synthetic construct
  misc_feature 1..23: note = /note="Description of Artificial Sequence: Synthetic oligonucleotide"
  source 1..23: mol_type = other RNA
tgtcccttttaagcaacctacag

SEQ ID NO: 539 - RNA, 23 nt, synthetic construct
  misc_feature 1..23: note = /note="Description of Artificial Sequence: Synthetic oligonucleotide"
  source 1..23: mol_type = other RNA
tgtcccttttaagcaacctacag

SEQ ID NO: 540 - RNA, 23 nt, synthetic construct
  misc_feature 1..23: note = /note="Description of Artificial Sequence: Synthetic oligonucleotide"
  source 1..23: mol_type = other RNA
ccttttaagcaacctacaggggc

SEQ ID NO: 541 - RNA, 23 nt, synthetic construct
  misc_feature 1..23: note = /note="Description of Artificial Sequence: Synthetic oligonucleotide"
  source 1..23: mol_type = other RNA
atactgtcccttttaagcaacct

SEQ ID NO: 542 - RNA, 23 nt, synthetic construct
  misc_feature 1..23: note = /note="Description of Artificial Sequence: Synthetic oligonucleotide"
  source 1..23: mol_type = other RNA
atactgtcccttttaagcaacct

SEQ ID NO: 543 - RNA, 23 nt, synthetic construct
  misc_feature 1..23: note = /note="Description of Artificial Sequence: Synthetic oligonucleotide"
  source 1..23: mol_type = other RNA
aatactgtcccttttaagcaacc

SEQ ID NO: 544 - RNA, 23 nt, synthetic construct
  misc_feature 1..23: note = /note="Description of Artificial Sequence: Synthetic oligonucleotide"
  source 1..23: mol_type = other RNA
aatactgtcccttttaagcaacc

SEQ ID NO: 545 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 546 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 547 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 548 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 549 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 550 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 551 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 552 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 553 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 554 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 555 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 556 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 557 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 558 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 559 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 560 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 561 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 562 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 563 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 564 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 565 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 566 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 14..17: mod_base = OTHER; note = uracil
  source 1..23: mol_type = other DNA
agaatactgtcccttttaagcaa

SEQ ID NO: 567 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 568 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 569 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 570 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 571 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 572 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 573 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 574 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 14..17: mod_base = OTHER; note = uracil
  source 1..23: mol_type = other DNA
agaatactgtcccttttaagcaa

SEQ ID NO: 575 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 576 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 577 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 578 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 579 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 14..17: mod_base = OTHER; note = uracil
  source 1..23: mol_type = other DNA
agaatactgtcccttttaagcaa

SEQ ID NO: 580 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15: mod_base = OTHER; note = uracil
  modified_base 17: mod_base = OTHER; note = uracil
  source 1..23: mol_type = other DNA
agaatactgtcccttttaagcaa

SEQ ID NO: 581 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 582 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 583 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 584 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 585 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 586 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 587 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 10: mod_base = OTHER; note = uracil
  modified_base 15..17: mod_base = OTHER; note = uracil
  source 1..23: mol_type = other DNA
agaatactgtcccttttaagcaa

SEQ ID NO: 588 - DNA, 23 bp, synthetic construct
  misc_feature 1..23: note = /note="Description of Combined DNA/RNA Molecule: Synthetic oligonucleotide"
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
agaatactgtcccttttaagcaa

SEQ ID NO: 589 - RNA, 23 nt, synthetic construct
  misc_feature 1..23: note = /note="Description of Artificial Sequence: Synthetic oligonucleotide"
  source 1..23: mol_type = other RNA
gagaatactgtcccttttaagca

SEQ ID NO: 590 - RNA, 23 nt, synthetic construct
  misc_feature 1..23: note = /note="Description of Artificial Sequence: Synthetic oligonucleotide"
  source 1..23: mol_type = other RNA
gcactgagaatactgtccctttt

SEQ ID NO: 591 - RNA, 23 nt, synthetic construct
  misc_feature 1..23: note = /note="Description of Artificial Sequence: Synthetic oligonucleotide"
  source 1..23: mol_type = other RNA
gtccagctttattgggaggccag

SEQ ID NO: 592 - RNA, 23 nt, synthetic construct
  misc_feature 1..23: note = /note="Description of Artificial Sequence: Synthetic oligonucleotide"
  source 1..23: mol_type = other RNA
tgtccagctttattgggaggcca

SEQ ID NO: 593 - RNA, 23 nt, synthetic construct
  misc_feature 1..23: note = /note="Description of Artificial Sequence: Synthetic oligonucleotide"
  source 1..23: mol_type = other RNA
ttgtccagctttattgggaggcc

SEQ ID NO: 594 - RNA, 23 nt, synthetic construct
  misc_feature 1..23: note = /note="Description of Artificial Sequence: Synthetic oligonucleotide"
  source 1..23: mol_type = other RNA
cttgtccagctttattgggaggc

SEQ ID NO: 595 - RNA, 23 nt, synthetic construct
  misc_feature 1..23: note = /note="Description of Artificial Sequence: Synthetic oligonucleotide"
  source 1..23: mol_type = other RNA
tcttgtccagctttattgggagg

SEQ ID NO: 596 - RNA, 23 nt, synthetic construct
  misc_feature 1..23: note = /note="Description of Artificial Sequence: Synthetic oligonucleotide"
  source 1..23: mol_type = other RNA
ttcttgtccagctttattgggag

SEQ ID NO: 597 - RNA, 23 nt, synthetic construct
  misc_feature 1..23: note = /note="Description of Artificial Sequence: Synthetic oligonucleotide"
  source 1..23: mol_type = other RNA
catagcagcttcttgtccagctt

SEQ ID NO: 598 - RNA, 23 nt, synthetic construct
  misc_feature 1..23: note = /note="Description of Artificial Sequence: Synthetic oligonucleotide"
  source 1..23: mol_type = other RNA
agagcccagcagcaaggatccct

SEQ ID NO: 599 - RNA, 23 nt, synthetic construct
  misc_feature 1..23: note = /note="Description of Artificial Sequence: Synthetic oligonucleotide"
  source 1..23: mol_type = other RNA
agagcccagcagcaaggatccct

SEQ ID NO: 600 - RNA, 23 nt, synthetic construct
  misc_feature 1..23: note = /note="Description of Artificial Sequence: Synthetic oligonucleotide"
  source 1..23: mol_type = other RNA
tggaggcttgttccatgtagccc

SEQ ID NO: 601 - RNA, 23 nt, synthetic construct
  misc_feature 1..23: note = /note="Description of Artificial Sequence: Synthetic oligonucleotide"
  source 1..23: mol_type = other RNA
tggaggcttgttccatgtagccc

SEQ ID NO: 602 - RNA, 23 nt, synthetic construct
  misc_feature 1..23: note = /note="Description of Artificial Sequence: Synthetic oligonucleotide"
  source 1..23: mol_type = other RNA
ttggaggcttgttccatgtagcc

SEQ ID NO: 603 - RNA, 23 nt, synthetic construct
  misc_feature 1..23: note = /note="Description of Artificial Sequence: Synthetic oligonucleotide"
  source 1..23: mol_type = other RNA
ttggaggcttgttccatgtagcc

SEQ ID NO: 604 - RNA, 23 nt, synthetic construct
  misc_feature 1..23: note = /note="Description of Artificial Sequence: Synthetic oligonucleotide"
  source 1..23: mol_type = other RNA
tcttggaggcttgttccatgtag

SEQ ID NO: 605 - RNA, 23 nt, synthetic construct
  misc_feature 1..23: note = /note="Description of Artificial Sequence: Synthetic oligonucleotide"
  source 1..23: mol_type = other RNA
tcttggaggcttgttccatgtag

SEQ ID NO: 606 - RNA, 23 nt, synthetic construct
  misc_feature 1..23: note = /note="Description of Artificial Sequence: Synthetic oligonucleotide"
  source 1..23: mol_type = other RNA
accgtcttggaggcttgttccat

SEQ ID NO: 607 - RNA, 23 nt, synthetic construct
  misc_feature 1..23: note = /note="Description of Artificial Sequence: Synthetic oligonucleotide"
  source 1..23: mol_type = other RNA
accgtcttggaggcttgttccat

SEQ ID NO: 608 - RNA, 23 nt, synthetic construct
  misc_feature 1..23: note = /note="Description of Artificial Sequence: Synthetic oligonucleotide"
  source 1..23: mol_type = other RNA
tggaccgtcttggaggcttgttc

SEQ ID NO: 609 - RNA, 23 nt, synthetic construct
  misc_feature 1..23: note = /note="Description of Artificial Sequence: Synthetic oligonucleotide"
  source 1..23: mol_type = other RNA
tggaccgtcttggaggcttgttc

SEQ ID NO: 610 - RNA, 23 nt, synthetic construct
  misc_feature 1..23: note = /note="Description of Artificial Sequence: Synthetic oligonucleotide"
  source 1..23: mol_type = other RNA
atcctggaccgtcttggaggctt

SEQ ID NO: 611 - RNA, 23 nt, synthetic construct
  misc_feature 1..23: note = /note="Description of Artificial Sequence: Synthetic oligonucleotide"
  source 1..23: mol_type = other RNA
atcctggaccgtcttggaggctt

SEQ ID NO: 612 - RNA, 23 nt, synthetic construct
  misc_feature 1..23: note = /note="Description of Artificial Sequence: Synthetic oligonucleotide"
  source 1..23: mol_type = other RNA
tgctccagtagcctttcagggat

SEQ ID NO: 613 - RNA, 23 nt, synthetic construct
  misc_feature 1..23: note = /note="Description of Artificial Sequence: Synthetic oligonucleotide"
  source 1..23: mol_type = other RNA
tgctccagtagcctttcagggat

SEQ ID NO: 614 - RNA, 23 nt, synthetic construct
  misc_feature 1..23: note = /note="Description of Artificial Sequence: Synthetic oligonucleotide"
  source 1..23: mol_type = other RNA
ttgctccagtagcctttcaggga

SEQ ID NO: 615 - RNA, 23 nt, synthetic construct
  misc_feature 1..23: note = /note="Description of Artificial Sequence: Synthetic oligonucleotide"
  source 1..23: mol_type = other RNA
ttgctccagtagcctttcaggga

SEQ ID NO: 616 - RNA, 23 nt, synthetic construct
  misc_feature 1..23: note = /note="Description of Artificial Sequence: Synthetic oligonucleotide"
  source 1..23: mol_type = other RNA
acttgctccagtagcctttcagg

SEQ ID NO: 617 - RNA, 23 nt, synthetic construct
  misc_feature 1..23: note = /note="Description of Artificial Sequence: Synthetic oligonucleotide"
  source 1..23: mol_type = other RNA
acttgctccagtagcctttcagg

SEQ ID NO: 618 - RNA, 23 nt, synthetic construct
  misc_feature 1..23: note = /note="Description of Artificial Sequence: Synthetic oligonucleotide"
  source 1..23: mol_type = other RNA
aacttgctccagtagcctttcag

SEQ ID NO: 619 - RNA, 23 nt, synthetic construct
  misc_feature 1..23: note = /note="Description of Artificial Sequence: Synthetic oligonucleotide"
  source 1..23: mol_type = other RNA
aacttgctccagtagcctttcag

SEQ ID NO: 620 - RNA, 23 nt, synthetic construct
  misc_feature 1..23: note = /note="Description of Artificial Sequence: Synthetic oligonucleotide"
  source 1..23: mol_type = other RNA
ctttattagggacagcatgttta

SEQ ID NO: 621 - RNA, 23 nt, synthetic construct
  misc_feature 1..23: note = /note="Description of Artificial Sequence: Synthetic oligonucleotide"
  source 1..23: mol_type = other RNA
tgagaacatactttccccttaaa

SEQ ID NO: 622 - RNA, 23 nt, synthetic construct
  misc_feature 1..23: note = /note="Description of Artificial Sequence: Synthetic oligonucleotide"
  source 1..23: mol_type = other RNA
tggaggaacaggcacatctgcaa

SEQ ID NO: 623 - RNA, 23 nt, synthetic construct
  misc_feature 1..23: note = /note="Description of Artificial Sequence: Synthetic oligonucleotide"
  source 1..23: mol_type = other RNA
tgaagacatgagaacatactttc

SEQ ID NO: 624 - RNA, 23 nt, synthetic construct
  misc_feature 1..23: note = /note="Description of Artificial Sequence: Synthetic oligonucleotide"
  source 1..23: mol_type = other RNA
agggacagcatgtttaggtgaga

SEQ ID NO: 625 - RNA, 23 nt, synthetic construct
  misc_feature 1..23: note = /note="Description of Artificial Sequence: Synthetic oligonucleotide"
  source 1..23: mol_type = other RNA
gagaacatactttccccttaaag

SEQ ID NO: 626 - RNA, 23 nt, synthetic construct
  misc_feature 1..23: note = /note="Description of Artificial Sequence: Synthetic oligonucleotide"
  source 1..23: mol_type = other RNA
atgtttaggtgagatctagggag

SEQ ID NO: 627 - RNA, 23 nt, synthetic construct
  misc_feature 1..23: note = /note="Description of Artificial Sequence: Synthetic oligonucleotide"
  source 1..23: mol_type = other RNA
cttatccagctttattagggaca

SEQ ID NO: 628 - RNA, 23 nt, synthetic construct
  misc_feature 1..23: note = /note="Description of Artificial Sequence: Synthetic oligonucleotide"
  source 1..23: mol_type = other RNA
tttattagggacagcatgtttag

SEQ ID NO: 629 - RNA, 23 nt, synthetic construct
  misc_feature 1..23: note = /note="Description of Artificial Sequence: Synthetic oligonucleotide"
  source 1..23: mol_type = other RNA
tcttatccagctttattagggac

SEQ ID NO: 630 - RNA, 23 nt, synthetic construct
  misc_feature 1..23: note = /note="Description of Artificial Sequence: Synthetic oligonucleotide"
  source 1..23: mol_type = other RNA
ttcttatccagctttattaggga

SEQ ID NO: 631 - RNA, 23 nt, synthetic construct
  misc_feature 1..23: note = /note="Description of Artificial Sequence: Synthetic oligonucleotide"
  source 1..23: mol_type = other RNA
tccccttaaagcaaccttcaggg

SEQ ID NO: 632 - RNA, 23 nt, synthetic construct
  misc_feature 1..23: note = /note="Description of Artificial Sequence: Synthetic oligonucleotide"
  source 1..23: mol_type = other RNA
atgagaacatactttccccttaa

SEQ ID NO: 633 - RNA, 23 nt, synthetic construct
  misc_feature 1..23: note = /note="Description of Artificial Sequence: Synthetic oligonucleotide"
  source 1..23: mol_type = other RNA
acagcagcttcttatccagcttt

SEQ ID NO: 634 - RNA, 23 nt, synthetic construct
  misc_feature 1..23: note = /note="Description of Artificial Sequence: Synthetic oligonucleotide"
  source 1..23: mol_type = other RNA
ttagggacagcatgtttaggtga

SEQ ID NO: 635 - RNA, 23 nt, synthetic construct
  misc_feature 1..23: note = /note="Description of Artificial Sequence: Synthetic oligonucleotide"
  source 1..23: mol_type = other RNA
agaacatactttccccttaaagc

SEQ ID NO: 636 - RNA, 23 nt, synthetic construct
  misc_feature 1..23: note = /note="Description of Artificial Sequence: Synthetic oligonucleotide"
  source 1..23: mol_type = other RNA
tgcaacacagaagtctcacgact

SEQ ID NO: 637 - RNA, 23 nt, synthetic construct
  misc_feature 1..23: note = /note="Description of Artificial Sequence: Synthetic oligonucleotide"
  source 1..23: mol_type = other RNA
acagaagtctcacgactcaatag

SEQ ID NO: 638 - RNA, 23 nt, synthetic construct
  misc_feature 1..23: note = /note="Description of Artificial Sequence: Synthetic oligonucleotide"
  source 1..23: mol_type = other RNA
ttatccagctttattagggacag

SEQ ID NO: 639 - RNA, 23 nt, synthetic construct
  misc_feature 1..23: note = /note="Description of Artificial Sequence: Synthetic oligonucleotide"
  source 1..23: mol_type = other RNA
aggcacatctgcaacacagaagt

SEQ ID NO: 640 - RNA, 23 nt, synthetic construct
  misc_feature 1..23: note = /note="Description of Artificial Sequence: Synthetic oligonucleotide"
  source 1..23: mol_type = other RNA
taaagcaaccttcaggggccacc

SEQ ID NO: 641 - RNA, 23 nt, synthetic construct
  misc_feature 1..23: note = /note="Description of Artificial Sequence: Synthetic oligonucleotide"
  source 1..23: mol_type = other RNA
atactttccccttaaagcaacct

SEQ ID NO: 642 - RNA, 23 nt, synthetic construct
  misc_feature 1..23: note = /note="Description of Artificial Sequence: Synthetic oligonucleotide"
  source 1..23: mol_type = other RNA
atctgcaacacagaagtctcacg

SEQ ID NO: 643 - RNA, 23 nt, synthetic construct
  misc_feature 1..23: note = /note="Description of Artificial Sequence: Synthetic oligonucleotide"
  source 1..23: mol_type = other RNA
aacagcagcttcttatccagctt

SEQ ID NO: 644 - RNA, 23 nt, synthetic construct
  misc_feature 1..23: note = /note="Description of Artificial Sequence: Synthetic oligonucleotide"
  source 1..23: mol_type = other RNA
gtttaggtgagatctagggaggg

SEQ ID NO: 645 - RNA, 23 nt, synthetic construct
  misc_feature 1..23: note = /note="Description of Artificial Sequence: Synthetic oligonucleotide"
  source 1..23: mol_type = other RNA
attagggacagcatgtttaggtg

SEQ ID NO: 646 - RNA, 23 nt, synthetic construct
  misc_feature 1..23: note = /note="Description of Artificial Sequence: Synthetic oligonucleotide"
  source 1..23: mol_type = other RNA
aagacatgagaacatactttccc

SEQ ID NO: 647 - RNA, 23 nt, synthetic construct
  misc_feature 1..23: note = /note="Description of Artificial Sequence: Synthetic oligonucleotide"
  source 1..23: mol_type = other RNA
cttaaagcaaccttcaggggcca

SEQ ID NO: 648 - RNA, 23 nt, synthetic construct
  misc_feature 1..23: note = /note="Description of Artificial Sequence: Synthetic oligonucleotide"
  source 1..23: mol_type = other RNA
ttaggtgagatctagggaggggt

SEQ ID NO: 649 - RNA, 23 nt, synthetic construct
  misc_feature 1..23: note = /note="Description of Artificial Sequence: Synthetic oligonucleotide"
  source 1..23: mol_type = other RNA
atccagctttattagggacagca

SEQ ID NO: 650 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 651 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 652 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 653 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 654 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 655 - RNA, 21 nt, synthetic construct
  misc_feature 1..21: note = /note="Description of Artificial Sequence: Synthetic oligonucleotide"
  source 1..21: mol_type = other RNA
gcttaaaagggacagtcttca

SEQ ID NO: 656 - RNA, 21 nt, synthetic construct
  misc_feature 1..21: note = /note="Description of Artificial Sequence: Synthetic oligonucleotide"
  source 1..21: mol_type = other RNA
gcttaaaagggacagtcttca

SEQ ID NO: 657 - RNA, 21 nt, synthetic construct
  misc_feature 1..21: note = /note="Description of Artificial Sequence: Synthetic oligonucleotide"
  source 1..21: mol_type = other RNA
gcttaaaagggacagtcttca

SEQ ID NO: 658 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 659 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 660 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 661 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 662 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 663 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 664 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 665 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 666 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 667 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 668 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 669 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 670 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 671 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 672 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 673 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 674 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 675 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 676 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 677 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtcttca

SEQ ID NO: 678 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtcttca

SEQ ID NO: 679 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtcttca

SEQ ID NO: 680 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 681 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 682 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 683 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 684 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 685 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 686 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 687 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 688 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 689 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 690 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 691 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 692 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 693 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 694 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 695 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattct

SEQ ID NO: 696 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 697 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 698 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 699 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 700 - RNA, 23 nt, synthetic construct
  misc_feature 1..23: note = /note="Description of Artificial Sequence: Synthetic oligonucleotide"
  source 1..23: mol_type = other RNA
agaatactgtcccttttaagcaa

SEQ ID NO: 701 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 702 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 703 - DNA, 23 bp, synthetic construct
  misc_feature 1..23: note = /note="Description of Combined DNA/RNA Molecule: Synthetic oligonucleotide"
  modified_base 1: mod_base = OTHER; note = uracil
  modified_base 5: mod_base = OTHER; note = uracil
  modified_base 8: mod_base = OTHER; note = uracil
  modified_base 15..17: mod_base = OTHER; note = uracil
  source 1..23: mol_type = other DNA
tgaatactgtcccttttaagcaa

SEQ ID NO: 704 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 705 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 706 - RNA, 21 nt, synthetic construct
  misc_feature 1..21: note = /note="Description of Artificial Sequence: Synthetic oligonucleotide"
  source 1..21: mol_type = other RNA
cccaataaagctggacaagaa

SEQ ID NO: 707 - RNA, 21 nt, synthetic construct
  misc_feature 1..21: note = /note="Description of Artificial Sequence: Synthetic oligonucleotide"
  source 1..21: mol_type = other RNA
cctcccaataaagctggacaa

SEQ ID NO: 708 - RNA, 21 nt, synthetic construct
  misc_feature 1..21: note = /note="Description of Artificial Sequence: Synthetic oligonucleotide"
  source 1..21: mol_type = other RNA
tcccaataaagctggacaaga

SEQ ID NO: 709 - RNA, 21 nt, synthetic construct
  misc_feature 1..21: note = /note="Description of Artificial Sequence: Synthetic oligonucleotide"
  source 1..21: mol_type = other RNA
ctcccaataaagctggacaag

SEQ ID NO: 710 - RNA, 23 nt, synthetic construct
  misc_feature 1..23: note = /note="Description of Artificial Sequence: Synthetic oligonucleotide"
  source 1..23: mol_type = other RNA
ttcttgtccagctttattgggag

SEQ ID NO: 711 - RNA, 23 nt, synthetic construct
  misc_feature 1..23: note = /note="Description of Artificial Sequence: Synthetic oligonucleotide"
  source 1..23: mol_type = other RNA
ttgtccagctttattgggaggcc

SEQ ID NO: 712 - RNA, 23 nt, synthetic construct
  misc_feature 1..23: note = /note="Description of Artificial Sequence: Synthetic oligonucleotide"
  source 1..23: mol_type = other RNA
tcttgtccagctttattgggagg

SEQ ID NO: 713 - RNA, 23 nt, synthetic construct
  misc_feature 1..23: note = /note="Description of Artificial Sequence: Synthetic oligonucleotide"
  source 1..23: mol_type = other RNA
cttgtccagctttattgggaggc

SEQ ID NO: 714 - RNA, 21 nt, synthetic construct
  misc_feature 1..21: note = /note="Description of Artificial Sequence: Synthetic oligonucleotide"
  source 1..21: mol_type = other RNA
cccaataaagctggacaagaa

SEQ ID NO: 715 - RNA, 21 nt, synthetic construct
  misc_feature 1..21: note = /note="Description of Artificial Sequence: Synthetic oligonucleotide"
  source 1..21: mol_type = other RNA
cctcccaataaagctggacaa

SEQ ID NO: 716 - RNA, 21 nt, synthetic construct
  misc_feature 1..21: note = /note="Description of Artificial Sequence: Synthetic oligonucleotide"
  source 1..21: mol_type = other RNA
tcccaataaagctggacaaga

SEQ ID NO: 717 - RNA, 21 nt, synthetic construct
  misc_feature 1..21: note = /note="Description of Artificial Sequence: Synthetic oligonucleotide"
  source 1..21: mol_type = other RNA
ctcccaataaagctggacaag

SEQ ID NO: 718 - RNA, 23 nt, synthetic construct
  misc_feature 1..23: note = /note="Description of Artificial Sequence: Synthetic oligonucleotide"
  source 1..23: mol_type = other RNA
ttcttgtccagctttattgggag

SEQ ID NO: 719 - RNA, 23 nt, synthetic construct
  misc_feature 1..23: note = /note="Description of Artificial Sequence: Synthetic oligonucleotide"
  source 1..23: mol_type = other RNA
ttgtccagctttattgggaggcc

SEQ ID NO: 720 - RNA, 23 nt, synthetic construct
  misc_feature 1..23: note = /note="Description of Artificial Sequence: Synthetic oligonucleotide"
  source 1..23: mol_type = other RNA
tcttgtccagctttattgggagg

SEQ ID NO: 721 - RNA, 23 nt, synthetic construct
  misc_feature 1..23: note = /note="Description of Artificial Sequence: Synthetic oligonucleotide"
  source 1..23: mol_type = other RNA
cttgtccagctttattgggaggc

SEQ ID NO: 722 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 723 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtattca

SEQ ID NO: 724 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtcttca

SEQ ID NO: 725 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagtcttca

SEQ ID NO: 726 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 727 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 728 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 729 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 730 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 731 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 732 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 733 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 734 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 735 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 736 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 737 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 738 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 739 - RNA, 21 nt, synthetic construct
  misc_feature 1..21: note = /note="Description of Artificial Sequence: Synthetic oligonucleotide"
  source 1..21: mol_type = other RNA
gcttaaaagggacagttttca

SEQ ID NO: 740 - RNA, 21 nt, synthetic construct
  misc_feature 1..21: note = /note="Description of Artificial Sequence: Synthetic oligonucleotide"
  source 1..21: mol_type = other RNA
gcttaaaagggacagttttca

SEQ ID NO: 741 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagttttca

SEQ ID NO: 742 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 743 - DNA, 21 bp, synthetic construct
  misc_feature 1..21: note = /note="Description of Combined DNA/RNA Molecule: Synthetic oligonucleotide"
  modified_base 3..4: mod_base = OTHER; note = uracil
  modified_base 16: mod_base = OTHER; note = uracil
  modified_base 18..19: mod_base = OTHER; note = uracil
  source 1..21: mol_type = other DNA
gcttaaaagggacagttttca

SEQ ID NO: 744 - RNA, 21 nt, synthetic construct
  misc_feature 1..21: note = /note="Description of Artificial Sequence: Synthetic oligonucleotide"
  source 1..21: mol_type = other RNA
gcttaaaagggacagtattca

SEQ ID NO: 745 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 746 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 747 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 748 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 749 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 750 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 751 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 752 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 753 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 754 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

SEQ ID NO: 755 - RNA, 23 nt, synthetic construct
  misc_feature 1..23: note = /note="Description of Artificial Sequence: Synthetic oligonucleotide"
  source 1..23: mol_type = other RNA
tgaatactgtcccttttaagcaa

(19) United States
(12) Patent Application Publication
(10) Pub. No.: US 2025/0051765 A1
(43) Pub. Date: Feb. 13, 2025

(54) APOLIPOPROTEIN C3 (APOC3) iRNA COMPOSITIONS AND METHODS OF USE THEREOF

(71) Applicant: Alnylam Pharmaceuticals, Inc., Cambridge, MA (US)

(72) Inventors: Kevin Fitzgerald, Brookline, MA (US); William Querbes, Boston, MA (US); James Butler, Lynnfield, MA (US); Stephanie Williams, Littleton, MA (US); Abigail Liebow, Somerville, MA (US); Gregory Hinkle, Plymouth, MA (US); Martin A. Maier, Belmont, MA (US); Stuart Milstein, Arlington, MA (US); Satyanarayana Kuchimanchi, Acton, MA (US); Muthiah Manoharan, Weston, MA (US)

(21) Appl. No.: 18/648,530

(22) Filed: Apr. 29, 2024

Related U.S. Application Data
(63) Continuation of application No. 17/741,550, filed on May 11, 2022, now abandoned.
Application No. 17/741,550 is a continuation of application No. 17/347,863, filed on Jun. 15, 2021, now Pat. No. 11,408,001.
Application No. 17/347,863 is a continuation of application No. 17/128,320, filed on Dec. 21, 2020, now Pat. No. 11,034,958.
Application No. 17/128,320 is a continuation of application No. 16/534,193, filed on Aug. 7, 2019, now Pat. No. 11,142,766.
Application No. 16/534,193 is a continuation of application No. 15/597,225, filed on May 17, 2017, now Pat. No. 10,407,679.
Application No. 15/597,225 is a continuation of application No. PCT/US2015/061065, filed on Nov. 17, 2015.
(60) Provisional application No. 62/136,159, filed on Mar. 20, 2015.
(60) Provisional application No. 62/080,941, filed on Nov. 17, 2014.

(51) Int. Cl. C12N 15/113; A61K 31/713; A61P 1/16; A61P 1/18; A61P 3/04; A61P 3/06; A61P 3/10; A61P 9/10; A61P 9/12; A61P 13/12; A61P 15/08; A61P 43/00

(52) U.S. Cl. CPC C12N 15/113; A61K 31/713; A61P 1/16; A61P 1/18; A61P 3/04; A61P 3/06; A61P 3/10; A61P 9/10; A61P 9/12; A61P 13/12; A61P 15/08; A61P 43/00; C12N 2310/14; C12N 2310/315; C12N 2310/321; C12N 2310/322; C12N 2310/335; C12N 2310/343; C12N 2310/3515

(57) ABSTRACT

The present invention relates to RNAi agents, e.g., double-stranded RNAi agents, targeting the apolipoprotein C3 (APOC3) gene, and methods of using such RNAi agents to inhibit expression of APOC3 and methods of treating subjects having an APOC3 associated disorder, e.g., hypertriglyceridemia.

### RELATED APPLICATIONS

This application is a continuation of U.S. patent application Ser. No. 17/741,550, filed on May 11, 2022, which is a continuation of U.S. patent application Ser. No. 17/347,863, filed on Jun. 15, 2021, now U.S. Pat. No. 11,408,001, issued on Aug. 9, 2022, which is a continuation of U.S. patent application Ser. No. 17/128,320, filed on Dec. 21, 2020, now U.S. Pat. No. 11,034,958, issued on Jun. 15, 2021, which is a continuation of U.S. patent application Ser. No. 16/534,193, filed on Aug. 7, 2019, now U.S. Pat. No. 11,142,766, issued on Oct. 12, 2021, which is a continuation application of U.S. patent application Ser. No. 15/597,225, filed on May 17, 2017, now U.S. Pat. No. 10,407,679, issued on Sep. 10, 2019, which is a 35 § U.S.C. 111(a) continuation application which claims the benefit of priority to PCT/US2015/061065, filed on Nov. 17, 2015, which claims priority to U.S. Provisional Application No. 62/080,941, filed on Nov. 17, 2014, and U.S. Provisional Application No. 62/136,159, filed on Mar. 20, 2015. The entire contents of each of the foregoing applications are hereby incorporated herein by reference.

### SEQUENCE LISTING

The instant application contains a Sequence Listing which has been submitted electronically in XML file format and is hereby incorporated by reference in its entirety. Said .XML copy, created on Apr. 4, 2024, is named “121301-02509.xml” and is 1,164,872 bytes in size.

### BACKGROUND OF THE INVENTION

Apolipoprotein C3 (APOC3) is a very low density lipoprotein (VLDL) and an important regulator of lipoprotein metabolism. In humans, APOC2 is encoded by the APOC3 gene that is located in a gene cluster together with the APOA1 and APOA4 genes on the long arm of chromosome 11. APOC3 is expressed in the liver and, to a lesser extent, in the intestines, as a small 99-amino acid protein. Following removal of the 20-amino-acid signal peptide in the endoplasmic reticulum, a mature ApoC3 protein of 79 amino acids is formed, which may be present as a non-glycosylated or a glycosylated isoform.

The primary role of APOC3 is as a regulator of lipolysis through non-competitive inhibition of endothelial bound lipoprotein lipase (LPL). LPL hydrolyses triacylglycerols in triacylglycerol (triglyceride)-rich lipoproteins (TRLs), releasing fatty acids into the plasma and transforming large triacylglycerol-rich particles into smaller triacylglycerol-depleted remnant lipoproteins. Individuals lacking APOC3 have low TRL levels, coupled with highly efficient lipolysis of triacylglycerols. Furthermore, mice in which the APOC3 gene has been genetically deleted were also shown to have low plasma triacylglycerol levels and efficient TRL catabolism. APOC3 also inhibits hepatic lipase (HL), a lipolytic enzyme with triacylglycerol lipase and phospholipase A1 activity that is synthesized in the liver. The inhibitory effect of APOC3 on HL further reduces the lipolysis and uptake of TRL remnants by the liver. APOC3 has also been shown to stimulate synthesis of very low density lipoproteins (VLDLs). It is believed that the underlying mechanisms associated with this effect of APOC3 may relate to the inhibition of proteasome mediated degradation of APOB, resulting in increased APOB synthesis and secretion, and increased synthesis of VLDL triacylglycerols. APOC3 may, therefore, play a key role in regulating VLDL output by the liver.

Cellular studies report that APOC3 may interfere with TRL and remnant binding to hepatic lipoprotein receptors. APOC3 can abolish APOB- and ApoE-mediated binding of lipoproteins to low density lipoprotein receptor (LDLR), either by masking or altering the conformation of APOB and APOE. The binding of chylomicrons and VLDL particles to the lipolysis-stimulated receptor (LSR) is also significantly inhibited by APOC3.

An increase in APOC3 levels induces the development of hypertriglyceridemia, or high (hyper-) blood levels (-emia) of triglycerides. Elevated levels of triglycerides are associated with a variety of diseases, including cardiovascular disease, atherosclerosis, non-alcoholic fatty liver disease, non-alcoholic steatohepatitis, polycystic ovary syndrome, kidney disease, obesity, type 2 diabetes mellitus (insulin resistance), hypertension and skin lesions (xanthomas). Very high triglyceride levels also increase the risk of acute pancreatitis. Therefore, regulating APOC3 metabolism may be an important new therapeutic approach to managing hypertriglyceridemia and the associated diseases.

Accordingly, there is a need in the art for regulators of APOC3 expression for treating apolipoprotein C3 associated disorders, such as hypertriglyceridemia.

### SUMMARY OF THE INVENTION

The present invention provides iRNA compositions which inhibit or reduce the expression of APOC3 gene. The gene may be within a cell, e.g., a cell within a subject, such as a human.

The present invention also provides methods and therapies for treating a subject having a disorder that would benefit from inhibiting or reducing the expression of an APOC3 gene, e.g., an apolipoprotein C3-associated disease, such as hypertriglyceridemia, using iRNA compositions which inhibit or reduce the expression of the APOC3 gene.

In some embodiments, the present invention provides a double stranded RNAi agent for inhibiting expression of apolipoprotein C3 (APOC3) in a cell, wherein the double stranded RNAi agent comprises a sense strand and an antisense strand forming a double-stranded region, wherein the sense strand comprises at least 15 contiguous nucleotides differing by no more than 3 nucleotides from the nucleotide sequence of SEQ ID NO:1, and the antisense strand comprises at least 15 contiguous nucleotides differing by no more than 3 nucleotides from the nucleotide sequence of SEQ ID NO:2,
wherein substantially all of the nucleotides of at least one strand are modified nucleotides, and wherein said sense strand is conjugated to a ligand attached at the 3′-terminus.

In certain aspects, all of the nucleotides of the sense strand and all of the nucleotides of the antisense strand are modified nucleotides. In one aspect, the sense strand and the antisense strand comprise a region of complementarity which comprises at least 15 contiguous nucleotides differing by no more than 3 nucleotides from any one of the sequences listed in Tables 4A, 4B, 5, 8, 9, 10, 11A, 11B, 12, and 13.

In some embodiments, at least one of the modified nucleotides is selected from the group consisting of a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate, and a terminal nucleotide linked to a cholesteryl derivative or a dodecanoic acid bisdecylamide group.

In one embodiment, substantially all of the nucleotides of the sense strand are modified. In another aspect, substantially all of the nucleotides of the antisense strand are modified. In yet another embodiment, substantially all of the nucleotides of the sense strand and substantially all of the nucleotides of the antisense strand are modified nucleotides. In one embodiment, all of the nucleotides of the sense strand are modified nucleotides. In another embodiment, all of the nucleotides of the antisense strand are modified nucleotides. In yet another embodiment, all of the nucleotides of the sense strand and all of the nucleotides of the antisense strand are modified nucleotides.

In one aspect, at least one strand comprises a 3′ overhang of at least 1 nucleotide. In another aspect, at least one strand comprises a 3′ overhang of at least 2 nucleotides.

In some embodiment, the present invention provides a double stranded RNAi agent capable of inhibiting the expression of apolipoprotein C3 (APOC3) in a cell, wherein the double stranded RNAi agent comprises a sense strand and an antisense strand forming a double stranded region, wherein the antisense strand comprises a region complementary to part of an mRNA encoding APOC3, wherein each strand is about 14 to about 30 nucleotides in length, wherein the double stranded RNAi agent is represented by formula (III):

sense: 5′nₚ-Nₐ—(XXX)ᵢ—N_{b}—YYY—N_{b}—(ZZZ)ⱼ—Nₐ-n_{q}3′

antisense: 3′nₚ′-Nₐ′—(X′X′X′)k-N_{b}′—Y′Y′Y′—N_{b}′—(Z′Z′Z′)ₗ—Nₐ′-n_{q}′5′ (III)
wherein:
i, j, k, and 1 are each independently 0 or 1;
p, p′, q, and q′ are each independently 0-6;
each Nₐ and Nₐ′ independently represents an oligonucleotide sequence comprising 0-25 nucleotides which are either modified or unmodified or combinations thereof, each sequence comprising at least two differently modified nucleotides;
each N_{b} and N_{b}′ independently represents an oligonucleotide sequence comprising 0-10 nucleotides which are either modified or unmodified or combinations thereof;
each nₚ, nₚ′, n_{q}, and n_{q}′, each of which may or may not be present, independently represents an overhang nucleotide;
XXX, YYY, ZZZ, X′X′X′, Y′Y′Y′, and Z′Z′Z′ each independently represent one motif of three identical modifications on three consecutive nucleotides;
modifications on N_{b} differ from the modification on Y and modifications on N_{b}′ differ from the modification on Y′; and
wherein the sense strand is conjugated to at least one ligand.

In a further embodiment, i is 0; j is 0; i is 1; j is 1; both i and j are 0; or both i and j are 1. In another further embodiment, k is 0; 1 is 0; k is 1; 1 is 1; both k and 1 are 0; or both k and 1 are 1. In another aspect, the YYY motif occurs at or near the cleavage site of the sense strand. In yet another aspect, the Y′Y′Y′ motif occurs at the 11, 12 and 13 positions of the antisense strand from the 5′-end.

In one embodiment, Y′ is 2′-O-methyl or 2′-fluoro.

In some aspects, formula (III) is represented by formula (IIIa):

sense: 5′nₚ-Nₐ—YYY—Nₐ-n_{q}3′

antisense: 3′n_{p′}—N_{a′}—Y′Y′Y′—N_{a′}-n_{q′}-5′ (IIIa).

In a further aspect, the double-stranded region is 15-30 nucleotide pairs in length. In another aspect, the double-stranded region is 17-23 nucleotide pairs in length. In another embodiment, the double-stranded region is 17-25 nucleotide pairs in length. In yet another embodiment, the double-stranded region is 23-27 nucleotide pairs in length. In a further aspect, the double-stranded region is 19-21 nucleotide pairs in length. In yet another aspect, the double-stranded region is 21-23 nucleotide pairs in length.

In one embodiment, each strand has 15-30 nucleotides. In a further embodiment, each strand has 19-30 nucleotides.

In one aspect, the modifications on the nucleotides are selected from the group consisting of the modifications as listed in Tables 5, 9, 10, 11B, 12, 13, and combinations thereof.

In some embodiments, the modifications on the nucleotides are 2′-O-methyl and 2′-fluoro modifications.

In some embodiments, the ligand is one or more GalNAc derivatives attached through a bivalent or trivalent branched linker. In a further embodiment, the ligand is

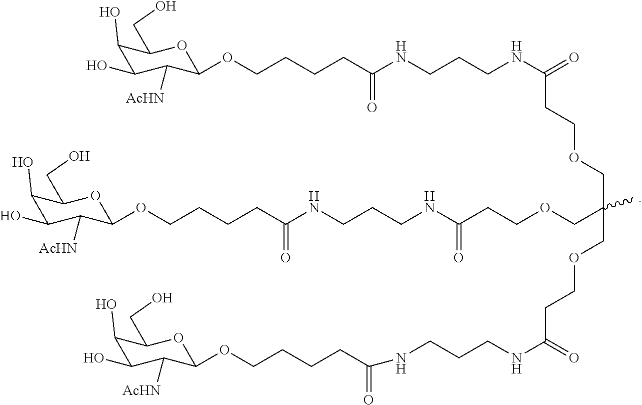 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some aspects, the ligand is attached to the 3′ end of the sense strand.

In certain embodiments, the the RNAi agent is conjugated to the ligand as shown in the following schematic

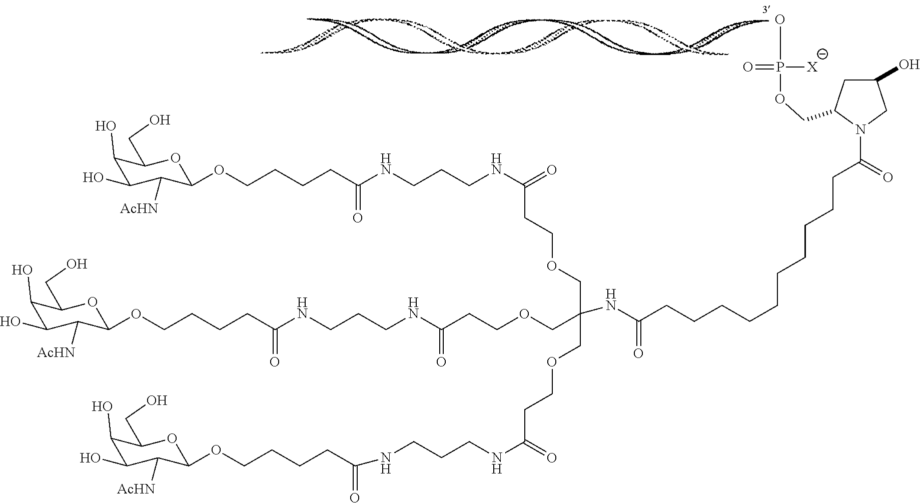

wherein X is O or S.

In some aspects, the RNAi agent further comprises at least one phosphorothioate or methylphosphonate internucleotide linkage. In a further aspect, the phosphorothioate or methylphosphonate internucleotide linkage is at the 3′-terminus of one strand. In another further aspect, the strand is the antisense strand. In yet another aspect, the strand is the sense strand.

In some embodiments, the phosphorothioate or methylphosphonate internucleotide linkage is at the 5′-terminus of one strand. In a further aspect, the strand is the antisense strand. In another further aspect, the strand is the sense strand.

In certain embodiments, the phosphorothioate or methylphosphonate internucleotide linkage is at the both the 5′- and 3′-terminus of one strand. In one embodiment, the strand is the antisense strand.

In some aspects, the RNAi agent comprises 6-8 phosphorothioate internucleotide linkages. In a further embodiment, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3′-terminus.

In some embodiments, the the base pair at the 1 position of the 5′-end of the antisense strand of the duplex is an AU base pair.

In some aspects, the Y nucleotides contain a 2′-fluoro modification. In a further aspect, the Y′ nucleotides contain a 2′-O-methyl modification.

In some embodiments, the sense strand has a total of 21 nucleotides and the antisense strand has a total of 23 nucleotides.

In some aspects, the RNAi agent is selected from the group of RNAi agents listed in any one of Tables 4A, 4B, 5, 8, 9, 10, 11A, 11B and 12.

In certain embodiments, the present invention also provides a double stranded RNAi agent capable of inhibiting the expression of apolipoprotein C3 (APOC3) in a cell, wherein the double stranded RNAi agent comprises a sense strand and an antisense strand forming a double-stranded region,
wherein the sense strand comprises 5′-GCUUAAAAGGGACAGUAUUCU-3′ (SEQ ID NO: 13), and the antisense strand comprises 5′-AGAAUACUGUCCCUUUUAAGCAA-3′ (SEQ ID NO: 14),
wherein substantially all of the nucleotides of the sense strand and substantially all of the nucleotides of the antisense strand are modified nucleotides,
wherein the sense strand is conjugated to a ligand attached at the 3′-terminus, and
wherein the ligand is one or more GalNAc derivatives attached through a bivalent or trivalent branched linker.

In other embodiments, the present invention also provides a double stranded RNAi agent capable of inhibiting the expression of apolipoprotein C3 (APOC3) in a cell, wherein the double stranded RNAi agent comprises a sense strand and an antisense strand forming a double-stranded region,
wherein the sense strand comprises 5′-GCUUAAAAGGGACAGUAUUCU-3′ (SEQ ID NO: 13), and the antisense strand comprises 5′-UGAAUACUGUCCCUUUUAAGCAA-3′ (SEQ ID NO: 15),
wherein substantially all of the nucleotides of the sense strand and substantially all of the nucleotides of the antisense strand are modified nucleotides,
wherein the sense strand is conjugated to a ligand attached at the 3′-terminus, and
wherein the ligand is one or more GalNAc derivatives attached through a bivalent or trivalent branched linker.

In certain embodiments, the present invention also provides a double stranded RNAi agent capable of inhibiting the expression of apolipoprotein C3 (APOC3) in a cell, wherein the double stranded RNAi agent comprises a sense strand and an antisense strand forming a double-stranded region,
wherein the sense strand comprises 5′-GCUUAAAAGGGACAGUAUUCA-3′ (SEQ ID NO:659), and the antisense strand comprises 5′-UGAAUACUGUCCCUUUUAAGCAA-3′ (SEQ ID NO:670),
wherein substantially all of the nucleotides of the sense strand and substantially all of the nucleotides of the antisense strand are modified nucleotides,
wherein the sense strand is conjugated to a ligand attached at the 3′-terminus, and
wherein the ligand is one or more GalNAc derivatives attached through a bivalent or trivalent branched linker.

In embodiment, all of the nucleotides of the sense strand are modified nucleotides. In one embodiment, all of the nucleotides of the antisense strand are modified nucleotides. In another embodiment, all of the nucleotides of the sense strand and all of the nucleotides of the antisense strand are modified nucleotides.

In a further aspect, at least one of the modified nucleotides is selected from the group consisting of a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate, and a terminal nucleotide linked to a cholesteryl derivative or a dodecanoic acid bisdecylamide group.

In one embodiment, the RNAi agent comprises no more than 10 nucleotides comprising 2′-fluoro modifications. In another embodiment, the RNAi agent comprises no more than 9 nucleotides comprising 2′-fluoro modifications. In another embodiment, the RNAi agent comprises no more than 8 nucleotides comprising 2′-fluoro modifications. In another embodiment, the RNAi agent comprises no more than 7 nucleotides comprising 2′-fluoro modifications. In another embodiment, the RNAi agent comprises no more than 6 nucleotides comprising 2′-fluoro modifications. In another embodiment, the RNAi agent comprises no more than 5 nucleotides comprising 2′-fluoro modifications. In yet another embodiment, the sense strand comprises no more than 4 nucleotides comprising 2′-fluoro modifications. In another embodiment, the sense strand comprises no more than 4 nucleotides comprising 2′-fluoro modifications. In another embodiment, the sense strand comprises no more than 3 nucleotides comprising 2′-fluoro modifications. In another embodiment, the sense strand comprises no more than 2 nucleotides comprising 2′-fluoro modifications. In another aspect, the antisense strand comprises no more than 6 nucleotides comprising 2′-fluoro modifications. In another embodiment, the antisense strand comprises no more than 5 nucleotides comprising 2′-fluoro modifications. In another embodiment, the the antisense strand comprises no more than 4 nucleotides comprising 2′-fluoro modifications. In another embodiment, the antisense strand comprises no more than 3 nucleotides comprising 2′-fluoro modifications. In yet another aspect, the antisense strand comprises no more than 2 nucleotides comprising 2′-fluoro modifications.

In one embodiment, the double-stranded RNAi agent of the invention further comprises a 5′-phosphate or a 5′-phosphate mimic at the 5′ nucleotide of the antisense strand. In another embodiment, the double-stranded RNAi agent further comprises a 5′-phosphate mimic at the 5′ nucleotide of the antisense strand. In a specific embodiment, the 5′-phosphate mimic is a 5′-vinyl phosphate (5′-VP).

In certain aspects, the ligand is

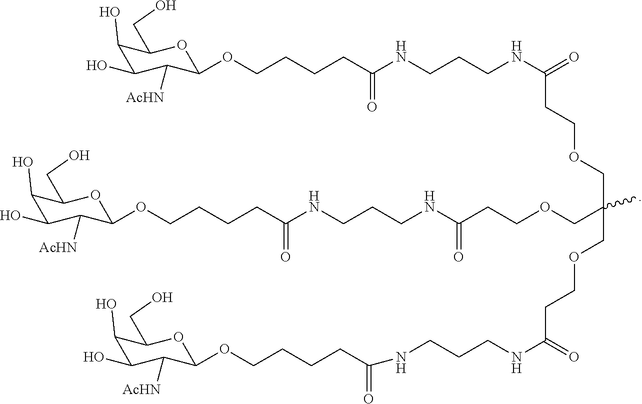 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent is conjugated to the ligand as shown in the following schematic

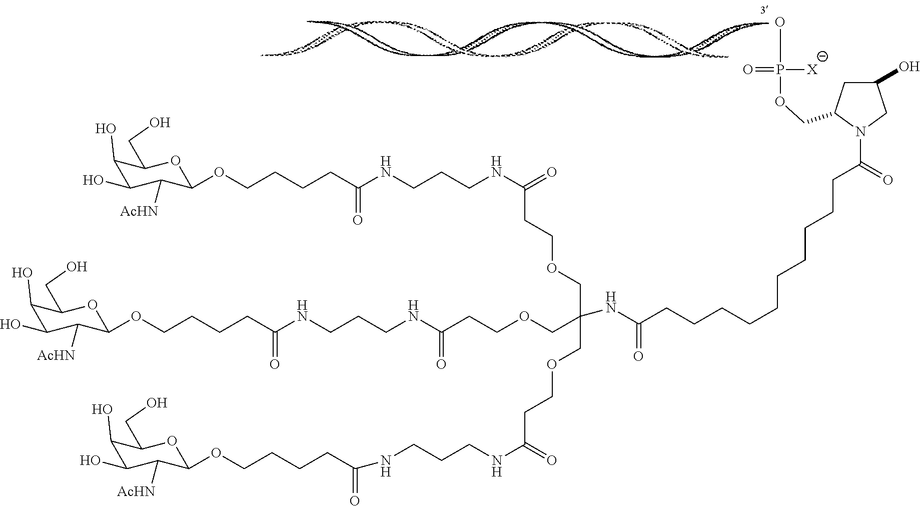

wherein X is O or S.

In some aspects, the present invention provides a double stranded RNAi agent comprising the RNAi sequences listed in any one of Tables 4A, 4B, 5, 8, 9, 10, 11A, 11B, 12, and 13.

In one embodiment, the RNAi agent is AD-57553 comprising the following sequence:

sense:
(SEQ ID NO: 16)
5′ GfscsUfuAfaAfaGfGfGfaCfaGfuAfuUfcUfL96 3′
antisense:
(SEQ ID NO: 17)
5′ asGfsaAfuAfcUfgUfcccUfuUfuAfaGfcsAfsa 3′.
In another embodiment, the RNAi agent is AD-65696
comprising the following sequence:
sense:
(SEQ ID NO: 18)
5′ GfscsUfuAfaAfaGfGfGfaCfaGfuAfuUfcUfL96 3′
antisense:
(SEQ ID NO: 19)
5′ VPusGfsaAfuAfcUfgUfcccUfuUfuAfaGfcsasa 3′.

In yet another aspect, the RNAi agent is AD-65703 comprising the following sequence:

sense:
(SEQ ID NO: 20)
5′ gscsuuaaAfaGfGfGfacaguauucaL96 3′
antisense:
(SEQ ID NO: 21)
5′ usGfsaauAfcUfGfucccUfuUfuaagcsasa 3′.

In yet another aspect, the RNAi agent is AD-65704 comprising the following sequence:

sense:
(SEQ ID NO: 22)
5′ gscsuuaaAfaGfGfGfacaguauucaL96 3′
antisense:
(SEQ ID NO: 23)
5′ usGfsaauacugucccUfuuuaagcsasa 3′.

In yet another aspect, the RNAi agent is AD-67221 comprising the following sequence:

sense:
(SEQ ID NO: 714)
5′ cscscaauAfaAfGfCfuggacaagaaL96 3′
antisense:
(SEQ ID NO: 718)
5′ usUfscuuGfuCfCfagcuUfuAfuugggsasg 3′

In one embodiment, the RNAi agent is AD-69535 comprising the following sequence:

sense:
(SEQ ID NO: 738)
5′ gscsuuaaaaGfgGfacaguauuca 3′
antisense:
(SEQ ID NO: 749)
5′ sGfsaauacugucCfcUfuuuaagcsasa 3′.

In another embodiment, the RNAi agent is AD-69541 comprising the following sequence:

sense:
(SQE ID NO: 744)
5′ gscsuuaaaaGfgGfacagu(Agn)uuca 3′
antisense:
(SEQ ID NO: 755)
5′ usGfsaauacugucCfcUfuuuaagcsasa 3′.

In certain embodiments, the present invention also provides a composition comprising a modified antisense polynucleotide agent, wherein the agent is capable of inhibiting the expression of APOC3 in a cell, and comprises a sequence complementary to a sense sequence selected from the group of the sequences listed in any one of Tables 4A, 4B, 5, 8, 9, 10, 11A, 11B, 12, and 13, wherein the polynucleotide is about 14 to about 30 nucleotides in length.

In some aspects, the present invention also provides a vector containing the double stranded RNAi agent as described herein. In other aspects, the present invention also provides a cell containing the double stranded RNAi agent as described herein.

In some embodiments, the present invention relates to a pharmaceutical composition comprising the double stranded RNAi agent, or the composition comprising a modified antisense polynucleotide agent, or the vector as described herein.

In certain aspects, the double stranded RNAi agent is present in an unbuffered solution. In a further aspect, the unbuffered solution is saline or water. In other aspects, the double stranded RNAi agent is present in a buffered solution. In a further embodiment, the buffer solution comprises acetate, citrate, prolamine, carbonate, or phosphate or any combination thereof. In a specific embodiment, the buffer solution is phosphate buffered saline (PBS).

In one embodiment, the present invention also provides a method of inhibiting apolipoprotein C3 (APOC3) expression in a cell, the method comprising:
(a) contacting the cell with the double stranded RNAi agent, or the composition comprising a modified antisense polynucleotide agent, the vector, or the pharmaceutical composition as described herein; and
(b) maintaining the cell produced in step (a) for a time sufficient to obtain degradation of the mRNA transcript of a APOC3 gene, thereby inhibiting expression of the APOC3 gene in the cell.

In one aspect, the cell is within a subject. In a further aspect, the subject is a human or a rabbit. In one embodiment, the subject suffers from an APOC3 associated disease.

In some embodiments, the APOC3 expression is inhibited by at least about 30%, about 40%, about 50%, about 60%, about 70%, about 80%, about 90%, about 95%, about 98% or about 100%.

In some aspects, the present invention provides a method of treating a subject having an apolipoprotein C3 (APOC3) associated disease, comprising administering to the subject a therapeutically effective amount of the double stranded RNAi agent, or the composition comprising a modified antisense polynucleotide agent, or the vector, or the pharmaceutical composition as described herein, thereby treating said subject.

In one embodiment, the APOC3 associated disease is hypertriglyceridemia. In another embodiment, the APOC3 associated disease is selected from the group consisting of non-alcoholic fatty liver disease, non-alcoholic steatohepatitis, polycystic ovary syndrome, kidney disease, obesity, type 2 diabetes mellitus (insulin resistance), hypertension, artherosclerosis and pancreatitis.

In some aspects, the double stranded RNAi agent is administered at a dose of about 0.01 mg/kg to about 10 mg/kg or about 0.5 mg/kg to about 50 mg/kg. In a further aspect, the double stranded RNAi agent is administered at a dose of about 10 mg/kg to about 30 mg/kg.

In another aspect, the double stranded RNAi agent is administered at a dose of about 3 mg/kg. In yet another aspect, the double stranded RNAi agent is administered at a dose of about 10 mg/kg.

In one embodiment, the double stranded RNAi agent is administered subcutaneously. In another embodiment, the double stranded RNAi agent is administered intravenously. In another embodiment, the double stranded RNAi agent is administered intramuscularly.

In some aspects, the RNAi agent is administered in two or more doses. In a further aspect, the RNAi agent is administered at intervals selected from the group consisting of once every about 12 hours, once every about 24 hours, once every about 48 hours, once every about 72 hours, and once every about 96 hours.

In certain embodiments, the methods of the invention further comprise administering to the subject an additional therapeutic agent. In a further embodiment, the additional therapeutic agent is selected from the group consisting of an HMG-CoA reductase inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, or fish oil.

### BRIEF DESCRIPTION OF THE DRAWINGS

FIG. 1 is a bar graph showing the relative amount of APOC3 mRNA in Hep3B cells after treatment with a single dose of 0.1 nM or 10 mM of the indicated iRNAs of the invention.

FIG. 2 is a bar graph showing the relative amount of APOC3 mRNA measured on day 5 in wild-type mice treated with 3, 10 and 30 mg/kg dose of GalNac-conjugated AD-57558.

FIG. 3 is a bar graph showing the measured levels of APOC3 mRNA measured in individual APOC3-AAV mice injected with AD-57553, AD-57547 and AD-58924.

FIG. 4 is a bar graph showing the group averages of the levels of APOC3 mRNA measured in APOC3-AAV mice injected with AD-57553, AD-57547 and AD-58924.

FIG. 5 is a bar graph showing the relative amount of APOC3 mRNA measured in APOC3-AAV mice previously injected with 10¹¹ genome copies of hAPOC3 AAV, followed by 1.25 mg/kg, 2.5 mg/kg and 5 mg/kg doses of AD-57553.

FIG. 6 is a bar graph showing the group averages of the relative amount of APOC3 mRNA measured in APOC3-AAV mice previously injected with 10¹¹ genome copies of hAPOC3 AAV, followed by 1.25 mg/kg, 2.5 mg/kg and 5 mg/kg doses of AD-57553.

FIG. 7A is a graph showing a 20-day time course of serum APOC3 protein measured in APOC3-AAV mice injected with 10¹¹ genome copies of hAPOC3 AAV, followed by 3 mg/kg dose of the indicated iRNAs of the invention.

FIG. 7B is a graph showing a 30-day time course of serum APOC3 protein measured in APOC3-AAV mice injected with 10¹¹ genome copies of hAPOC3 AAV, followed by 3 mg/kg dose of the indicated iRNAs of the invention.

FIG. 8 is a bar graph showing the amount of serum APOC3 protein measured on day 10 in APOC3-AAV mice injected with 10¹¹ genome copies of hAPOC3 AAV, followed by 3 mg/kg dose of the indicated iRNAs of the invention.

FIG. 9 is a bar graph showing the amount of serum APOC3 protein measured on day 20 in APOC3-AAV mice injected with 10¹¹ genome copies of hAPOC3 AAV, followed by 3 mg/kg dose of the indicated iRNAs of the invention.

FIG. 10 is a time course showing the amount of APOC3 protein measured in APOC3-AAV mice injected with 10¹¹ genome copies of hAPOC3 AAV, followed by 3 mg/kg dose of the indicated iRNAs of the invention.

FIG. 11 is a schematic showing the dosing schedule Q2W×4 used for multi-dose studies with AD-57553, AD-65696, AD-65699, AD-65703 and AD-65704.

FIG. 12A is a time course showing the amount of APOC3 protein measured in APOC3-AAV mice injected with 10¹¹ genome copies of hAPOC3 AAV, followed by four 0.3 mg/kg doses of the indicated iRNAs of the invention administered according to the dosing schedule shown in FIG. 11.

FIG. 12B is a time course showing the amount of APOC3 protein measured in APOC3-AAV mice injected with 10¹¹ genome copies of hAPOC3 AAV, followed by four 1 mg/kg doses of the indicated iRNAs of the invention administered according to the dosing schedule shown in FIG. 11.

FIG. 12C is a time course showing the amount of APOC3 protein measured in APOC3-AAV mice injected with 10¹¹ genome copies of hAPOC3 AAV, followed by four 3 mg/kg doses of the indicated iRNAs of the invention administered according to the dosing schedule shown in FIG. 11.

FIG. 13 is a bar graph showing the relative amounts of APOC3 protein measured on day 14 in APOC3-AAV mice injected with 10¹¹ genome copies of hAPOC3 AAV, followed by single doses of 0.3 mg/kg, 1 mg/kg and 3 mg/kg of AD-65704.

FIG. 14 is a bar graph showing the relative amounts of APOC3 protein measured 20 days after last dose in APOC3-AAV mice injected with 10¹¹ genome copies of hAPOC3 AAV, followed by multiple doses of 0.3 mg/kg, 1 mg/kg and 3 mg/kg of AD-65704, administered according to the dosing schedule shown in FIG. 11.

FIG. 15 is a time course showing the amount of APOC3 protein measured in APOC3-AAV mice injected with 10¹¹ genome copies of hAPOC3 AAV, followed by 1 mg/kg dose of the indicated iRNAs of the invention.

FIG. 16A is a bar graph showing the relative amounts of APOC3 protein measured on day 10 in APOC3-AAV mice injected with 10¹¹ genome copies of hAPOC3 AAV, followed by a 1 mg/kg single dose of the indicated iRNAs of the invention.

FIG. 16B is a bar graph showing the relative amounts of APOC3 protein measured on day 24 in APOC3-AAV mice injected with 10¹¹ genome copies of hAPOC3 AAV, followed by a 1 mg/kg single dose of the indicated iRNAs of the invention.

FIG. 17A is a bar graph showing the relative amounts of serum APOC3 protein measured on day 14 in APOC3-AAV mice injected with 10¹¹ genome copies of hAPOC3 AAV, followed by a single 1 mg/kg dose of the indicated iRNAs.

FIG. 17B is a graph showing the amount of serumAPOC3 protein at days 0, 14, 28, and 42 relative to pre-dose levels measured in APOC3-AAV mice injected with 10¹¹ genome copies of hAPOC3 AAV, followed by a single 1 mg/kg dose of the indicated iRNAs.

FIG. 18A is a graph showing the amounts, relative to pre-dose on day −7, of serum APOC3 protein measured on days 1, 8, 11, 15, 22, 29, 36, 43, 57, 64, and 71 in Cynomolgus monkeys following a single 1 mg/kg weekly dose of AD-65704 for 8 weeks (QW×8).

FIG. 18B is a graph showing the amounts, relative to pre-dose on day −7, of serum APOC3 protein measured on days 1, 8, 11, 15, 22, 29, and 36 in Cynomolgus monkeys following a single 1 mg/kg dose of AD-65704.

FIG. 18C is a graph showing the amount, relative to pre-dose on day −7, of liver APOC3 mRNA on day 64 in Cynomolgus monkeys following a single 1 mg/kg weekly dose of AD-65704 for 5 weeks (q1w×5) and the amount, relative to pre-dose on day −7, of liver APOC3 mRNA on day 12 in Cynomolgus monkeys following a single 1 mg/kg dose of AD-65704.

FIG. 19A is a graph showing the amounts, relative to pre-dose on day −7, of serum APOC3 protein measured on days 1, 8, 11, 15, 22, 29, and 36 in Cynomolgus monkeys following a single 1 mg/kg dose of the indicated iRNAs.

FIG. 19B is a bar graph showing the amounts, relative to pre-dose on day −7, of liver APOC3 mRNA measured on day 12 in Cynomolgus monkeys following a single 1 mg/kg dose of the indicated iRNAs.

FIG. 20A is a graph showing the amounts, relative to pre-dose on day −7, of serum APOC3 mRNA measured on days 1, 8, 11, 15, 22, 29, 36, 43, 50, 57, 64, and 71 in Cynomolgus monkeys following a single 1 mg/kg dose of the indicated iRNAs and subsequent administration of a single subcutaneous 3 mg/kg dose of the same agent on day 36.

FIG. 20B is a bar graph showing the amounts, relative to pre-dose on day −7, of liver APOC3 mRNA measured on day 12 in Cynomolgus monkeys following a single 1 mg/kg dose of the indicated iRNAs on day 1 followed by a single 3 mg/kg dose of the same iRNA agent on day 36.

### DETAILED DESCRIPTION OF THE INVENTION

The present invention provides iRNA agents, e.g., double-stranded iRNA agents, and compositions that reduce or inhibit the expression of an APOC3 gene. The gene may be within a cell, e.g., a cell within a subject, such as a human.

The present invention also provides methods for treating a subject having a disorder that would benefit from inhibiting or reducing the expression of an APOC3, e.g., an apolipoprotein C3 associated disease or disorder, such as hypertriglyceridemia, using iRNA compositions which inhibit or reduce the expression of the APOC3 gene.

The iRNAs of the invention include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an APOC3 gene. The use of these iRNAs enables the targeted degradation of mRNAs of the APOC3 gene in cells. Very low dosages of the iRNAs of the invention, in particular, can specifically and efficiently mediate RNA interference (RNAi), resulting in significant inhibition of expression of the APOC3 gene. Using in vitro and in vivo assays, the present inventors have demonstrated that iRNAs targeting the APOC3 gene can mediate RNAi, resulting in significant inhibition of expression of APOC3 gene and in reduced levels of APOC3 protein. The present inventors have also demonstrated that iRNAs targeting an APOC3 gene can reduce the symptoms associated with an apolipoprotein C3 associated disorder, e.g., lower triglyceride levels. Thus, methods and compositions including these iRNAs are useful for treating a subject having an apolipoprotein C3 associated disorder, such as hypertriglyceridemia.

The following detailed description discloses how to make and use compositions containing iRNAs to inhibit the expression of APOC3 gene as well as compositions, uses, and methods for treating subjects having diseases and disorders that would benefit from inhibition and/or reduction of the expression of APOC3.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the term “APOC3” refers to the well-known gene that encodes apolipoprotein C3, as well as to its protein product, also known in the art as HALP2 or APOCIII.

The term “APOC3” includes human APOC3, the amino acid and complete coding sequence of which may be found in for example, GenBank Accession No. GI:4557322 (NM_000040.1; SEQ ID NO:1); Macaca fascicularis APOC3, the amino acid and complete coding sequence of which may be found in for example, GenBank Accession No. GI:544489959 (XM_05579730.1, SEQ ID NO:3); Macaca mulatta APOC3, the amino acid and complete coding sequence of which may be found in for example, GenBank Accession No. GI:297269260 (XM_001090312.2; SEQ ID NO: 5); mouse (Mus musculus) APOC3, the amino acid and complete coding sequence of which may be found in for example, GenBank Accession No. GI:577019555 (NM_023114.4, SEQ ID NO:7); rat (Rattus norvegicus) APOC3, the amino acid and complete coding sequence of which may be found in for example, GenBank Accession No. GI:402534545 (NM_012501.2, SEQ ID NO:9); and rabbit (Oryctolagus cuniculus), GenBank Accession No. GI:655601498 (XM_002708371.2, SEQ ID NO:11).

Additional examples of APOC3 mRNA sequences are readily available through publicly available databases, e.g., GenBank, UniProt, OMIM, and the Macaca genome project web site.

The term “APOC3,” as used herein, also refers to naturally occurring DNA sequence variations of the APOC3 gene, such as a single nucleotide polymorphism (SNP) in the APOC3 gene. Exemplary SNPs in the APOC3 DNA sequence may be found through the dbSNP database available at www.ncbi.nlm.nih.gov/projects/SNP/. Non-limiting examples of sequence variations within the APOC3 gene include, for example, the two variations rs2854116 and rs2854117, described in Petersen, K. F. et al., (2010), N. Engl. J. Med. 362(12):1082-1089, the entire contents of which are incorporated herein by reference.

As used herein, “target sequence” refers to a contiguous portion of the nucleotide sequence of an mRNA molecule formed during the transcription of an APOC3 gene, including mRNA that is a product of RNA processing of a primary transcription product. In one embodiment, the target portion of the sequence will be at least long enough to serve as a substrate for iRNA-directed cleavage at or near that portion of the nucleotide sequence of an mRNA molecule formed during the transcription of an APOC3 gene.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety (see, e.g., Table 3). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of APOC3 in a cell, e.g., a cell within a subject, such as a mammalian subject.

In one embodiment, an RNAi agent of the invention includes a single stranded RNA that interacts with a target RNA sequence, e.g., an APOC3 target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes these dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (ssRNA) (the antisense strand of an siRNA duplex) generated within a cell and which promotes the formation of a RISC complex to effect silencing of the target gene, i.e., an APOC3 gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an “iRNA” for use in the compositions, uses, and methods of the invention is a double-stranded RNA and is referred to herein as a “double stranded RNAi agent,” “double-stranded RNA (dsRNA) molecule,” “dsRNA agent,” “RNAi agent”, “RNAi”, or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., an APOC3 gene. In some embodiments of the invention, a double-stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, and/or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “RNAi agent” for the purposes of this specification and claims.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, at least 3, at least 4, at least 5, at least 6, at least 7, at least 8, at least 9, at least 10, at least 20, at least 23 or more unpaired nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA, each strand of which comprises 20-30 nucleotides that interacts with a target RNA sequence, e.g., an APOC3 target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188).

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end and/or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end and/or the 5′-end. In another embodiment, one or more of the nucleotides in the overhang is replaced with a nucleoside thiophosphate.

“Blunt” or “blunt end” means that there are no unpaired nucleotides at that end of the double stranded RNAi agent, i.e., no nucleotide overhang. A “blunt ended” RNAi agent is a dsRNA that is double-stranded over its entire length, i.e., no nucleotide overhang at either end of the molecule. The RNAi agents of the invention include RNAi agents with nucleotide overhangs at one end (i.e., agents with one overhang and one blunt end) or with nucleotide overhangs at both ends.

The term “antisense strand” or “guide strand” refers to the strand of an iRNA, e.g., a dsRNA, which includes a region that is substantially complementary to a target sequence, e.g., a APOC3 mRNA. As used herein, the term “region of complementarity” refers to the region on the antisense strand that is substantially complementary to a sequence, for example a target sequence, e.g., an APOC3 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the iRNA.

The term “sense strand,” or “passenger strand” as used herein, refers to the strand of an iRNA that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding APOC3). For example, a polynucleotide is complementary to at least a part of an APOC3 mRNA if the sequence is substantially complementary to a non-interrupted portion of an mRNA encoding APOC3.

Accordingly, in some embodiments, the antisense polynucleotides disclosed herein are fully complementary to the target APOC3 sequence. In other embodiments, the sense strand polynucleotides and/or the antisense polynucleotides disclosed herein are substantially complementary to the target APOC3 sequence and comprise a contiguous nucleotide sequence which is at least about 80% complementary over its entire length to the equivalent region of the nucleotide sequence of any one of SEQ ID NOs:1-12, or a fragment of any one of SEQ ID NOs:1-12, such as about 85%, about 86%, about 87%, about 88%, about 89%, about 90%, about % 91%, about 92%, about 93%, about 94%, about 95%, about 96%, about 97%, about 98%, or about 99% complementary.

In one embodiment, an RNAi agent of the invention includes a sense strand that is substantially complementary to an antisense polynucleotide which is complementary to a target APOC3 sequence, and wherein the sense strand polynucleotide comprises a contiguous nucleotide sequence which is at least about 80% complementary over its entire length to the equivalent region of the nucleotide sequence of any one of SEQ ID NOs:1-12, or a fragment of any one of SEQ ID NOs:1-12, such as about 85%, about 86%, about 87%, about 88%, about 89%, about 90%, about % 91%, about 92%, about 93%, about 94%, about 95%, about 96%, about 97%, about 98%, or about 99% complementary. In another embodiment, an RNAi agent of the invention includes an antisense strand that is substantially complementary to the target APOC3 sequence and comprise a contiguous nucleotide sequence which is at least about 80% complementary over its entire length to the equivalent region of the nucleotide sequence of any one of SEQ ID NOs:1-12, or a fragment of any one of SEQ ID NOs:1-12, such as about 85%, about 86%, about 87%, about 88%, about 89%, about 90%, about % 91%, about 92%, about 93%, about 94%, about 95%, about 96%, about 97%, about 98%, or about 99% complementary.

In general, the majority of nucleotides of each strand are ribonucleotides, but as described in detail herein, each or both strands can also include one or more non-ribonucleotides, e.g., a deoxyribonucleotide and/or a modified nucleotide. In addition, an “iRNA” may include ribonucleotides with chemical modifications. Such modifications may include all types of modifications disclosed herein or known in the art. Any such modifications, as used in an iRNA molecule, are encompassed by “iRNA” for the purposes of this specification and claims.

In one aspect of the invention, an agent for use in the methods and compositions of the invention is a single-stranded antisense nucleic acid molecule that inhibits a target mRNA via an antisense inhibition mechanism. The single-stranded antisense nucleic acid molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense nucleic acid molecule may be about 15 to about 30 nucleotides in length and have a sequence that is complementary to a target sequence. For example, the single-stranded antisense nucleic acid molecule may comprise a sequence that is at least about 15, 16, 17, 18, 19, 20, or more contiguous nucleotides from any one of the antisense sequences described herein.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). In an embodiment, the subject is a human, such as a human being treated or assessed for a disease, disorder or condition that would benefit from reduction in APOC3 expression; a human at risk for a disease, disorder or condition that would benefit from reduction in APOC3 expression; a human having a disease, disorder or condition that would benefit from reduction in APOC3 expression; and/or human being treated for a disease, disorder or condition that would benefit from reduction in APOC3 expression as described herein.

As used herein, the terms “treating” or “treatment” refer to a beneficial or desired result including, but not limited to, alleviation or amelioration of one or more symptoms associated with unwanted or excessive APOC3 expression, e.g., hypertriglyceridemia (or high triglyceride levels). Such symptoms may include, e.g., skin symptoms (e.g., eruptive xanthoma); eye abnormalities (e.g., lipemia retinalis); hepatosplenomegaly (enlargement of the liver and spleen); neurological symptoms; or attacks of abdominal pain that may be mild episodes of pancreatitis. Other symptoms associated with unwanted or excessive APOC3 expression may also include any symptom of a disease, disorder or condition that may be caused by, be associated with, or be a consequence of hypertriglyceridemia, e.g., non-alcoholic fatty liver disease, non-alcoholic steatohepatitis, polycystic ovary syndrome, kidney disease, obesity, type 2 diabetes mellitus (insulin resistance), artherosclerosis, cardiovascular disease or pancreatitis. “Treatment” can also mean prolonging survival as compared to expected survival in the absence of treatment.

The term “lower” in the context of the level of APOC3 in a subject or a disease marker or symptom refers to a statistically significant decrease in such level. The decrease can be, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more and is preferably down to a level accepted as within the range of normal for an individual without such disorder.

As used herein, “prevention” or “preventing,” when used in reference to a disease, disorder or condition thereof, may be treated or ameliorated by a reduction in expression of an APOC3 gene, refers to a reduction in the likelihood that a subject will develop a symptom associated with such a disease, disorder, or condition, e.g., a symptom of unwanted or excessive APOC3 expression, such as hypertriglyceridemia. The likelihood of developing, e.g., hypertriglyceridemia, is reduced, for example, when an individual having one or more risk factors for hypertriglyceridemia either fails to develop hypertriglyceridemia or develops hypertriglyceridemia with less severity relative to a population having the same risk factors and not receiving treatment as described herein. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

As used herein, the term “apolipoprotein C3-associated disease” or “APOC3-associated disease,” is a disease, disorder or a condition that is caused by, or is associated with, unwanted or excessive APOC3 expression. The term “APOC3-associated disease” includes a disease, disorder or condition that may be treated or ameliorated by a reduction in APOC3 expression. The term APOC3-associated disease” includes hypertriglyceridemia, or a high triglyceride levels.

The levels of triglycerides in a serum of a subject, e.g., a human subject, that may be indicative of hypertriglyceridemia are described in Oh, R. C. et al., (2007) American Family Physician, 75(9):1366-1371. Specifically, hypertriglyceridemia may be associated with “borderline-high serum triglyceride levels” (i.e., 150 to 199 mg per dL or 1.70 to 2.25 mmol per L); “high serum triglyceride levels” (i.e., 200 to 499 mg per dL or 2.26 to 5.64 mmol per L); or “very high triglyceride levels” (i.e., 500 mg per dL or higher (or 5.65 mmol per L or higher)

In one embodiment, an APOC3-associated disease is primary hypertriglyceridemia. “Primary triglyceridemia” results from environmental or genetic causes (e.g., a result of no obvious underlying medical cause). Exemplary diseases characterized as primary hypertriglyceridemias include, but are not limited to, familial chylomicronemia (hyperlipoproteinemia type I), primary mixed hyperlipidemia (type 5), familial hypertriglyceridemia (hyperlipoproteinemia type 4), familial combined hyperlipoproteinemia (type 2B) and familial dysbetalipoproteinemia (hyperlipoproteinemia type 3).

In another embodiment, an APOC3-associated disease is secondary hypertriglyceridemia. “Secondary triglyceridemia” is caused by, or be associated with, other underlying disorders and conditions. Such disorders and/or conditions include, e.g., obesity, metabolic syndrome, diabetes, fatty liver, alcohol use, renal disease, pregnancy, nonalcoholic fatty liver disorder, hypothyroidism, paraproteinemias (such as hypergammaglobulinemia in macroglobulinemia, myeloma, lymphoma and lymphocytic leukemias), autoimmune disorders (such as systemic lupus erythematosis), intake of medications (such as antiretroviral drugs, including ritonavir and lopinavir, and antipsychotic medications, including clozapine and olanzapine), see G. Yuan et al., (2007) Canadian Medical Association Journal, 176(8):1113-1120.

Any disorder that may be a cause of hypertriglyceridemia (e.g., secondary hypertriglyceridemia) or that may be a consequence of hypertriglyceridemia (e.g., primary or secondary hypertriglyceridemia) is encompassed by the term “APOC3-associated disease”. Non-limiting examples of APOC3-associated diseases include metabolic disorders, e.g., non-alcoholic fatty liver disease, non-alcoholic steatohepatitis, polycystic ovary syndrome, kidney disease, obesity, type 2 diabetes mellitus (insulin resistance); hypertension; cardiovascular disorders, e.g., artherosclerosis; and pancreatitis, e.g., acute pancreatitis.

### II. iRNAs of the Invention

The present invention provides iRNAs which inhibit the expression of an APOC3 gene. In one embodiment, the iRNA agent includes double-stranded ribonucleic acid (dsRNA) molecules for inhibiting the expression of an APOC3 gene in a cell, such as a cell within a subject, e.g., a mammal, such as a human having an APOC3-associated disease, e.g., hypertriglyceridemia. The dsRNA includes an antisense strand having a region of complementarity which is complementary to at least a part of an mRNA formed in the expression of an APOC3 gene. The region of complementarity is about 30 nucleotides or less in length (e.g., about 30, 29, 28, 27, 26, 25, 24, 23, 22, 21, 20, 19, or 18 nucleotides or less in length). Upon contact with a cell expressing the APOC3 gene, the iRNA inhibits the expression of the APOC3 gene (e.g., a human, a primate, a non-primate, or a bird APOC3 gene) by at least about 10% as assayed by, for example, a PCR or branched DNA (bDNA)-based method, or by a protein-based method, such as by immunofluorescence analysis, using, for example, Western Blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an APOC3 gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is between 15 and 30 base pairs in length, e.g., between, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity to the target sequence is between 15 and 30 nucleotides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

In some embodiments, the dsRNA is between about 15 and about 20 nucleotides in length, or between about 25 and about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 9 to 36 base pairs, e.g., about 10-36, 11-36, 12-36, 13-36, 14-36, 15-36, 9-35, 10-35, 11-35, 12-35, 13-35, 14-35, 15-35, 9-34, 10-34, 11-34, 12-34, 13-34, 14-34, 15-34, 9-33, 10-33, 11-33, 12-33, 13-33, 14-33, 15-33, 9-32, 10-32, 11-32, 12-32, 13-32, 14-32, 15-32, 9-31, 10-31, 11-31, 12-31, 13-32, 14-31, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target APOC3 expression is not generated in the target cell by cleavage of a larger dsRNA.

A dsRNA as described herein can further include one or more single-stranded nucleotide overhangs e.g., 1, 2, 3, or 4 nucleotides. dsRNAs having at least one nucleotide overhang can have unexpectedly superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

A dsRNA can be synthesized by standard methods known in the art as further discussed below, e.g., by use of an automated DNA synthesizer, such as are commercially available from, for example, Biosearch, Applied Biosystems, Inc.

iRNA compounds of the invention may be prepared using a two-step procedure. First, the individual strands of the double-stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

In one aspect, a dsRNA of the invention includes at least two nucleotide sequences, a sense sequence and an anti-sense sequence. The sense strand and the corresponding antisense strand are each selected from the group of sequences provided in any one of Tables 4A, 4B, 5, 8, 9, 10, 11A, 11B, 12, and 13. In this aspect, one of the two sequences is complementary to the other of the two sequences, with one of the sequences being substantially complementary to a sequence of an mRNA generated in the expression of an APOC3 gene. As such, in this aspect, a dsRNA will include two oligonucleotides, where one oligonucleotide is described as the sense strand in any one of Tables 4A, 4B, 5, 8, 9, 10, 11A, 11B, 12, and 13, and the second oligonucleotide is described as the corresponding antisense strand of the sense strand in any one of Tables 4A, 4B, 5, 8, 9, 10, 11A, 11B, 12, and 13. In one embodiment, the substantially complementary sequences of the dsRNA are contained on separate oligonucleotides. In another embodiment, the substantially complementary sequences of the dsRNA are contained on a single oligonucleotide.

It will be understood that, although some of the sequences in Tables 4A, 4B, 5, 8, 9, 10, 11A, 11B, 12, and 13, are described as modified and/or conjugated sequences, the RNA of the iRNA of the invention e.g., a dsRNA of the invention, may comprise any one of the sequences set forth in Tables 4A, 4B, 5, 8, 9, 10, 11A, 11B, 12, and 13 that is un-modified, un-conjugated, and/or modified and/or conjugated differently than described therein.

The skilled person is well aware that dsRNAs having a duplex structure of between about 20 and 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in any one of Tables 4A, 4B, 5, 8, 9, 10, 11A, 11B, 12, and 13, dsRNAs described herein can include at least one strand of a length of minimally 21 nucleotides. It can be reasonably expected that shorter duplexes having one of the sequences of any one of Tables 4A, 4B, 5, 8, 9, 10, 11A, 11B, 12, and 13 minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences of any one of Tables 4A, 4B, 5, 8, 9, 10, 11A, 11B, 12, and 13, and differing in their ability to inhibit the expression of a APOC3 gene by not more than about 5, 10, 15, 20, 25, or 30% inhibition from a dsRNA comprising the full sequence, are contemplated to be within the scope of the present invention.

In addition, the RNAs provided in any one of Tables 4A, 4B, 5, 8, 9, 10, 11A, 11B, 12, and 13 identify a site(s) in a APOC3 transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 15 contiguous nucleotides from one of the sequences provided in any one of Tables 4A, 4B, 5, 8, 9, 10, 11A, 11B, 12, and 13 coupled to additional nucleotide sequences taken from the region contiguous to the selected sequence in a APOC3 gene.

While a target sequence is generally about 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, while the sequences identified, for example, in any one of Tables 4A, 4B, 5, 8, 9, 10, 11A, 11B, 12, and 13 represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified, e.g., in any one of Tables 4A, 4B, 5, 8, 9, 10, 11A, 11B, 12, and 13, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of iRNAs based on those target sequences in an inhibition assay as known in the art and/or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch is not located in the center of the region of complementarity. If the antisense strand of the iRNA contains mismatches to the target sequence, it is preferable that the mismatch be restricted to be within the last 5 nucleotides from either the 5′- or 3′-end of the region of complementarity. For example, for a 23 nucleotide iRNA agent the strand which is complementary to a region of an APOC3 gene, generally does not contain any mismatch within the central 13 nucleotides. The methods described herein or methods known in the art can be used to determine whether an iRNA containing a mismatch to a target sequence is effective in inhibiting the expression of an APOC3 gene. Consideration of the efficacy of iRNAs with mismatches in inhibiting expression of an APOC3 gene is important, especially if the particular region of complementarity in an APOC3 gene is known to have polymorphic sequence variation within the population.

### III. Modified iRNAs of the Invention

In one embodiment, the RNA of the iRNA of the invention e.g., a dsRNA, is un-modified, and does not comprise, e.g., chemical modifications and/or conjugations known in the art and described herein. In another embodiment, the RNA of an iRNA of the invention, e.g., a dsRNA, is chemically modified to enhance stability or other beneficial characteristics. In certain embodiments of the invention, substantially all of the nucleotides of an iRNA of the invention are modified. In other embodiments of the invention, all of the nucleotides of an iRNA of the invention are modified nucleotides. iRNAs of the invention in which “substantially all of the nucleotides are modified” are largely but not wholly modified and can include not more than 5, 4, 3, 2, or 1 unmodified nucleotides.

In some aspects of the invention, substantially all of the nucleotides of an iRNA of the invention are modified and the iRNA agents comprise no more than 10 nucleotides comprising 2′-fluoro modifications (e.g., no more than 9 2′-fluoro modifications, no more than 8 2′-fluoro modifications, no more than 7 2′-fluoro modifications, no more than 6 2′-fluoro modifications, no more than 5 2′-fluoro modifications, no more than 4 2′-fluoro modifications, no more than 5 2′-fluoro modifications, no more than 4 2′-fluoro modifications, no more than 3 2′-fluoro modifications, or no more than 2 2′-fluoro modifications). For example, in some embodiments, the sense strand comprises no more than 4 nucleotides comprising 2′-fluoro modifications (e.g., no more than 3 2′-fluoro modifications, or no more than 2 2′-fluoro modifications). In other embodiments, the antisense strand comprises no more than 6 nucleotides comprising 2′-fluoro modifications (e.g., no more than 5 2′-fluoro modifications, no more than 4 2′-fluoro modifications, no more than 4 2′-fluoro modifications, or no more than 2 2′-fluoro modifications). In other aspects of the invention, all of the nucleotides of an iRNA of the invention are modified and the iRNA agents comprise no more than 10 nucleotides comprising 2′-fluoro modifications (e.g., no more than 9 2′-fluoro modifications, no more than 8 2′-fluoro modifications, no more than 7 2′-fluoro modifications, no more than 6 2′-fluoro modifications, no more than 5 2′-fluoro modifications, no more than 4 2′-fluoro modifications, no more than 5 2′-fluoro modifications, no more than 4 2′-fluoro modifications, no more than 3 2′-fluoro modifications, or no more than 2 2′-fluoro modifications).

The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of iRNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′-position: OH; F; O—, S—, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)_{·n}OCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as deoxy-thymine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

The RNA of an iRNA can also be modified to include one or more bicyclic sugar moities. A “bicyclic sugar” is a furanosyl ring modified by the bridging of two atoms. A“bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)-O-2′ (and analogs thereof; see e.g., U.S. Pat. No. 8,278,283); 4′-CH2 N(OCH3)-2′ (and analogs thereof; see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2 C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426). The entire contents of each of the foregoing are hereby incorporated herein by reference.

Additional representative U.S. patents and US patenttent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and 3-D-ribofuranose (see WO 99/14226).

The RNA of an iRNA can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-O-2′ bridge. In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

One or more of the nucleotides of an iRNA of the invention may also include a hydroxymethyl substituted nucleotide. A “hydroxymethyl substituted nucleotide” is an acyclic 2′-3′-seco-nucleotide, also referred to as an “unlocked nucleic acid” (“UNA”) modification

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

A. Modified iRNAs Comprising Motifs of the Invention

In certain aspects of the invention, the double-stranded RNAi agents of the invention include agents with chemical modifications as disclosed, for example, in WO 2013/075035, filed on Nov. 16, 2012, the entire contents of which are incorporated herein by reference. As shown herein and in PCT Publication No. WO 2013/075035, a superior result may be obtained by introducing one or more motifs of three identical modifications on three consecutive nucleotides into a sense strand and/or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense and/or antisense strand. The RNAi agent may be optionally conjugated with a GalNAc derivative ligand, for instance on the sense strand. The resulting RNAi agents present superior gene silencing activity.

More specifically, it has been surprisingly discovered that when the sense strand and antisense strand of the double-stranded RNAi agent are completely modified to have one or more motifs of three identical modifications on three consecutive nucleotides at or near the cleavage site of at least one strand of an RNAi agent, the gene silencing activity of the RNAi agent was superiorly enhanced.

Accordingly, the invention provides double-stranded RNAi agents capable of inhibiting the expression of a target gene (i.e., apolipoprotein C3 (APOC3) gene) in vivo. The RNAi agent comprises a sense strand and an antisense strand. Each strand of the RNAi agent may range from 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as an “RNAi agent.” The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (preferably GalNAc3).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region region which is at least 25 nucleotides in length, and the second strand is sufficiently complemenatary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adajacent to the first motif or is separated by at least one or more nucleotides. When the motifs are immediately adjacent to each other then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motifs of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In one embodiment, every nucleotide in the sense strand and antisense strand of the RNAi agent, including the nucleotides that are part of the motifs, may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In one embodiment, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In one embodiment, the Nₐ and/or N_{b} comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In one embodiment, the RNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 5′-3′ of the strand within the duplex region. As another example, the alternating motif in the sense strand may start with “AABBAABB” from 5′-3′ of the strand and the alternating motif in the antisenese strand may start with “BBAABBAA” from 5′-3′ of the strand within the duplex region, so that there is a complete or partial shift of the modification patterns between the sense strand and the antisense strand.

In one embodiment, the RNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand and/or antisense strand interrupts the initial modification pattern present in the sense strand and/or antisense strand. This interruption of the modification pattern of the sense and/or antisense strand by introducing one or more motifs of three identical modifications on three consecutive nucleotides to the sense and/or antisense strand surprisingly enhances the gene silencing activity to the target gene.

In one embodiment, when the motif of three identical modifications on three consecutive nucleotides is introduced to any of the strands, the modification of the nucleotide next to the motif is a different modification than the modification of the motif. For example, the portion of the sequence containing the motif is “ . . . NₐYYYN_{b} . . . ,” where “Y” represents the modification of the motif of three identical modifications on three consecutive nucleotide, and “Nₐ” and “N_{b}” represent a modification to the nucleotide next to the motif “YYY” that is different than the modification of Y, and where Nₐ and N_{b} can be the same or different modifications. Alternatively, Nₐ and/or N_{b} may be present or absent when there is a wing modification present.

The RNAi agent may further comprise at least one phosphorothioate or methylphosphonate internucleotide linkage. The phosphorothioate or methylphosphonate internucleotide linkage modification may occur on any nucleotide of the sense strand or antisense strand or both strands in any position of the strand. For instance, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-standed RNAi agent comprises 6-8phosphorothioate internucleotide linkages. In one embodiment, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3′-terminus.

In one embodiment, the RNAi comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, and/or the 5′end of the antisense strand.

In one embodiment, the 2 nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the RNAi agent may additionally have two phosphorothioate internucleotide linkages between the terminal three nucleotides at both the 5′-end of the sense strand and at the 5′-end of the antisense strand.

In one embodiment, the RNAi agent comprises mismatch(es) with the target, within the duplex, or combinations thereof. The mistmatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, J:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense and/or antisense strand.

In one embodiment, the sense strand sequence may be represented by formula (I):

5′nₚ-Nₐ—(XXX)ᵢ-N_{b}—YYY—N_{b}—(ZZZ)ⱼ-Nₐ-n_{q}3′ (I)
wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein N_{b} and Y do not have the same modification; and
XXX, YYY and ZZZ each independently represent one motif of three identical modifications on three consecutive nucleotides. Preferably YYY is all 2′-F modified nucleotides.

In one embodiment, the Nₐ and/or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11, 12 or 11, 12, 13) of—the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

5′nₚ-Nₐ—YYY—N_{b}—ZZZ—Nₐ-n_{q}3′ (Ib);

5′nₚ-Nₐ—XXX—N_{b}—YYY—Nₐ-n_{q}3′ (Ic); or

5′nₚ-Nₐ—XXX—N_{b}—YYY—N_{b}—ZZZ—Nₐ-n_{q}3′ (Id).

When the sense strand is represented by formula (Ib), N_{b} represents an oligonucleotide sequence comprising 0-10, 0-7, 0-5, 0-4, 0-2 or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides. Each of X, Y and Z may be the same or different from each other.

In other embodiments, i is 0 and j is 0, and the sense strand may be represented by the formula:

5′nₚ-Nₐ—YYY—Nₐ-n_{q}3′ (Ia).

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

5′n_{q}′-Nₐ′—(Z′Z′Z′)k-N_{b}′—Y′Y′Y′—N_{b}′—(X′X′X′)ₗ-N′ₐ-nₚ′3′ (II)
wherein:
k and l are each independently 0 or 1;
p′ and q′ are each independently 0-6;
each N_{a′} independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide;
wherein N_{b}′ and Y′ do not have the same modification; and
X′X′X′, Y′Y′Y′ and Z′Z′Z′ each independently represent one motif of three identical modifications on three consecutive nucleotides.

In one embodiment, the Nₐ′ and/or N_{b}′ comprise modifications of alternating pattern.

The Y′Y′Y′ motif occurs at or near the cleavage site of the antisense strand. For example, when the RNAi agent has a duplex region of 17-23nucleotidein length, the Y′Y′Y′ motif can occur at positions 9, 10, 11; 10, 11, 12; 11, 12, 13; 12, 13, 14; or 13, 14, 15 of the antisense strand, with the count starting from the 1^{st} nucleotide, from the 5′-end; or optionally, the count starting at the 1^{st} paired nucleotide within the duplex region, from the 5′-end. Preferably, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and 1 are 1.

The antisense strand can therefore be represented by the following formulas:

5′n_{q′}-Nₐ′—Z′Z′Z′—N_{b}′—Y′Y′Y′—Nₐ′-n_{p′}3′ (IIb);

5′n_{q′}-Nₐ′—Y′Y′Y′—N_{b}′—X′X′X′-n_{p′}3′ (IIc); or

5′n_{q′}-Nₐ′—Z′Z′Z′—N_{b}′—Y′Y′Y′—N_{b}′—X′X′X′—Nₐ′-n_{p′}3′ (IId).

When the antisense strand is represented by formula (IIb), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and 1 is 0 and the antisense strand may be represented by the formula:

5′nₚ′-Nₐ′—Y′Y′Y′—Nₐ′-n_{q}′3 (Ia).

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1^{st} nucleotide from the 5′-end, or optionally, the count starting at the 1^{st} paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the RNAi agents for use in the methods of the invention may comprise a sense strand and an antisense strand, each strand having 14 to 30 nucleotides, the RNAi duplex represented by formula (III):

sense: 5′nₚ-Nₐ—(XXX)ᵢ—N_{b}—YYY—N_{b}—(ZZZ)ⱼ—Nₐ-n_{q}3′

antisense: 3′nₚ′-Nₐ′—(X′X′X′)ₖ—N_{b}′—Y′Y′Y′—N_{b}′—(Z′Z′Z′)ₗ—Nₐ′-n_{q}′5′ (III)
wherein:
i, j, k, and 1 are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein each nₚ′, nₚ, n_{q}′, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and 1 are 0; or both k and 1 are 1.

Exemplary combinations of the sense strand and antisense strand forming a RNAi duplex include the formulas below:

5′nₚ-Nₐ—YYY—Nₐ-n_{q}3′

3′nₚ′—Nₐ′—Y′Y′Y′—Nₐ′n_{q}′5′ (IIIa)

5′nₚ-Nₐ—YYY—N_{b}—ZZZ—Nₐ-n_{q}3′

3′nₚ′-Nₐ′—Y′Y′Y′—N_{b}′—Z′Z′Z′—Nₐ′n_{q}′₅′ (IIIb)

5′nₚ-Nₐ—XXX—N_{b}—YYY—Nₐ-n_{q}3′

3′nₚ′-Nₐ′—X′X′X′-N_{b}′—Y′Y′Y′—Nₐ′-n_{q}′5′ (IIIc)

5′nₚ-Nₐ—XXX—N_{b}—YYY—N_{b}—ZZZ—Nₐ-n_{q}3′

3′nₚ′—Nₐ′—X′X′X′—N_{b}′—Y′Y′Y′—N_{b}′—Z′Z′Z′—Nₐ-n_{q}′5′ (IIId)

When the RNAi agent is represented by formula (IIIa), each Nₐ independently represents an oligonucleotide sequence comprising 2-20, 2-15, or 2-10 modified nucleotides.

When the RNAi agent is represented by formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as formula (IIIc), each N_{b}, N_{b}′ independently represents an oligonucleotide sequence comprising 0-10, 0-7, 0-10, 0-7, 0-5, 0-4, 0-2 or 0 modified nucleotides. Each Nₐ independently represents an oligonucleotide sequence comprising 2-20, 2-15, or 2-10 modified nucleotides.

When the RNAi agent is represented as formula (IIId), each N_{b}, N_{b}′ independently represents an oligonucleotide sequence comprising 0-10, 0-7, 0-10, 0-7, 0-5, 0-4, 0-2 or 0modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern.

Each of X, Y and Z in formulas (III), (IIIa), (IIIb), (IIIc), and (IIId) may be the same or different from each other.

When the RNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the RNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the RNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In one embodiment, the modification on the Y nucleotide is different than the modification on the Y′ nucleotide, the modification on the Z nucleotide is different than the modification on the Z′ nucleotide, and/or the modification on the X nucleotide is different than the modification on the X′ nucleotide.

In one embodiment, when the RNAi agent is represented by formula (IIId), the Nₐ modifications are 2′-O-methyl or 2′-fluoro modifications. In another embodiment, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet another embodiment, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more GalNAc derivatives attached through a bivalent or trivalent branched linker (described below). In another embodiment, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, when the RNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric RNAi agents that can be used in the methods of the invention. Such publications include WO2007/091269, U.S. Pat. No. 7,858,769, WO2010/141511, WO2007/117686, WO2009/014887 and WO2011/031520 the entire contents of each of which are hereby incorporated herein by reference.

As described in more detail below, the RNAi agent that contains conjugations of one or more carbohydrate moieties to a RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In certain specific embodiments, the RNAi agent for use in the methods of the invention is an agent selected from the group of agents listed in any one of Tables 4A, 4B, 5, 8, 9, 10, 11A, 11B, 12, and 13. These agents may further comprise a ligand.

### IV. iRNAs Conjugated to Ligands

Another modification of the RNA of an iRNA of the invention involves chemically linking to the RNA one or more ligands, moieties or conjugates that enhance the activity, cellular distribution or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment, a ligand alters the distribution, targeting or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucoseamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In one embodiment, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

### B. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 24). An RFGF analogue (e.g., amino acid sequence AALLPVLLAAP (SEQ ID NO: 25) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 26) and the Drosophila Antennapedia protein (RQIKIWFQNRRMKWKK (SEQ ID NO: 27) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, an iRNA oligonucleotide further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include AGT and above (e.g., AGT, C6, C7, or C8) sugars; di- and trisaccharides include sugars having two or three monosaccharide units (e.g., AGT, C6, C7, or C8).

In one embodiment, a carbohydrate conjugate for use in the compositions and methods of the invention is a monosaccharide. In one embodiment, the monosaccharide is an N-acetylgalactosamine, such as

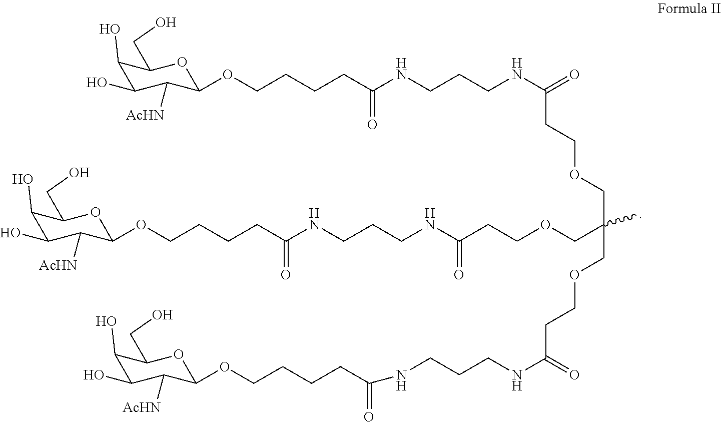 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In another embodiment, a carbohydrate conjugate for use in the compositions and methods of the invention is selected from the group consisting of:

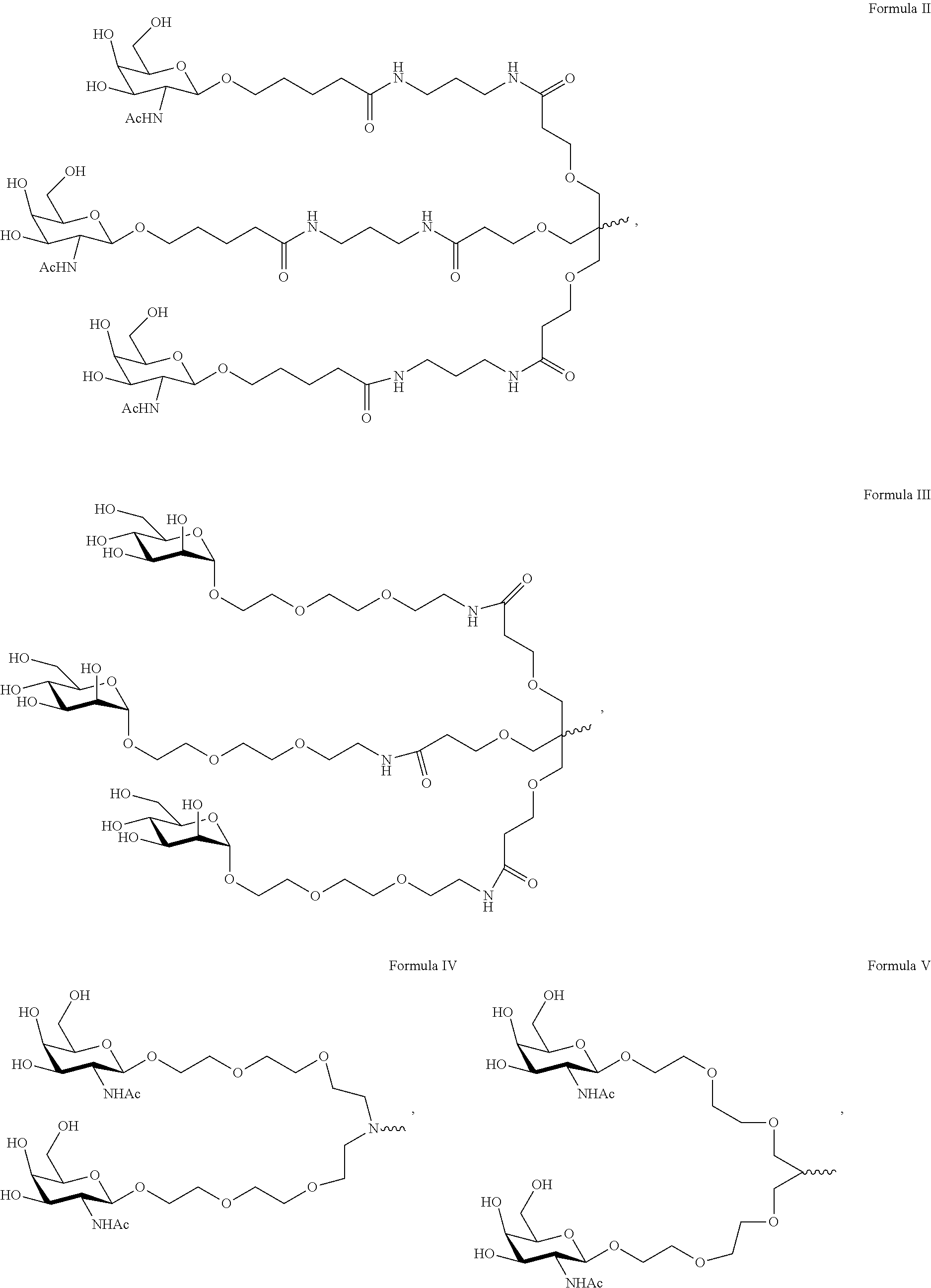

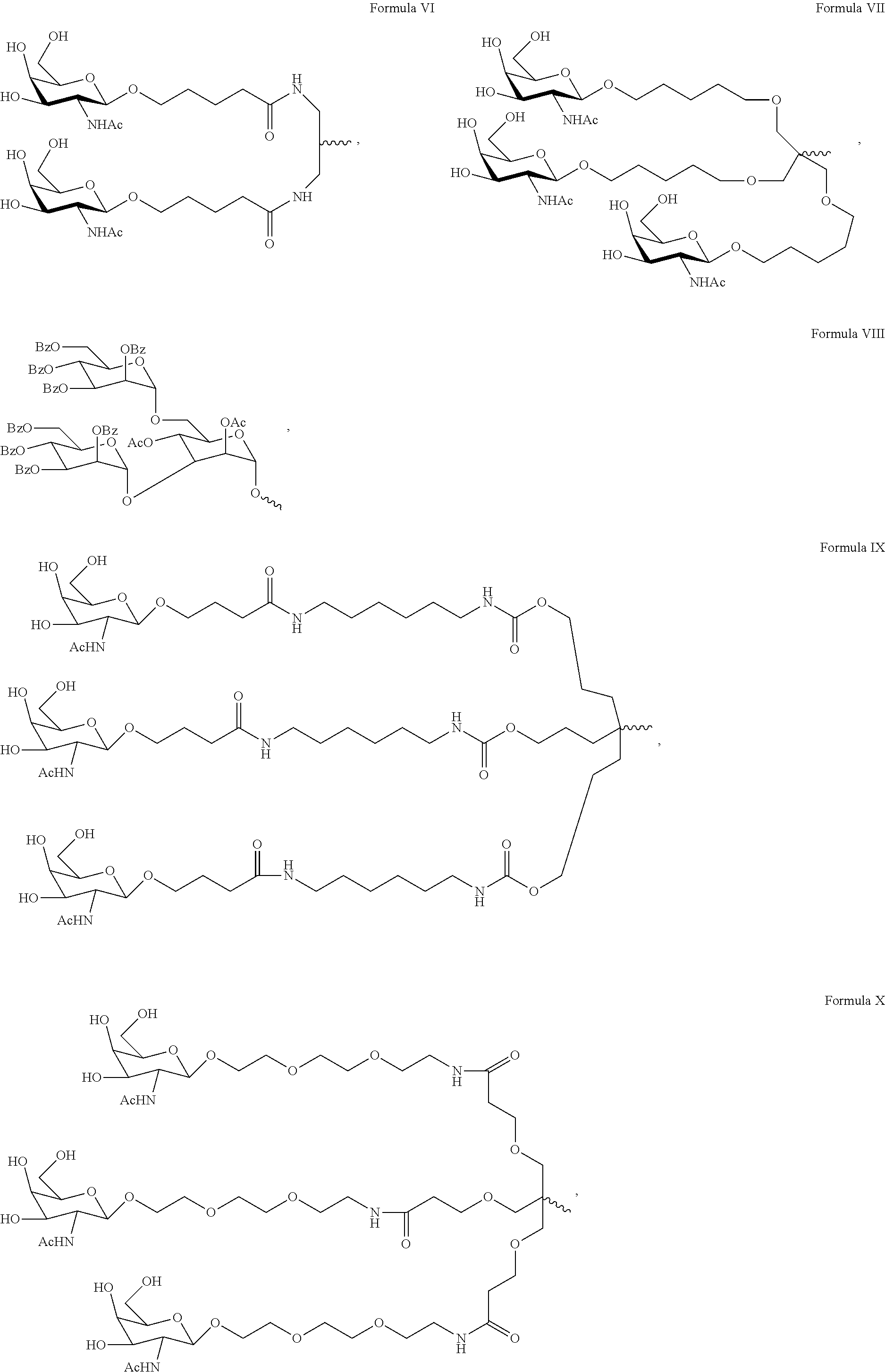

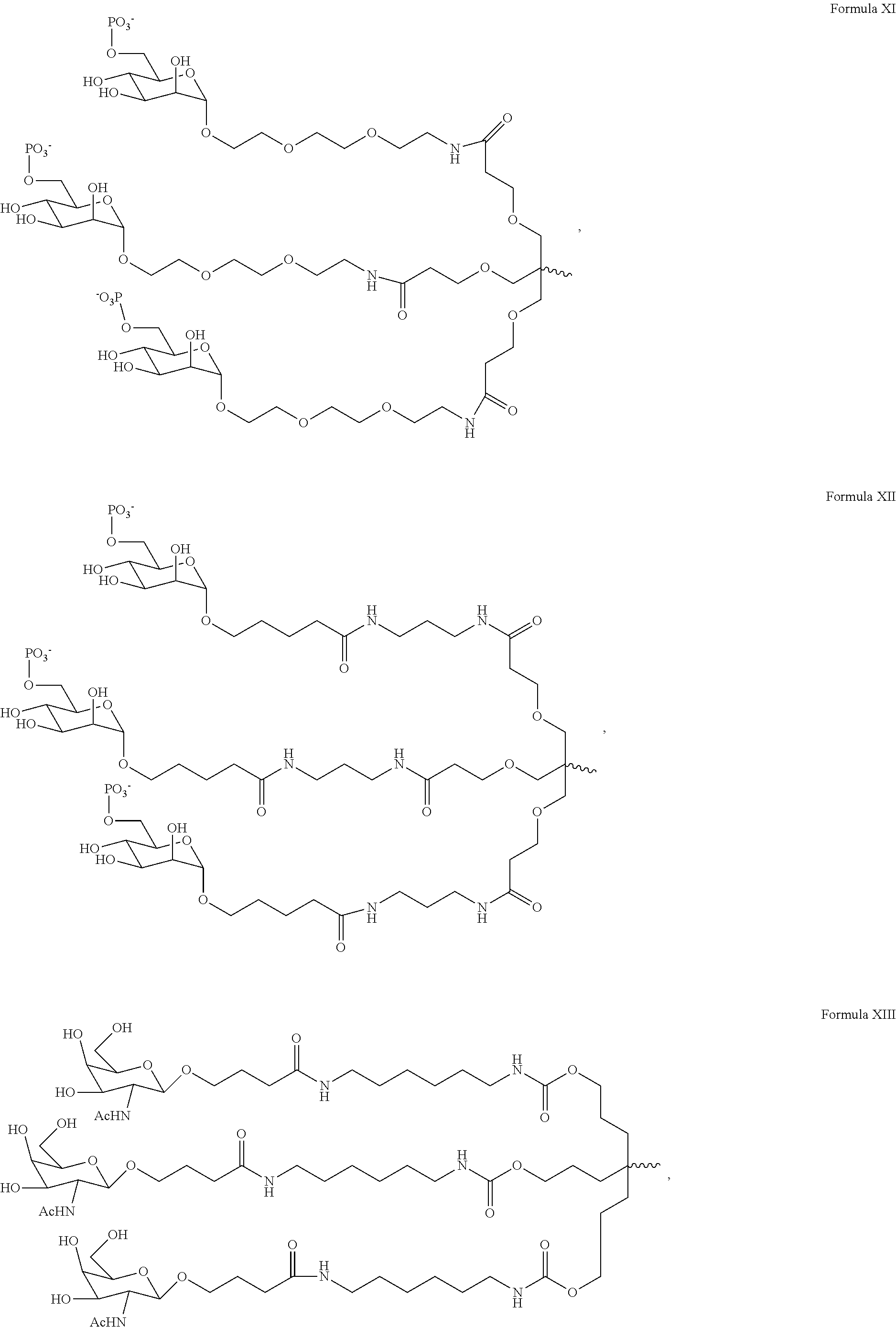

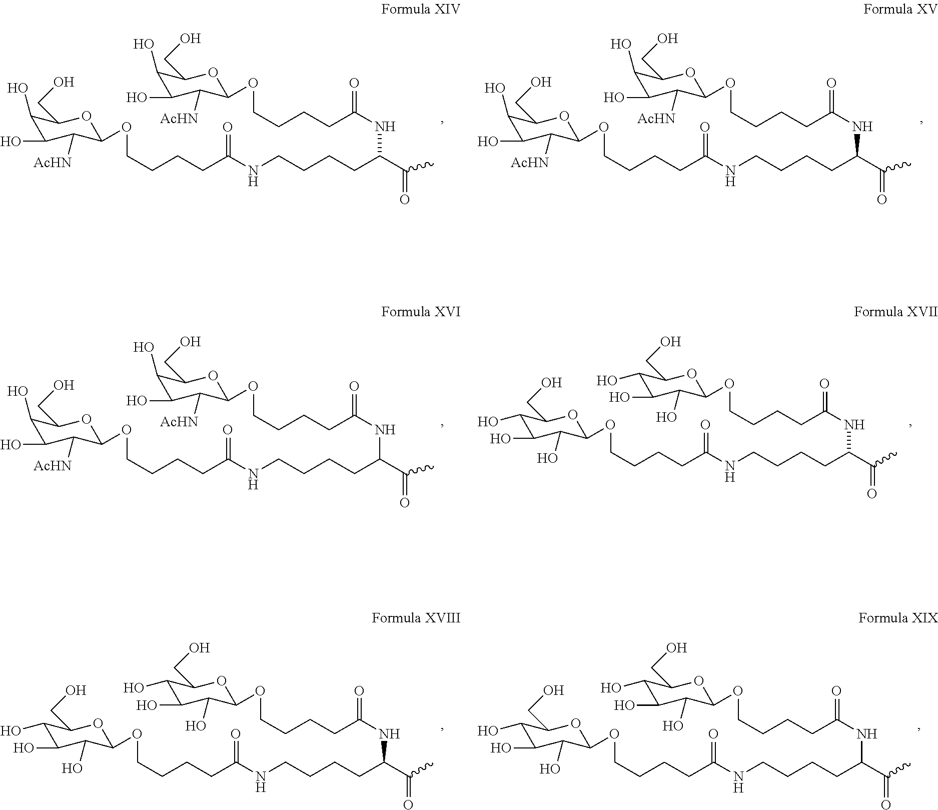

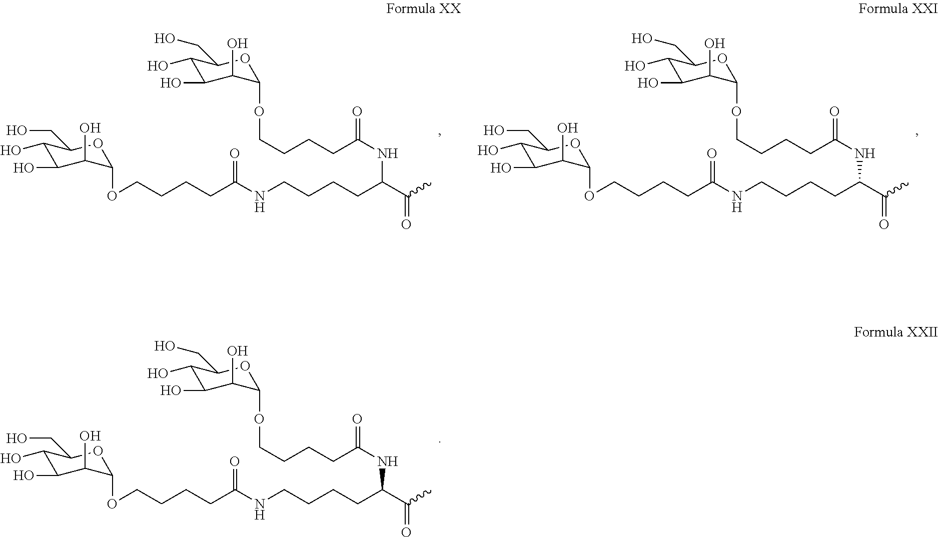

Another representative carbohydrate conjugate for use in the embodiments described herein includes, but is not limited to,

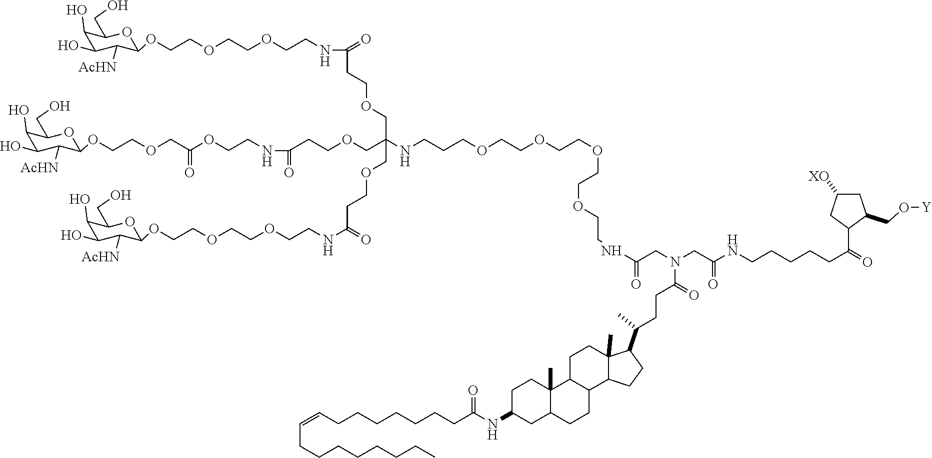

(Formula XXIII), when one of X or Y is an oligonucleotide, the other is a hydrogen.

In some embodiments, the carbohydrate conjugate further comprises one or more additional ligands as described above, such as, but not limited to, a PK modulator and/or a cell permeation peptide.

Additional carbohydrate conjugates suitable for use in the present invention include those described in PCT Publication Nos. WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-16, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

i. Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In another embodiment, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, —O—P(S)(H)—S—. A preferred embodiment is —O—P(O)(OH)—O—. These candidates can be evaluated using methods analogous to those described above.

iii. Acid Cleavable Linking Groups

In another embodiment, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula—NHCHRAC(O)NHCHRBC(O)—, where RA and RB are the R groups of the two adjacent amino acids. These candidates can be evaluated using methods analogous to those described above.

In one embodiment, an iRNA of the invention is conjugated to a carbohydrate through a linker. Non-limiting examples of iRNA carbohydrate conjugates with linkers of the compositions and methods of the invention include, but are not limited to,

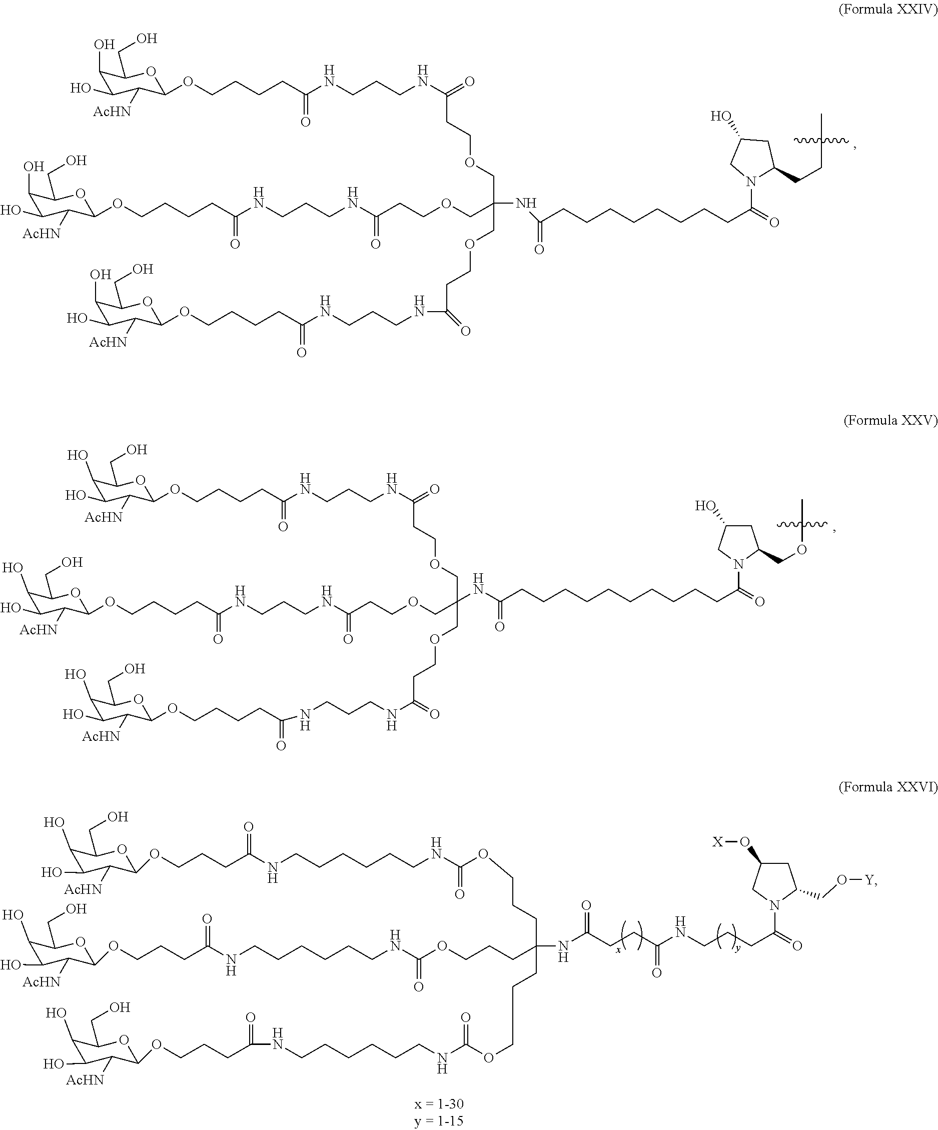

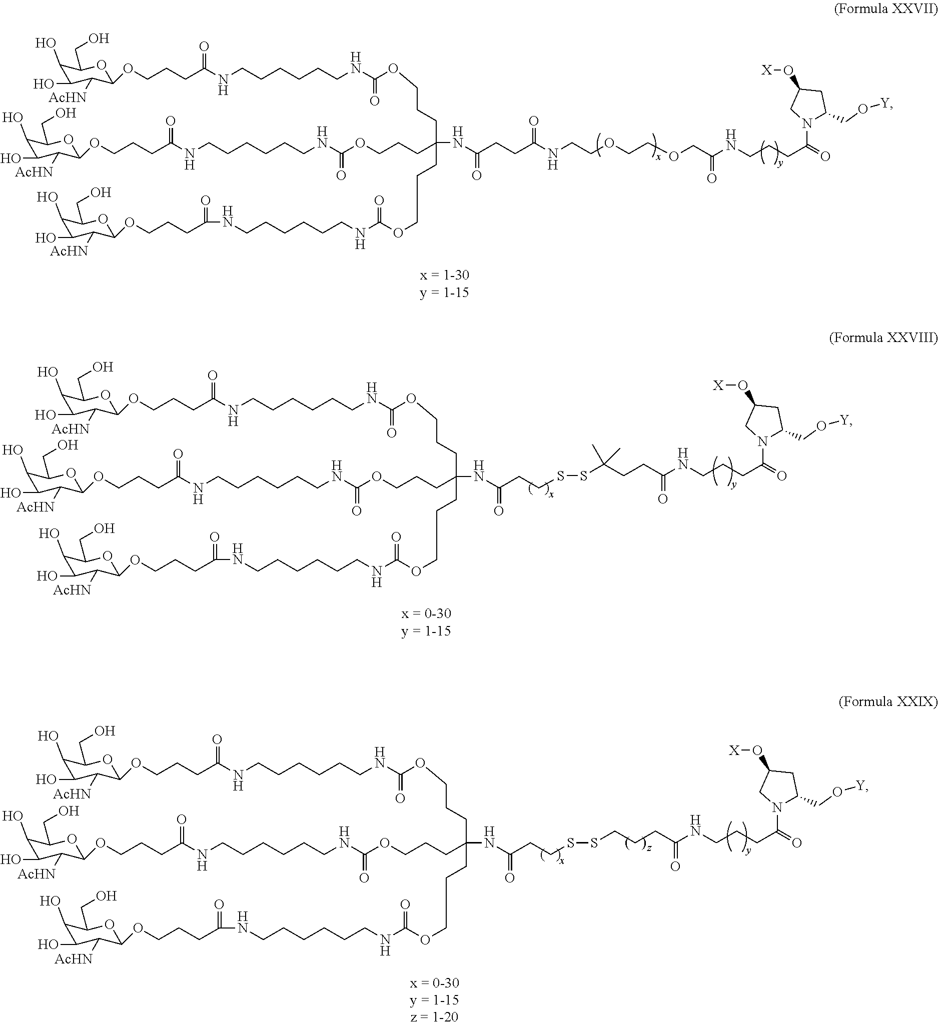

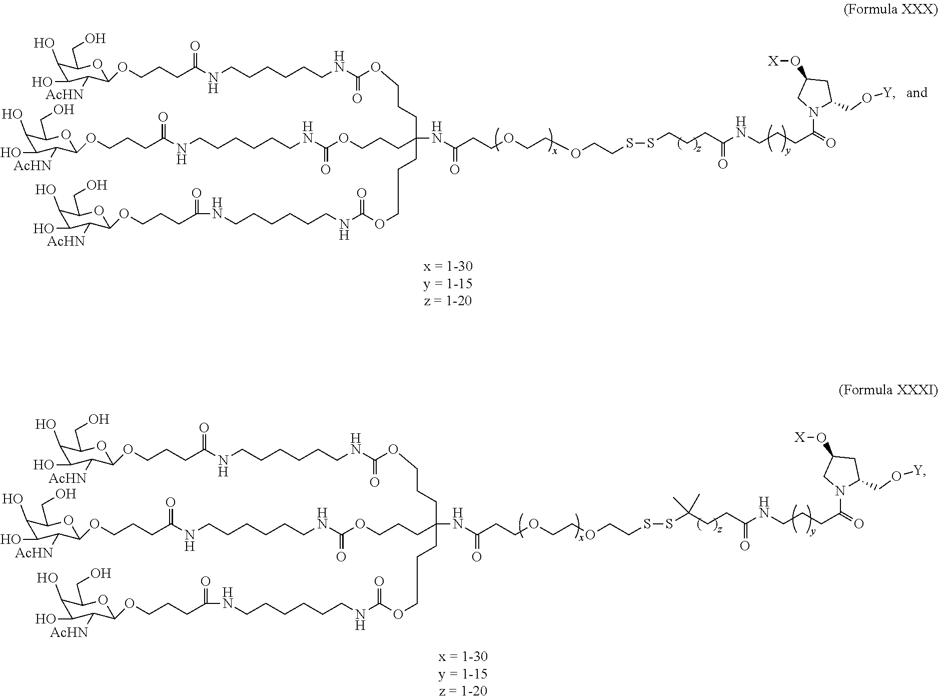

when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In one embodiment, a dsRNA of the invention is conjugated to a bivalent or trivalent branched linker selected from the group of structures shown in any of formula (XXXII)-(XXXV):

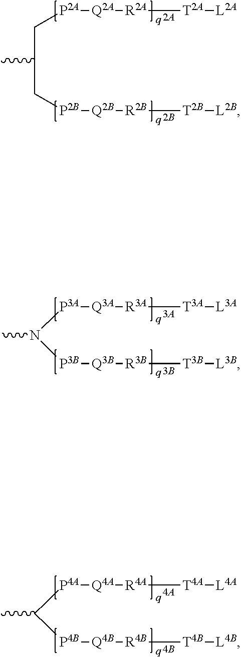

wherein:
q2A, q2B, q3A, q3B, q4A, q4B, q5A, q5B and q5C represent independently for each occurrence 0-20 and wherein the repeating unit can be the same or different; P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O); R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—

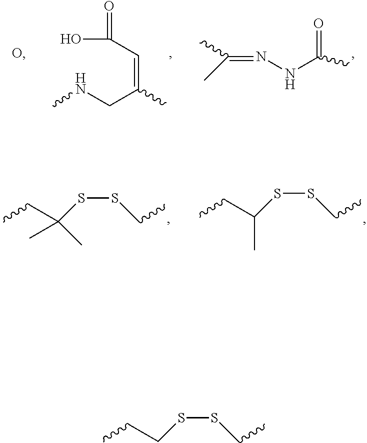

L^{2A}, L^{2B}, L^{3A}, L^{3B}, L^{4A}, L^{4B}, L^{5A}, L^{5B}, and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XXXV):

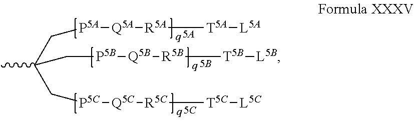

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNAs, which contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, and/or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### V. Delivery of an iRNA of the Invention

The delivery of an iRNA of the invention to a cell e.g., a cell within a subject, such as a human subject (e.g., a subject in need thereof, such as a subject having an APOC3-associated disease) can be achieved in a number of different ways. For example, delivery may be performed by contacting a cell with an iRNA of the invention either in vitro or in vivo. In vivo delivery may also be performed directly by administering a composition comprising an iRNA, e.g., a dsRNA, to a subject. Alternatively, in vivo delivery may be performed indirectly by administering one or more vectors that encode and direct the expression of the iRNA. These alternatives are discussed further below.

In general, any method of delivering a nucleic acid molecule (in vitro or in vivo) can be adapted for use with an iRNA of the invention (see e.g., Akhtar S. and Julian R L. (1992) Trends Cell. Biol. 2(5):139-144 and WO94/02595, which are incorporated herein by reference in their entireties). For in vivo delivery, factors to consider in order to deliver an iRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when an iRNA is administered locally. For example, intraocular delivery of a VEGF dsRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA composition to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O., et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H., et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

A. Vector Encoded iRNAs of the Invention

iRNA targeting the APOC3 gene can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

iRNA expression plasmids can be transfected into target cells as a complex with cationic lipid carriers (e.g., Oligofectamine) or non-cationic lipid-based carriers (e.g., Transit-TKO™). Multiple lipid transfections for iRNA-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are further described below.

Vectors useful for the delivery of an iRNA will include regulatory elements (promoter, enhancer, etc.) sufficient for expression of the iRNA in the desired target cell or tissue. The regulatory elements can be chosen to provide either constitutive or regulated/inducible expression.

Expression of the iRNA can be precisely regulated, for example, by using an inducible regulatory sequence that is sensitive to certain physiological regulators, e.g., circulating glucose levels, or hormones (Docherty et al., 1994, FASEB J. 8:20-24). Such inducible expression systems, suitable for the control of dsRNA expression in cells or in mammals include, for example, regulation by ecdysone, by estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the iRNA transgene.

Viral vectors that contain nucleic acid sequences encoding an iRNA can be used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an iRNA are cloned into one or more vectors, which facilitate delivery of the nucleic acid into a patient. More detail about retroviral vectors can be found, for example, in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, which are herein incorporated by reference.

Adenoviruses are also contemplated for use in delivery of iRNAs of the invention. Adenoviruses are especially attractive vehicles, e.g., for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). A suitable AV vector for expressing an iRNA featured in the invention, a method for constructing the recombinant AV vector, and a method for delivering the vector into target cells, are described in Xia H et al. (2002), Nat. Biotech. 20: 1006-1010.

Adeno-associated virus (AAV) vectors may also be used to delivery an iRNA of the invention (Walsh et al., Proc. Soc. Exp. Biol. Med. 204:289-300 (1993); U.S. Pat. No. 5,436,146). In one embodiment, the iRNA can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosures of which are herein incorporated by reference.

Another viral vector suitable for delivery of an iRNA of the invention is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes; see, e.g., Rabinowitz J E et al. (2002), J Virol 76:791-801, the entire disclosure of which is herein incorporated by reference.

The pharmaceutical preparation of a vector can include the vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### VI. Pharmaceutical Compositions of the Invention

The present invention also includes pharmaceutical compositions and formulations which include the iRNAs of the invention. In one embodiment, provided herein are pharmaceutical compositions containing an iRNA, as described herein, and a pharmaceutically acceptable carrier. The pharmaceutical compositions containing the iRNA are useful for treating a disease or disorder associated with the expression or activity of an APOC3 gene. Such pharmaceutical compositions are formulated based on the mode of delivery. One example is compositions that are formulated for systemic administration via parenteral delivery, e.g., by subcutaneous (SC) or intravenous (IV) delivery. Another example is compositions that are formulated for direct delivery into the brain parenchyma, e.g., by infusion into the brain, such as by continuous pump infusion. The pharmaceutical compositions of the invention may be administered in dosages sufficient to inhibit expression of an APOC3 gene. In general, a suitable dose of an iRNA of the invention will be in the range of about 0.001 to about 200.0 milligrams per kilogram body weight of the recipient per day, generally in the range of about 1 to 50 mg per kilogram body weight per day. For example, the dsRNA can be administered at about 0.01 mg/kg, about 0.05 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 20 mg/kg, about 30 mg/kg, about 40 mg/kg, or about 50 mg/kg per single dose.

For example, the dsRNA may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of this invention.

In another embodiment, the dsRNA is administered at a dose of about 0.1 to about 50 mg/kg, about 0.25 to about 50 mg/kg, about 0.5 to about 50 mg/kg, about 0.75 to about 50 mg/kg, about 1 to about 50 mg/kg, about 1.5 to about 50 mg/kg, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/kg, about 1.5 to about 45 mg/kg, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/kg, about 1.5 to about 40 mg/kg, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/kg, about 1.5 to about 30 mg/kg, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/kg, about 1.5 to about 20 mg/kg, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also intended to be part of this invention.

For example, the dsRNA may be administered at a dose of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of this invention.

In another embodiment, the dsRNA is administered at a dose of about 0.5 to about 50 mg/kg, about 0.75 to about 50 mg/kg, about 1 to about 50 mg/kg, about 1.5 to about 50 mg/kg, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.5 to about 45 mg/kg, about 0.75 to about 45 mg/kg, about 1 to about 45 mg/kg, about 1.5 to about 45 mg/kg, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.5 to about 40 mg/kg, about 0.75 to about 40 mg/kg, about 1 to about 40 mg/kg, about 1.5 to about 40 mg/kg, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.5 to about 30 mg/kg, about 0.75 to about 30 mg/kg, about 1 to about 30 mg/kg, about 1.5 to about 30 mg/kg, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.5 to about 20 mg/kg, about 0.75 to about 20 mg/kg, about 1 to about 20 mg/kg, about 1.5 to about 20 mg/kg, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. In one embodiment, the dsRNA is administered at a dose of about 10 mg/kg to about 30 mg/kg. Values and ranges intermediate to the recited values are also intended to be part of this invention.

For example, subjects can be administered, e.g., subcutaneously or intravenously, a single therapeutic amount of iRNA, such as about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, or about 50 mg/kg. Values and ranges intermediate to the recited values are also intended to be part of this invention.

In some embodiments, subjects are administered, e.g., subcutaneously or intravenously, multiple doses of a therapeutic amount of iRNA, such as a dose about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, or about 50 mg/kg. A multi-dose regimine may include administration of a therapeutic amount of iRNA daily, such as for two days, three days, four days, five days, six days, seven days, or longer.

In other embodiments, subjects are administered, e.g., subcutaneously or intravenously, a repeat dose of a therapeutic amount of iRNA, such as a dose about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, or about 50 mg/kg. A repeat-dose regimine may include administration of a therapeutic amount of iRNA on a regular basis, such as every other day, every third day, every fourth day, twice a week, once a week, every other week, or once a month.

In certain embodiments, for example, when a composition of the invention comprises a dsRNA as described herein and a lipid, subjects can be administered a therapeutic amount of iRNA, such as about 0.01 mg/kg to about 5 mg/kg, about 0.01 mg/kg to about 10 mg/kg, about 0.05 mg/kg to about 5 mg/kg, about 0.05 mg/kg to about 10 mg/kg, about 0.1 mg/kg to about 5 mg/kg, about 0.1 mg/kg to about 10 mg/kg, about 0.2 mg/kg to about 5 mg/kg, about 0.2 mg/kg to about 10 mg/kg, about 0.3 mg/kg to about 5 mg/kg, about 0.3 mg/kg to about 10 mg/kg, about 0.4 mg/kg to about 5 mg/kg, about 0.4 mg/kg to about 10 mg/kg, about 0.5 mg/kg to about 5 mg/kg, about 0.5 mg/kg to about 10 mg/kg, about 1 mg/kg to about 5 mg/kg, about 1 mg/kg to about 10 mg/kg, about 1.5 mg/kg to about 5 mg/kg, about 1.5 mg/kg to about 10 mg/kg, about 2 mg/kg to about about 2.5 mg/kg, about 2 mg/kg to about 10 mg/kg, about 3 mg/kg to about 5 mg/kg, about 3 mg/kg to about 10 mg/kg, about 3.5 mg/kg to about 5 mg/kg, about 4 mg/kg to about 5 mg/kg, about 4.5 mg/kg to about 5 mg/kg, about 4 mg/kg to about 10 mg/kg, about 4.5 mg/kg to about 10 mg/kg, about 5 mg/kg to about 10 mg/kg, about 5.5 mg/kg to about 10 mg/kg, about 6 mg/kg to about 10 mg/kg, about 6.5 mg/kg to about 10 mg/kg, about 7 mg/kg to about 10 mg/kg, about 7.5 mg/kg to about 10 mg/kg, about 8 mg/kg to about 10 mg/kg, about 8.5 mg/kg to about 10 mg/kg, about 9 mg/kg to about 10 mg/kg, or about 9.5 mg/kg to about 10 mg/kg. Values and ranges intermediate to the recited values are also intended to be part of this invention.

For example, the dsRNA may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of this invention.

In certain embodiments of the invention, for example, when a double-stranded RNAi agent includes a modification (e.g., one or more motifs of three identical modifications on three consecutive nucleotides), including one such motif at or near the cleavage site of the agent, six phosphorothioate linkages, and a ligand, such an agent is administered at a dose of about 0.01 to about 0.5 mg/kg, about 0.01 to about 0.4 mg/kg, about 0.01 to about 0.3 mg/kg, about 0.01 to about 0.2 mg/kg, about 0.01 to about 0.1 mg/kg, about 0.01 mg/kg to about 0.09 mg/kg, about 0.01 mg/kg to about 0.08 mg/kg, about 0.01 mg/kg to about 0.07 mg/kg, about 0.01 mg/kg to about 0.06 mg/kg, about 0.01 mg/kg to about 0.05 mg/kg, about 0.02 to about 0.5 mg/kg, about 0.02 to about 0.4 mg/kg, about 0.02 to about 0.3 mg/kg, about 0.02 to about 0.2 mg/kg, about 0.02 to about 0.1 mg/kg, about 0.02 mg/kg to about 0.09 mg/kg, about 0.02 mg/kg to about 0.08 mg/kg, about 0.02 mg/kg to about 0.07 mg/kg, about 0.02 mg/kg to about 0.06 mg/kg, about 0.02 mg/kg to about 0.05 mg/kg, about 0.03 to about 0.5 mg/kg, about 0.03 to about 0.4 mg/kg, about 0.03 to about 0.3 mg/kg, about 0.03 to about 0.2 mg/kg, about 0.03 to about 0.1 mg/kg, about 0.03 mg/kg to about 0.09 mg/kg, about 0.03 mg/kg to about 0.08 mg/kg, about 0.03 mg/kg to about 0.07 mg/kg, about 0.03 mg/kg to about 0.06 mg/kg, about 0.03 mg/kg to about 0.05 mg/kg, about 0.04 to about 0.5 mg/kg, about 0.04 to about 0.4 mg/kg, about 0.04 to about 0.3 mg/kg, about 0.04 to about 0.2 mg/kg, about 0.04 to about 0.1 mg/kg, about 0.04 mg/kg to about 0.09 mg/kg, about 0.04 mg/kg to about 0.08 mg/kg, about 0.04 mg/kg to about 0.07 mg/kg, about 0.04 mg/kg to about 0.06 mg/kg, about 0.05 to about 0.5 mg/kg, about 0.05 to about 0.4 mg/kg, about 0.05 to about 0.3 mg/kg, about 0.05 to about 0.2 mg/kg, about 0.05 to about 0.1 mg/kg, about 0.05 mg/kg to about 0.09 mg/kg, about 0.05 mg/kg to about 0.08 mg/kg, or about 0.05 mg/kg to about 0.07 mg/kg. Values and ranges intermediate to the foregoing recited values are also intended to be part of this invention, e.g., the RNAi agent may be administered to the subject at a dose of about 0.015 mg/kg to about 0.45 mg/kg.

For example, the RNAi agent, e.g., RNAi agent in a pharmaceutical composition, may be administered at a dose of about 0.01 mg/kg, 0.0125 mg/kg, 0.015 mg/kg, 0.0175 mg/kg, 0.02 mg/kg, 0.0225 mg/kg, 0.025 mg/kg, 0.0275 mg/kg, 0.03 mg/kg, 0.0325 mg/kg, 0.035 mg/kg, 0.0375 mg/kg, 0.04 mg/kg, 0.0425 mg/kg, 0.045 mg/kg, 0.0475 mg/kg, 0.05 mg/kg, 0.0525 mg/kg, 0.055 mg/kg, 0.0575 mg/kg, 0.06 mg/kg, 0.0625 mg/kg, 0.065 mg/kg, 0.0675 mg/kg, 0.07 mg/kg, 0.0725 mg/kg, 0.075 mg/kg, 0.0775 mg/kg, 0.08 mg/kg, 0.0825 mg/kg, 0.085 mg/kg, 0.0875 mg/kg, 0.09 mg/kg, 0.0925 mg/kg, 0.095 mg/kg, 0.0975 mg/kg, 0.1 mg/kg, 0.125 mg/kg, 0.15 mg/kg, 0.175 mg/kg, 0.2 mg/kg, 0.225 mg/kg, 0.25 mg/kg, 0.275 mg/kg, 0.3 mg/kg, 0.325 mg/kg, 0.35 mg/kg, 0.375 mg/kg, 0.4 mg/kg, 0.425 mg/kg, 0.45 mg/kg, 0.475 mg/kg, or about 0.5 mg/kg. Values intermediate to the foregoing recited values are also intended to be part of this invention.

The pharmaceutical composition can be administered by intravenous infusion over a period of time, such as over a 5, 6, 7, 8, 9, 10, 11, 12, 13, 14, 15, 16, 17, 18, 19, 20, and 21, 22, 23, 24, or about a 25 minute period. The administration may be repeated, for example, on a regular basis, such as weekly, biweekly (i.e., every two weeks) for one month, two months, three months, four months or longer. After an initial treatment regimen, the treatments can be administered on a less frequent basis. For example, after administration weekly or biweekly for three months, administration can be repeated once per month, for six months or a year or longer.

The pharmaceutical composition can be administered once daily, or the iRNA can be administered as two, three, or more sub-doses at appropriate intervals throughout the day or even using continuous infusion or delivery through a controlled release formulation. In that case, the iRNA contained in each sub-dose must be correspondingly smaller in order to achieve the total daily dosage. The dosage unit can also be compounded for delivery over several days, e.g., using a conventional sustained release formulation which provides sustained release of the iRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments of the invention, a single dose of the pharmaceutical compositions of the invention is administered once per week. In other embodiments of the invention, a single dose of the pharmaceutical compositions of the invention is administered bi-monthly.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNAs encompassed by the invention can be made using conventional methodologies or on the basis of in vivo testing using an appropriate animal model, as described elsewhere herein.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The iRNA can be delivered in a manner to target a particular tissue, such as the liver (e.g., the hepatocytes of the liver).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the iRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). iRNAs featured in the invention can be encapsulated within liposomes or can form complexes thereto, in particular to cationic liposomes. Alternatively, iRNAs can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof). Topical formulations are described in detail in U.S. Pat. No. 6,747,014, which is incorporated herein by reference.

A. iRNA Formulations Comprising Membranous Molecular Assemblies

An iRNA for use in the compositions and methods of the invention can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the iRNA composition. The lipophilic material isolates the aqueous interior from an aqueous exterior, which typically does not include the iRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the iRNA are delivered into the cell where the iRNA can specifically bind to a target RNA and can mediate iRNA. In some cases the liposomes are also specifically targeted, e.g., to direct the iRNA to particular cell types.

A liposome containing an iRNA agent can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The iRNA agent preparation is then added to the micelles that include the lipid component. The cationic groups on the lipid interact with the iRNA agent and condense around the iRNA agent to form a liposome. After condensation, the detergent is removed, e.g., by dialysis, to yield a liposomal preparation of iRNA agent.

If necessary a carrier compound that assists in condensation can be added during the condensation reaction, e.g., by controlled addition. For example, the carrier compound can be a polymer other than a nucleic acid (e.g., spermine or spermidine). pH can also adjusted to favor condensation.

Methods for producing stable polynucleotide delivery vehicles, which incorporate a polynucleotide/cationic lipid complex as structural components of the delivery vehicle, are further described in, e.g., WO 96/37194, the entire contents of which are incorporated herein by reference. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986). Microfluidization can be used when consistently small (50 to 200 nm) and relatively uniform aggregates are desired (Mayhew, et al. Biochim. Biophys. Acta 775:169, 1984). These methods are readily adapted to packaging iRNA agent preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap nucleic acids rather than complex with it. Since both the nucleic acid and the lipid are similarly charged, repulsion rather than complex formation occurs. Nevertheless, some nucleic acid is entrapped within the aqueous interior of these liposomes. pH-sensitive liposomes have been used to deliver nucleic acids encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al. S.T.P. Pharma. Sci., 1994, 4(6) 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M}1, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M}1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver iRNA agents to macrophages.

Further advantages of liposomes include: liposomes obtained from natural phospholipids are biocompatible and biodegradable; liposomes can incorporate a wide range of water and lipid soluble drugs; liposomes can protect encapsulated iRNA agents in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of iRNA agent (see, e.g., Felgner, P. L. et al., Proc. Natl. Acad. Sci., USA 8:7413-7417, 1987 and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer iRNA agent into the skin. In some implementations, liposomes are used for delivering iRNA agent to epidermal cells and also to enhance the penetration of iRNA agent into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with iRNA agent are useful for treating a dermatological disorder.

Liposomes that include iRNA can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include iRNA agent can be delivered, for example, subcutaneously by infection in order to deliver iRNA agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in “Pharmaceutical Dosage Forms”, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms”, Marcel Dekker, Inc., New York, N.Y., 1988, p. 285).

The iRNA for use in the methods of the invention can also be provided as micellar formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C22 alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### B. Lipid Particles

iRNAs, e.g., dsRNAs of in the invention may be fully encapsulated in a lipid formulation, e.g., a LNP, or other nucleic acid-lipid particle.

As used herein, the term “LNP” refers to a stable nucleic acid-lipid particle. LNPs typically contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

The cationic lipid can be, for example, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof. The cationic lipid can comprise from about 20 mol % to about 50 mol % or about 40 mol % of the total lipid present in the particle.

In another embodiment, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

In one embodiment, the lipid-siRNA particle includes 40% 2, 2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (Ci₅). The conjugated lipid that prevents aggregation of particles can be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the lipidoid ND98-4HCl (MW 1487) (see U.S. patent application Ser. No. 12/056,230, filed Mar. 26, 2008, which is incorporated herein by reference), Cholesterol (Sigma-Aldrich), and PEG-Ceramide C16 (Avanti Polar Lipids) can be used to prepare lipid-dsRNA nanoparticles (i.e., LNP01 particles). Stock solutions of each in ethanol can be prepared as follows: ND98, 133 mg/ml; Cholesterol, 25 mg/ml, PEG-Ceramide C16, 100 mg/ml. The ND98, Cholesterol, and PEG-Ceramide C16 stock solutions can then be combined in a, e.g., 42:48:10 molar ratio. The combined lipid solution can be mixed with aqueous dsRNA (e.g., in sodium acetate pH 5) such that the final ethanol concentration is about 35-45% and the final sodium acetate concentration is about 100-300 mM. Lipid-dsRNA nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

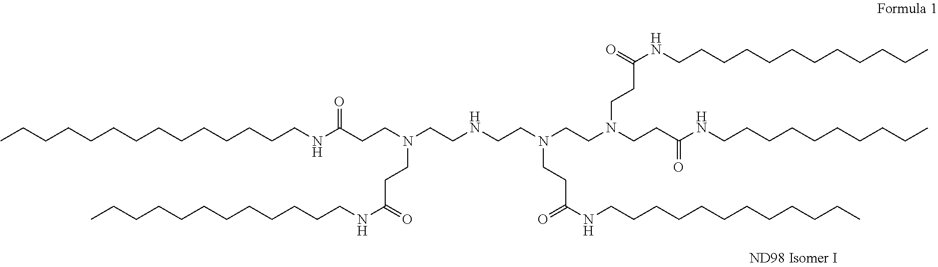 [SMILES: CCCCCCCCCCCCCCNC(=O)CCN(CCNCCN(CCC(=O)NCCCCCCCCCCCC)CCN(CCC(=O)NCCCCCCCCCC)CCC(=O)NCCCCCCCCCCCC)CCC(=O)NCCCCCCCCCCCC]

LNP01 formulations are described, e.g., in International Application Publication No. WO 2008/042973, which is hereby incorporated by reference.

Additional exemplary lipid-dsRNA formulations are described in Table 1.

TABLE 1
Exemplary lipid-dsRNA formulations.

| | | cationic lipid/non-cationic |
|---|---|---|
| | | lipid/cholesterol/PEG-lipid conjugate |
| | Ionizable/Cationic Lipid | Lipid:siRNA ratio |
| SNALP- | 1,2-Dilinolenyloxy-N,N- | DLinDMA/DPPC/Cholesterol/PEG- |
| 1 | dimethylaminopropane (DLinDMA) | cDMA |
| | | (57.1/7.1/34.4/1.4) |
| | | lipid:siRNA~7:1 |
| 2-XTC | 2,2-Dilinoleyl-4-dimethylaminoethyl- | XTC/DPPC/Cholesterol/PEG-cDMA |
| | [1,3]-dioxolane (XTC) | 57.1/7.1/34.4/1.4 |
| | | lipid:siRNA~7:1 |
| LNP05 | 2,2-Dilinoleyl-4-dimethylaminoethyl- | XTC/DSPC/Cholesterol/PEG-DMG |
| | [1,3]-dioxolane (XTC) | 57.5/7.5/31.5/3.5 |
| | | lipid:siRNA~6:1 |
| LNP06 | 2,2-Dilinoleyl-4-dimethylaminoethyl- | XTC/DSPC/Cholesterol/PEG-DMG |
| | [1,3]-dioxolane (XTC) | 57.5/7.5/31.5/3.5 |
| | | lipid:siRNA~11:1 |
| LNP07 | 2,2-Dilinoleyl-4-dimethylaminoethyl- | XTC/DSPC/Cholesterol/PEG-DMG |
| | [1,3]-dioxolane (XTC) | 60/7.5/31/1.5, |
| | | lipid:siRNA~6:1 |
| LNP08 | 2,2-Dilinoleyl-4-dimethylaminoethyl- | XTC/DSPC/Cholesterol/PEG-DMG |
| | [1,3]-dioxolane (XTC) | 60/7.5/31/1.5, |
| | | lipid:siRNA~11:1 |
| LNP09 | 2,2-Dilinoleyl-4-dimethylaminoethyl- | XTC/DSPC/Cholesterol/PEG-DMG |
| | [1,3]-dioxolane (XTC) | 50/10/38.5/1.5 |
| | | Lipid:siRNA 10:1 |
| LNP10 | (3aR,5s,6aS)-N,N-dimethyl-2,2- | ALN100/DSPC/Cholesterol/PEG-DMG |
| | di((9Z,12Z)-octadeca-9,12- | 50/10/38.5/1.5 |
| | dienyl)tetrahydro-3aH- | Lipid:siRNA 10:1 |
| | cyclopenta[d][1,3]dioxol-5-amine | |
| | (ALN100) | |
| LNP11 | (6Z,9Z,28Z,31Z)-heptatriaconta- | MC-3/DSPC/Cholesterol/PEG-DMG |
| | 6,9,28,31-tetraen-19-yl 4- | 50/10/38.5/1.5 |
| | (dimethylamino)butanoate (MC3) | Lipid:siRNA 10:1 |
| LNP12 | 1,1′-(2-(4-(2-((2-(bis(2- | Tech G1/DSPC/Cholesterol/PEG-DMG |
| | hydroxydodecyl)amino)ethyl)(2- | 50/10/38.5/1.5 |
| | hydroxydodecyl)amino)ethyl)piperazin- | Lipid:siRNA 10:1 |
| | 1-yl)ethylazanediyl)didodecan-2-ol | |
| | (Tech G1) | |
| LNP13 | XTC | XTC/DSPC/Chol/PEG-DMG |
| | | 50/10/38.5/1.5 |
| | | Lipid:siRNA: 33:1 |
| LNP14 | MC3 | MC3/DSPC/Chol/PEG-DMG |
| | | 40/15/40/5 |
| | | Lipid:siRNA: 11:1 |
| LNP15 | MC3 | MC3/DSPC/Chol/PEG-DSG/GalNAc- |
| | | PEG-DSG |
| | | 50/10/35/4.5/0.5 |
| | | Lipid:siRNA: 11:1 |
| LNP16 | MC3 | MC3/DSPC/Chol/PEG-DMG |
| | | 50/10/38.5/1.5 |
| | | Lipid:siRNA: 7:1 |
| LNP17 | MC3 | MC3/DSPC/Chol/PEG-DSG |
| | | 50/10/38.5/1.5 |
| | | Lipid:siRNA: 10:1 |
| LNP18 | MC3 | MC3/DSPC/Chol/PEG-DMG |
| | | 50/10/38.5/1.5 |
| | | Lipid:siRNA: 12:1 |
| LNP19 | MC3 | MC3/DSPC/Chol/PEG-DMG |
| | | 50/10/35/5 |
| | | Lipid:siRNA: 8:1 |
| LNP20 | MC3 | MC3/DSPC/Chol/PEG-DPG |
| | | 50/10/38.5/1.5 |
| | | Lipid:siRNA: 10:1 |
| LNP21 | C12-200 | C12-200/DSPC/Chol/PEG-DSG |
| | | 50/10/38.5/1.5 |
| | | Lipid:siRNA: 7:1 |
| LNP22 | XTC | XTC/DSPC/Chol/PEG-DSG |
| | | 50/10/38.5/1.5 |
| | | Lipid:siRNA: 10:1 |

DSPC: distearoylphosphatidylcholine
DPPC: dipalmitoylphosphatidylcholine
PEG-DMG: PEG-didimyristoyl glycerol (C14-PEG, or PEG-C14) (PEG with avg mol wt of 2000)
PEG-DSG: PEG-distyryl glycerol (C18-PEG, or PEG-C18) (PEG with avg mol wt of 2000)
PEG-cDMA: PEG-carbamoyl-1,2-dimyristyloxypropylamine (PEG with avg mol wt of 2000)
SNALP (1,2-Dilinolenyloxy-N,N-dimethylaminopropane (DLinDMA)) comprising formulations are described in International Publication No. WO2009/127060, filed Apr. 15, 2009, which is hereby incorporated by reference.
XTC comprising formulations are described, e.g., in U.S. Provisional Ser. No. 61/148,366, filed Jan. 29, 2009; U.S. Provisional Ser. No. 61/156,851, filed Mar. 2, 2009; U.S. Provisional Ser. No. filed Jun. 10, 2009; U.S. Provisional Ser. No. 61/228,373, filed Jul. 24, 2009; U.S. Provisional Ser. No. 61/239,686, filed Sep. 3, 2009, and International Application No. PCT/US2010/022614, filed Jan. 29, 2010, which are hereby incorporated by reference.
MC3 comprising formulations are described, e.g., in U.S. Publication No. 2010/0324120, filed Jun. 10, 2010, the entire contents of which are hereby incorporated by reference.
ALNY-100 comprising formulations are described, e.g., International patent application number PCT/US09/63933, filed on Nov. 10, 2009, which is hereby incorporated by reference.
C12-200 comprising formulations are described in U.S. Provisional Ser. No. 61/175,770, filed May 5, 2009 and International Application No. PCT/US10/33777, filed May 5, 2010, which are hereby incorporated by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the invention are administered in conjunction with one or more penetration enhancer surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, US Publn. No. 20030027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions can be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

### C. Additional Formulations

i. Emulsions

The compositions of the present invention can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present invention, the compositions of iRNAs and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (P0310), hexaglycerol pentaoleate (P0500), decaglycerol monocaprate (MCA750), decaglycerol monooleate (M0750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or iRNAs. Microemulsions have also been effective in the transdermal delivery of active components in both cosmetic and pharmaceutical applications. It is expected that the microemulsion compositions and formulations of the present invention will facilitate the increased systemic absorption of iRNAs and nucleic acids from the gastrointestinal tract, as well as improve the local cellular uptake of iRNAs and nucleic acids.

Microemulsions of the present invention can also contain additional components and additives such as sorbitan monostearate (Grill 3), Labrasol, and penetration enhancers to improve the properties of the formulation and to enhance the absorption of the iRNAs and nucleic acids of the present invention. Penetration enhancers used in the microemulsions of the present invention can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

An iRNA agent of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p. 92). Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of iRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, NY, 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C1-20 alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al., Enhancement in Drug Delivery, CRC Press, Danvers, MA, 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, NY, 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, NY, 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of iRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, MA, 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of iRNAs through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of iRNAs at the cellular level can also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, CA), Lipofectamine 2000™ (Invitrogen; Carlsbad, CA), 293fectin™ (Invitrogen; Carlsbad, CA), Cellfectin™ (Invitrogen; Carlsbad, CA), DMRIE-C™ (Invitrogen; Carlsbad, CA), FreeStyle™ MAX (Invitrogen; Carlsbad, CA), Lipofectamine™ 2000 CD (Invitrogen; Carlsbad, CA), Lipofectamine™ (Invitrogen; Carlsbad, CA), iRNAMAX (Invitrogen; Carlsbad, CA), Oligofectamine™ (Invitrogen; Carlsbad, CA), Optifect™ (Invitrogen; Carlsbad, CA), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, WI), TransFast™ Transfection Reagent (Promega; Madison, WI), Tfx™-20 Reagent (Promega; Madison, WI), Tfx™_50 Reagent (Promega; Madison, WI), DreamFect™ (OZ Biosciences; Marseille, France), EcoTransfect (OZ Biosciences; Marseille, France), TransPass^{a} D1 Transfection Reagent (New England Biolabs; Ipswich, MA, USA), LyoVec™/LipoGen™ (Invitrogen; San Diego, CA, USA), PerFectin Transfection Reagent (Genlantis; San Diego, CA, USA), NeuroPORTER Transfection Reagent (Genlantis; San Diego, CA, USA), GenePORTER Transfection reagent (Genlantis; San Diego, CA, USA), GenePORTER 2 Transfection reagent (Genlantis; San Diego, CA, USA), Cytofectin Transfection Reagent (Genlantis; San Diego, CA, USA), BaculoPORTER Transfection Reagent (Genlantis; San Diego, CA, USA), TroganPORTER™ transfection Reagent (Genlantis; San Diego, CA, USA), RiboFect (Bioline; Taunton, MA, USA), PlasFect (Bioline; Taunton, MA, USA), UniFECTOR (B-Bridge International; Mountain View, CA, USA), SureFECTOR (B-Bridge International; Mountain View, CA, USA), or HiFect™ (B-Bridge International, Mountain View, CA, USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

v. Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

vi. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

vii. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions can contain substances which increase the viscosity of the suspension including, for example, sodium carboxymethylcellulose, sorbitol and/or dextran. The suspension can also contain stabilizers.

In some embodiments, pharmaceutical compositions featured in the invention include (a) one or more iRNA compounds and (b) one or more agents which function by a non-iRNA mechanism and which are useful in treating an APOC3-associated disorder. Examples of such agents include, but are not limited to an anti-inflammatory agent, anti-steatosis agent, anti-viral, and/or anti-fibrosis agent. In addition, other substances commonly used to protect the liver, such as silymarin, can also be used in conjunction with the iRNAs described herein. Other agents useful for treating liver diseases include telbivudine, entecavir, and protease inhibitors such as telaprevir and other disclosed, for example, in Tung et al., U.S. Application Publication Nos. 2005/0148548, 2004/0167116, and 2003/0144217; and in Hale et al., U.S. Application Publication No. 2004/0127488.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured herein in the invention lies generally within a range of circulating concentrations that include the ED50 with little or no toxicity. The dosage can vary within this range depending upon the dosage form employed and the route of administration utilized. For any compound used in the methods featured in the invention, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the iRNAs featured in the invention can be administered in combination with other known agents effective in treatment of pathological processes mediated by APOC3 expression. In any event, the administering physician can adjust the amount and timing of iRNA administration on the basis of results observed using standard measures of efficacy known in the art or described herein.

### VII. Methods of the Invention

The present invention provides therapeutic and prophylactic methods which include administering to a subject having, or prone to developing, an APOC3-associated disease, disorder, and/or condition (e.g., hypertriglyceridemia), pharmaceutical compositions comprising an iRNA agent, or vector comprising an iRNA of the invention.

In one aspect, the present invention provides methods of treating a subject having a disorder that would benefit from reduction in APOC3 expression, e.g., hypertriglyceridemia and other APOC-3 associated diseases, e.g., non-alcoholic fatty liver disease, non-alcoholic steatohepatitis, polycystic ovary syndrome, kidney disease, obesity, type 2 diabetes mellitus (insulin resistance); hypertension; cardiovascular disorders, e.g., artherosclerosis; and pancreatitis, e.g., acute pancreatitis

The treatment methods (and uses) of the invention include administering to the subject, e.g., a human, a therapeutically effective amount of an iRNA agent targeting an APOC3 gene or a pharmaceutical composition comprising an iRNA agent targeting an APOC3 gene, thereby treating the subject having a disorder that would benefit from reduction in APOC3 expression.

In one aspect, the invention provides methods of preventing at least one symptom in a subject having a disorder that would benefit from reduction in APOC3 expression, e.g., a APOC3-associated disease, such as hypertriglyceridemia and other diseases that may be caused by, be associated with, or be a consequence of hypertriglyceridemia. The latter diseases include, but are not limited to, non-alcoholic fatty liver disease, non-alcoholic steatohepatitis, polycystic ovary syndrome, kidney disease, obesity, type 2 diabetes mellitus (insulin resistance), artherosclerosis, cardiovascular disease or pancreatitis. The methods include administering to the subject a therapeutically effective amount of the iRNA agent, e.g., dsRNA, or vector of the invention, thereby preventing at least one symptom in the subject having a disorder that would benefit from reduction in APOC3 expression.

In another aspect, the present invention provides uses of a therapeutically effective amount of an iRNA agent of the invention for treating a subject, e.g., a subject that would benefit from a reduction and/or inhibition of APOC3 expression.

In a further aspect, the present invention provides uses of an iRNA agent, e.g., a dsRNA, of the invention targeting an APOC3 gene or pharmaceutical composition comprising an iRNA agent targeting an APOC3 gene in the manufacture of a medicament for treating a subject, e.g., a subject that would benefit from a reduction and/or inhibition of APOC3 expression, such as a subject having a disorder that would benefit from reduction in APOC3 expression, e.g., a APOC3-associated disease, such as hypertriglyceridemia and other diseases that may be caused by, be associated with, or be a consequence of hypertriglyceridemia. The latter diseases may include, but are not limited to, non-alcoholic fatty liver disease, non-alcoholic steatohepatitis, polycystic ovary syndrome, kidney disease, obesity, type 2 diabetes mellitus (insulin resistance), artherosclerosis, cardiovascular disease or pancreatitis.

In another aspect, the invention provides uses of an iRNA, e.g., a dsRNA, of the invention for preventing at least one symptom in a subject suffering from a disorder that would benefit from a reduction and/or inhibition of APOC3 expression, such as a APOC3-associated disease, e.g., hypertriglyceridemia and other diseases that may be caused by, be associated with, or be a consequence of hypertriglyceridemia. The latter diseases may include, but are not limited to, non-alcoholic fatty liver disease, non-alcoholic steatohepatitis, polycystic ovary syndrome, kidney disease, obesity, type 2 diabetes mellitus (insulin resistance), artherosclerosis, cardiovascular disease or pancreatitis.

In a further aspect, the present invention provides uses of an iRNA agent of the invention in the manufacture of a medicament for preventing at least one symptom in a subject suffering from a disorder that would benefit from a reduction and/or inhibition of APOC3 expression, such as an APOC3-associated disease, e.g., hypertriglyceridemia and other diseases that may be caused by, be associated with, or be a consequence of hypertriglyceridemia. The latter diseases may include, but are not limited to, non-alcoholic fatty liver disease, non-alcoholic steatohepatitis, polycystic ovary syndrome, kidney disease, obesity, type 2 diabetes mellitus (insulin resistance), artherosclerosis, cardiovascular disease or pancreatitis.

In one embodiment, an iRNA agent targeting APOC3 is administered to a subject having a APOC3-associated disease such that APOC3 levels, e.g., in a cell, tissue, blood or other tissue or fluid of the subject are reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 62%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more when the dsRNA agent is administered to the subject.

The methods and uses of the invention include administering a composition described herein such that expression of the target APOC3 gene is decreased, such as for about 1, 2, 3, 4 5, 6, 7, 8, 12, 16, 18, 24, 28, 32, 36, 40, 44, 48, 52, 56, 60, 64, 68, 72, 76, or about 80 hours. In one embodiment, expression of the target APOC3 gene is decreased for an extended duration, e.g., at least about two, three, four, five, six, seven days or more, e.g., about one week, two weeks, three weeks, or about four weeks or longer.

Administration of the dsRNA according to the methods and uses of the invention may result in a reduction of the severity, signs, symptoms, and/or markers of such diseases or disorders in a patient with an APOC3-associated disease. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of hypertriglyceridemia may be assessed, for example, by periodic monitoring of blood triglyceride levels. Comparison of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an iRNA targeting APOC3 or pharmaceutical composition thereof, “effective against” an APOC3-associated disease indicates that administration in a clinically appropriate manner results in a beneficial effect for at least a statistically significant fraction of patients, such as improvement of symptoms, a cure, a reduction in disease, extension of life, improvement in quality of life, or other effect generally recognized as positive by medical doctors familiar with treating a APOC3-associated disease and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given iRNA drug or formulation of that drug can also be judged using an experimental animal model for the given disease as known in the art. When using an experimental animal model, efficacy of treatment is evidenced when a statistically significant reduction in a marker or symptom is observed. Suitable animal models of an APOC3-associated disease include any animal models that have, e.g., hypertriglyceridemia. Such animal models include, e.g., transgenic mice expressing the human apolipoprotein C2 (APOC2) gene (such as mice of the strain B6;CBA-Tg(APOC2)2Bres/J or B6.Cg-Tg(APOC2)2Bres/J available from the Jackson Laboratory in Bar Harbor, Maine); transgenic mice expressing the human apopoprotein C3 (APOC3) gene (such as mice of the strain B6;CBA-Tg(APOC3)3707Bres/J, available from the Jackson Laboratory); homozygous fatty liver dystrophy (fid) mice (such as mice of the strain C3H/HeJ-Lpin1^{fld-2J}/J, available from the Jackson Laboratory); mice homozygous for the ENU-induced missense mutation called Sec61a1^{Y344H} (such as mice of the strain C57BL/6J-Sec61a1^{m1Gek}/J, available from the Jackson Laboratory); diet-induced obese mice (such as mice of the strain C57BL/6J fed 60% kcal fat diet, available from the Jackson Laboratory or mice of the strain C57BL/6NTac fed 60% kcal fat diet, available from Taconic); and transgenic C57Bl/6 mice overexpressing the human ApoC3 gene under liver-specific TBG promotor or transgenic C57Bl/6 mice as described herein.

In certain embodiments, an experimental animal model suitable for testing the efficacy of an iRNA or a formulation comprising an iRNA of the invention includes a rabbit. Exemplary rabbit models include, e.g., a Watanabe heritable hyperlipidemic (WHHL) rabbit. The WHHL rabbit is an animal model for hypercholesterolemia due to a deficiency of low-density lipoprotein (LDL) receptors. The characteristics of the WHHL rabbit and the history of studies conducted using the WHHL rabbits are described, e.g., by Shiomi, M. and Ito, T., Artherosclerosis (2009), 207(1):1-7, the entire contents of which are hereby incorporated herein by reference. WHHL rabbits exhibit increased cholesterol and triglyceride levels as shown below:

| Plasma Lipid (mg/dL) | Normal Rabbits | WHHL Rabbits |
|---|---|---|
| Total cholesterol | 41 ± 10 | 810 ± 110 |
| Triglyceride | 34 ± 19 | 417 ± 117 |

In certain embodiments, a WHHL rabbit may be a preferred animal model for studying inhibition of APOC3 expression because the WHHL rabbit exhibits a more human-like lipid profile than other animal models and may contribute to the understanding of the relationship between ApoC3 knock-down and lowering of triglycerides and may inform dosing for the studies involving non-human primates. The comparison of enzyme and lipoprotein profiles among various animal species is presented in Table 2 below.

TABLE 2
Differences in Enzymes and Lipoprotein Profiles Among Animal Species

| Enzyme or Lipoprotein | Mice and Rats | Human | WHHL Rabbit |
|---|---|---|---|
| Main plasma | HDL or VLDL | LDL | LDL |
| lipoproteins | ApoB-48 & | ApoB-100 | ApoB-100 |
| ApoB on VLDL | apoB-100 | | |
| ApoB Editing enzyme | Ileum and liver | Ileum | ileum |
| CETP | None | Yes | Yes |
| Hepatic lipase | In plasma | Binding to cell | Binding to cell |
| | | membrane | membrane |
| Atherosclerosis | Resistance | Susceptible | Spontaneous |
| | Lipid- | Various lesions | Various lesions |
| | rich/Collagen- | | |
| | poor | | |
| Hypocholesterolemic | No effect or | Effective | Effective |
| effects of statins | elevation | | |

Other exemplary rabbit models that may be suitable for testing the efficacy of an iRNA or a formulation comprising an iRNA of the invention include, e.g., a diet induced obese rabbit. Diet induced obese rabbits have been previously described in the literature by, e.g., Taylor and Fan, Front. Biosci. (1997), 2:298-308; Carroll et al., Am. J. Physiol. (1996), 271:H373-8; Antic et al., Am. J. Hypertens. (2000), 13:556-9; Carroll et al., Acta Physiol. Scand. (2004), 181:183-91; and Rong et al., Arterioscler. Thromb. Vasc. Biol. (1999), 19:2179-88, the entire contents of which are incorporated herein by reference.

Subjects can be administered a therapeutic amount of iRNA, such as about 0.01 mg/kg, 0.02 mg/kg, 0.03 mg/kg, 0.04 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg dsRNA, 2.6 mg/kg dsRNA, 2.7 mg/kg dsRNA, 2.8 mg/kg dsRNA, 2.9 mg/kg dsRNA, 3.0 mg/kg dsRNA, 3.1 mg/kg dsRNA, 3.2 mg/kg dsRNA, 3.3 mg/kg dsRNA, 3.4 mg/kg dsRNA, 3.5 mg/kg dsRNA, 3.6 mg/kg dsRNA, 3.7 mg/kg dsRNA, 3.8 mg/kg dsRNA, 3.9 mg/kg dsRNA, 4.0 mg/kg dsRNA, 4.1 mg/kg dsRNA, 4.2 mg/kg dsRNA, 4.3 mg/kg dsRNA, 4.4 mg/kg dsRNA, 4.5 mg/kg dsRNA, 4.6 mg/kg dsRNA, 4.7 mg/kg dsRNA, 4.8 mg/kg dsRNA, 4.9 mg/kg dsRNA, 5.0 mg/kg dsRNA, 5.1 mg/kg dsRNA, 5.2 mg/kg dsRNA, 5.3 mg/kg dsRNA, 5.4 mg/kg dsRNA, 5.5 mg/kg dsRNA, 5.6 mg/kg dsRNA, 5.7 mg/kg dsRNA, 5.8 mg/kg dsRNA, 5.9 mg/kg dsRNA, 6.0 mg/kg dsRNA, 6.1 mg/kg dsRNA, 6.2 mg/kg dsRNA, 6.3 mg/kg dsRNA, 6.4 mg/kg dsRNA, 6.5 mg/kg dsRNA, 6.6 mg/kg dsRNA, 6.7 mg/kg dsRNA, 6.8 mg/kg dsRNA, 6.9 mg/kg dsRNA, 7.0 mg/kg dsRNA, 7.1 mg/kg dsRNA, 7.2 mg/kg dsRNA, 7.3 mg/kg dsRNA, 7.4 mg/kg dsRNA, 7.5 mg/kg dsRNA, 7.6 mg/kg dsRNA, 7.7 mg/kg dsRNA, 7.8 mg/kg dsRNA, 7.9 mg/kg dsRNA, 8.0 mg/kg dsRNA, 8.1 mg/kg dsRNA, 8.2 mg/kg dsRNA, 8.3 mg/kg dsRNA, 8.4 mg/kg dsRNA, 8.5 mg/kg dsRNA, 8.6 mg/kg dsRNA, 8.7 mg/kg dsRNA, 8.8 mg/kg dsRNA, 8.9 mg/kg dsRNA, 9.0 mg/kg dsRNA, 9.1 mg/kg dsRNA, 9.2 mg/kg dsRNA, 9.3 mg/kg dsRNA, 9.4 mg/kg dsRNA, 9.5 mg/kg dsRNA, 9.6 mg/kg dsRNA, 9.7 mg/kg dsRNA, 9.8 mg/kg dsRNA, 9.9 mg/kg dsRNA, 9.0 mg/kg dsRNA, 10 mg/kg dsRNA, 15 mg/kg dsRNA, 20 mg/kg dsRNA, 25 mg/kg dsRNA, 30 mg/kg dsRNA, 35 mg/kg dsRNA, 40 mg/kg dsRNA, 45 mg/kg dsRNA, or about 50 mg/kg dsRNA. Values and ranges intermediate to the recited values are also intended to be part of this invention.

In certain embodiments, for example, when a composition of the invention comprises a dsRNA as described herein and a lipid, subjects can be administered a therapeutic amount of iRNA, such as about 0.01 mg/kg to about 5 mg/kg, about 0.01 mg/kg to about 10 mg/kg, about 0.05 mg/kg to about 5 mg/kg, about 0.05 mg/kg to about 10 mg/kg, about 0.1 mg/kg to about 5 mg/kg, about 0.1 mg/kg to about 10 mg/kg, about 0.2 mg/kg to about 5 mg/kg, about 0.2 mg/kg to about 10 mg/kg, about 0.3 mg/kg to about 5 mg/kg, about 0.3 mg/kg to about 10 mg/kg, about 0.4 mg/kg to about 5 mg/kg, about 0.4 mg/kg to about 10 mg/kg, about 0.5 mg/kg to about 5 mg/kg, about 0.5 mg/kg to about 10 mg/kg, about 1 mg/kg to about 5 mg/kg, about 1 mg/kg to about 10 mg/kg, about 1.5 mg/kg to about 5 mg/kg, about 1.5 mg/kg to about 10 mg/kg, about 2 mg/kg to about 2.5 mg/kg, about 2 mg/kg to about 10 mg/kg, about 3 mg/kg to about 5 mg/kg, about 3 mg/kg to about 10 mg/kg, about 3.5 mg/kg to about 5 mg/kg, about 4 mg/kg to about 5 mg/kg, about 4.5 mg/kg to about 5 mg/kg, about 4 mg/kg to about 10 mg/kg, about 4.5 mg/kg to about 10 mg/kg, about 5 mg/kg to about 10 mg/kg, about 5.5 mg/kg to about 10 mg/kg, about 6 mg/kg to about 10 mg/kg, about 6.5 mg/kg to about 10 mg/kg, about 7 mg/kg to about 10 mg/kg, about 7.5 mg/kg to about 10 mg/kg, about 8 mg/kg to about 10 mg/kg, about 8.5 mg/kg to about 10 mg/kg, about 9 mg/kg to about 10 mg/kg, or about 9.5 mg/kg to about 10 mg/kg. Values and ranges intermediate to the recited values are also intended to be part of this invention.

For example, the dsRNA may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of this invention.

In other embodiments, for example, when a composition of the invention comprises a dsRNA as described herein and an N-acetylgalactosamine, subjects can be administered a therapeutic amount of iRNA, such as a dose of about 0.1 to about 50 mg/kg, about 0.25 to about 50 mg/kg, about 0.5 to about 50 mg/kg, about 0.75 to about 50 mg/kg, about 1 to about 50 mg/kg, about 1.5 to about 50 mg/kg, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/kg, about 1.5 to about 45 mg/kg, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/kg, about 1.5 to about 40 mg/kg, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/kg, about 1.5 to about 30 mg/kg, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/kg, about 1.5 to about 20 mg/kg, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. In one embodiment, when a composition of the invention comprises a dsRNA as described herein and an N-acetylgalactosamine, subjects can be administered a therapeutic amount of about 10 to about 30 mg/kg of dsRNA. Values and ranges intermediate to the recited values are also intended to be part of this invention.

For example, subjects can be administered a therapeutic amount of iRNA, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, or about 50 mg/kg. Values and ranges intermediate to the recited values are also intended to be part of this invention.

In certain embodiments of the invention, for example, when a double-stranded RNAi agent includes a modification (e.g., one or more motifs of three identical modifications on three consecutive nucleotides), including one such motif at or near the cleavage site of the agent, six phosphorothioate linkages, and a ligand, such an agent is administered at a dose of about 0.01 to about 0.5 mg/kg, about 0.01 to about 0.4 mg/kg, about 0.01 to about 0.3 mg/kg, about 0.01 to about 0.2 mg/kg, about 0.01 to about 0.1 mg/kg, about 0.01 mg/kg to about 0.09 mg/kg, about 0.01 mg/kg to about 0.08 mg/kg, about 0.01 mg/kg to about 0.07 mg/kg, about 0.01 mg/kg to about 0.06 mg/kg, about 0.01 mg/kg to about 0.05 mg/kg, about 0.02 to about 0.5 mg/kg, about 0.02 to about 0.4 mg/kg, about 0.02 to about 0.3 mg/kg, about 0.02 to about 0.2 mg/kg, about 0.02 to about 0.1 mg/kg, about 0.02 mg/kg to about 0.09 mg/kg, about 0.02 mg/kg to about 0.08 mg/kg, about 0.02 mg/kg to about 0.07 mg/kg, about 0.02 mg/kg to about 0.06 mg/kg, about 0.02 mg/kg to about 0.05 mg/kg, about 0.03 to about 0.5 mg/kg, about 0.03 to about 0.4 mg/kg, about 0.03 to about 0.3 mg/kg, about 0.03 to about 0.2 mg/kg, about 0.03 to about 0.1 mg/kg, about 0.03 mg/kg to about 0.09 mg/kg, about 0.03 mg/kg to about 0.08 mg/kg, about 0.03 mg/kg to about 0.07 mg/kg, about 0.03 mg/kg to about 0.06 mg/kg, about 0.03 mg/kg to about 0.05 mg/kg, about 0.04 to about 0.5 mg/kg, about 0.04 to about 0.4 mg/kg, about 0.04 to about 0.3 mg/kg, about 0.04 to about 0.2 mg/kg, about 0.04 to about 0.1 mg/kg, about 0.04 mg/kg to about 0.09 mg/kg, about 0.04 mg/kg to about 0.08 mg/kg, about 0.04 mg/kg to about 0.07 mg/kg, about 0.04 mg/kg to about 0.06 mg/kg, about 0.05 to about 0.5 mg/kg, about 0.05 to about 0.4 mg/kg, about 0.05 to about 0.3 mg/kg, about 0.05 to about 0.2 mg/kg, about 0.05 to about 0.1 mg/kg, about 0.05 mg/kg to about 0.09 mg/kg, about 0.05 mg/kg to about 0.08 mg/kg, or about 0.05 mg/kg to about 0.07 mg/kg. Values and ranges intermediate to the foregoing recited values are also intended to be part of this invention, e.g., the RNAi agent may be administered to the subject at a dose of about 0.015 mg/kg to about 0.45 mg/kg.

For example, the RNAi agent, e.g., RNAi agent in a pharmaceutical composition, may be administered at a dose of about 0.01 mg/kg, 0.0125 mg/kg, 0.015 mg/kg, 0.0175 mg/kg, 0.02 mg/kg, 0.0225 mg/kg, 0.025 mg/kg, 0.0275 mg/kg, 0.03 mg/kg, 0.0325 mg/kg, 0.035 mg/kg, 0.0375 mg/kg, 0.04 mg/kg, 0.0425 mg/kg, 0.045 mg/kg, 0.0475 mg/kg, 0.05 mg/kg, 0.0525 mg/kg, 0.055 mg/kg, 0.0575 mg/kg, 0.06 mg/kg, 0.0625 mg/kg, 0.065 mg/kg, 0.0675 mg/kg, 0.07 mg/kg, 0.0725 mg/kg, 0.075 mg/kg, 0.0775 mg/kg, 0.08 mg/kg, 0.0825 mg/kg, 0.085 mg/kg, 0.0875 mg/kg, 0.09 mg/kg, 0.0925 mg/kg, 0.095 mg/kg, 0.0975 mg/kg, 0.1 mg/kg, 0.125 mg/kg, 0.15 mg/kg, 0.175 mg/kg, 0.2 mg/kg, 0.225 mg/kg, 0.25 mg/kg, 0.275 mg/kg, 0.3 mg/kg, 0.325 mg/kg, 0.35 mg/kg, 0.375 mg/kg, 0.4 mg/kg, 0.425 mg/kg, 0.45 mg/kg, 0.475 mg/kg, or about 0.5 mg/kg. Values intermediate to the foregoing recited values are also intended to be part of this invention.

The iRNA can be administered by intravenous infusion over a period of time, such as over a 5, 6, 7, 8, 9, 10, 11, 12, 13, 14, 15, 16, 17, 18, 19, 20, 21, 22, 23, 24, or about a 25 minute period. The administration may be repeated, for example, on a regular basis, such as weekly, biweekly (i.e., every two weeks) for one month, two months, three months, four months or longer. After an initial treatment regimen, the treatments can be administered on a less frequent basis. For example, after administration weekly or biweekly for three months, administration can be repeated once per month, for six months or a year or longer.

Administration of the iRNA can reduce APOC3 levels, e.g., in a cell, tissue, blood, urine or other compartment of the patient by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

Before administration of a full dose of the iRNA, patients can be administered a smaller dose, such as a 5% infusion, and monitored for adverse effects, such as an allergic reaction. In another example, the patient can be monitored for unwanted immunostimulatory effects, such as increased cytokine (e.g., TNF-alpha or INF-alpha) levels.

Owing to the inhibitory effects on APOC3 expression, a composition according to the invention or a pharmaceutical composition prepared therefrom can enhance the quality of life.

An iRNA of the invention may be administered in “naked” form, where the modified or unmodified iRNA agent is directly suspended in aqueous or suitable buffer solvent, as a “free iRNA.” A free iRNA is administered in the absence of a pharmaceutical composition. The free iRNA may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the iRNA can be adjusted such that it is suitable for administering to a subject.

Alternatively, an iRNA of the invention may be administered as a pharmaceutical composition, such as a dsRNA liposomal formulation.

Subjects that would benefit from a reduction and/or inhibition of APOC3 gene expression are those having a APOC3-associated disease or disorder as described herein.

Treatment of a subject that would benefit from a reduction and/or inhibition of APOC3 gene expression includes therapeutic and prophylactic treatment.

The invention further provides methods and uses of an iRNA agent or a pharmaceutical composition thereof for treating a subject that would benefit from reduction and/or inhibition of APOC3 expression, e.g., a subject having a APOC3-associated disease, in combination with other pharmaceuticals and/or other therapeutic methods, e.g., with known pharmaceuticals and/or known therapeutic methods, such as, for example, those which are currently employed for treating these diseases. For example, in certain embodiments, an iRNA targeting APOC3 is administered in combination with, e.g., an additional agent useful in treating an APOC3-associated disease.

Examples of additional therapeutic agents include those known to treat hypertriglyceridemia and other diseases that are caused by, associated with or are a consequence of hypertriglyceridemia. For example, an iRNA featured in the invention can be administered with, e.g., an HMG-CoA reductase inhibitor (e.g., a statin), a fibrate, a bile acid sequestrant, niacin, an antiplatelet agent, an angiotensin converting enzyme inhibitor, an angiotensin II receptor antagonist (e.g., losartan potassium, such as Merck & Co.'s Cozaar®), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant (e.g., AGI-1067, from Atherogenics), a glycoprotein IIb/IIIa inhibitor, aspirin or an aspirin-like compound, an IBAT inhibitor (e.g., S-8921, from Shionogi), a squalene synthase inhibitor, a monocyte chemoattractant protein (MCP)-I inhibitor, or fish oil. Exemplary HMG-CoA reductase inhibitors include atorvastatin (Pfizer's Lipitor®/Tahor/Sortis/Torvast/Cardyl), pravastatin (Bristol-Myers Squibb's Pravachol, Sankyo's Mevalotin/Sanaprav), simvastatin (Merck's Zocor®/Sinvacor, Boehringer Ingelheim's Denan, Banyu's Lipovas), lovastatin (Merck's Mevacor/Mevinacor, Bexal's Lovastatina, Cepa; Schwarz Pharma's Liposcler), fluvastatin (Novartis' Lescol®/Locol/Lochol, Fujisawa's Cranoc, Solvay's Digaril), cerivastatin (Bayer's Lipobay/GlaxoSmithKline's Baycol), rosuvastatin (AstraZeneca's Crestor®), and pitivastatin (itavastatin/risivastatin) (Nissan Chemical, Kowa Kogyo, Sankyo, and Novartis). Exemplary fibrates include, e.g., bezafibrate (e.g., Roche's Befizal®/Cedur®/Bezalip®, Kissei's Bezatol), clofibrate (e.g., Wyeth's Atromid-S®), fenofibrate (e.g., Fournier's Lipidil/Lipantil, Abbott's Tricor®, Takeda's Lipantil, generics), gemfibrozil (e.g., Pfizer's Lopid/Lipur) and ciprofibrate (Sanofi-Synthelabo's Modalim®). Exemplary bile acid sequestrants include, e.g., cholestyramine (Bristol-Myers Squibb's Questran® and Questran Light™), colestipol (e.g., Pharmacia's Colestid), and colesevelam (Genzyme/Sankyo's WelChol™). Exemplary niacin therapies include, e.g., immediate release formulations, such as Aventis' Nicobid, Upsher-Smith's Niacor, Aventis' Nicolar, and Sanwakagaku's Perycit. Niacin extended release formulations include, e.g., Kos Pharmaceuticals' Niaspan and Upsher-Smith's SIo-Niacin. Exemplary antiplatelet agents include, e.g., aspirin (e.g., Bayer's aspirin), clopidogrel (Sanofi-Synthelabo/Bristol-Myers Squibb's Plavix), and ticlopidine (e.g., Sanofi-Synthelabo's Ticlid and Daiichi's Panaldine). Other aspirin-like compounds useful in combination with a dsRNA targeting APOC3 include, e.g., Asacard (slow-release aspirin, by Pharmacia) and Pamicogrel (Kanebo/Angelini Ricerche/CEPA). Exemplary angiotensin-converting enzyme inhibitors include, e.g., ramipril (e.g., Aventis' Altace) and enalapril (e.g., Merck & Co.'s Vasotec). Exemplary acyl CoA cholesterol acetyltransferase (AC AT) inhibitors include, e.g., avasimibe (Pfizer), eflucimibe (BioMsrieux Pierre Fabre/Eli Lilly), CS-505 (Sankyo and Kyoto), and SMP-797 (Sumito). Exemplary cholesterol absorption inhibitors include, e.g., ezetimibe (Merck/Schering-Plough Pharmaceuticals Zetia®) and Pamaqueside (Pfizer). Exemplary CETP inhibitors include, e.g., Torcetrapib (also called CP-529414, Pfizer), JTT-705 (Japan Tobacco), and CETi-I (Avant Immunotherapeutics). Exemplary microsomal triglyceride transfer protein (MTTP) inhibitors include, e.g., implitapide (Bayer), R-103757 (Janssen), and CP-346086 (Pfizer). Other exemplary cholesterol modulators include, e.g., NO-1886 (Otsuka/TAP Pharmaceutical), CI-1027 (Pfizer), and WAY-135433 (Wyeth-Ayerst).

Exemplary bile acid modulators include, e.g., HBS-107 (Hisamitsu/Banyu), Btg-511 (British Technology Group), BARI-1453 (Aventis), 5-8921 (Shionogi), SD-5613 (Pfizer), and AZD-7806 (AstraZeneca). Exemplary peroxisome proliferation activated receptor (PPAR) agonists include, e.g., tesaglitazar (AZ-242) (AstraZeneca), Netoglitazone (MCC-555) (Mitsubishi/Johnson & Johnson), GW-409544 (Ligand Pharniaceuticals/GlaxoSmithKline), GW-501516 (Ligand Pharmaceuticals/GlaxoSmithKline), LY-929 (Ligand Pharmaceuticals and Eli Lilly), LY-465608 (Ligand Pharmaceuticals and Eli Lilly), LY-518674 (Ligand Pharmaceuticals and Eli Lilly), and MK-767 (Merck and Kyorin). Exemplary gene-based therapies include, e.g., AdGWEGF 121.10 (GenVec), ApoAl (UCB Pharma/Groupe Fournier), EG-004 (Trinam) (Ark Therapeutics), and ATP-binding cassette transporter-A1 (ABCA1) (CV Therapeutics/Incyte, Aventis, Xenon). Exemplary Glycoprotein Ilb/IIIa inhibitors include, e.g., roxifiban (also called DMP754, Bristol-Myers Squibb), Gantofiban (Merck KGaA/Yamanouchi), and Cromafiban (Millennium Pharmaceuticals). Exemplary squalene synthase inhibitors include, e.g., BMS-1884941 (Bristol-Myers Squibb), CP-210172 (Pfizer), CP-295697 (Pfizer), CP-294838 (Pfizer), and TAK-475 (Takeda). An exemplary MCP-I inhibitor is, e.g., RS-504393 (Roche Bioscience). The anti-atherosclerotic agent BO-653 (Chugai Pharmaceuticals), and the nicotinic acid derivative Nyclin (Yamanouchi Pharmacuticals) are also appropriate for administering in combination with a dsRNA featured in the invention. Exemplary combination therapies suitable for administration with a dsRNA targeting APOC3 include, e.g., advicor (Niacin/lovastatin from Kos Pharmaceuticals), amlodipine/atorvastatin (Pfizer), and ezetimibe/simvastatin (e.g., Vytorin® 10/10, 10/20, 10/40, and 10/80 tablets by Merck/Schering-Plough Pharmaceuticals). Agents for treating hypertriglyceridemia, and suitable for administration in combination with a dsRNA targeting APOC3 include, e.g., lovastatin, niacin Altoprev® Extended-Release Tablets (Andrx Labs), lovastatin Caduet® Tablets (Pfizer), amlodipine besylate, atorvastatin calcium Crestor® Tablets (AstraZeneca), rosuvastatin calcium Lescol® Capsules (Novartis), fluvastatin sodium Lescol® (Reliant, Novartis), fluvastatin sodium Lipitor® Tablets (Parke-Davis), atorvastatin calcium Lofibra® Capsules (Gate), Niaspan Extended-Release Tablets (Kos), niacin Pravachol Tablets (Bristol-Myers Squibb), pravastatin sodium TriCor® Tablets (Abbott), fenofibrate Vytorin® 10/10 Tablets (Merck/Schering-Plough Pharmaceuticals), ezetimibe, simvastatin WelChol™ Tablets (Sankyo), colesevelam hydrochloride Zetia® Tablets (Schering), ezetimibe Zetia® Tablets (Merck/Schering-Plough Pharmaceuticals), and ezetimibe Zocor® Tablets (Merck).

In one embodiment, an iRNA agent is administered in combination with an ezetimibe/simvastatin combination (e.g., Vytorin® (Merck/Schering-Plough Pharmaceuticals)). In one embodiment, the iRNA agent is administered to the patient, and then the additional therapeutic agent is administered to the patient (or vice versa). In another embodiment, the iRNA agent and the additional therapeutic agent are administered at the same time.

The iRNA agent and an additional therapeutic agent and/or treatment may be administered at the same time and/or in the same combination, e.g., parenterally, or the additional therapeutic agent can be administered as part of a separate composition or at separate times and/or by another method known in the art or described herein.

The present invention also provides methods of using an iRNA agent of the invention and/or a composition containing an iRNA agent of the invention to reduce and/or inhibit APOC3 expression in a cell. In other aspects, the present invention provides an iRNA of the invention and/or a composition comprising an iRNA of the invention for use in reducing and/or inhibiting APOC3 expression in a cell. In yet other aspects, use of an iRNA of the invention and/or a composition comprising an iRNA of the invention for the manufacture of a medicament for reducing and/or inhibiting APOC3 expression in a cell are provided.

The methods and uses include contacting the cell with an iRNA, e.g., a dsRNA, of the invention and maintaining the cell for a time sufficient to obtain degradation of the mRNA transcript of an APOC3 gene, thereby inhibiting expression of the APOC3 gene in the cell.

Reduction in gene expression can be assessed by any methods known in the art. For example, a reduction in the expression of APOC3 may be determined by determining the mRNA expression level of APOC3 using methods routine to one of ordinary skill in the art, e.g., Northern blotting, qRT-PCR, by determining the protein level of APOC3 using methods routine to one of ordinary skill in the art, such as Western blotting, immunological techniques, flow cytometry methods, ELISA, and/or by determining a biological activity of APOC3.

In the methods and uses of the invention the cell may be contacted in vitro or in vivo, i.e., the cell may be within a subject.

A cell suitable for treatment using the methods of the invention may be any cell that expresses an APOC3 gene. A cell suitable for use in the methods and uses of the invention may be a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell, e.g., a human liver cell.

APOC3 expression may be inhibited in the cell by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

The in vivo methods and uses of the invention may include administering to a subject a composition containing an iRNA, where the iRNA includes a nucleotide sequence that is complementary to at least a part of an RNA transcript of the APOC3 gene of the mammal to be treated. When the organism to be treated is a human, the composition can be administered by any means known in the art including, but not limited to subcutaneous, intravenous, oral, intraperitoneal, or parenteral routes, including intracranial (e.g., intraventricular, intraparenchymal and intrathecal), intramuscular, transdermal, airway (aerosol), nasal, rectal, and topical (including buccal and sublingual) administration. In certain embodiments, the compositions are administered by subcutaneous or intravenous infusion or injection.

In some embodiments, the administration is via a depot injection. A depot injection may release the iRNA in a consistent way over a prolonged time period. Thus, a depot injection may reduce the frequency of dosing needed to obtain a desired effect, e.g., a desired inhibition of APOC3, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the iRNA to the liver.

The mode of administration may be chosen based upon whether local or systemic treatment is desired and based upon the area to be treated. The route and site of administration may be chosen to enhance targeting.

In one aspect, the present invention also provides methods for inhibiting the expression of an APOC3 gene in a mammal, e.g., a human. The present invention also provides a composition comprising an iRNA, e.g., a dsRNA, that targets an APOC3 gene in a cell of a mammal for use in inhibiting expression of the APOC3 gene in the mammal. In another aspect, the present invention provides use of an iRNA, e.g., a dsRNA, that targets an APOC3 gene in a cell of a mammal in the manufacture of a medicament for inhibiting expression of the APOC3 gene in the mammal.

The methods and uses include administering to the mammal, e.g., a human, a composition comprising an iRNA, e.g., a dsRNA, that targets an APOC3 gene in a cell of the mammal and maintaining the mammal for a time sufficient to obtain degradation of the mRNA transcript of the APOC3 gene, thereby inhibiting expression of the APOC3 gene in the mammal.

Reduction in gene expression can be assessed in peripheral blood sample of the iRNA-administered subject by any methods known it the art, e.g. qRT-PCR, described herein. Reduction in protein production can be assessed by any methods known it the art and by methods, e.g., ELISA or Western blotting, described herein. In one embodiment, a puncture liver biopsy sample serves as the tissue material for monitoring the reduction in APOC3 gene and/or protein expression. In another embodiment, a blood sample serves as the tissue material for monitoring the reduction in APOC3 gene and/or protein expression.

In one embodiment, verification of RISC medicated cleavage of target in vivo following administration of iRNA agent is done by performing 5′-RACE or modifications of the protocol as known in the art (Lasham A et al., (2010) Nucleic Acid Res., 38 (3) p-e19) (Zimmermann et al. (2006) Nature 441: 111-4).

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Materials and Methods

The following materials and methods were used in the Examples.

### Source of Reagents

Where the source of a reagent is not specifically given herein, such reagent can be obtained from any supplier of reagents for molecular biology at a quality/purity standard for application in molecular biology.

siRNA Synthesis

APOC3 siRNA sequences were synthesized at 1 μmol scale on Mermade 192 synthesizer (BioAutomation) using the solid support mediated phosphoramidite chemistry. The solid support was controlled pore glass (500 Å) loaded with custom GalNAc ligand or universal solid support (AM biochemical). Ancillary synthesis reagents, 2′-F and 2′-O-Methyl RNA and deoxy phosphoramidites were obtained from Thermo-Fisher (Milwaukee, WI) and Hongene (China). 2′F, 2′-O-Methyl, GNA (glycol nucleic acids), 5′phosphate and abasic modifications were introduced employing the corresponding phosphoramidites. Synthesis of 3′ GalNAc conjugated single strands was performed on a GalNAc modified CPG support. Custom CPG universal solid support was used for the synthesis of antisense single strands. Coupling time for all phosphoramidites (100 mM in acetonitrile) was 5 min employing 5-Ethylthio-1H-tetrazole (ETT) as activator (0.6 M in acetonitrile). Phosphorothioate linkages were generated using a 50 mM solution of 3-((Dimethylamino-methylidene) amino)-3H-1, 2, 4-dithiazole-3-thione (DDTT, obtained from Chemgenes (Wilmington, MA, USA) in anhydrous acetonitrile/pyridine (1:1 v/v). Oxidation time was 3 minutes. All sequences were synthesized with final removal of the DMT group (“DMT off”).

APOC3 sequences for in vitro screening assays were initially synthesized using ‘TOFFEE-6PS’ motif. In TOFFEE-6PS design, sense strands are made of 21 nucleotides in length, with GalNAc ligand at the 3′end, two phosporothioates at the 5′end and a triplet of 2′F nucleotides at positions 9, 10 and 11. The antisense sequences in TOFFEE-6PS design are 23 nucleotides in length; contain a 3 nucleotide triplet of 2′-OMe nucleotides at positions 11, 12 and 13 with two phosphorothioates at 3′ and 5′ ends respectively.

Upon completion of the solid phase synthesis, oligoribonucleotides were cleaved from the solid support and deprotected in sealed 96 deep well plates using 200 μL Aqueous Methylamine reagent at 60° C. for 20 minutes. For sequences containing 2′ ribo residues (2′-OH) that are protected with tert-butyl dimethyl silyl (TBDMS) group, a second step deprotection was performed using TEA.3HF (triethylamine trihydro fluoride) reagent. To the methylamine deprotection solution, 200 μL of dimethyl sulfoxide (DMSO) and 300 μl TEA.3HF reagent was added, and the solution was incubated for additional 20 minutes at 60° C. At the end of cleavage and deprotection step, the synthesis plate was allowed to come to room temperature and was precipitated by addition of 1 mL of acetontile:ethanol mixture (9:1). The plates were cooled at −80° C. for 2 hrs, and the superanatant was decanted carefully with the aid of a multi channel pipette. The oligonucleotide pellet was re-suspended in 20 mM NaOAc buffer and were desalted using a 5 mL HiTrap size exclusion column (GE Healthcare) on an AKTA Purifier System equipped with an A905 autosampler and a Frac 950 fraction collector. Desalted samples were collected in 96 well plates. Samples from each sequence were analyzed by LC-MS to confirm the identity, UV (260 nm) for quantification and a selected set of samples by IEX chromatography to determine purity.

Annealing of APOC3 single strands was performed on a Tecan liquid handling robot. Equimolar mixture of sense and antisense single strands were combined and annealed in 96 well plates. After combining the complementary single strands, the 96 well plate was sealed tightly and heated in an oven at 100° C. for 10 minutes and allowed to come slowly to room temperature over a period 2-3 hours. The concentration of each duplex was normalized to 10 μM in 1×PBS and then submitted for in vitro screening assays.

### Cell Culture and 96-Well Transfections

Hep3B cells (ATCC, Manassas, VA) were grown to near confluence at 37° C. in an atmosphere of 5% CO₂ in RPMI (ATCC) supplemented with 10% FBS, streptomycin, and glutamine (ATCC) before being released from the plate by trypsinization. Transfection was carried out by adding 14.8 μl of Opti-MEM plus 0.2 μl of Lipofectamine RNAiMax per well (Invitrogen, Carlsbad CA. cat #13778-150) to 5 μl of siRNA duplexes per well into a 96-well plate and incubated at room temperature for 15 minutes. 80 μl of complete growth media without antibiotic containing ˜2×10⁴ Hep3B cells were then added to the siRNA mixture. Cells were incubated for either 24 or 120 hours prior to RNA purification. Single dose experiments were performed at 10 nM and 0.1 nM final duplex concentration and response experiments were done over a range of doses from 10 nM to 36 fM final duplex concentration over 8, 6-fold dilutions.

Total RNA Isolation Using DYNABEADS mRNA Isolation Kit (Invitrogen, Part #: 610-12)

Cells were harvested and lysed in 150 μl of Lysis/Binding Buffer then mixed for 5 minute at 850 rpm using an Eppendorf Thermomixer (the mixing speed was the same throughout the process). Ten microliters of magnetic beads and 80 μl Lysis/Binding Buffer mixture were added to a round bottom plate and mixed for 1 minute. Magnetic beads were captured using magnetic stand and the supernatant was removed without disturbing the beads. After removing supernatant, the lysed cells were added to the remaining beads and mixed for 5 minutes. After removing supernatant, magnetic beads were washed 2 times with 150 μl Wash Buffer A and mixed for 1 minute. Beads were capture again and supernatant removed. Beads were then washed with 150 μl Wash Buffer B, captured and supernatant was removed. Beads were next washed with 150 μl Elution Buffer, captured and supernatant removed. Beads were allowed to dry for 2 minutes. After drying, 50 μl of Elution Buffer was added and mixed for 5 minutes at 70° C. Beads were captured on magnet for 5 minutes. 40 μl of supernatant was removed and added to another 96 well plate.

cDNA Synthesis Using ABI High Capacity cDNA Reverse Transcription Kit (Applied Biosystems, Foster City, CA, Cat #4368813)

A master mix of 2 μl 10× Buffer, 0.8 μl 25×dNTPs, 2 μl Random primers, 1 μl Reverse Transcriptase, 1 μl RNase inhibitor and 3.2 μl of H₂O per reaction were added into 10 μl total RNA. cDNA was generated using a Bio-Rad C-1000 or S-1000 thermal cycler (Hercules, CA) through the following steps: 25° C. 10 min, 37° C. 120 min, 85° C. 5 sec, 4° C. hold.

### Real Time PCR

2 μl of cDNA were added to a master mix containing 0.5 μl GAPDH TaqMan Probe (Applied Biosystems Cat #4326317E), 0.5 μl ApoC3 TaqMan probe (Applied Biosystems cat #Hs00163644_ml) and 5 μl Lightcycler 480 probe master mix (Roche Cat #04887301001) per well in a 384 well plates (Roche cat #04887301001). Real time PCR was done in a LightCycler480 Real Time PCR system (Roche) using the ΔΔCt(RQ) assay. Each duplex was tested in two independent transfections and each transfection was assayed in duplicate, unless otherwise noted in the summary tables.

To calculate relative fold change, real time data were analyzed using the ΔΔCt method and normalized to assays performed with cells transfected with 10 nM AD-1955, or mock transfected cells. IC₅₀s were calculated using a 4 parameter fit model using XLFit and normalized to cells transfected with AD-1955 or naïve cells.

The sense and antisense sequences of AD-1955 are:

SENSE:
(SEQ ID NO: 28)
cuuAcGcuGAGuAcuucGAdTsdT
ANTISENSE:
(SEQ ID NO: 29)
UCGAAGuACUcAGCGuAAGdTsdT

TABLE 3
Abbreviations of nucleotide monomers used in nucleic acid sequence
representation. It will be understood that, unless otherwise indicated, these monomers, when
present in an oligonucleotide, are mutually linked by 5′-3′-phosphodiester bonds.

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol Hyp- |
| | (GalNAc-alkyl)3 |
| dT | 2′-deoxythymidine-3′-phosphate |
| VP | Vinyl phosphate |
| Agn | Adenosine-glycol nucleic acid (GNA) |
| Tgn | Thymidine-glycol nucleic acid (GNA) S-Isomer |
| Y44 | 2-hydroxymethyl-tetrahydrofurane-5-phosphate |
| dG | 2′-deoxyguanosine-3′-phosphate |

### Example 1. iRNA Synthesis

Generation of the Initial Set of iRNA Agents Targeting APOC3

A set of iRNAs targeting the human APOC3, “apolipoprotein C-III” (human: NCBI refseqID NM_000040.1), as well as tox-species APOC3 orthologs (cynomolgus monkey: XM_005579730; rhesus monkey, XM_001090312.2; mouse: NM_023114; rat, NM_012501) were designed using custom R and Python scripts. The human APOC3 REFSEQ mRNA has a length of 533 bases. The rationale and method for the set of siRNA designs is as follows: the predicted efficacy for every potential 19mer iRNA from position 47 through position 533 was determined with a linear model derived the direct measure of mRNA knockdown from more than 20,000 distinct iRNA designs targeting a large number of vertebrate genes. Subsets of the APOC3 siRNAs were designed with perfect or near-perfect matches between human, cynomolgus and rhesus monkey. A further subset was designed with perfect or near-perfect matches to mouse and rat APOC3 orthologs. For each strand of the iRNA, a custom Python script was used in a brute force search to measure the number and positions of mismatches between the siRNA and all potential alignments in the target species transcriptome. Extra weight was given to mismatches in the seed region, defined here as positions 2-9 of the antisense oligonucleotide, as well the cleavage site of the iRNA, defined here as positions 10-11 of the antisense oligonucleotide. The relative weight of the mismatches was 2.8; 1.2:1 for seed mismatches, cleavage site, and other positions up through antisense position 19. Mismatches in the first position were ignored. A specificity score was calculated for each strand by summing the value of each weighted mismatch. Preference was given to siRNAs whose antisense score in human and cynomolgus monkey was >=3.0 and predicted efficacy was >=70% knockdown of the APOC3 transcript.

A series of iRNA duplexes with sequences designed as described above were synthesized, and conjugated with a trivalent GalNAc at the 3-end of the sense strand using the techniques described above. The sequences of these duplexes are shown in Tables 4A and 4B. These same sequences were also synthesized with various nucleotide modifications and conjugated with a trivalent GalNAc. The sequences of the modified duplexes are shown in Table 5.

TABLE 4A
APOC3 Unmodified Sequences Based on NM_000040.1.

| | Sense | Sense Sequence | SEQ | Position in | Antisense | Antisense Sequence | SEQ | Position in |
|---|---|---|---|---|---|---|---|---|
| Duplex ID | Strand ID | (5′ to 3′) | ID NO: | NM_000040.1 | Strand ID | (5′ to 3′) | ID NO: | NM_000040.1 |
| AD- | A-117251.1 | ACCAAGACCGCCAAGGAUGCA | 30 | 164-184 | A-117252.1 | UGCAUCCUUGGCGGUCUUGGUGG | 133 | 162-184 |
| 57501.1 | | | | | | | | |
| AD- | A-117323.1 | ACCAAGACCGCCAAGGAUGCA | 31 | 164-184 | A-117324.1 | UGCAUCCUUGGCGGUCUUGGUGG | 134 | 162-184 |
| 57537.1 | | | | | | | | |
| AD- | A-117271.1 | CAAGACCGCCAAGGAUGCACU | 32 | 166-186 | A-117272.1 | AGUGCAUCCUUGGCGGUCUUGGU | 135 | 164-186 |
| 57512.1 | | | | | | | | |
| AD- | A-117343.1 | CAAGACCGCCAAGGAUGCACU | 33 | 166-186 | A-117344.1 | AGUGCAUCCUUGGCGGUCUUGGU | 136 | 164-186 |
| 57548.1 | | | | | | | | |
| AD- | A-117249.1 | CCGAUGGCUUCAGUUCCCUGA | 34 | 237-257 | A-117250.1 | UCAGGGAACUGAAGCCAUCGGUC | 137 | 235-257 |
| 57496.1 | | | | | | | | |
| AD- | A-117321.1 | CCGAUGGCUUCAGUUCCCUGA | 35 | 237-257 | A-117322.1 | UCAGGGAACUGAAGCCAUCGGUC | 138 | 235-257 |
| 57532.1 | | | | | | | | |
| AD- | A-117247.1 | CGAUGGCUUCAGUUCCCUGAA | 36 | 238-258 | A-117248.1 | UUCAGGGAACUGAAGCCAUCGGU | 139 | 236-258 |
| 57491.1 | | | | | | | | |
| AD- | A-117319.1 | CGAUGGCUUCAGUUCCCUGAA | 37 | 238-258 | A-117320.1 | UUCAGGGAACUGAAGCCAUCGGU | 140 | 236-258 |
| 57527.1 | | | | | | | | |
| AD- | A-117327.1 | AGACUACUGGAGCACCGUUAA | 38 | 259-279 | A-117328.1 | UUAACGGUGCUCCAGUAGUCUUU | 141 | 257-279 |
| 57547.1 | | | | | | | | |
| AD- | A-117255.1 | AGACUACUGGAGCACCGUUAA | 39 | 259-279 | A-117256.1 | UUAACGGUGCUCCAGUAGUCUUU | 142 | 257-279 |
| 57511.1 | | | | | | | | |
| AD- | A-117365.1 | CUACUGGAGCACCGUUAAGGA | 40 | 262-282 | A-117366.1 | UCCUUAACGGUGCUCCAGUAGUC | 143 | 260-282 |
| 57561.1 | | | | | | | | |
| AD- | A-117293.1 | CUACUGGAGCACCGUUAAGGA | 41 | 262-282 | A-117294.1 | UCCUUAACGGUGCUCCAGUAGUC | 144 | 260-282 |
| 57525.1 | | | | | | | | |
| AD- | A-117275.1 | ACUGGAGCACCGUUAAGGACA | 42 | 264-284 | A-117276.1 | UGUCCUUAACGGUGCUCCAGUAG | 145 | 262-284 |
| 57520.1 | | | | | | | | |
| AD- | A-117347.1 | ACUGGAGCACCGUUAAGGACA | 43 | 264-284 | A-117348.1 | UGUCCUUAACGGUGCUCCAGUAG | 146 | 262-284 |
| 57556.1 | | | | | | | | |
| AD- | A-117283.1 | CUGGAGCACCGUUAAGGACAA | 44 | 265-285 | A-117284.1 | UUGUCCUUAACGGUGCUCCAGUA | 147 | 263-285 |
| 57503.1 | | | | | | | | |
| AD- | A-117355.1 | CUGGAGCACCGUUAAGGACAA | 45 | 265-285 | A-117356.1 | UUGUCCUUAACGGUGCUCCAGUA | 148 | 263-285 |
| 57539.1 | | | | | | | | |
| AD- | A-117337.1 | GGAGCACCGUUAAGGACAAGU | 46 | 267-287 | A-117338.1 | ACUUGUCCUUAACGGUGCUCCAG | 149 | 265-287 |
| 57533.1 | | | | | | | | |
| AD- | A-117265.1 | GGAGCACCGUUAAGGACAAGU | 47 | 267-287 | A-117266.1 | ACUUGUCCUUAACGGUGCUCCAG | 150 | 265-287 |
| 57497.1 | | | | | | | | |
| AD- | A-117281.1 | GAGCACCGUUAAGGACAAGUU | 48 | 268-288 | A-117282.1 | AACUUGUCCUUAACGGUGCUCCA | 151 | 266-288 |
| 57498.1 | | | | | | | | |
| AD- | A-117353.1 | GAGCACCGUUAAGGACAAGUU | 49 | 268-288 | A-117354.1 | AACUUGUCCUUAACGGUGCUCCA | 152 | 266-288 |
| 57534.1 | | | | | | | | |
| AD- | A-117253.1 | GUGGCUGCCUGAGACCUCAAU | 50 | 335-355 | A-117254.1 | AUUGAGGUCUCAGGCAGCCACGG | 153 | 333-355 |
| 57506.1 | | | | | | | | |
| AD- | A-117325.1 | GUGGCUGCCUGAGACCUCAAU | 51 | 335-355 | A-117326.1 | AUUGAGGUCUCAGGCAGCCACGG | 154 | 333-355 |
| 57542.1 | | | | | | | | |
| AD- | A-117261.1 | GCCUGAGACCUCAAUACCCCA | 52 | 341-361 | A-117262.1 | UGGGGUAUUGAGGUCUCAGGCAG | 155 | 339-361 |
| 57523.1 | | | | | | | | |
| AD- | A-117333.1 | GCCUGAGACCUCAAUACCCCA | 53 | 341-361 | A-117334.1 | UGGGGUAUUGAGGUCUCAGGCAG | 156 | 339-361 |
| 57559.1 | | | | | | | | |
| AD- | A-119685.1 | GGCUGCCUGAGACCUCAAUAC | 54 | 337-357 | A-119686.1 | GUAUUGAGGUCUCAGGCAGCCAC | 157 | 335-357 |
| 58915.1 | | | | | | | | |
| AD- | A-117269.1 | CCUGAGACCUCAAUACCCCAA | 55 | 342-362 | A-117270.1 | UUGGGGUAUUGAGGUCUCAGGCA | 158 | 340-362 |
| 57507.1 | | | | | | | | |
| AD- | A-117341.1 | CCUGAGACCUCAAUACCCCAA | 56 | 342-362 | A-117342.1 | UUGGGGUAUUGAGGUCUCAGGCA | 159 | 340-362 |
| 57543.1 | | | | | | | | |
| AD- | A-119690.1 | GCUGCCUGAGACCUCAAUACC | 57 | 338-358 | A-119691.1 | GGUAUUGAGGUCUCAGGCAGCCA | 160 | 336-358 |
| 58921.1 | | | | | | | | |
| AD- | A-119698.1 | CUGCCUGAGACCUCAAUACCC | 58 | 339-359 | A-119699.1 | GGGUAUUGAGGUCUCAGGCAGCC | 161 | 337-359 |
| 58913.1 | | | | | | | | |
| AD- | A-117267.1 | GACCUCAAUACCCCAAGUCCA | 59 | 347-367 | A-117268.1 | UGGACUUGGGGUAUUGAGGUCUC | 162 | 345-367 |
| 57502.1 | | | | | | | | |
| AD- | A-117339.1 | GACCUCAAUACCCCAAGUCCA | 60 | 347-367 | A-117340.1 | UGGACUUGGGGUAUUGAGGUCUC | 163 | 345-367 |
| 57538.1 | | | | | | | | |
| AD- | A-119692.1 | UGAGACCUCAAUACCCCAAGU | 61 | 344-364 | A-119693.1 | ACUUGGGGUAUUGAGGUCUCAGG | 164 | 342-364 |
| 58923.1 | | | | | | | | |
| AD- | A-119683.1 | AUCUCCAGGGCUGCCCCUGUA | 62 | 405-425 | A-119684.1 | UACAGGGGCAGCCCUGGAGAUUG | 165 | 403-425 |
| 58912.1 | | | | | | | | |
| AD- | A-117273.1 | GCUGCCCCUGUAGGUUGCUUA | 63 | 414-434 | A-117274.1 | UAAGCAACCUACAGGGGCAGCCC | 166 | 412-434 |
| 57516.1 | | | | | | | | |
| AD- | A-117345.1 | GCUGCCCCUGUAGGUUGCUUA | 64 | 414-434 | A-117346.1 | UAAGCAACCUACAGGGGCAGCCC | 167 | 412-434 |
| 57552.1 | | | | | | | | |
| AD- | A-117287.1 | UGCCCCUGUAGGUUGCUUAAA | 65 | 416-436 | A-117288.1 | UUUAAGCAACCUACAGGGGCAGC | 168 | 414-436 |
| 57513.1 | | | | | | | | |
| AD- | A-117359.1 | UGCCCCUGUAGGUUGCUUAAA | 66 | 416-436 | A-117360.1 | UUUAAGCAACCUACAGGGGCAGC | 169 | 414-436 |
| 57549.1 | | | | | | | | |
| AD- | A-117259.1 | GCCCCUGUAGGUUGCUUAAAA | 67 | 417-437 | A-117260.1 | UUUUAAGCAACCUACAGGGGCAG | 170 | 415-437 |
| 57519.1 | | | | | | | | |
| AD- | A-117331.1 | GCCCCUGUAGGUUGCUUAAAA | 68 | 417-437 | A-117332.1 | UUUUAAGCAACCUACAGGGGCAG | 171 | 415-437 |
| 57555.1 | | | | | | | | |
| AD- | A-117367.2 | CUGCCCCUGUAGGUUGCUUAA | 69 | 415-435 | A-119687.1 | UUAAGCAACCUACAGGGGCAGCC | 172 | 413-435 |
| 58917.1 | | | | | | | | |
| AD- | A-117279.1 | CUGUAGGUUGCUUAAAAGGGA | 70 | 421-441 | A-117280.1 | UCCCUUUUAAGCAACCUACAGGG | 173 | 419-441 |
| 57493.1 | | | | | | | | |
| AD- | A-117351.1 | CUGUAGGUUGCUUAAAAGGGA | 71 | 421-441 | A-117352.1 | UCCCUUUUAAGCAACCUACAGGG | 174 | 419-441 |
| 57529.1 | | | | | | | | |
| AD- | A-119669.1 | CCCCUGUAGGUUGCUUAAAAG | 72 | 418-438 | A-119670.1 | CUUUUAAGCAACCUACAGGGGCA | 175 | 416-438 |
| 58914.1 | | | | | | | | |
| AD- | A-117291.1 | GUAGGUUGCUUAAAAGGGACA | 73 | 423-443 | A-117292.1 | UGUCCCUUUUAAGCAACCUACAG | 176 | 421-443 |
| 57521.1 | | | | | | | | |
| AD- | A-117363.1 | GUAGGUUGCUUAAAAGGGACA | 74 | 423-443 | A-117364.1 | UGUCCCUUUUAAGCAACCUACAG | 177 | 421-443 |
| 57557.1 | | | | | | | | |
| AD- | A-119681.1 | CCCUGUAGGUUGCUUAAAAGG | 75 | 419-439 | A-119682.1 | CCUUUUAAGCAACCUACAGGGGC | 178 | 417-439 |
| 58926.1 | | | | | | | | |
| AD- | A-117257.1 | GUUGCUUAAAAGGGACAGUAU | 76 | 427-447 | A-117258.1 | AUACUGUCCCUUUUAAGCAACCU | 179 | 425-447 |
| 57515.1 | | | | | | | | |
| AD- | A-117329.1 | GUUGCUUAAAAGGGACAGUAU | 77 | 427-447 | A-117330.1 | AUACUGUCCCUUUUAAGCAACCU | 180 | 425-447 |
| 57551.1 | | | | | | | | |
| AD- | A-117357.1 | UUGCUUAAAAGGGACAGUAUU | 78 | 428-448 | A-117358.1 | AAUACUGUCCCUUUUAAGCAACC | 181 | 426-448 |
| 57544.1 | | | | | | | | |
| AD- | A-117285.1 | UUGCUUAAAAGGGACAGUAUU | 79 | 428-448 | A-117286.1 | AAUACUGUCCCUUUUAAGCAACC | 182 | 426-448 |
| 57508.1 | | | | | | | | |
| AD- | A-117289.1 | GCUUAAAAGGGACAGUAUUCU | 80 | 430-450 | A-117290.1 | AGAAUACUGUCCCUUUUAAGCAA | 183 | 428-450 |
| 57517.1 | | | | | | | | |
| AD- | A-117361.1 | GCUUAAAAGGGACAGUAUUCU | 81 | 430-450 | A-117362.1 | AGAAUACUGUCCCUUUUAAGCAA | 184 | 428-450 |
| 57553.1 | | | | | | | | |
| AD- | A-129548.4 | GCUUAAAAGGGACAGUAUUCU | 82 | 430-450 | A-129547.4 | AGAAUACUGUCCCUUUUAAGCAA | 185 | 428-450 |
| 64805.1 | | | | | | | | |
| AD- | A- | GCUUAAAAGGGACAGUAUUCU | 83 | 430-450 | A-129547.3 | AGAAUACUGUCCCUUUUAAGCAA | 186 | 428-450 |
| 64793.1 | 117361.24 | | | | | | | |
| AD- | A-129548.3 | GCUUAAAAGGGACAGUAUUCU | 84 | 430-450 | A- | AGAAUACUGUCCCUUUUAAGCAA | 187 | 428-450 |
| 64799.1 | | | | | 129546.18 | | | |
| AD- | A- | GCUUAAAAGGGACAGUAUUCU | 85 | 430-450 | A- | AGAAUACUGUCCCUUUUAAGCAA | 188 | 428-450 |
| 64787.1 | 117361.23 | | | | 129546.17 | | | |
| AD- | A- | GCUUAAAAGGGACAGUAUUCU | 86 | 430-450 | A-129565.2 | AGAAUACUGUCCCUUUUAAGCAA | 189 | 428-450 |
| 64813.1 | 117361.27 | | | | | | | |
| AD- | A-129554.4 | GCUUAAAAGGGACAGUAUUCU | 87 | 430-450 | A- | AGAAUACUGUCCCUUUUAAGCAA | 190 | 428-450 |
| 64794.1 | | | | | 129546.24 | | | |
| AD- | A-129559.2 | GCUUAAAAGGGACAGUTUUCU | 88 | 430-450 | A- | AGAAUACUGUCCCUUUUAAGCAA | 191 | 428-450 |
| 64824.1 | | | | | 129546.29 | | | |
| AD- | A- | GCUUAAAAGGGACAGUAUUCU | 89 | 430-450 | A-129567.2 | AGAAUACUGUCCCUUUUAAGCAA | 192 | 428-450 |
| 64825.1 | 117361.29 | | | | | | | |
| AD- | A- | GCUUAAAAGGGACAGUAUUCU | 90 | 430-450 | A-129566.3 | AGAAUACUGUCCCUUUUAAGCAA | 193 | 428-450 |
| 64819.1 | 117361.28 | | | | | | | |
| AD- | A-129552.2 | GCUUAAAAUGGACAGUAUUCU | 91 | 430-450 | A- | AGAAUACUGUCCCUUUUAAGCAA | 194 | 428-450 |
| 64828.1 | | | | | 129546.22 | | | |
| AD- | A-129561.2 | GCUUAAAAGGGACAGUCUUCU | 92 | 430-450 | A- | AGAAUACUGUCCCUUUUAAGCAA | 195 | 428-450 |
| 64789.1 | | | | | 129546.31 | | | |
| AD- | A- | GCUUAAAAGGGACAGUAUUCU | 93 | 430-450 | A-129564.3 | AGAAUACUGUCCCUUUUAAGCAA | 196 | 428-450 |
| 64807.1 | 117361.26 | | | | | | | |
| AD- | A-129557.4 | GCUUAAAAGGGACAGUTUUCU | 94 | 430-450 | A- | AGAAUACUGUCCCUUUUAAGCAA | 197 | 428-450 |
| 64812.1 | | | | | 129546.27 | | | |
| AD- | A-129562.2 | GCUUAAAAGGGACAGUUUUCU | 95 | 430-450 | A- | AGAAUACUGUCCCUUUUAAGCAA | 198 | 428-450 |
| 64795.1 | | | | | 129546.32 | | | |
| AD- | A-129554.6 | GCUUAAAAGGGACAGUAUUCU | 96 | 430-450 | A-129572.6 | AGAAUACUGUCCCTUUUAAGCAA | 199 | 428-450 |
| 64804.1 | | | | | | | | |
| AD- | A-129550.8 | GCUUAAAAGGGACAGUAUUCU | 97 | 430-450 | A-129566.4 | AGAAUACUGUCCCUUUUAAGCAA | 200 | 428-450 |
| 64827.1 | | | | | | | | |
| AD- | A-129553.5 | GCUUAAAAGGGACAGTAUUCU | 98 | 430-450 | A- | AGAAUACUGUCCCUUUUAAGCAA | 201 | 428-450 |
| 64788.1 | | | | | 129546.23 | | | |
| AD- | A-129554.5 | GCUUAAAAGGGACAGUAUUCU | 99 | 430-450 | A-129571.6 | AGAAUACUGTCCCTUUUAAGCAA | 202 | 428-450 |
| 64832.1 | | | | | | | | |
| AD- | A-129553.6 | GCUUAAAAGGGACAGTAUUCU | 100 | 430-450 | A-129571.7 | AGAAUACUGTCCCTUUUAAGCAA | 203 | 428-450 |
| 64792.1 | | | | | | | | |
| AD- | A- | GCUUAAAAGGGACAGUAUUCU | 101 | 430-450 | A-129576.2 | AGAAUACUGTCCCUUUUAAGCAA | 204 | 428-450 |
| 64831.1 | 117361.38 | | | | | | | |
| AD- | A- | GCUUAAAAGGGACAGUAUUCU | 102 | 430-450 | A-129574.2 | AGAAUACTGUCCCTUUUAAGCAA | 205 | 428-450 |
| 64820.1 | 117361.36 | | | | | | | |
| AD- | A-129557.6 | GCUUAAAAGGGACAGUTUUCU | 103 | 430-450 | A-129572.8 | AGAAUACUGUCCCTUUUAAGCAA | 206 | 428-450 |
| 64816.1 | | | | | | | | |
| AD- | A-129549.2 | GCUUAAAAGGGACAGUAUUCU | 104 | 430-450 | A- | AGAAUACUGUCCCUUUUAAGCAA | 207 | 428-450 |
| 64811.1 | | | | | 129546.19 | | | |
| AD- | A-129550.7 | GCUUAAAAGGGACAGUAUUCU | 105 | 430-450 | A-129564.4 | AGAAUACUGUCCCUUUUAAGCAA | 208 | 428-450 |
| 64821.1 | | | | | | | | |
| AD- | A- | GCUUAAAAGGGACAGUAUUCU | 106 | 430-450 | A-129572.5 | AGAAUACUGUCCCTUUUAAGCAA | 209 | 428-450 |
| 64808.1 | 117361.34 | | | | | | | |
| AD- | A-129553.7 | GCUUAAAAGGGACAGTAUUCU | 107 | 430-450 | A-129572.7 | AGAAUACUGUCCCTUUUAAGCAA | 210 | 428-450 |
| 64810.1 | | | | | | | | |
| AD- | A-129550.5 | GCUUAAAAGGGACAGUAUUCU | 108 | 430-450 | A- | AGAAUACUGUCCCUUUUAAGCAA | 211 | 428-450 |
| 64817.1 | | | | | 129546.20 | | | |
| AD- | A- | GCUUAAAAGGGACAGUAUUCU | 109 | 430-450 | A-129578.2 | AGAAUACUGUCCCUUUUAAGCAA | 212 | 428-450 |
| 64797.1 | 117361.40 | | | | | | | |
| AD- | A-129560.2 | GCUUAAAAGGGACAGUGUUCU | 110 | 430-450 | A- | AGAAUACUGUCCCUUUUAAGCAA | 213 | 428-450 |
| 64829.1 | | | | | 129546.30 | | | |
| AD- | A- | GCUUAAAAGGGACAGUAUUCU | 111 | 430-450 | A-129571.5 | AGAAUACUGTCCCTUUUAAGCAA | 214 | 428-450 |
| 64802.1 | 117361.33 | | | | | | | |
| AD- | A-129557.5 | GCUUAAAAGGGACAGUTUUCU | 112 | 430-450 | A-129571.8 | AGAAUACUGTCCCTUUUAAGCAA | 215 | 428-450 |
| 64798.1 | | | | | | | | |
| AD- | A-129550.6 | GCUUAAAAGGGACAGUAUUCU | 113 | 430-450 | A-129581.2 | AGAAUACUGUCCCUUUUAAGCAA | 216 | 428-450 |
| 64815.1 | | | | | | | | |
| AD- | A- | GCUUAAAAGGGACAGUAUUCU | 114 | 430-450 | A-129577.2 | AGAAUACUGTCCCUUUUAAGCAA | 217 | 428-450 |
| 64791.1 | 117361.39 | | | | | | | |
| AD- | A- | GCUUAAAAGGGACAGUAUUCU | 115 | 430-450 | A-129573.2 | AGAAUACUGUCCCTUTUAAGCAA | 218 | 428-450 |
| 64814.1 | 117361.35 | | | | | | | |
| AD- | A-129555.2 | GCUUAAAAGGGACAGUATUCU | 116 | 430-450 | A- | AGAAUACUGUCCCUUUUAAGCAA | 219 | 428-450 |
| 64800.1 | | | | | 129546.25 | | | |
| AD- | A-129551.2 | GCUUAAAAGGGACAGUAUUCU | 117 | 430-450 | A- | AGAAUACUGUCCCUUUUAAGCAA | 220 | 428-450 |
| 64823.1 | | | | | 129546.21 | | | |
| AD- | A-129558.2 | GCUUAAAAGGGACAGUAUUCU | 118 | 430-450 | A- | AGAAUACUGUCCCUUUUAAGCAA | 221 | 428-450 |
| 64818.1 | | | | | 129546.28 | | | |
| AD- | A-129556.2 | GCUUAAAAGGGACAGUAUUCU | 119 | 430-450 | A- | AGAAUACUGUCCCUUUUAAGCAA | 222 | 428-450 |
| 64806.1 | | | | | 129546.26 | | | |
| AD- | A- | GCUUAAAAGGGACAGUAUUCU | 120 | 430-450 | A-129580.3 | AGAAUACUGTCCCTUUUAAGCAA | 223 | 428-450 |
| 64809.1 | 117361.42 | | | | | | | |
| AD- | A-129553.8 | GCUUAAAAGGGACAGTAUUCU | 121 | 430-450 | A-129580.4 | AGAAUACUGTCCCTUUUAAGCAA | 224 | 428-450 |
| 64822.1 | | | | | | | | |
| AD- | A- | GCUUAAAAGGGACAGUAUUCU | 122 | 430-450 | A-129570.2 | AGAAUACUGUCCCTUUUAAGCAA | 225 | 428-450 |
| 64796.1 | 117361.32 | | | | | | | |
| AD- | A- | GCUUAAAAGGGACAGUAUUCU | 123 | 430-450 | A-129569.2 | AGAAUACUGTCCCTUUUAAGCAA | 226 | 428-450 |
| 64790.1 | 117361.31 | | | | | | | |
| AD- | A-119675.1 | CUUAAAAGGGACAGUAUUCUC | 124 | 431-451 | A-119676.1 | GAGAAUACUGUCCCUUUUAAGCA | 227 | 429-451 |
| 58920.1 | | | | | | | | |
| AD- | A-119694.1 | AAGGGACAGUAUUCUCAGUGC | 125 | 436-456 | A-119695.1 | GCACUGAGAAUACUGUCCCUUUU | 228 | 434-456 |
| 58925.1 | | | | | | | | |
| AD- | A-119696.1 | GGCCUCCCAAUAAAGCUGGAC | 126 | 499-519 | A-119697.1 | GUCCAGCUUUAUUGGGAGGCCAG | 229 | 497-519 |
| 58927.1 | | | | | | | | |
| AD- | A-119688.1 | GCCUCCCAAUAAAGCUGGACA | 127 | 500-520 | A-119689.1 | UGUCCAGCUUUAUUGGGAGGCCA | 230 | 498-520 |
| 58919.1 | | | | | | | | |
| AD- | A-119679.1 | CCUCCCAAUAAAGCUGGACAA | 128 | 501-521 | A-119680.1 | UUGUCCAGCUUUAUUGGGAGGCC | 231 | 499-521 |
| 58924.1 | | | | | | | | |
| AD- | A-119671.1 | CUCCCAAUAAAGCUGGACAAG | 129 | 502-522 | A-119672.1 | CUUGUCCAGCUUUAUUGGGAGGC | 232 | 500-522 |
| 58916.1 | | | | | | | | |
| AD- | A-119677.1 | UCCCAAUAAAGCUGGACAAGA | 130 | 503-523 | A-119678.1 | UCUUGUCCAGCUUUAUUGGGAGG | 233 | 501-523 |
| 58922.1 | | | | | | | | |
| AD- | A-119667.1 | CCCAAUAAAGCUGGACAAGAA | 131 | 504-524 | A-119668.1 | UUCUUGUCCAGCUUUAUUGGGAG | 234 | 502-524 |
| 58911.1 | | | | | | | | |
| AD- | A-119673.1 | GCUGGACAAGAAGCUGCUAUG | 132 | 513-533 | A-119674.1 | CAUAGCAGCUUCUUGUCCAGCUU | 235 | 511-533 |
| 58918.1 | | | | | | | | |

TABLE 4B
APOC3 Unmodified Sequences Based on NM_023114.3

| Duplex | Sense | Sense Sequence | SEQ | Position in | Antisense | Antisense Sequence | SEQ | Position in |
|---|---|---|---|---|---|---|---|---|
| Name | Oligo Name | (5′ to 3′) | ID NO: | NM_023114.3 | Oligo Name | (5′ to 3′) | ID NO: | NM_023114.3 |
| AD- | A- | GGAUCCUUGCUGCUGGGCUC | 236 | 125-145 | A- | AGAGCCCAGCAGCAAGGAUCCCU | 288 | 123-145 |
| 57526.1 | 117309.1 | U | | | 117310.1 | | | |
| AD- | A- | GGAUCCUUGCUGCUGGGCUC | 237 | 125-145 | A- | AGAGCCCAGCAGCAAGGAUCCCU | 289 | 123-145 |
| 57562.1 | 117381.1 | U | | | 117382.1 | | | |
| AD- | A- | GCUACAUGGAACAAGCCUCC | 238 | 153-173 | A- | UGGAGGCUUGUUCCAUGUAGCCC | 290 | 151-173 |
| 57504.1 | 117299.1 | A | | | 117300.1 | | | |
| AD- | A- | GCUACAUGGAACAAGCCUCC | 239 | 153-173 | A- | UGGAGGCUUGUUCCAUGUAGCCC | 291 | 151-173 |
| 57540.1 | 117371.1 | A | | | 117372.1 | | | |
| AD- | A- | CUACAUGGAACAAGCCUCCA | 240 | 154-174 | A- | UUGGAGGCUUGUUCCAUGUAGCC | 292 | 152-174 |
| 57522.1 | 117307.1 | A | | | 117308.1 | | | |
| AD- | A- | CUACAUGGAACAAGCCUCCA | 241 | 154-174 | A- | UUGGAGGCUUGUUCCAUGUAGCC | 293 | 152-174 |
| 57558.1 | 117379.1 | A | | | 117380.1 | | | |
| AD- | A- | ACAUGGAACAAGCCUCCAAG | 242 | 156-176 | A- | UCUUGGAGGCUUGUUCCAUGUAG | 294 | 154-176 |
| 57500.1 | 117313.1 | A | | | 117314.1 | | | |
| AD- | A- | ACAUGGAACAAGCCUCCAAG | 243 | 156-176 | A- | UCUUGGAGGCUUGUUCCAUGUAG | 295 | 154-176 |
| 57536.1 | 117385.1 | A | | | 117386.1 | | | |
| AD- | A- | GGAACAAGCCUCCAAGACGG | 244 | 160-180 | A- | ACCGUCUUGGAGGCUUGUUCCAU | 296 | 158-180 |
| 57554.1 | 117377.1 | U | | | 117378.1 | | | |
| AD- | A- | GGAACAAGCCUCCAAGACGG | 245 | 160-180 | A- | ACCGUCUUGGAGGCUUGUUCCAU | 297 | 158-180 |
| 57518.1 | 117305.1 | U | | | 117306.1 | | | |
| AD- | A- | ACAAGCCUCCAAGACGGUCC | 246 | 163-183 | A- | UGGACCGUCUUGGAGGCUUGUUC | 298 | 161-183 |
| 57545.1 | 117373.1 | A | | | 117374.1 | | | |
| AD- | A- | ACAAGCCUCCAAGACGGUCC | 247 | 163-183 | A- | UGGACCGUCUUGGAGGCUUGUUC | 299 | 161-183 |
| 57509.1 | 117301.1 | A | | | 117302.1 | | | |
| AD- | A- | GCCUCCAAGACGGUCCAGGA | 248 | 167-187 | A- | AUCCUGGACCGUCUUGGAGGCUU | 300 | 165-187 |
| 57499.1 | 117297.1 | U | | | 117298.1 | | | |
| AD- | A- | GCCUCCAAGACGGUCCAGGA | 249 | 167-187 | A- | AUCCUGGACCGUCUUGGAGGCUU | 301 | 165-187 |
| 57535.1 | 117369.1 | U | | | 117370.1 | | | |
| AD- | A- | CCCUGAAAGGCUACUGGAGC | 250 | 258-278 | A- | UGCUCCAGUAGCCUUUCAGGGAU | 302 | 256-278 |
| 57505.1 | 117315.1 | A | | | 117316.1 | | | |
| AD- | A- | CCCUGAAAGGCUACUGGAGC | 251 | 258-278 | A- | UGCUCCAGUAGCCUUUCAGGGAU | 303 | 256-278 |
| 57541.1 | 117387.1 | A | | | 117388.1 | | | |
| AD- | A- | CCUGAAAGGCUACUGGAGCA | 252 | 259-279 | A- | UUGCUCCAGUAGCCUUUCAGGGA | 304 | 257-279 |
| 57510.1 | 117317.1 | A | | | 117318.1 | | | |
| AD- | A- | CCUGAAAGGCUACUGGAGCA | 253 | 259-279 | A- | UUGCUCCAGUAGCCUUUCAGGGA | 305 | 257-279 |
| 57546.1 | 117389.1 | A | | | 117390.1 | | | |
| AD- | A- | UGAAAGGCUACUGGAGCAA | 254 | 261-281 | A- | ACUUGCUCCAGUAGCCUUUCAGG | 306 | 259-281 |
| 57495.1 | 117311.1 | GU | | | 117312.1 | | | |
| AD- | A- | UGAAAGGCUACUGGAGCAA | 255 | 261-281 | A- | ACUUGCUCCAGUAGCCUUUCAGG | 307 | 259-281 |
| 57531.1 | 117383.1 | GU | | | 117384.1 | | | |
| AD- | A- | GAAAGGCUACUGGAGCAAG | 256 | 262-282 | A- | AACUUGCUCCAGUAGCCUUUCAG | 308 | 260-282 |
| 57514.1 | 117303.1 | UU | | | 117304.1 | | | |
| AD- | A- | GAAAGGCUACUGGAGCAAG | 257 | 262-282 | A- | AACUUGCUCCAGUAGCCUUUCAG | 309 | 260-282 |
| 57550.1 | 117375.1 | UU | | | 117376.1 | | | |
| AD | A- | AACAUGCUGUCCCUAAUAAA | 258 | 348-368 | A- | CUUUAUUAGGGACAGCAUGUUUA | 310 | 346-368 |
| 58954.1 | 119758.1 | G | | | 119759.1 | | | |
| AD- | A- | UAAGGGGAAAGUAUGUUCU | 259 | 350-370 | A- | UGAGAACAUACUUUCCCCUUAAA | 311 | 348-370 |
| 58939.1 | 119720.1 | CA | | | 119721.1 | | | |
| AD- | A- | GCAGAUGUGCCUGUUCCUCC | 260 | 352-372 | A- | UGGAGGAACAGGCACAUCUGCAA | 312 | 350-372 |
| 58949.1 | 119740.1 | A | | | 119741.1 | | | |
| AD | A- | AAGUAUGUUCUCAUGUCUU | 261 | 367-387 | A- | UGAAGACAUGAGAACAUACUUUC | 313 | 365-387 |
| 58936.1 | 119750.1 | CA | | | 119751.1 | | | |
| AD | A- | UCACCUAAACAUGCUGUCCC | 262 | 373-393 | A- | AGGGACAGCAUGUUUAGGUGAGA | 314 | 371-393 |
| 58940.1 | 119736.1 | U | | | 119737.1 | | | |
| AD | A- | UUAAGGGGAAAGUAUGUUC | 263 | 374-394 | A- | GAGAACAUACUUUCCCCUUAAAG | 315 | 372-394 |
| 58945.1 | 119738.1 | UC | | | 119739.1 | | | |
| AD- | A- | CCCUAGAUCUCACCUAAACA | 264 | 377-397 | A- | AUGUUUAGGUGAGAUCUAGGGAG | 316 | 375-397 |
| 58937.1 | 119766.1 | U | | | 119767.1 | | | |
| AD- | A- | UCCCUAAUAAAGCUGGAUA | 265 | 393-413 | A- | CUUAUCCAGCUUUAUUAGGGACA | 317 | 391-413 |
| 58955.1 | 119712.1 | AG | | | 119713.1 | | | |
| AD- | A- | AAACAUGCUGUCCCUAAUAA | 266 | 395-415 | A- | UUUAUUAGGGACAGCAUGUUUAG | 318 | 393-415 |
| 58951.1 | 119710.1 | A | | | 119711.1 | | | |
| AD- | A- | CCCUAAUAAAGCUGGAUAA | 267 | 409-429 | A- | UCUUAUCCAGCUUUAUUAGGGAC | 319 | 407-429 |
| 58947.1 | 119708.1 | GA | | | 119709.1 | | | |
| AD- | A- | CCUAAUAAAGCUGGAUAAG | 268 | 416-436 | A- | UUCUUAUCCAGCUUUAUUAGGGA | 320 | 414-436 |
| 58933.1 | 119702.1 | AA | | | 119703.1 | | | |
| AD- | A- | CUGAAGGUUGCUUUAAGGG | 269 | 417-437 | A- | UCCCCUUAAAGCAACCUUCAGGG | 321 | 415-437 |
| 58931.1 | 119748.1 | GA | | | 119749.1 | | | |
| AD- | A- | AAGGGGAAAGUAUGUUCUC | 270 | 423-443 | A- | AUGAGAACAUACUUUCCCCUUAA | 322 | 421-443 |
| 58938.1 | 119704.1 | AU | | | 119705.1 | | | |
| AD- | A- | AGCUGGAUAAGAAGCUGCU | 271 | 430-450 | A- | ACAGCAGCUUCUUAUCCAGCUUU | 323 | 428-450 |
| 58957.1 | 119744.1 | GU | | | 119745.1 | | | |
| AD- | A- | ACCUAAACAUGCUGUCCCUA | 272 | 435-455 | A- | UUAGGGACAGCAUGUUUAGGUGA | 324 | 433-455 |
| 58958.1 | 119760.1 | A | | | 119761.1 | | | |
| AD- | A- | UUUAAGGGGAAAGUAUGUU | 273 | 452-472 | A- | AGAACAUACUUUCCCCUUAAAGC | 325 | 450-472 |
| 58930.1 | 119732.1 | CU | | | 119733.1 | | | |
| AD- | A- | UCGUGAGACUUCUGUGUUG | 274 | 453-473 | A- | UGCAACACAGAAGUCUCACGACU | 326 | 451-473 |
| 58932.1 | 119764.1 | CA | | | 119765.1 | | | |
| AD- | A- | AUUGAGUCGUGAGACUUCU | 275 | 457-477 | A- | ACAGAAGUCUCACGACUCAAUAG | 327 | 455-477 |
| 58961.1 | 119746.1 | GU | | | 119747.1 | | | |
| AD- | A- | GUCCCUAAUAAAGCUGGAU | 276 | 463-483 | A- | UUAUCCAGCUUUAUUAGGGACAG | 328 | 461-483 |
| 58952.1 | 119726.1 | AA | | | 119727.1 | | | |
| AD- | A- | UUCUGUGUUGCAGAUGUGC | 277 | 465-485 | A- | AGGCACAUCUGCAACACAGAAGU | 329 | 463-485 |
| 58946.1 | 119754.1 | CU | | | 119755.1 | | | |
| AD- | A- | UGGCCCCUGAAGGUUGCUUU | 278 | 468-488 | A- | UAAAGCAACCUUCAGGGGCCACC | 330 | 466-488 |
| 58956.1 | 119728.1 | A | | | 119729.1 | | | |
| AD- | A- | GUUGCUUUAAGGGGAAAGU | 279 | 470-490 | A- | AUACUUUCCCCUUAAAGCAACCU | 331 | 468-490 |
| 58929.1 | 119716.1 | AU | | | 119717.1 | | | |
| AD- | A- | UGAGACUUCUGUGUUGCAG | 280 | 473-493 | A- | AUCUGCAACACAGAAGUCUCACG | 332 | 471-493 |
| 58948.1 | 119724.1 | AU | | | 119725.1 | | | |
| AD- | A- | GCUGGAUAAGAAGCUGCUG | 281 | 475-495 | A- | AACAGCAGCUUCUUAUCCAGCUU | 333 | 473-495 |
| 58935.1 | 119734.1 | UU | | | 119735.1 | | | |
| AD- | A- | CUCCCUAGAUCUCACCUAAA | 282 | 476-496 | A- | GUUUAGGUGAGAUCUAGGGAGGG | 334 | 474-496 |
| 58944.1 | 119722.1 | C | | | 119723.1 | | | |
| AD- | A- | CCUAAACAUGCUGUCCCUAA | 283 | 480-500 | A- | AUUAGGGACAGCAUGUUUAGGUG | 335 | 478-500 |
| 58959.1 | 119714.1 | U | | | 119715.1 | | | |
| AD- | A- | GAAAGUAUGUUCUCAUGUC | 284 | 490-510 | A- | AAGACAUGAGAACAUACUUUCCC | 336 | 488-510 |
| 58960.1 | 119730.1 | UU | | | 119731.1 | | | |
| AD- | A- | GCCCCUGAAGGUUGCUUUAA | 285 | 497-517 | A- | CUUAAAGCAACCUUCAGGGGCCA | 337 | 495-517 |
| 58928.1 | 119700.1 | G | | | 119701.1 | | | |
| AD- | A- | CCCUCCCUAGAUCUCACCUA | 286 | 501-521 | A- | UUAGGUGAGAUCUAGGGAGGGGU | 338 | 499-521 |
| 58950.1 | 119756.1 | A | | | 119757.1 | | | |
| AD- | A- | CUGUCCCUAAUAAAGCUGGA | 287 | 506-526 | A- | AUCCAGCUUUAUUAGGGACAGCA | 339 | 504-526 |
| 58962.1 | 119762.1 | U | | | 119763.1 | | | |

TABLE 5
APOC3 Modified Sequences

| Duplex | Sense Oligo | | SEQ ID | Antis Oligo | | SEQ ID |
|---|---|---|---|---|---|---|
| Name | Name | Sense Sequence (5′ to 3′) | NO: | Name | Antisense Sequence (5′ to 3′) | NO: |
| AD-57501.1 | A-117251.1 | AfcCfaAfgAfcCfGfCfcAfaGfgAfuGfcAfL96 | 340 | A-117252.1 | uGfcAfuCfcUfuGfgcgGfuCfuUfgGfusGfsg | 495 |
| AD-57537.1 | A-117323.1 | AfscsCfaAfgAfcCfGfCfcAfaGfgAfuGfcAfL96 | 341 | A-117324.1 | usGfscAfuCfcUfuGfgcgGfuCfuUfgGfusGfsg | 496 |
| AD-57512.1 | A-117271.1 | CfaAfgAfcCfgCfCfAfaGfgAfuGfcAfcUfL96 | 342 | A-117272.1 | aGfuGfcAfuCfcUfuggCfgGfuCfuUfgsGfsu | 497 |
| AD-57548.1 | A-117343.1 | CfsasAfgAfcCfgCfCfAfaGfgAfuGfcAfcUfL96 | 343 | A-117344.1 | asGfsuGfcAfuCfcUfuggCfgGfuCfuUfgsGfsu | 498 |
| AD-57496.1 | A-117249.1 | CfcGfaUfgGfcUfUfCfaGfuUfcCfcUfgAfL96 | 344 | A-117250.1 | uCfaGfgGfaAfcUfgaaGfcCfaUfcGfgsUfsc | 499 |
| AD-57532.1 | A-117321.1 | CfscsGfaUfgGfcUfUfCfaGfuUfcCfcUfgAfL96 | 345 | A-117322.1 | usCfsaGfgGfaAfcUfgaaGfcCfaUfcGfgsUfsc | 500 |
| AD-57491.1 | A-117247.1 | CfgAfuGfgCfuUfCfAfgUfuCfcCfuGfaAfL96 | 346 | A-117248.1 | uUfcAfgGfgAfaCfugaAfgCfcAfuCfgsGfsu | 501 |
| AD-57527.1 | A-117319.1 | CfsgsAfuGfgCfuUfCfAfgUfuCfcCfuGfaAfL96 | 347 | A-117320.1 | usUfscAfgGfgAfaCfugaAfgCfcAfuCfgsGfsu | 502 |
| AD-57547.1 | A-117327.1 | AfsgsAfcUfaCfuGfGfAfgCfaCfcGfuUfaAfL96 | 348 | A-117328.1 | usUfsaAfcGfgUfgCfuccAfgUfaGfuCfusUfsu | 503 |
| AD-57511.1 | A-117255.1 | AfgAfcUfaCfuGfGfAfgCfaCfcGfuUfaAfL96 | 349 | A-117256.1 | uUfaAfcGfgUfgCfuccAfgUfaGfuCfusUfsu | 504 |
| AD-57561.1 | A-117365.1 | CfsusAfcUfgGfaGfCfAfcCfgUfuAfaGfgAfL96 | 350 | A-117366.1 | usCfscUfuAfaCfgGfugcUfcCfaGfuAfgsUfsc | 505 |
| AD-57525.1 | A-117293.1 | CfuAfcUfgGfaGfCfAfcCfgUfuAfaGfgAfL96 | 351 | A-117294.1 | uCfcUfuAfaCfgGfugcUfcCfaGfuAfgsUfsc | 506 |
| AD-57520.1 | A-117275.1 | AfcUfgGfaGfcAfCfCfgUfuAfaGfgAfcAfL96 | 352 | A-117276.1 | uGfuCfcUfuAfaCfgguGfcUfcCfaGfusAfsg | 507 |
| AD-57556.1 | A-117347.1 | AfscsUfgGfaGfcAfCfCfgUfuAfaGfgAfcAfL96 | 353 | A-117348.1 | usGfsuCfcUfuAfaCfgguGfcUfcCfaGfusAfsg | 508 |
| AD-57503.1 | A-117283.1 | CfuGfgAfgCfaCfCfGfuUfaAfgGfaCfaAfL96 | 354 | A-117284.1 | uUfgUfcCfuUfaAfcggUfgCfuCfcAfgsUfsa | 509 |
| AD-57539.1 | A-117355.1 | CfsusGfgAfgCfaCfCfGfuUfaAfgGfaCfaAfL96 | 355 | A-117356.1 | usUfsgUfcCfuUfaAfcggUfgCfuCfcAfgsUfsa | 510 |
| AD-57533.1 | A-117337.1 | GfsgsAfgCfaCfcGfUfUfaAfgGfaCfaAfgUfL96 | 356 | A-117338.1 | asCfsuUfgUfcCfuUfaacGfgUfgCfuCfcsAfsg | 511 |
| AD-57497.1 | A-117265.1 | GfgAfgCfaCfcGfUfUfaAfgGfaCfaAfgUfL96 | 357 | A-117266.1 | aCfuUfgUfcCfuUfaacGfgUfgCfuCfcsAfsg | 512 |
| AD-57498.1 | A-117281.1 | GfaGfcAfcCfgUfUfAfaGfgAfcAfaGfuUfL96 | 358 | A-117282.1 | aAfcUfuGfuCfcUfuaaCfgGfuGfcUfcsCfsa | 513 |
| AD-57534.1 | A-117353.1 | GfsasGfcAfcCfgUfUfAfaGfgAfcAfaGfuUfL96 | 359 | A-117354.1 | asAfscUfuGfuCfcUfuaaCfgGfuGfcUfcsCfsa | 514 |
| AD-57506.1 | A-117253.1 | GfuGfgCfuGfcCfUfGfaGfaCfcUfcAfaUfL96 | 360 | A-117254.1 | aUfuGfaGfgUfcUfcagGfcAfgCfcAfcsGfsg | 515 |
| AD-57542.1 | A-117325.1 | GfsusGfgCfuGfcCfUfGfaGfaCfcUfcAfaUfL96 | 361 | A-117326.1 | asUfsuGfaGfgUfcUfcagGfcAfgCfcAfcsGfsg | 516 |
| AD-57523.1 | A-117261.1 | GfcCfuGfaGfaCfCfUfcAfaUfaCfcCfcAfL96 | 362 | A-117262.1 | uGfgGfgUfaUfuGfaggUfcUfcAfgGfcsAfsg | 517 |
| AD-57559.1 | A-117333.1 | GfscsCfuGfaGfaCfCfUfcAfaUfaCfcCfcAfL96 | 363 | A-117334.1 | usGfsgGfgUfaUfuGfaggUfcUfcAfgGfcsAfsg | 518 |
| AD-58915.1 | A-119685.1 | GfsgsCfuGfcCfuGfAfGfaCfcUfcAfaUfaCfL96 | 364 | A-119686.1 | gsUfsaUfuGfaGfgUfcucAfgGfcAfgCfcsasc | 519 |
| AD-57507.1 | A-117269.1 | CfcUfgAfgAfcCfUfCfaAfuAfcCfcCfaAfL96 | 365 | A-117270.1 | uUfgGfgGfuAfuUfgagGfuCfuCfaGfgsCfsa | 520 |
| AD-57543.1 | A-117341.1 | CfscsUfgAfgAfcCfUfCfaAfuAfcCfcCfaAfL96 | 366 | A-117342.1 | usUfsgGfgGfuAfuUfgagGfuCfuCfaGfgsCfsa | 521 |
| AD-58921.1 | A-119690.1 | GfscsUfgCfcUfgAfGfAfcCfuCfaAfuAfcCfL96 | 367 | A-119691.1 | gsGfsuAfuUfgAfgGfucuCfaGfgCfaGfcscsa | 522 |
| AD-58913.1 | A-119698.1 | CfsusGfcCfuGfaGfAfCfcUfcAfaUfaCfcCfL96 | 368 | A-119699.1 | gsGfsgUfaUfuGfaGfgucUfcAfgGfcAfgscsc | 523 |
| AD-57502.1 | A-117267.1 | GfaCfcUfcAfaUfAfCfcCfcAfaGfuCfcAfL96 | 369 | A-117268.1 | uGfgAfcUfuGfgGfguaUfuGfaGfgUfcsUfsc | 524 |
| AD-57538.1 | A-117339.1 | GfsasCfcUfcAfaUfAfCfcCfcAfaGfuCfcAfL96 | 370 | A-117340.1 | usGfsgAfcUfuGfgGfguaUfuGfaGfgUfcsUfsc | 525 |
| AD-58923.1 | A-119692.1 | UfsgsAfgAfcCfuCfAfAfuAfcCfcCfaAfgUfL96 | 371 | A-119693.1 | asCfsuUfgGfgGfuAfuugAfgGfuCfuCfasgsg | 526 |
| AD-58912.1 | A-119683.1 | AfsusCfuCfcAfgGfGfCfuGfcCfcCfuGfuAfL96 | 372 | A-119684.1 | usAfscAfgGfgGfcAfgccCfuGfgAfgAfususg | 527 |
| AD-57516.1 | A-117273.1 | GfcUfgCfcCfcUfGfUfaGfgUfuGfcUfuAfL96 | 373 | A-117274.1 | uAfaGfcAfaCfcUfacaGfgGfgCfaGfcsCfsc | 528 |
| AD-57552.1 | A-117345.1 | GfscsUfgCfcCfcUfGfUfaGfgUfuGfcUfuAfL96 | 374 | A-117346.1 | usAfsaGfcAfaCfcUfacaGfgGfgCfaGfcsCfsc | 529 |
| AD-57513.1 | A-117287.1 | UfgCfcCfcUfgUfAfGfgUfuGfcUfuAfaAfL96 | 375 | A-117288.1 | uUfuAfaGfcAfaCfcuaCfaGfgGfgCfasGfsc | 530 |
| AD-57549.1 | A-117359.1 | UfsgsCfcCfcUfgUfAfGfgUfuGfcUfuAfaAfL96 | 376 | A-117360.1 | usUfsuAfaGfcAfaCfcuaCfaGfgGfgCfasGfsc | 531 |
| AD-57519.1 | A-117259.1 | GfcCfcCfuGfuAfGfGfuUfgCfuUfaAfaAfL96 | 377 | A-117260.1 | uUfuUfaAfgCfaAfccuAfcAfgGfgGfcsAfsg | 532 |
| AD-57555.1 | A-117331.1 | GfscsCfcCfuGfuAfGfGfuUfgCfuUfaAfaAfL96 | 378 | A-117332.1 | usUfsuUfaAfgCfaAfccuAfcAfgGfgGfcsAfsg | 533 |
| AD-58917.1 | A-117367.2 | CfsusGfcCfcCfuGfUfAfgGfuUfgCfuUfaAfL96 | 379 | A-119687.1 | usUfsaAfgCfaAfcCfuacAfgGfgGfcAfgscsc | 534 |
| AD-57493.1 | A-117279.1 | CfuGfuAfgGfuUfGfCfuUfaAfaAfgGfgAfL96 | 380 | A-117280.1 | uCfcCfuUfuUfaAfgcaAfcCfuAfcAfgsGfsg | 535 |
| AD-57529.1 | A-117351.1 | CfsusGfuAfgGfuUfGfCfuUfaAfaAfgGfgAfL96 | 381 | A-117352.1 | usCfscCfuUfuUfaAfgcaAfcCfuAfcAfgsGfsg | 536 |
| AD-58914.1 | A-119669.1 | CfscsCfcUfgUfaGfGfUfuGfcUfuAfaAfaGfL96 | 382 | A-119670.1 | csUfsuUfuAfaGfcAfaccUfaCfaGfgGfgscsa | 537 |
| AD-57521.1 | A-117291.1 | GfuAfgGfuUfgCfUfUfaAfaAfgGfgAfcAfL96 | 383 | A-117292.1 | uGfuCfcCfuUfuUfaagCfaAfcCfuAfcsAfsg | 538 |
| AD-57557.1 | A-117363.1 | GfsusAfgGfuUfgCfUfUfaAfaAfgGfgAfcAfL96 | 384 | A-117364.1 | usGfsuCfcCfuUfuUfaagCfaAfcCfuAfcsAfsg | 539 |
| AD-58926.1 | A-119681.1 | CfscsCfuGfuAfgGfUfUfgCfuUfaAfaAfgGfL96 | 385 | A-119682.1 | csCfsuUfuUfaAfgCfaacCfuAfcAfgGfgsgsc | 540 |
| AD-57515.1 | A-117257.1 | GfuUfgCfuUfaAfAfAfgGfgAfcAfgUfaUfL96 | 386 | A-117258.1 | aUfaCfuGfuCfcCfuuuUfaAfgCfaAfcsCfsu | 541 |
| AD-57551.1 | A-117329.1 | GfsusUfgCfuUfaAfAfAfgGfgAfcAfgUfaUfL96 | 387 | A-117330.1 | asUfsaCfuGfuCfcCfuuuUfaAfgCfaAfcsCfsu | 542 |
| AD-57544.1 | A-117357.1 | UfsusGfcUfuAfaAfAfGfgGfaCfaGfuAfuUfL96 | 388 | A-117358.1 | asAfsuAfcUfgUfcCfcuuUfuAfaGfcAfasCfsc | 543 |
| AD-57508.1 | A-117285.1 | UfuGfcUfuAfaAfAfGfgGfaCfaGfuAfuUfL96 | 389 | A-117286.1 | aAfuAfcUfgUfcCfcuuUfuAfaGfcAfasCfsc | 544 |
| AD-57517.1 | A-117289.1 | GfcUfuAfaAfaGfGfGfaCfaGfuAfuUfcUfL96 | 390 | A-117290.1 | aGfaAfuAfcUfgUfcccUfuUfuAfaGfcsAfsa | 545 |
| AD-57553.1 | A-117361.1 | GfscsUfuAfaAfaGfGfGfaCfaGfuAfuUfcUfL96 | 391 | A-117362.1 | asGfsaAfuAfcUfgUfcccUfuUfuAfaGfcsAfsa | 546 |
| AD-64805.1 | A-129548.4 | Y44GfscsUfuAfaAfaGfGfGfaCfaGfuAfuUfcUfL96 | 392 | A-129547.4 | PasGfsaAfuAfcUfgUfcccUfuUfuAfaGfcsasa | 547 |
| AD-64793.1 | A-117361.24 | GfscsUfuAfaAfaGfGfGfaCfaGfuAfuUfcUfL96 | 393 | A-129547.3 | PasGfsaAfuAfcUfgUfcccUfuUfuAfaGfcsasa | 548 |
| AD-64799.1 | A-129548.3 | Y44GfscsUfuAfaAfaGfGfGfaCfaGfuAfuUfcUfL96 | 394 | A-129546.18 | asGfsaAfuAfcUfgUfcccUfuUfuAfaGfcsasa | 549 |
| AD-64787.1 | A-117361.23 | GfscsUfuAfaAfaGfGfGfaCfaGfuAfuUfcUfL96 | 395 | A-129546.17 | asGfsaAfuAfcUfgUfcccUfuUfuAfaGfcsasa | 550 |
| AD-64813.1 | A-117361.27 | GfscsUfuAfaAfaGfGfGfaCfaGfuAfuUfcUfL96 | 396 | A-129565.2 | asGfsaauAfcUfgucccUfuuuaagcsasa | 551 |
| AD-64794.1 | A-129554.4 | gscsuuaaaaggdGacagu(Agn)uucuL96 | 397 | A-129546.24 | asGfsaAfuAfcUfgUfcccUfuUfuAfaGfcsasa | 552 |
| AD-64824.1 | A-129559.2 | gscsuuaaaaggdGdAcagudTuucuL96 | 398 | A-129546.29 | asGfsaAfuAfcUfgUfcccUfuUfuAfaGfcsasa | 553 |
| AD-64825.1 | A-117361.29 | GfscsUfuAfaAfaGfGfGfaCfaGfuAfuUfcUfL96 | 399 | A-129567.2 | asGfsaauacugucccUfuuuaagcsasa | 554 |
| AD-64819.1 | A-117361.28 | GfscsUfuAfaAfaGfGfGfaCfaGfuAfuUfcUfL96 | 400 | A-129566.3 | asGfsaauAfcugucccUfuUfuaagcsasa | 555 |
| AD-64828.1 | A-129552.2 | gscsuuaaaaugdGacaguauucuL96 | 401 | A-129546.22 | asGfsaAfuAfcUfgUfcccUfuUfuAfaGfcsasa | 556 |
| AD-64789.1 | A-129561.2 | gscsuuaaaaggdGacagucuucuL96 | 402 | A-129546.31 | asGfsaAfuAfcUfgUfcccUfuUfuAfaGfcsasa | 557 |
| AD-64807.1 | A-117361.26 | GfscsUfuAfaAfaGfGfGfaCfaGfuAfuUfcUfL96 | 403 | A-129564.3 | asGfsaauAfcUfGfucccUfuUfuaagcsasa | 558 |
| AD-64812.1 | A-129557.4 | gscsuuaaaaggdGacagudTuucuL96 | 404 | A-129546.27 | asGfsaAfuAfcUfgUfcccUfuUfuAfaGfcsasa | 559 |
| AD-64795.1 | A-129562.2 | gscsuuaaaaggdGacaguuuucuL96 | 405 | A-129546.32 | asGfsaAfuAfcUfgUfcccUfuUfuAfaGfcsasa | 560 |
| AD-64804.1 | A-129554.6 | gscsuuaaaaggdGacagu(Agn)uucuL96 | 406 | A-129572.6 | asdGsaaudAcugucccdTuuuaagcsasa | 561 |
| AD-64827.1 | A-129550.8 | gscsuuaaAfagGfGfacaguauucuL96 | 407 | A-129566.4 | asGfsaauAfcugucccUfuUfuaagcsasa | 562 |
| AD-64788.1 | A-129553.5 | gscsuuaaaaggdGacag(Tgn)auucuL96 | 408 | A-129546.23 | asGfsaAfuAfcUfgUfcccUfuUfuAfaGfcsasa | 563 |
| AD-64832.1 | A-129554.5 | gscsuuaaaaggdGacagu(Agn)uucuL96 | 409 | A-129571.6 | asdGsaauacugdTcccdTuuuaagcsasa | 564 |
| AD-64792.1 | A-129553.6 | gscsuuaaaaggdGacag(Tgn)auucuL96 | 410 | A-129571.7 | asdGsaauacugdTcccdTuuuaagcsasa | 565 |
| AD-64831.1 | A-117361.38 | GfscsUfuAfaAfaGfGfGfaCfaGfuAfuUfcUfL96 | 411 | A-129576.2 | asdGsaauacugdTcccUuuuaagcsasa | 566 |
| AD-64820.1 | A-117361.36 | GfscsUfuAfaAfaGfGfGfaCfaGfuAfuUfcUfL96 | 412 | A-129574.2 | asdGsaaudAcdTgucccdTuuuaagcsasa | 567 |
| AD-64816.1 | A-129557.6 | gscsuuaaaaggdGacagudTuucuL96 | 413 | A-129572.8 | asdGsaaudAcugucccdTuuuaagcsasa | 568 |
| AD-64811.1 | A-129549.2 | gscsuuAfaAfagGfGfacaguauucuL96 | 414 | A-129546.19 | asGfsaAfuAfcUfgUfcccUfuUfuAfaGfcsasa | 569 |
| AD-64821.1 | A-129550.7 | gscsuuaaAfagGfGfacaguauucuL96 | 415 | A-129564.4 | asGfsaauAfcUfGfucccUfuUfuaagcsasa | 570 |
| AD-64808.1 | A-117361.34 | GfscsUfuAfaAfaGfGfGfaCfaGfuAfuUfcUfL96 | 416 | A-129572.5 | asdGsaaudAcugucccdTuuuaagcsasa | 571 |
| AD-64810.1 | A-129553.7 | gscsuuaaaaggdGacag(Tgn)auucuL96 | 417 | A-129572.7 | asdGsaaudAcugucccdTuuuaagcsasa | 572 |
| AD-64817.1 | A-129550.5 | gscsuuaaAfagGfGfacaguauucuL96 | 418 | A-129546.20 | asGfsaAfuAfcUfgUfcccUfuUfuAfaGfcsasa | 573 |
| AD-64797.1 | A-117361.40 | GfscsUfuAfaAfaGfGfGfaCfaGfuAfuUfcUfL96 | 419 | A-129578.2 | asGsaaudAcugucccUuuuaagcsasa | 574 |
| AD-64829.1 | A-129560.2 | gscsuuaaaaggdGacaguguucuL96 | 420 | A-129546.30 | asGfsaAfuAfcUfgUfcccUfuUfuAfaGfcsasa | 575 |
| AD-64802.1 | A-117361.33 | GfscsUfuAfaAfaGfGfGfaCfaGfuAfuUfcUfL96 | 421 | A-129571.5 | asdGsaauacugdTcccdTuuuaagcsasa | 576 |
| AD-64798.1 | A-129557.5 | gscsuuaaaaggdGacagudTuucuL96 | 422 | A-129571.8 | asdGsaauacugdTcccdTuuuaagcsasa | 577 |
| AD-64815.1 | A-129550.6 | gscsuuaaAfagGfGfacaguauucuL96 | 423 | A-129581.2 | asGfsaauAfcugUfcccUfuUfuaagcsasa | 578 |
| AD-64791.1 | A-117361.39 | GfscsUfuAfaAfaGfGfGfaCfaGfuAfuUfcUfL96 | 424 | A-129577.2 | asGsaauacugdTcccUuuuaagcsasa | 579 |
| AD-64814.1 | A-117361.35 | GfscsUfuAfaAfaGfGfGfaCfaGfuAfuUfcUfL96 | 425 | A-129573.2 | asdGsaauacugucccdTudTuaagcsasa | 580 |
| AD-64800.1 | A-129555.2 | gscsuuaaaaggdGacagua(Tgn)ucuL96 | 426 | A-129546.25 | asGfsaAfuAfcUfgUfcccUfuUfuAfaGfcsasa | 581 |
| AD-64823.1 | A-129551.2 | gscsuuaaaaggdGacaguauucuL96 | 427 | A-129546.21 | asGfsaAfuAfcUfgUfcccUfuUfuAfaGfcsasa | 582 |
| AD-64818.1 | A-129558.2 | gscsuuaaaaggdGdAcagudAuucuL96 | 428 | A-129546.28 | asGfsaAfuAfcUfgUfcccUfuUfuAfaGfcsasa | 583 |
| AD-64806.1 | A-129556.2 | gscsuuaaaaggdGacagudAuucuL96 | 429 | A-129546.26 | asGfsaAfuAfcUfgUfcccUfuUfuAfaGfcsasa | 584 |
| AD-64809.1 | A-117361.42 | GfscsUfuAfaAfaGfGfGfaCfaGfuAfuUfcUfL96 | 430 | A-129580.3 | PasgsaauacugdTcccdTuuuaagcsasa | 585 |
| AD-64822.1 | A-129553.8 | gscsuuaaaaggdGacag(Tgn)auucuL96 | 431 | A-129580.4 | PasgsaauacugdTcccdTuuuaagcsasa | 586 |
| AD-64796.1 | A-117361.32 | GfscsUfuAfaAfaGfGfGfaCfaGfuAfuUfcUfL96 | 432 | A-129570.2 | asgsaaudAcugucccdTuuuaagcsasa | 587 |
| AD-64790.1 | A-117361.31 | GfscsUfuAfaAfaGfGfGfaCfaGfuAfuUfcUfL96 | 433 | A-129569.2 | asgsaauacugdTcccdTuuuaagcsasa | 588 |
| AD-58920.1 | A-119675.1 | CfsusUfaAfaAfgGfGfAfcAfgUfaUfuCfuCfL96 | 434 | A-119676.1 | gsAfsgAfaUfaCfuGfuccCfuUfuUfaAfgscsa | 589 |
| AD-58925.1 | A-119694.1 | AfsasGfgGfaCfaGfUfAfuUfcUfcAfgUfgCfL96 | 435 | A-119695.1 | gsCfsaCfuGfaGfaAfuacUfgUfcCfcUfususu | 590 |
| AD-58927.1 | A-119696.1 | GfsgsCfcUfcCfcAfAfUfaAfaGfcUfgGfaCfL96 | 436 | A-119697.1 | gsUfscCfaGfcUfuUfauuGfgGfaGfgCfcsasg | 591 |
| AD-58919.1 | A-119688.1 | GfscsCfuCfcCfaAfUfAfaAfgCfuGfgAfcAfL96 | 437 | A-119689.1 | usGfsuCfcAfgCfuUfuauUfgGfgAfgGfcscsa | 592 |
| AD-58924.1 | A-119679.1 | CfscsUfcCfcAfaUfAfAfaGfcUfgGfaCfaAfL96 | 438 | A-119680.1 | usUfsgUfcCfaGfcUfuuaUfuGfgGfaGfgscsc | 593 |
| AD-58916.1 | A-119671.1 | CfsusCfcCfaAfuAfAfAfgCfuGfgAfcAfaGfL96 | 439 | A-119672.1 | csUfsuGfuCfcAfgCfuuuAfuUfgGfgAfgsgsc | 594 |
| AD-58922.1 | A-119677.1 | UfscsCfcAfaUfaAfAfGfcUfgGfaCfaAfgAfL96 | 440 | A-119678.1 | usCfsuUfgUfcCfaGfcuuUfaUfuGfgGfasgsg | 595 |
| AD-58911.1 | A-119667.1 | CfscsCfaAfuAfaAfGfCfuGfgAfcAfaGfaAfL96 | 441 | A-119668.1 | usUfscUfuGfuCfcAfgcuUfuAfuUfgGfgsasg | 596 |
| AD-58918.1 | A-119673.1 | GfscsUfgGfaCfaAfGfAfaGfcUfgCfuAfuGfL96 | 442 | A-119674.1 | csAfsuAfgCfaGfcUfucuUfgUfcCfaGfcsusu | 597 |
| AD-57526.1 | A-117309.1 | GfgAfuCfcUfuGfCfUfgCfuGfgGfcUfcUfL96 | 443 | A-117310.1 | aGfaGfcCfcAfgCfagcAfaGfgAfuCfcsCfsu | 598 |
| AD-57562.1 | A-117381.1 | GfsgsAfuCfcUfuGfCfUfgCfuGfgGfcUfcUfL96 | 444 | A-117382.1 | asGfsaGfcCfcAfgCfagcAfaGfgAfuCfcsCfsu | 599 |
| AD-57504.1 | A-117299.1 | GfcUfaCfaUfgGfAfAfcAfaGfcCfuCfcAfL96 | 445 | A-117300.1 | uGfgAfgGfcUfuGfuucCfaUfgUfaGfcsCfsc | 600 |
| AD-57540.1 | A-117371.1 | GfscsUfaCfaUfgGfAfAfcAfaGfcCfuCfcAfL96 | 446 | A-117372.1 | usGfsgAfgGfcUfuGfuucCfaUfgUfaGfcsCfsc | 601 |
| AD-57522.1 | A-117307.1 | CfuAfcAfuGfgAfAfCfaAfgCfcUfcCfaAfL96 | 447 | A-117308.1 | uUfgGfaGfgCfuUfguuCfcAfuGfuAfgsCfsc | 602 |
| AD-57558.1 | A-117379.1 | CfsusAfcAfuGfgAfAfCfaAfgCfcUfcCfaAfL96 | 448 | A-117380.1 | usUfsgGfaGfgCfuUfguuCfcAfuGfuAfgsCfsc | 603 |
| AD-57500.1 | A-117313.1 | AfcAfuGfgAfaCfAfAfgCfcUfcCfaAfgAfL96 | 449 | A-117314.1 | uCfuUfgGfaGfgCfuugUfuCfcAfuGfusAfsg | 604 |
| AD-57536.1 | A-117385.1 | AfscsAfuGfgAfaCfAfAfgCfcUfcCfaAfgAfL96 | 450 | A-117386.1 | usCfsuUfgGfaGfgCfuugUfuCfcAfuGfusAfsg | 605 |
| AD-57554.1 | A-117377.1 | GfsgsAfaCfaAfgCfCfUfcCfaAfgAfcGfgUfL96 | 451 | A-117378.1 | asCfscGfuCfuUfgGfaggCfuUfgUfuCfcsAfsu | 606 |
| AD-57518.1 | A-117305.1 | GfgAfaCfaAfgCfCfUfcCfaAfgAfcGfgUfL96 | 452 | A-117306.1 | aCfcGfuCfuUfgGfaggCfuUfgUfuCfcsAfsu | 607 |
| AD-57545.1 | A-117373.1 | AfscsAfaGfcCfuCfCfAfaGfaCfgGfuCfcAfL96 | 453 | A-117374.1 | usGfsgAfcCfgUfcUfuggAfgGfcUfuGfusUfsc | 608 |
| AD-57509.1 | A-117301.1 | AfcAfaGfcCfuCfCfAfaGfaCfgGfuCfcAfL96 | 454 | A-117302.1 | uGfgAfcCfgUfcUfuggAfgGfcUfuGfusUfsc | 609 |
| AD-57499.1 | A-117297.1 | GfcCfuCfcAfaGfAfCfgGfuCfcAfgGfaUfL96 | 455 | A-117298.1 | aUfcCfuGfgAfcCfgucUfuGfgAfgGfcsUfsu | 610 |
| AD-57535.1 | A-117369.1 | GfscsCfuCfcAfaGfAfCfgGfuCfcAfgGfaUfL96 | 456 | A-117370.1 | asUfscCfuGfgAfcCfgucUfuGfgAfgGfcsUfsu | 611 |
| AD-57505.1 | A-117315.1 | CfcCfuGfaAfaGfGfCfuAfcUfgGfaGfcAfL96 | 457 | A-117316.1 | uGfcUfcCfaGfuAfgccUfuUfcAfgGfgsAfsu | 612 |
| AD-57541.1 | A-117387.1 | CfscsCfuGfaAfaGfGfCfuAfcUfgGfaGfcAfL96 | 458 | A-117388.1 | usGfscUfcCfaGfuAfgccUfuUfcAfgGfgsAfsu | 613 |
| AD-57510.1 | A-117317.1 | CfcUfgAfaAfgGfCfUfaCfuGfgAfgCfaAfL96 | 459 | A-117318.1 | uUfgCfuCfcAfgUfagcCfuUfuCfaGfgsGfsa | 614 |
| AD-57546.1 | A-117389.1 | CfscsUfgAfaAfgGfCfUfaCfuGfgAfgCfaAfL96 | 460 | A-117390.1 | usUfsgCfuCfcAfgUfagcCfuUfuCfaGfgsGfsa | 615 |
| AD-57495.1 | A-117311.1 | UfgAfaAfgGfcUfAfCfuGfgAfgCfaAfgUfL96 | 461 | A-117312.1 | aCfuUfgCfuCfcAfguaGfcCfuUfuCfasGfsg | 616 |
| AD-57531.1 | A-117383.1 | UfsgsAfaAfgGfcUfAfCfuGfgAfgCfaAfgUfL96 | 462 | A-117384.1 | asCfsuUfgCfuCfcAfguaGfcCfuUfuCfasGfsg | 617 |
| AD-57514.1 | A-117303.1 | GfaAfaGfgCfuAfCfUfgGfaGfcAfaGfuUfL96 | 463 | A-117304.1 | aAfcUfuGfcUfcCfaguAfgCfcUfuUfcsAfsg | 618 |
| AD-57550.1 | A-117375.1 | GfsasAfaGfgCfuAfCfUfgGfaGfcAfaGfuUfL96 | 464 | A-117376.1 | asAfscUfuGfcUfcCfaguAfgCfcUfuUfcsAfsg | 619 |
| AD-58954.1 | A-119758.1 | AfsasCfaUfgCfuGfUfCfcCfuAfaUfaAfaGfL96 | 465 | A-119759.1 | csUfsuUfaUfuAfgGfgacAfgCfaUfgUfususa | 620 |
| AD-58939.1 | A-119720.1 | UfsasAfgGfgGfaAfAfGfuAfuGfuUfcUfcAfL96 | 466 | A-119721.1 | usGfsaGfaAfcAfuAfcuuUfcCfcCfuUfasasa | 621 |
| AD-58949.1 | A-119740.1 | GfscsAfgAfuGfuGfCfCfuGfuUfcCfuCfcAfL96 | 467 | A-119741.1 | usGfsgAfgGfaAfcAfggcAfcAfuCfuGfcsasa | 622 |
| AD-58936.1 | A-119750.1 | AfsasGfuAfuGfuUfCfUfcAfuGfuCfuUfcAfL96 | 468 | A-119751.1 | usGfsaAfgAfcAfuGfagaAfcAfuAfcUfususc | 623 |
| AD-58940.1 | A-119736.1 | UfscsAfcCfuAfaAfCfAfuGfcUfgUfcCfcUfL96 | 469 | A-119737.1 | asGfsgGfaCfaGfcAfuguUfuAfgGfuGfasgsa | 624 |
| AD-58945.1 | A-119738.1 | UfsusAfaGfgGfgAfAfAfgUfaUfgUfuCfuCfL96 | 470 | A-119739.1 | gsAfsgAfaCfaUfaCfuuuCfcCfcUfuAfasasg | 625 |
| AD-58937.1 | A-119766.1 | CfscsCfuAfgAfuCfUfCfaCfcUfaAfaCfaUfL96 | 471 | A-119767.1 | asUfsgUfuUfaGfgUfgagAfuCfuAfgGfgsasg | 626 |
| AD-58955.1 | A-119712.1 | UfscsCfcUfaAfuAfAfAfgCfuGfgAfuAfaGfL96 | 472 | A-119713.1 | csUfsuAfuCfcAfgCfuuuAfuUfaGfgGfascsa | 627 |
| AD-58951.1 | A-119710.1 | AfsasAfcAfuGfcUfGfUfcCfcUfaAfuAfaAfL96 | 473 | A-119711.1 | usUfsuAfuUfaGfgGfacaGfcAfuGfuUfusasg | 628 |
| AD-58947.1 | A-119708.1 | CfscsCfuAfaUfaAfAfGfcUfgGfaUfaAfgAfL96 | 474 | A-119709.1 | usCfsuUfaUfcCfaGfcuuUfaUfuAfgGfgsasc | 629 |
| AD-58933.1 | A-119702.1 | CfscsUfaAfuAfaAfGfCfuGfgAfuAfaGfaAfL96 | 475 | A-119703.1 | usUfscUfuAfuCfcAfgcuUfuAfuUfaGfgsgsa | 630 |
| AD-58931.1 | A-119748.1 | CfsusGfaAfgGfuUfGfCfuUfuAfaGfgGfgAfL96 | 476 | A-119749.1 | usCfscCfcUfuAfaAfgcaAfcCfuUfcAfgsgsg | 631 |
| AD-58938.1 | A-119704.1 | AfsasGfgGfgAfaAfGfUfaUfgUfuCfuCfaUfL96 | 477 | A-119705.1 | asUfsgAfgAfaCfaUfacuUfuCfcCfcUfusasa | 632 |
| AD-58957.1 | A-119744.1 | AfsgsCfuGfgAfuAfAfGfaAfgCfuGfcUfgUfL96 | 478 | A-119745.1 | asCfsaGfcAfgCfuUfcuuAfuCfcAfgCfususu | 633 |
| AD-58958.1 | A-119760.1 | AfscsCfuAfaAfcAfUfGfcUfgUfcCfcUfaAfL96 | 479 | A-119761.1 | usUfsaGfgGfaCfaGfcauGfuUfuAfgGfusgsa | 634 |
| AD-58930.1 | A-119732.1 | UfsusUfaAfgGfgGfAfAfaGfuAfuGfuUfcUfL96 | 480 | A-119733.1 | asGfsaAfcAfuAfcUfuucCfcCfuUfaAfasgsc | 635 |
| AD-58932.1 | A-119764.1 | UfscsGfuGfaGfaCfUfUfcUfgUfgUfuGfcAfL96 | 481 | A-119765.1 | usGfscAfaCfaCfaGfaagUfcUfcAfcGfascsu | 636 |
| AD-58961.1 | A-119746.1 | AfsusUfgAfgUfcGfUfGfaGfaCfuUfcUfgUfL96 | 482 | A-119747.1 | asCfsaGfaAfgUfcUfcacGfaCfuCfaAfusasg | 637 |
| AD-58952.1 | A-119726.1 | GfsusCfcCfuAfaUfAfAfaGfcUfgGfaUfaAfL96 | 483 | A-119727.1 | usUfsaUfcCfaGfcUfuuaUfuAfgGfgAfcsasg | 638 |
| AD-58946.1 | A-119754.1 | UfsusCfuGfuGfuUfGfCfaGfaUfgUfgCfcUfL96 | 484 | A-119755.1 | asGfsgCfaCfaUfcUfgcaAfcAfcAfgAfasgsu | 639 |
| AD-58956.1 | A-119728.1 | UfsgsGfcCfcCfuGfAfAfgGfuUfgCfuUfuAfL96 | 485 | A-119729.1 | usAfsaAfgCfaAfcCfuucAfgGfgGfcCfascsc | 640 |
| AD-58929.1 | A-119716.1 | GfsusUfgCfuUfuAfAfGfgGfgAfaAfgUfaUfL96 | 486 | A-119717.1 | asUfsaCfuUfuCfcCfcuuAfaAfgCfaAfcscsu | 641 |
| AD-58948.1 | A-119724.1 | UfsgsAfgAfcUfuCfUfGfuGfuUfgCfaGfaUfL96 | 487 | A-119725.1 | asUfscUfgCfaAfcAfcagAfaGfuCfuCfascsg | 642 |
| AD-58935.1 | A-119734.1 | GfscsUfgGfaUfaAfGfAfaGfcUfgCfuGfuUfL96 | 488 | A-119735.1 | asAfscAfgCfaGfcUfucuUfaUfcCfaGfcsusu | 643 |
| AD-58944.1 | A-119722.1 | CfsusCfcCfuAfgAfUfCfuCfaCfcUfaAfaCfL96 | 489 | A-119723.1 | gsUfsuUfaGfgUfgAfgauCfuAfgGfgAfgsgsg | 644 |
| AD-58959.1 | A-119714.1 | CfscsUfaAfaCfaUfGfCfuGfuCfcCfuAfaUfL96 | 490 | A-119715.1 | asUfsuAfgGfgAfcAfgcaUfgUfuUfaGfgsusg | 645 |
| AD-58960.1 | A-119730.1 | GfsasAfaGfuAfuGfUfUfcUfcAfuGfuCfuUfL96 | 491 | A-119731.1 | asAfsgAfcAfuGfaGfaacAfuAfcUfuUfcscsc | 646 |
| AD-58928.1 | A-119700.1 | GfscsCfcCfuGfaAfGfGfuUfgCfuUfuAfaGfL96 | 492 | A-119701.1 | csUfsuAfaAfgCfaAfccuUfcAfgGfgGfcscsa | 647 |
| AD-58950.1 | A-119756.1 | CfscsCfuCfcCfuAfGfAfuCfuCfaCfcUfaAfL96 | 493 | A-119757.1 | usUfsaGfgUfgAfgAfucuAfgGfgAfgGfgsgsu | 648 |
| AD-58962.1 | A-119762.1 | CfsusGfuCfcCfuAfAfUfaAfaGfcUfgGfaUfL96 | 494 | A-119763.1 | asUfscCfaGfcUfuUfauuAfgGfgAfcAfgscsa | 649 |

### Example 2. In vitro testing of siRNA Sequences

Table 6 shows the results of a single dose screen in Hep3B cells using the selected modified APOC3 iRNAs. The data are expressed as percent of APOC3 mRNA remaining in the cells transfected with iRNAs relative to APOC3 mRNA remaining in the cells transfected with the AD-1955 non-targeting control.

TABLE 6
Results of APOC3 single dose screen.

| Duplex Name | Avg. 10 nM | Avg. 0.1 nM | SD 10 nM | SD 0.1 nM |
|---|---|---|---|---|
| AD-57517.1 | 1.2 | 3.9 | 0.8 | 3.2 |
| AD-57544.1 | 2.4 | 18.2 | 1.1 | 14.2 |
| AD-57515.1 | 2.5 | 7.4 | 2.0 | 6.0 |
| AD-57551.1 | 2.8 | 19.4 | 2.2 | 14.8 |
| AD-57553.1 | 3.0 | 11.2 | 2.1 | 10.7 |
| AD-57498.1 | 3.4 | 19.3 | 2.4 | 16.0 |
| AD-57508.1 | 6.1 | 11.8 | 7.8 | 7.8 |
| AD-57523.1 | 6.2 | 39.7 | 1.2 | 20.8 |
| AD-57519.1 | 7.7 | 29.5 | 1.1 | 17.9 |
| AD-57561.1 | 8.0 | 50.7 | 2.3 | 31.0 |
| AD-57502.1 | 9.0 | 27.2 | 2.0 | 14.6 |
| AD-57547.1 | 9.8 | 39.4 | 4.3 | 21.9 |
| AD-57511.1 | 10.6 | 31.0 | 3.0 | 19.6 |
| AD-57493.1 | 10.9 | 42.0 | 6.0 | 22.1 |
| AD-57555.1 | 11.1 | 42.0 | 5.7 | 25.3 |
| AD-57503.1 | 12.9 | 36.9 | 3.2 | 23.5 |
| AD-57496.1 | 13.6 | 56.3 | 4.0 | 20.9 |
| AD-57559.1 | 13.7 | 66.1 | 5.7 | 37.5 |
| AD-57513.1 | 14.2 | 46.7 | 4.8 | 31.5 |
| AD-57534.1 | 15.5 | 46.5 | 17.3 | 23.7 |
| AD-57491.1 | 15.6 | 35.6 | 6.8 | 14.6 |
| AD-57512.1 | 15.8 | 48.0 | 5.7 | 29.6 |
| AD-57525.1 | 16.5 | 24.7 | 19.7 | 14.7 |
| AD-57539.1 | 17.8 | 54.8 | 10.7 | 21.0 |
| AD-57538.1 | 20.1 | 57.8 | 13.2 | 26.3 |
| AD-57529.1 | 20.5 | 62.3 | 9.7 | 29.7 |
| AD-57527.1 | 22.5 | 55.4 | 13.3 | 29.8 |
| AD-57521.1 | 25.8 | 42.2 | 24.4 | 22.2 |
| AD-57501.1 | 25.9 | 66.6 | 8.4 | 25.3 |
| AD-57548.1 | 26.1 | 63.0 | 12.2 | 27.2 |
| AD-57516.1 | 29.2 | 55.2 | 10.4 | 29.8 |
| AD-57533.1 | 29.7 | 58.9 | 22.8 | 29.8 |
| AD-57532.1 | 30.4 | 62.2 | 29.3 | 18.0 |
| AD-57497.1 | 30.5 | 57.2 | 6.0 | 31.2 |
| AD-57549.1 | 31.8 | 57.2 | 16.3 | 34.2 |
| AD-57506.1 | 34.3 | 55.5 | 9.0 | 30.7 |
| AD-57557.1 | 35.3 | 62.9 | 22.0 | 27.7 |
| AD-57520.1 | 37.6 | 52.7 | 17.7 | 26.5 |
| AD-57556.1 | 38.1 | 59.9 | 18.3 | 25.7 |
| AD-57505.1 | 39.8 | 63.2 | 19.2 | 18.5 |
| AD-57542.1 | 41.4 | 60.0 | 6.0 | 24.4 |
| AD-57552.1 | 41.4 | 62.8 | 13.3 | 25.8 |
| AD-57537.1 | 45.4 | 61.7 | 20.4 | 22.4 |
| AD-57541.1 | 58.3 | 72.5 | 13.8 | 26.0 |
| AD-57495.1 | 59.5 | 68.5 | 5.2 | 27.7 |
| AD-57507.1 | 64.2 | 62.0 | 2.4 | 33.1 |
| AD-57510.1 | 67.2 | 62.2 | 0.4 | 25.1 |
| AD-57522.1 | 67.5 | 67.4 | 15.5 | 30.6 |
| AD-57504.1 | 69.1 | 68.1 | 7.8 | 23.3 |
| AD-57546.1 | 72.1 | 69.8 | 9.6 | 30.4 |
| AD-57543.1 | 73.0 | 68.5 | 0.1 | 29.1 |
| AD-57558.1 | 75.6 | 68.3 | 11.9 | 27.2 |
| AD-57545.1 | 82.5 | 66.4 | 1.6 | 24.4 |
| AD-57509.1 | 83.4 | 71.2 | 2.1 | 31.0 |
| AD-57514.1 | 85.2 | 69.9 | 2.8 | 29.2 |
| AD-57550.1 | 85.2 | 64.3 | 3.0 | 19.0 |
| AD-57540.1 | 86.1 | 67.3 | 11.5 | 27.7 |
| AD-57500.1 | 86.3 | 73.3 | 2.7 | 31.3 |
| AD-57499.1 | 89.1 | 73.9 | 2.2 | 28.2 |
| AD-57536.1 | 90.1 | 75.6 | 13.4 | 35.8 |
| AD-57554.1 | 93.0 | 67.1 | 0.2 | 27.3 |
| AD-57518.1 | 95.0 | 68.0 | 1.5 | 21.7 |
| AD-57526.1 | 96.5 | 88.5 | 3.9 | 38.7 |
| AD-57531.1 | 99.8 | 73.6 | 28.0 | 30.1 |
| AD-57535.1 | 101.3 | 72.0 | 12.9 | 32.7 |
| AD-57562.1 | 103.6 | 81.2 | 8.6 | 38.6 |
| AD-58925.1 | 11.0 | 32.2 | 0.6 | 1.1 |
| AD-58911.1 | 12.1 | 25.2 | 0.4 | 0.9 |
| AD-58924.1 | 14.0 | 26.9 | 0.8 | 2.3 |
| AD-58933.1 | 15.1 | 43.8 | 0.4 | 1.1 |
| AD-58922.1 | 15.3 | 25.0 | 1.0 | 1.8 |
| AD-58916.1 | 23.0 | 57.6 | 0.9 | 0.5 |
| AD-58935.1 | 23.3 | 59.5 | 0.4 | ND |
| AD-58920.1 | 23.6 | 59.5 | 0.6 | ND |
| AD-58918.1 | 26.6 | 57.3 | 0.6 | 1.2 |
| AD-58917.1 | 29.4 | 71.9 | 1.1 | 3.5 |
| AD-58914.1 | 31.0 | 58.5 | 1.3 | 3.4 |
| AD-58919.1 | 31.8 | 83.8 | 1.9 | 1.6 |
| AD-58913.1 | 32.4 | 68.6 | 1.5 | 2.1 |
| AD-58957.1 | 36.0 | 81.2 | 1.4 | 0.8 |
| AD-58923.1 | 42.8 | 86.5 | 2.9 | 1.5 |
| AD-58915.1 | 48.4 | 77.7 | 0.8 | 2.6 |
| AD-58927.1 | 57.0 | 92.6 | 1.6 | 1.4 |
| AD-58962.1 | 57.9 | 96.7 | 5.5 | 3.7 |
| AD-58921.1 | 63.9 | 95.8 | 2.5 | 2.8 |
| AD-58926.1 | 65.1 | 88.1 | 2.0 | 2.5 |
| AD-58947.1 | 69.4 | 93.6 | 3.4 | 3.8 |
| AD-58928.1 | 74.5 | 91.3 | 5.3 | 0.0 |
| AD-58931.1 | 77.2 | 96.6 | 6.2 | 2.0 |
| AD-58950.1 | 82.4 | 98.3 | 1.1 | 1.0 |
| AD-58936.1 | 89.2 | 102.0 | 2.3 | 1.3 |
| AD-58956.1 | 93.0 | 102.4 | 1.4 | ND |
| AD-58912.1 | 93.9 | 98.0 | 3.1 | 3.4 |
| AD-58951.1 | 97.0 | 103.5 | 2.7 | 1.4 |
| AD-58949.1 | 97.3 | 110.5 | 1.8 | ND |
| AD-58955.1 | 97.4 | 103.8 | 3.7 | 1.3 |
| AD-58948.1 | 97.9 | 101.6 | 7.9 | 3.6 |
| AD-58937.1 | 98.7 | 89.4 | 4.8 | 2.2 |
| AD-58932.1 | 99.4 | 97.3 | 5.8 | ND |
| AD-58946.1 | 100.8 | 97.9 | 2.0 | ND |
| AD-58944.1 | 106.5 | 83.9 | 4.7 | 4.2 |
| AD-58954.1 | 108.7 | 95.1 | 3.5 | 1.5 |
| AD-58940.1 | 109.0 | 97.7 | ND | 0.3 |
| AD-58961.1 | 110.4 | 111.3 | ND | 6.0 |
| AD-58952.1 | 110.7 | 96.0 | ND | 1.5 |
| AD-58945.1 | 114.5 | 101.5 | ND | 1.5 |
| AD-58929.1 | 115.0 | 102.9 | ND | ND |
| AD-58958.1 | 115.7 | 118.1 | ND | ND |
| AD-58938.1 | 118.5 | 92.4 | ND | 2.7 |
| AD-58939.1 | 119.3 | 98.7 | ND | 0.8 |
| AD-58960.1 | 120.6 | 101.2 | ND | ND |
| AD-58930.1 | 123.7 | 108.6 | ND | 1.2 |
| AD-58959.1 | 133.2 | 123.3 | ND | 3.2 |
| AD-64805.1 | 4.4 | 18.8 | 2.2 | 11.0 |
| AD-64793.1 | 6.1 | 23.9 | 3.2 | 9.6 |
| AD-64799.1 | 6.0 | 51.4 | 3.3 | 10.8 |
| AD-64787.1 | 8.2 | 45.0 | 3.8 | 24.0 |
| AD-64813.1 | 9.1 | 45.5 | 4.9 | 16.0 |
| AD-64794.1 | 8.7 | 50.2 | 2.1 | 15.9 |
| AD-64824.1 | 9.6 | 57.5 | 2.2 | 5.4 |
| AD-64825.1 | 12.7 | 60.3 | 8.9 | 14.2 |
| AD-64819.1 | 12.2 | 62.4 | 5.1 | 13.6 |
| AD-64828.1 | 16.9 | 49.2 | 6.5 | 6.7 |
| AD-64789.1 | 21.8 | 39.7 | 6.1 | 5.7 |
| AD-64807.1 | 20.0 | 48.7 | 9.8 | 17.5 |
| AD-64812.1 | 18.2 | 59.3 | 6.1 | 19.9 |
| AD-64795.1 | 19.9 | 54.2 | 6.3 | 10.1 |
| AD-64804.1 | 18.0 | 59.4 | 6.0 | 7.6 |
| AD-64827.1 | 17.3 | 67.3 | 8.8 | 17.7 |
| AD-64788.1 | 15.9 | 69.2 | 3.4 | 11.6 |
| AD-64832.1 | 19.0 | 61.4 | 6.8 | 7.5 |
| AD-64792.1 | 24.6 | 54.6 | 7.6 | 6.2 |
| AD-64831.1 | 20.7 | 63.8 | 4.2 | 9.0 |
| AD-64820.1 | 19.6 | 73.9 | 7.7 | 7.2 |
| AD-64816.1 | 26.7 | 52.8 | 6.1 | 6.5 |
| AD-64811.1 | 15.4 | 97.5 | 4.2 | 17.6 |
| AD-64821.1 | 18.7 | 79.8 | 4.6 | 11.5 |
| AD-64808.1 | 27.6 | 58.4 | 12.0 | 8.9 |
| AD-64810.1 | 30.8 | 55.7 | 10.1 | 7.2 |
| AD-64817.1 | 20.0 | 91.2 | 9.1 | 8.4 |
| AD-64797.1 | 26.1 | 67.1 | 3.2 | 15.3 |
| AD-64829.1 | 25.5 | 75.8 | 10.3 | 18.4 |
| AD-64802.1 | 34.2 | 63.9 | 11.0 | 10.1 |
| AD-64798.1 | 34.5 | 66.9 | 18.0 | 8.3 |
| AD-64815.1 | 29.3 | 75.2 | 6.0 | 12.4 |
| AD-64791.1 | 38.6 | 72.1 | 7.6 | 13.5 |
| AD-64814.1 | 35.7 | 87.6 | 14.9 | 11.8 |
| AD-64800.1 | 37.9 | 96.0 | 10.2 | 13.8 |
| AD-64823.1 | 33.1 | 110.5 | 7.7 | 9.3 |
| AD-64818.1 | 41.3 | 120.3 | 8.0 | 10.5 |
| AD-64806.1 | 52.1 | 103.8 | 13.8 | 28.7 |
| AD-64809.1 | 84.7 | 81.4 | 16.3 | 22.4 |
| AD-64822.1 | 98.0 | 89.6 | 26.0 | 6.2 |
| AD-64796.1 | 103.4 | 95.4 | 20.8 | 10.5 |
| AD-64790.1 | 131.8 | 84.5 | 46.9 | 8.5 |

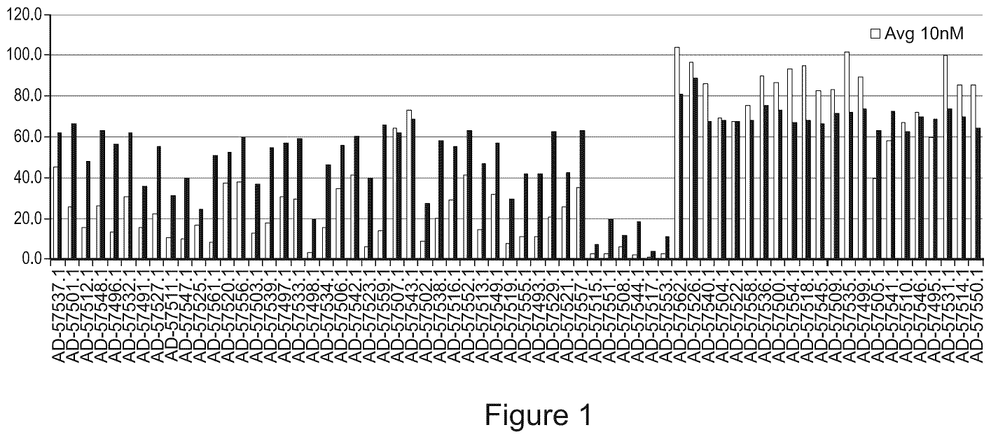

Table 7 shows the dose response of Hep3B cells transfected with the indicated cyno/human cross reactive modified APOC3 iRNAs. The indicated IC₅₀ values represent the IC₅₀ values relative to untreated cells. The results of a single dose screen (Avg. 10 nM blue bars and Avg. 0.1 nM red bars) for APOC3 iRNAs are shown in FIG. 1. Based on the results of the screen, three iRNAs (AD-57553.1, AD-57547.1 and AD-58924.1) were selected for further in vivo testing.

TABLE 7

| APOC3 Dose Response Screen | |
|---|---|
| | IC50 |
| Duplex ID | (nM) |
| AD-57553.1 | 0.012 |
| AD-57544.1 | 0.022 |
| AD-57551.1 | 0.021 |
| AD-57547.1 | 0.044 |
| AD-57555.1 | 0.075 |
| AD-57534.1 | 0.142 |
| AD-57549.1 | 0.871 |
| AD-57527.1 | 0.209 |
| AD-57533.1 | 1.425 |
| AD-57538.1 | 0.177 |
| AD-57559.1 | 0.395 |
| AD-58925.1 | 0.13 |
| AD-58911.1 | 0.09 |
| AD-58924.1 | 0.08 |
| AD-58933.1 | 0.39 |
| AD-58922.1 | 0.09 |
| AD-58916.1 | 0.40 |
| AD-58935.1 | 0.62 |
| AD-58920.1 | 0.30 |
| AD-58918.1 | 0.97 |
| AD-58917.1 | 1.78 |
| AD-58914.1 | 3.20 |

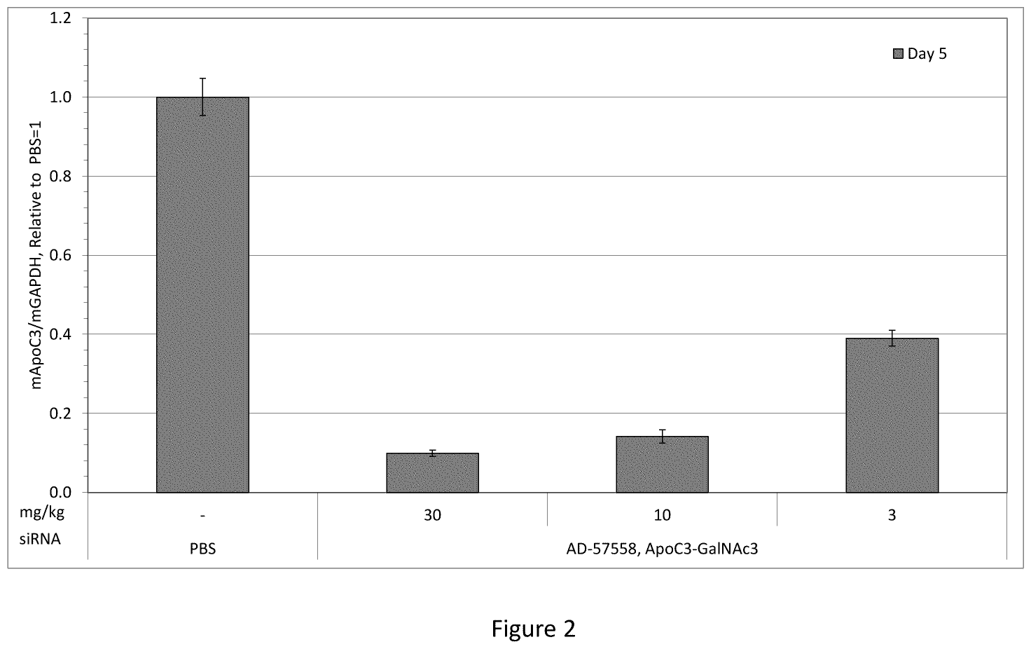

Example 3. In vivo testing of AD-57558 siRNA Sequence in Wild-Type Mice The rodent-specific AD-57558-GalNAc3 sequence was tested in wild-type mice for its ability to inhibit the expression of APOC3 and to reduce serum lipids. AD-57558-GalNAc3 was administered cubsutaneously at 3 mg/kg, 10 mg/kg or 30 mg/kg as a single dose, with PBS used as a negative control. The expression of APOC3 was measured 5 days after dosing by RT-PCR. The results, shown in FIG. 2, indicate that AD-57558-GalNAc3 is able to reduce the expression of APOC3 by about 60% (for a 3 mg/kg dose), about 80% (for a 10 mg/kg dose) and about 85% (for a 30 mg/kg dose).

### Example 4. Generation and Characterization of a Mouse Model of APOC3 Overexpression

To enable ApoC3 GalNAc conjugate lead finding in vivo with compounds that do not cross-react with the rodent APOC3 gene, a system for overexpression of the human APOC3 gene in the liver of C57Bl/6 mice was employed. An adeno-associated virus serotype 8 (AAV8) encapsidated AAV2 vector genome expressing human APOC3 under the liver-specific TBG promoter was generated. Model characterization studies were performed to identify optimal conditions, including the number of viral genome copies (GC) and time needed for high, durable expression of human ApoC3 in AAV-transduced mice.

Further testing was carried out using the dosing of 10¹¹ genome copies of the AAV2 vector per mouse. The levels of APOC3 mRNA in the liver of AAV-hApoC3 mice were measured by RT-PCR 1.5 weeks, 6.5 weeks, 8 weeks and 16 weeks after administration of the hAPOC3 AAV vector. The results of RT-PCR (data not shown) indicate that high expression levels of the human APOC3 gene were achieved in mice within 10 days following administration of 10¹¹ genome copies of hAPOC3 AAV and were sustained for at least 6 months with little animal-to-animal variability.

### Example 5. Testing Potential Lead iRNAs in a Mouse Model of APOC3 Overexpression

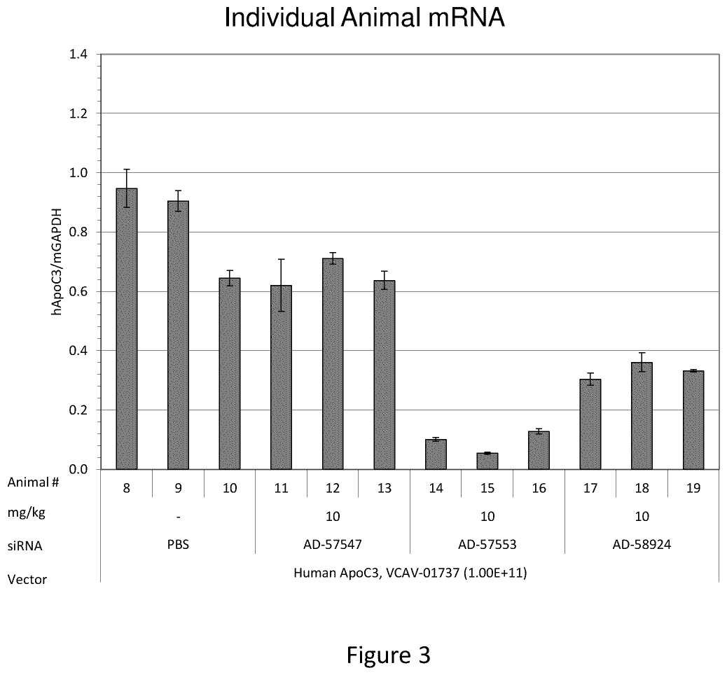

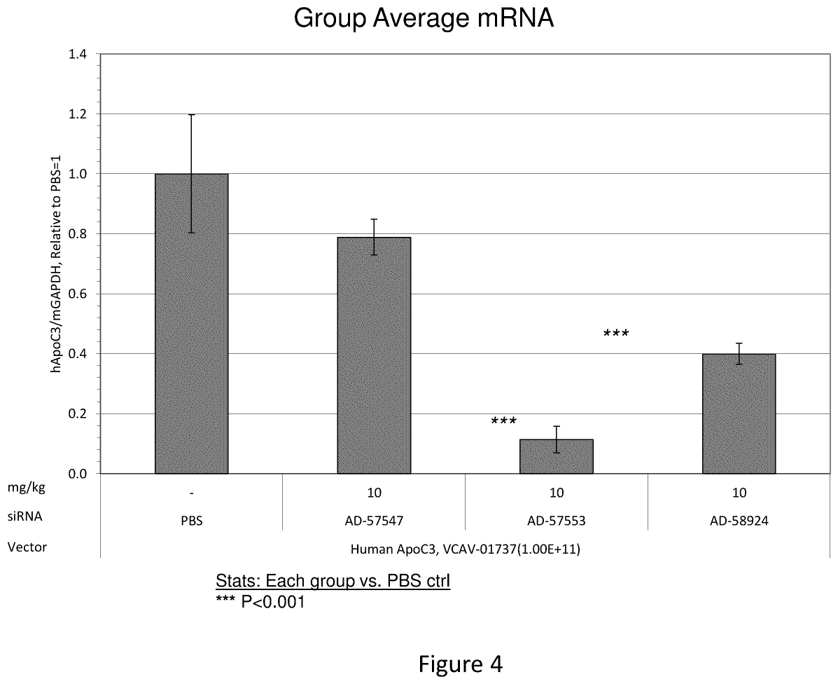

Once characterized, the APOC3-AAV mouse model system of APOC3 overexpression was used for in vivo screening of AD-57553, AD-57547 and AD-58924 that were identified based on their potency in vitro. For single high dose screen experiments, APOC3-AAV mice previously injected with 10¹¹ genome copies of hAPOC3 AAV were administered a single 10 mg/kg dose of AD-57553, AD-57547 and AD-58924 or PBS (as control), and APOC3 mRNA was subsequently measured. The results are presented in FIGS. 3 and 4. Specifically, FIG. 3 shows the levels of APOC3 mRNA measured in individual APOC3-AAV mice injected with AD-57553, AD-57547 and AD-58924 or PBS, while FIG. 4 shows group mRNA averages. The data indicate that AD-57553 is the most effective in inhibiting APOC3 expression.

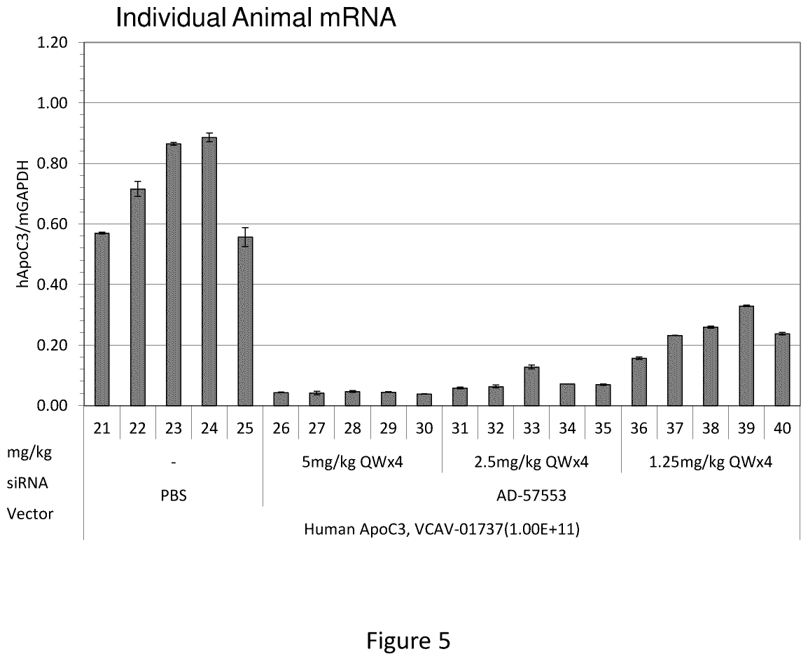

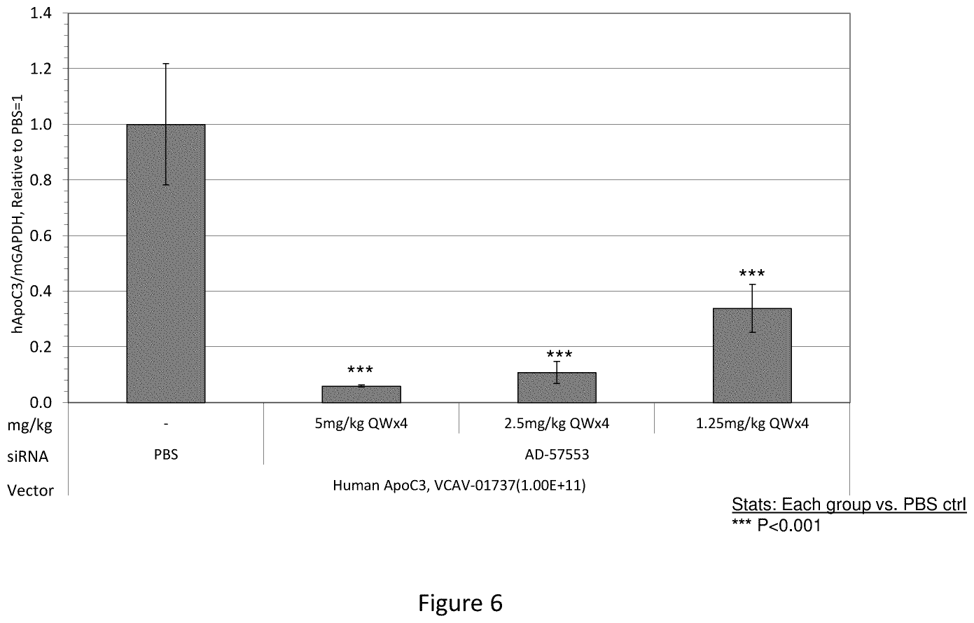

AD-57553 was selected for further in vivo testing in dose response and multi-dose studies. For the dose response experiments, APOC3-AAV mice previously injected with 10¹¹ genome copies of hAPOC3 AAV were administered 1.25 mg/kg, 2.5 mg/kg and 5 mg/kg doses weekly for 4 weeks, and the APOC3 mRNA levels were subsequently measured. The results are presented in FIGS. 5 and 6. Specifically, FIG. 5 shows the levels of APOC3 mRNA measured in individual APOC3-AAV mice injected with AD-57553, while FIG. 6 shows group mRNA averages. The data show an increasing inhibition of APOC3 mRNA expression with the increasing dose of AD-57553.

### Example 6. Generation and In Vivo Testing of Additional iRNA Sequences Based on AD-57553

From the initial round of SAR optimization of iRNA chemistry, 10 additional iRNA sequences were generated based on the AD-57553 lead sequence by introducing changes in 2′F, 2′OMe and 5′P modifications. The additional iRNAs are presented in Tables 8 and 9 below.

TABLE 8
Additional iRNA Sequences (Unmodified)

| Duplex | Sense Oligo | Sense Sequence | SEQ | Antisense | Antisense Sequence | SEQ |
|---|---|---|---|---|---|---|
| Name | Name | (5′ to 3′) | ID NO: | OligoName | (5′ to 3′) | ID NO: |
| AD- | A-117361.2 | GCUUAAAAGGGACAGUA | 650 | A-117362.2 | AGAAUACUGUCCCUUUUAA | 661 |
| 57553.3 | | UUCU | | | GCAA | |
| AD- | A-117361.2 | GCUUAAAAGGGACAGUA | 651 | A-130731.1 | UGAAUACUGUCCCUUUUAA | 662 |
| 65696.1 | | UUCU | | | GCAA | |
| AD- | A-130732.1 | GCUUAAAAGGGACAGUA | 652 | A-130734.1 | UGAAUACUGUCCCUUUUAA | 663 |
| 65697.1 | | UUCA | | | GCAA | |
| AD- | A-130732.1 | GCUUAAAAGGGACAGUA | 653 | A-130735.1 | UGAAUACUGUCCCUUUUAA | 664 |
| 65698.1 | | UUCA | | | GCAA | |
| AD- | A-130732.1 | GCUUAAAAGGGACAGUA | 654 | A-130736.1 | UGAAUACUGTCCCTUUUAA | 665 |
| 65699.1 | | UUCA | | | GCAA | |
| AD- | A-130733.1 | GCUUAAAAGGGACAGUC | 655 | A-130734.1 | UGAAUACUGUCCCUUUUAA | 666 |
| 65700.1 | | UUCA | | | GCAA | |
| AD- | A-130733.1 | GCUUAAAAGGGACAGUC | 656 | A-130735.1 | UGAAUACUGUCCCUUUUAA | 667 |
| 65701.1 | | UUCA | | | GCAA | |
| AD- | A-130733.1 | GCUUAAAAGGGACAGUC | 657 | A-130736.1 | UGAAUACUGTCCCTUUUAA | 668 |
| 65702.1 | | UUCA | | | GCAA | |
| AD- | A-130737.1 | GCUUAAAAGGGACAGUA | 658 | A-130734.1 | UGAAUACUGUCCCUUUUAA | 669 |
| 65703.1 | | UUCA | | | GCAA | |
| AD- | A-130737.1 | GCUUAAAAGGGACAGUA | 659 | A-130735.1 | UGAAUACUGUCCCUUUUAA | 670 |
| 65704.1 | | UUCA | | | GCAA | |
| AD- | A-130737.1 | GCUUAAAAGGGACAGUA | 660 | A-130736.1 | UGAAUACUGTCCCTUUUAA | 671 |
| 65705.1 | | UUCA | | | GCAA | |

TABLE 9
Additional iRNA Sequences (Modified)

| | Sense | | SEQ | sense | | SEQ |
|---|---|---|---|---|---|---|
| Duplex | Oligo | Sense Sequence | ID | Oligo- | Antisense Sequence | ID |
| Name | Name | (5′ to 3′) | NO: | Name | (5′ to 3′) | NO: |
| AD- | A- | GfscsUfuAfaAfaGfGfGfaCfa | 672 | A- | asGfsaAfuAfcUfgUfcccUfuUf | 683 |
| 57553.3 | 117361.2 | GfuAfuUfcUfL96 | | 117362.2 | uAfaGfcsAfsa | |
| AD- | A- | GfscsUfuAfaAfaGfGfGfaCfa | 673 | A- | VPusGfsaAfuAfcUfgUfcccUfu | 684 |
| 65696.1 | 117361.2 | GfuAfuUfcUfL96 | | 130731.1 | UfuAfaGfcsasa | |
| AD- | A- | gscsuuaaaaggdGacagu(Agn)u | 674 | A- | usGfsaauAfcUfGfucccUfuUfu | 685 |
| 65697.1 | 130732.1 | ucaL96 | | 130734.1 | aagcsasa | |
| AD- | A- | gscsuuaaaaggdGacagu(Agn)u | 675 | A- | usGfsaauacugucccUfuuuaagc | 686 |
| 65698.1 | 130732.1 | ucaL96 | | 130735.1 | sasa | |
| AD- | A- | gscsuuaaaaggdGacagu(Agn)u | 676 | A- | usdGsaauacugdTcccdTuuuaag | 687 |
| 65699.1 | 130732.1 | ucaL96 | | 130736.1 | csasa | |
| AD- | A- | gscsuuaaaaggdGacagucuuca | 677 | A- | usGfsaauAfcUfGfucccUfuUfu | 688 |
| 65700.1 | 130733.1 | L96 | | 130734.1 | aagcsasa | |
| AD- | A- | gscsuuaaaaggdGacagucuuca | 678 | A- | usGfsaauacugucccUfuuuaagc | 689 |
| 65701.1 | 130733.1 | L96 | | 130735.1 | sasa | |
| AD- | A- | gscsuuaaaaggdGacagucuuca | 679 | A- | usdGsaauacugdTcccdTuuuaag | 690 |
| 65702.1 | 130733.1 | L96 | | 130736.1 | csasa | |
| AD- | A- | gscsuuaaAfaGfGfGfacaguauu | 680 | A- | usGfsaauAfcUfGfucccUfuUfu | 691 |
| 65703.1 | 130737.1 | caL96 | | 130734.1 | aagcsasa | |
| AD- | A- | gscsuuaaAfaGfGfGfacaguauu | 681 | A- | usGfsaauacugucccUfuuuaagc | 692 |
| 65704.1 | 130737.1 | caL96 | | 130735.1 | sasa | |
| AD- | A- | gscsuuaaAfaGfGfGfacaguauu | 682 | A- | usdGsaauacugdTcccdTuuuaag | 693 |
| 65705.1 | 130737.1 | caL96 | | 130736.1 | csasa | |

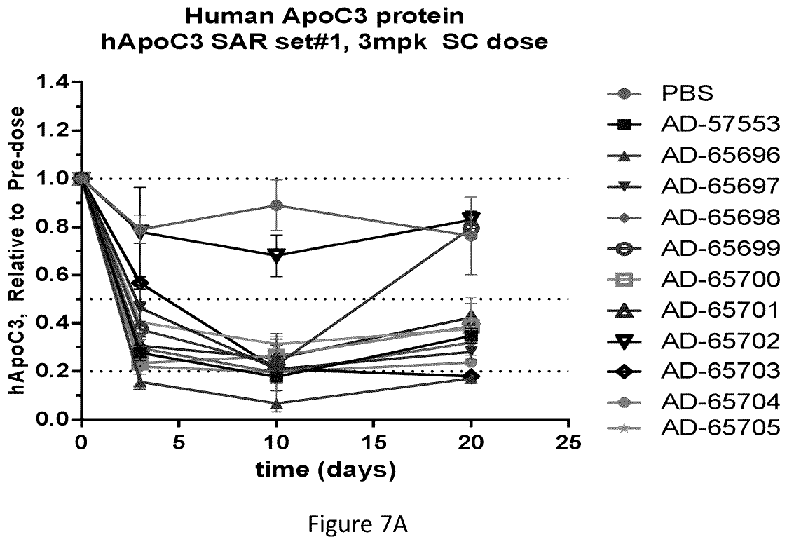

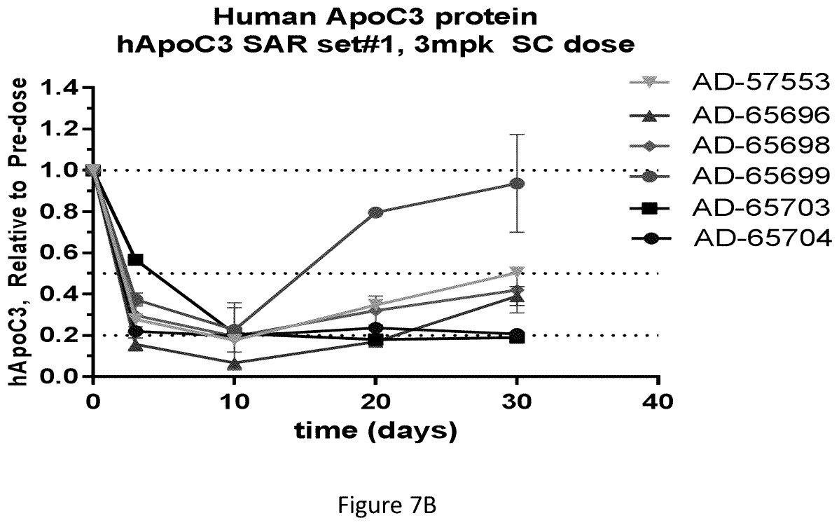

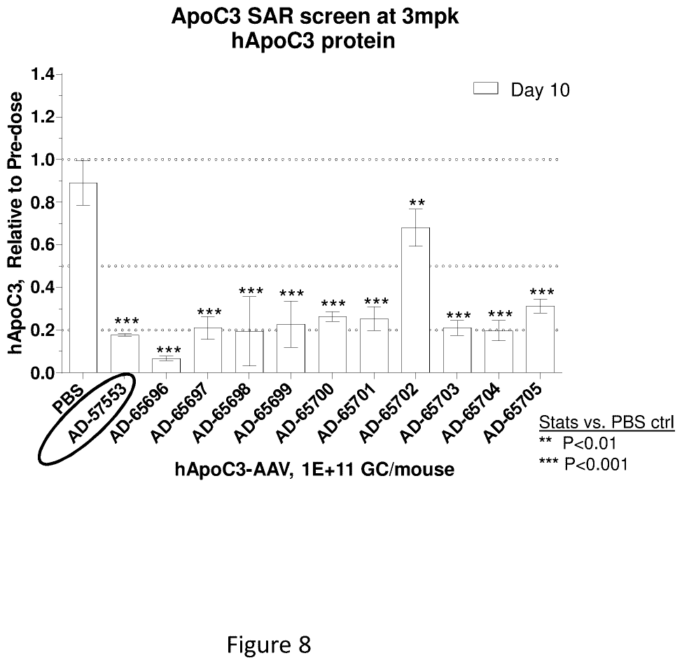

The additional sequences were tested in APOC3-AAV mice for their ability to inhibit the expression of the APOC3 protein. Specifically, APOC3-AAV mice previously injected with 10¹¹ genome copies of hAPOC3 AAV were administered a single dose of 3 mg/kg of the indicated modified iRNA, and the resulting levels of circulating serum APOC3 protein were measured on days 5, 10 and 20 after dosing. FIG. 7A presents the time course for up to 20 days for the levels of serum APOC3 protein measured for each tested iRNA sequence and FIG. 7B presents the time course for up to 30 days for the levels of serum APOC3 protein for six selected iRNA sequences. FIGS. 8 and 9 represent the data for each tested iRNA sequence for days 10 and 20, respectively. The data indicate that the most active iRNA sequences, such as AD-65704, are able to achieve about 80% knockdown of serum APOC3 protein on days 10 and 20.

### Example 7. Testing New Lead iRNAs in a Mouse Model of APOC3 Overexpression

AD-57553, AD-65696, AD-65703 and AD-65704 were selected for follow-on studies in order to test the effect of fluorine content and of vinyl phosphate on the ability of the iRNA agent to inhibit the expression of APOC3 protein in vivo. Table 9 above shows the modified sequences of AD-57553, AD-65696, AD-65703 and AD-65704, and Table 10 below contains a brief explanation of the modifications present in each strand.

TABLE 10
Modified Sequences of Selected iRNAs Used for In Vivo Experiments

| | | SEQ | | SEQ | |
|---|---|---|---|---|---|
| Duplex | Sense Sequence | ID | Antisense Sequence | ID | |
| Name | (5′ to 3′) | NO: | (5′ to 3′) | NO: | Explanation of Modifications |
| AD- | GfscsUfuAfaAfaGfGfGfa | 694 | asGfsaAfuAfcUfgUfcccU | 700 | Contains 6 phosphorothioates |
| 57553.3 | CfaGfuAfuUfcUfL96 | | fuUfuAfaGfcsAfsa | | |
| AD- | GfscsUfuAfaAfaGfGfGfa | 695 | VPusGfsaAfuAfcUfgUfcc | 701 | Contains 6 phosphorothioates |
| 65696.1 | CfaGfuAfuUfcUfL96 | | cUfuUfuAfaGfcsasa | | and a vinyl phosphate (VP) on |
| | | | | | the antisense strand |
| AD- | gscsuuaaaaggdGacagu | 696 | usGfsaauacugucccUfuuua | 702 | Ultra low fluorine content; |
| 65698.1 | (Agn)uucaL96 | | agcsasa | | no 2′F on the sense strand, |
| | | | | | two 2′Fs on the antisense |
| | | | | | strand |
| AD- | gscsuuaaaaggdGacagu | 697 | usdGsaauacugdTcccdTuu | 703 | No fluorine content; no 2F′s |
| 65699.1 | (Agn)uucaL96 | | uaagcsasa | | on either sense or antisense |
| | | | | | strand |
| AD- | gscsuuaaAfaGfGfGfaca | 698 | usGfsaauAfcUfGfucccUf | 704 | Low fluorine content; 10 total |
| 65703.1 | guauucaL96 | | uUfuaagcsasa | | 2′Fs on the sense and |
| | | | | | antisense strands |
| AD- | gscsuuaaAfaGfGfGfaca | 699 | usGfsaauacugucccUfuuua | 705 | Low fluorine content: 6 total |
| 65704.1 | guauucaL96 | | agcsasa | | 2′Fs on the sense and |
| | | | | | antisense strands |

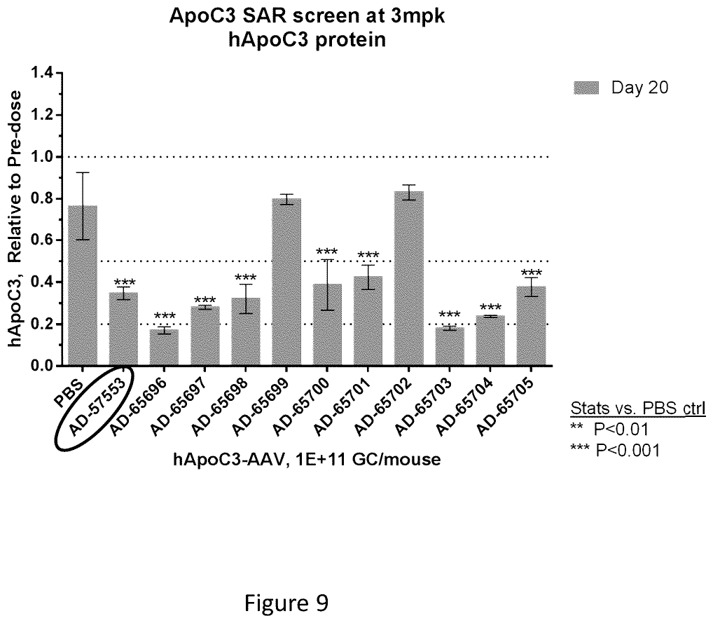

The ability of the iRNAs to inhibit the expression of the human APOC3 protein in vivo was tested in the APOC3-AAV mouse model system of APOC3 overexpression. For a single dose screen study, APOC3-AAV mice previously injected with 10¹¹ genome copies of APOC3 AAV were administered a single 3 mg/kg dose of AD-57553, AD-65696, AD-65703 and AD-65704 or PBS (as control), and the levels of APOC3 protein relative to pre-dose were measured at days 0, 3, 10, 20, 30, 41, 55 and 70 post-dose. FIG. 10 presents the data for all four tested iRNAs. The data in FIG. 10 indicate that iRNAs with low 2′F content (AD-65703 and AD-65704) are capable of sustaining an ˜80% of knock-down of APOC3 for 30 days. The data also demonstrate that it takes 55-70 days for the APOC3 levels to rebound to those observed in the control (PBS) group following a single 3 mg/kg dose.

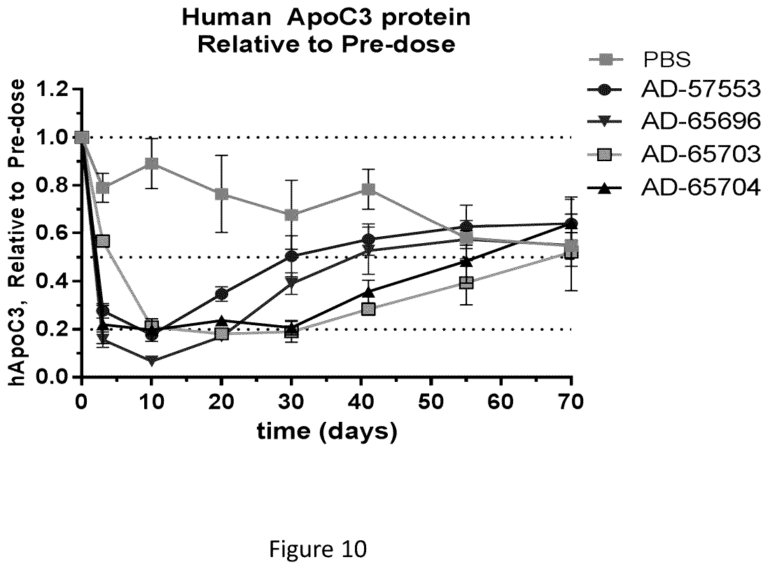

AD-57553, AD-65696, AD-65699, AD-65703 and AD-65704 were used for further in vivo testing in dose response and multi-dose studies. For the dose response experiments, APOC3-AAV mice previously injected with 10¹¹ genome copies of APOC3 AAV received four subcutaneous doses of 0.3 mg/kg, 1 mg/kg and 3 mg/kg of each iRNA (Q2W×4 dosing schedule). The animals were bled at days 0, 7, 14, 21, 35, 49, 62 and 77, and the amount of APOC3 was evaluated. The dosing schedule used in the experiment is depicted schematically in FIG. 11.

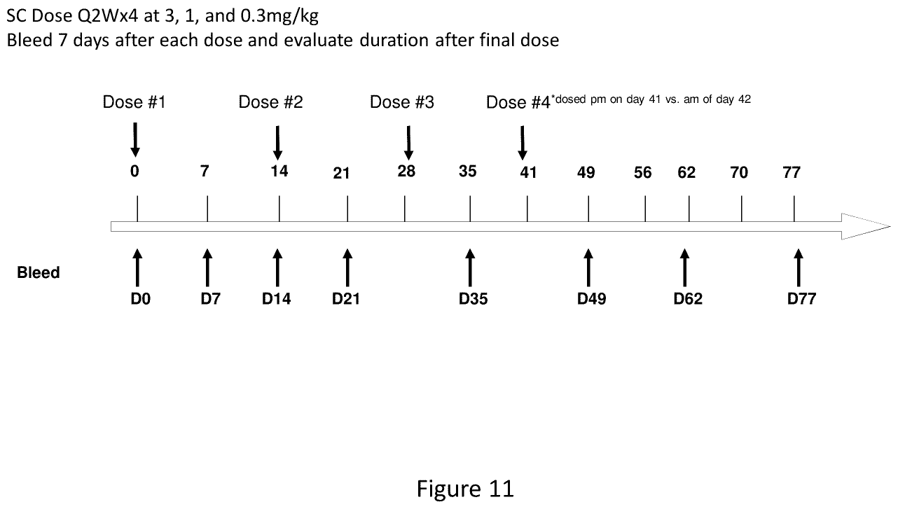

The time courses for the 0.3 mg/kg, 1 mg/kg and 3 mg/kg doses are shown in FIGS. 12A, 12B and 12C, respectively. The data in FIGS. 12A-C demonstrate that at a 0.3 mg/kg dose each of the tested iRNAs is able to inhibit the expression of APOC3 protein up to 50% relative to pre-dose measurement. The data also demonstrate that at the 1 mg/kg and 3 mg/kg doses, AD-65699, which contains no 2′F modifications, is less effective than the other tested iRNAs at inhibiting the expression of APOC3. The data further show that up to a 94% decrease in the relative APOC3 levels is achieved after a 3 mg/kg dose of AD-57553, AD-65696, AD-65703 and AD-65704 is administered to the animals. This knock-down in the levels of APOC3 is sustained for at least 3 weeks following the last dose. Two of the tested iRNAs, AD-65696 and AD-65704 were able to sustain ˜80% inhibition of APOC3 protein for at least 5 weeks following the last dose.

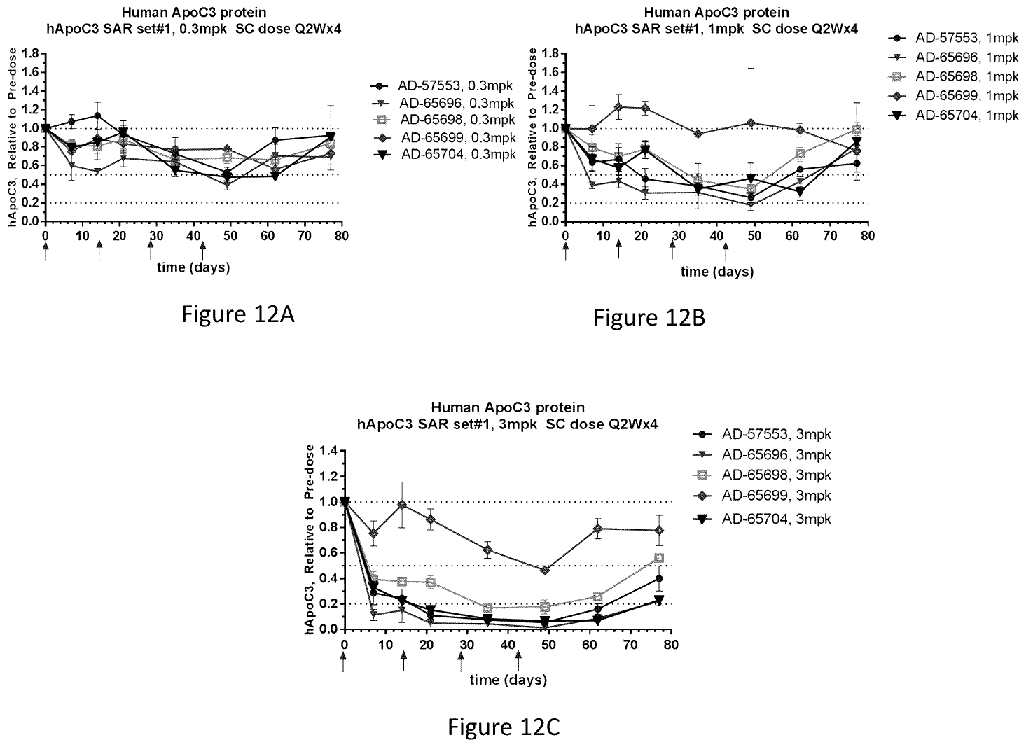

One of the selected lead sequences, AD-65704, containing 6 2′F modifications on the sense and antisense strand, was tested in a single-dose titration study using the APOC3-AAV mouse model system of APOC3 overexpression. For dose screen experiments, APOC3-AAV mice previously injected with 10¹¹ genome copies of hAPOC3 AAV were administered a single dose of 0.3 mg/kg, 1 mg/kg or 3 mg/kg of AD-65704 or PBS (as control), and the levels of APOC3 protein relative to pre-dose were measured after 14 days. The results, presented in FIG. 13, indicate that the dose effective to achieve 80% inhibition of the expression of APOC3 (ED80) is ˜3 mg/kg, while the dose effective to achieve 40% inhibition of the expression of APOC3 (ED₄₀) is 1 mg/kg.

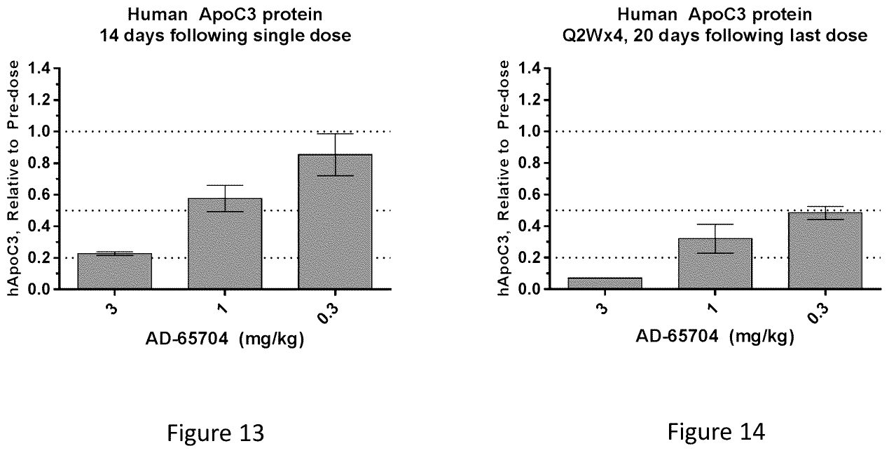

For dose titration experiments, APOC3-AAV mice previously injected with 10¹¹ genome copies of APOC3 AAV were subcutaneously administered AD-65704 at a dose of 0.3 mg/kg, 1 mg/kg or 3 mg/kg. For each dose level, a total of four doses were administered, one every other week (Q2W×4 administration). FIG. 14 shows the amount of APOC3 protein, relative to pre-dose, measured 20 days following the last dose. The data indicate that the dose effective to achieve 90% inhibition of the expression of APOC3 (ED₉₀) is 3 mg/kg; the dose effective to achieve 70% inhibition of the expression of APOC3 (ED₇₀) is 1 mg/kg; and the dose effective to achieve 50% inhibition of the expression of APOC3 (ED₅₀) is achieved at 0.3 mg/kg.

The results presented in this example (FIGS. 10-14) demonstrate that iRNAs with low fluorine content (AD-65703 and AD-65704) achieve ˜80% knock-down of APOC3 that is sustained for at least 30 days when administered as a single 3 mg/kg dose (see FIG. 10). Administration of four 3 mg/kg doses every two weeks achieves up to 94% lowering of APOC3 levels, with over 90% of knock-down sustained for at least 3 weeks (see FIG. 12C).

The results for an iRNA with ultra low fluorine content (AD-65698) indicate that this iRNA is able to achieve up to 83% knock-down with four doses of 3 mg/kg administered every two weeks, with 75% of the knock-down sustained for 3 weeks following the last dose (see FIG. 12C). The results for an iRNA with no fluorine content (AD-65699) indicate that with a single dose of 3 mg/kg, this iRNA is able to achieve a knock-down that is very short-lived. Four 3 mg/kg doses administered every two weeks can increase the knock-down to up to 50%, with APOC3 levels returning to baseline within 2 weeks after administration of the final dose (see FIG. 12C).

Addition of VP on the 5′ of the antisense strand results in a boost in the ability of the iRNA to inhibit the expression of APOC3. This is evident from the 94% knock-down of APOC3 observed after a single 3 mg/kg dose of VP-containing AD-65696, as compared to ˜80% knock-down observed for the parent iRNA AD-57553 without VP (see FIG. 10). Multi-dosing experiments with AD-65696 resulted in up to 99% knock-down of APOC3 with 3 mg/kg, ˜80% knock-down with 1 mg/kg and in >50% knock-down with 0.3 mg/kg doses (see FIGS. 12A-C).

### Example 8. Identification of iRNAs That Cross-React with Rabbit APOC3

The iRNA agents shown in Table 7 were analyzed for their ability to cross-react with rabbit APOC3. It was determined based on the number of mismatches to rabbit APOC3 that AD-58911, AD-58924, AD-58922 and AD-58916 were cross-reactive with rabbit APOC3.

The four rabbit cross-reactive sequences were modified to contain a total of 10 2′F modifications on the sense and antisense strand resulting in iRNAs AD-67221, AD-67222, AD-67223, and AD-67224. The unmodified and modified sequences for AD-67221, AD-67222, AD-67223, and AD-67224 are presented, respectively, in Tables 11A and 11B below.

TABLE 11A
Unmodified iRNAs That Cross-React to Rabbit APOC3.

| | | | | SEQ |
|---|---|---|---|---|
| | Sense Sequence | SEQ | Antisense Sequence | ID |
| Duplex Name | (5′ to 3′) | ID NO: | (5′ to 3′) | NO: |
| AD-67221.1 | CCCAAUAAAGCUGGACAAGAA | 706 | UUCUUGUCCAGCUUUAUUGGGAG | 710 |
| AD-67222.1 | CCUCCCAAUAAAGCUGGACAA | 707 | UUGUCCAGCUUUAUUGGGAGGCC | 711 |
| AD-67223.1 | UCCCAAUAAAGCUGGACAAGA | 708 | UCUUGUCCAGCUUUAUUGGGAGG | 712 |
| AD-67224.1 | CUCCCAAUAAAGCUGGACAAG | 709 | CUUGUCCAGCUUUAUUGGGAGGC | 713 |

TABLE 11B
Modified iRNAs That Cross-React to Rabbit APOC3.

| | Sense Sequence | SEQ | Antisense Sequence | SEQ |
|---|---|---|---|---|
| Duplex Name | (5′ to 3′) | ID NO: | (5′ to 3′) | ID NO: |
| AD-67221.1 | cscscaauAfaAfGfCfuggacaagaaL96 | 714 | usUfscuuGfuCfCfagcuUfuAfuugggsasg | 718 |
| AD-67222.1 | cscsucccAfaUfAfAfagcuggacaaL96 | 715 | usUfsgucCfaGfCfuuuaUfuGfggaggscsc | 719 |
| AD-67223.1 | uscsccaaUfaAfAfGfcuggacaagaL96 | 716 | usCfsuugUfcCfAfgcuuUfaUfugggasgsg | 720 |
| AD-67224.1 | csuscccaAfuAfAfAfgcuggacaagL96 | 717 | csUfsuguCfcAfGfcuuuAfuUfgggagsgsc | 721 |

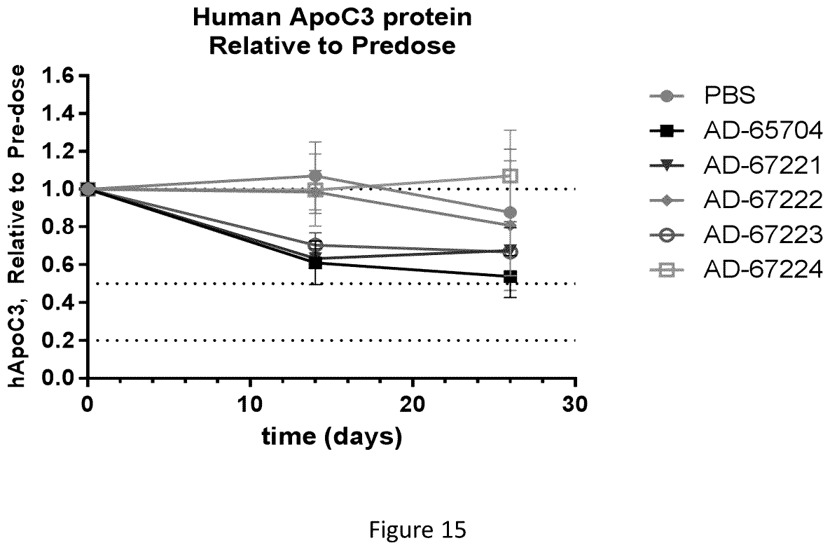

These iRNAs were tested in a single-dose study using the APOC3-AAV mouse model system of APOC3 overexpression. APOC3-AAV mice previously injected with 10¹¹ genome copies of hAPOC3 AAV were administered a single dose of 1 mg/kg of AD-65704, AD-67221, AD-67222, AD-67223, AD-67224, or PBS (as control), and the levels of APOC3 protein relative to pre-dose were measured on days 14 and 26 post dose. The results, presented in FIG. 15, indicate that at day 14, iRNAs AD-67222 and AD-67224 were not active at 1 mg/kg, AD-67223 showed 30% inhibition of APOC3 protein, and AD-67221 was comparable to AD-65704 with ˜40% inhibition of APOC3 protein at 1 mg/kg dose. While initial silencing of APOC3 protein is similar between AD-65704 and AD-67221 at day 14, the level of activity at day 26 indicates that AD-65704 is more durable than AD-67221, achieving 46% and 33% inhibition of APOC3 protein, respectively.

### Example 9. Testing the Effect of Vinyl Phosphate Modification

The aim of this study was to test the effect of vinyl phosphate (VP) and 2′F modifications on the antisense strand on the ability of iRNA to inhibit the expression of APOC3. The iRNAs used in the study are summarized in Table 12 below.

TABLE 12
iRNAs used for the VP study

| | | SEQ | | SEQ |
|---|---|---|---|---|
| Duplex | | ID | | ID |
| Name | Sense Sequence (5′ to 3′) | NO: | Antisense Sequence (5′ to 3′) | NO: |
| AD-65698 | gscsuuaaaaggdGacagu(Agn)uucaL96 | 722 | usGfsaauacugucccUfuuuaagcsasa | 728 |
| AD-66239 | gscsuuaaaaggdGacagu(Agn)uucaL96 | 723 | VPusGfsaAfuAfcUfgUfcccUfuUfuAfaGfcsasa | 729 |
| AD-65701 | gscsuuaaaaggdGacagucuucaL96 | 724 | usGfsaauacugucccUfuuuaagcsasa | 730 |
| AD-66240 | gscsuuaaaaggdGacagucuucaL96 | 725 | VPusGfsaAfuAfcUfgUfcccUfuUfuAfaGfcsasa | 731 |
| AD-65704 | gscsuuaaAfaGfGfGfacaguauucaL96 | 726 | usGfsaauacugucccUfuuuaagcsasa | 732 |
| AD-66241 | gscsuuaaAfaGfGfGfacaguauucaL96 | 727 | VPusGfsaAfuAfcUfgUfcccUfuUfuAfaGfcsasa | 733 |

iRNA agents AD-65698 and AD-66239 have the same sense strand but different antisense strands. Similarly, duplexes AD-65701 and AD-66240 have the same sense strand, but different antisense strands. iRNA agents AD-65704 and AD-66241 contain the same sense strand, but different antisense strands. The antisense strands for AD-65698, AD-65701 and AD-65704 are the same and contain four nucleotides containing phosphorothioates and two nucleotides containing a 2′-fluoro modification. The antisense strands for AD-66239, AD-66240 and AD-66241 are the same and contain four nucleotides containing phosphorothioates, nine nucleotides containing a 2′-fluoro modification and one vinyl phosphate at the 5′end of the antisense strand.

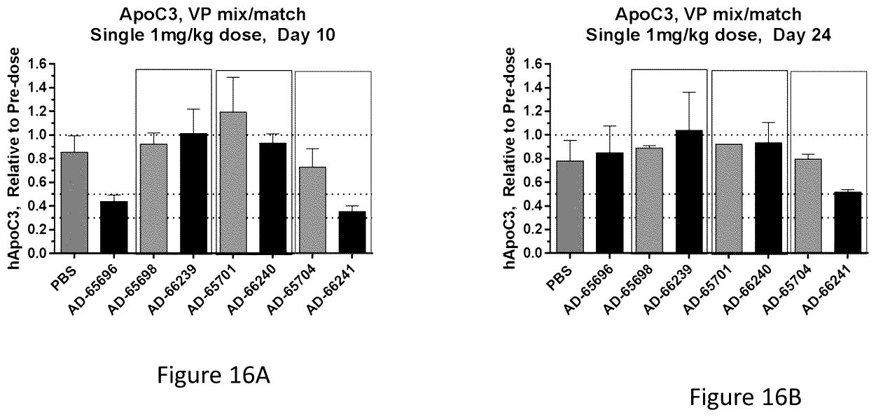

APOC3-AAV mice previously injected with 10¹¹ genome copies of APOC3 AAV were administered a single 1 mg/kg dose of each iRNA listed in Table 11 or PBS (as control), and the levels of APOC3 protein relative to pre-dose were measured at days 10 and 24 post-dose. The data for day 10 is shown in FIG. 16A, and the data for day 24 is shown in FIG. 16B.

The results demonstrate that mixing and matching of a VP modification on the antisense strand with low fluorine content of the sense strand resulted in a similar boost in activity, however, was not able to improve duration over pairing of the sense strand with antisense strands without the VP modification.

### Example 10. In Vivo Testing of Additional iRNA Sequences Based on AD-65704

SAR optimization of AD-65704 chemistry generated 10 additional iRNA sequences (Table 13).

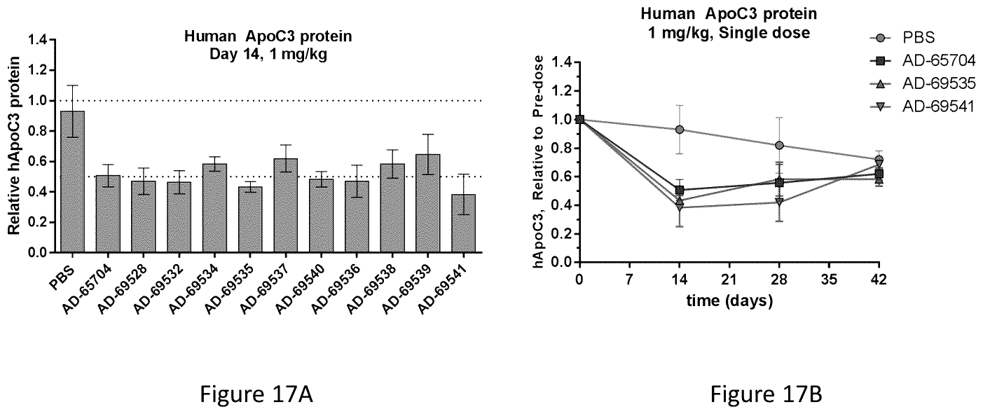

These iRNAs were tested in a single-dose study using the APOC3-AAV mouse model system of APOC3 overexpression. Briefly, two weeks after injecting APOC3-AAV mice with 10¹¹ genome copies of hAPOC3 AAV, the mice (n=3) were subcutaneously administered a single 1 mg/kg dose of the iRNA agents or PBS (as control). The serum levels of APOC3 protein relative to pre-dose levels were measured by ELISA assay on days 14, 28, and 42 post dose. The results, presented in FIGS. 17A and 17B, indicate that at day 14, all of the tested iRNAs were active at 1 mg/kg.

TABLE 13
Modified iRNA Sequences

| Duplex | | SEQ | | SEQ |
|---|---|---|---|---|
| Name | Sense Sequence (5′ to 3′) | ID NO: | Antisense Sequence (5′ to 3′) | ID NO: |
| AD-65704.1 | gscsuuaaAfaGfGfGfacaguauucaL96 | 734 | usGfsaauacugucccUfuuuaagcsasa | 745 |
| AD-69528.1 | gscsuuaaAfaGfGfGfacaguauucaL96 | 735 | usGfsaauAfcUfgucccUfuUfuaagcsasa | 746 |
| AD-69532.1 | gscsuuaaAfaGfgGfacaguauucaL96 | 736 | usGfsaauacugucCfcUfuuuaagcsasa | 747 |
| AD-69534.1 | gscsuuaaaaGfgGfacaguauucaL96 | 737 | usGfsaauacugucccUfuuuaagcsasa | 748 |
| AD-69535.1 | gscsuuaaaaGfgGfacaguauucaL96 | 738 | usGfsaauacugucCfcUfuuuaagcsasa | 749 |
| AD-69537.1 | gscsuuaaaaGfgGfacaguuuucaL96 | 739 | usGfsaauacugucccUfuuuaagcsasa | 750 |
| AD-69540.1 | gscsuuaaaaGfgGfacaguuuucaL96 | 740 | usGfsaauacugucCfcUfuuuaagcsasa | 751 |
| AD-69536.1 | gscsuuaaaaGfgGfacagudTuucaL96 | 741 | usGfsaauacugucccUfuuuaagcsasa | 752 |
| AD-69538.1 | gscsuuaaaaGfgGfacagu(Agn)uucaL96 | 742 | usGfsaauacugucccUfuuuaagcsasa | 753 |
| AD-69539.1 | gscsuuaaaaGfgGfacagudTuucaL96 | 743 | usGfsaauacugucCfcUfuuuaagcsasa | 754 |
| AD-69541.1 | gscsuuaaaaGfgGfacagu(Agn)uucaL96 | 744 | usGfsaauacugucCfcUfuuuaagcsasa | 755 |

### Example 11. In Vivo Testing of iRNA Agents in Non-Human Primates

Based on the results described in Example 10, three iRNA agents, AD-65704, AD-69535 and AD-69541, were selected for evaluation in non-human primtaes.

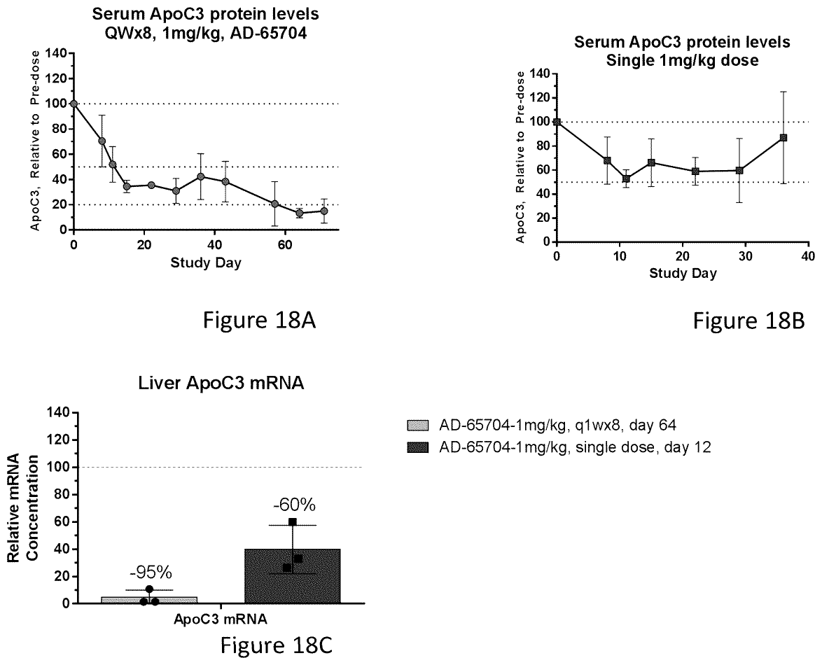

Single and multi-dosing experiments were performed in Cynomolgus monkeys. In one set of experiments, naïve male Cynomolgus monkeys (n=3) were subcutaneously administered a single weekly dose of 1 mg/kg dose of AD-65704 on day 1, or naïve male Cynomolgus monkeys (n=3) were subcutaneously administered a once weekly 1 mg/kg dose of AD-65704 on days 1, 8, 15, 22, 29, 36, 43, and 50. Serum was collected on days −7, −1, 1, 8, 11, 15, 22, 29, 36, 43, 57, 64, and 71. The level of Cynomolgus ApoC3 protein was determined by ELISA. Liver biopsies were performed on days −7, 12, 30, and 64 and the level of ApoC3 mRNA was determined. The results of the single-dose study are depecieted in FIGS. 18A and 18B and demonstrate that once weekly administration of 1 mg/kg of AD-65704 achieves >80% lowering of total serum ApoC3 and up to 50% lowering of total ApoC3 protein (18B). The data also demonstrate that once weekly administration of 1 mg/kg of AD-65704 achieves a lowering of liver ApoC3 mRNA by 60% as compared to the predose level. As depicted in FIG. 18C, once weekly dosing of 1 mg/kg of AD-65704 lowers the level of ApoC3 mRNA by 95% relative to the predose level.

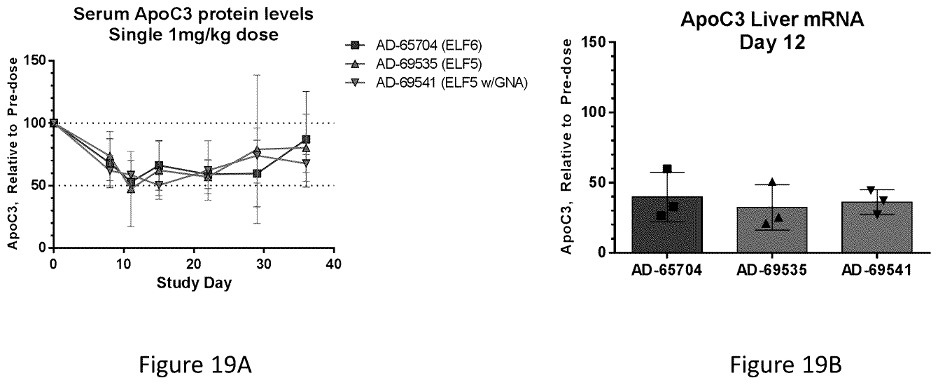

In another set of experiments, naïve male Cynomolgus monkeys (n=3) were subcutaneously administered a single weekly dose of 1 mg/kg dose of AD-65704, AD-69535, or AD-69541 on day 1. Serum was collected on days −7, −1, 1, 8, 11, 15, 22, 29, and 36. The level of Cynomolgus ApoC3 protein was determined by ELISA. Liver biopsies were performed on days −7, 12, 30, and 64 and the level of ApoC3 mRNA was determined. The data demonstrate that a single 1 mg/kg dose of all three of the iRNA agents tested lowers ApoC3 protein levels to about 50% of the baseline level (ED_{50 protein}=about 1 mg/kg) (19A). As demonstrated in FIG. 19B, a single 1 mg/kg dose of AD-65704 lowers ApoC3 mRNA by 60% relative to pre-dose levels, AD-69535 lowers ApoC3 mRNA by 68% relative to pre-dose levels, and AD-69541 lowers ApoC3 mRNA by 64% relative to pre-dose levels (ED_{50 mRNA}<1 mg/kg).

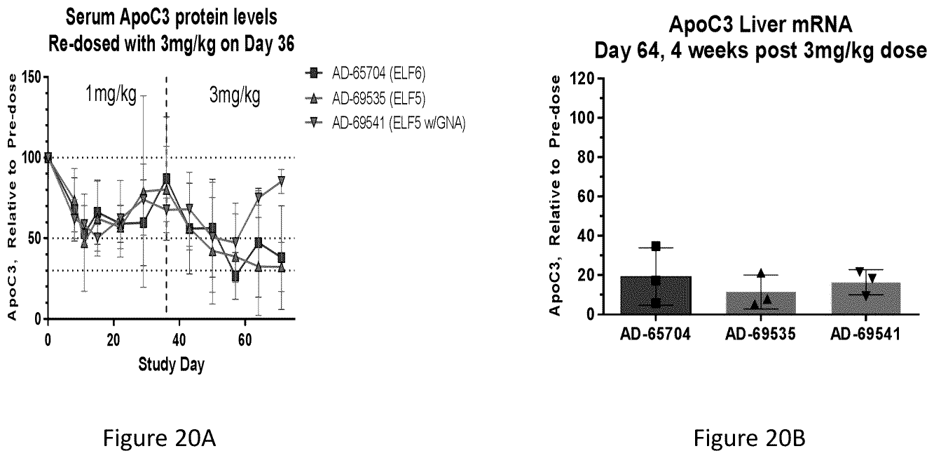

An additional multi-dose study was performed in Cynomolgus monkeys with AD-65704, AD-69535, and AD-69541. Naïve male Cynomolgus monkeys were subcutaneously administered a single 1 mg/kg dose AD-65704, AD-69535, or AD-69541 on day 1 and were subsequently administered a single subcutaneous 3 mg/kg dose of the same agent on day 36. N=3/group. Serum was collected on days −7, −1, 1, 8, 11, 15, 22, 29, 36, 43, 50, 57, 64, and 71. The level of Cynomolgus ApoC3 protein was determined by ELISA. Liver biopsies were performed on days −7, 12, 30, and 64 and the level of ApoC3 mRNA was determined. FIG. 20A demonstrates that administration of a 3 mg/kg dose of AD-65704 or AD-69535 lowers the level of ApoC3 protein by about 70% relative to pre-dose levels (ED_{70 protein=about} 3 mg/kg) and FIG. 20B demonstrates that, at day 64 post-administration of a 3 mg/kg dose of AD-65704, the level of ApoC3 mRNA is lowered by 81% relative to pre-dose levels, that at day 64 post-administration of a 3 mg/kg dose of AD-69535, the level of ApoC3 mRNA is lowered by 88% relative to pre-dose levels, and that at day 64 post-administration of a 3 mg/kg dose of AD-69541 the level of ApoC3 mRNA is lowered by 84% relative to pre-dose levels (ED_{80 mRNA}≤3 mg/kg).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A double stranded RNAi agent for inhibiting expression of apolipoprotein C3 (APOC3) in a cell, wherein said double stranded RNAi agent comprises a sense strand and an antisense strand forming a double-stranded region, wherein said sense strand comprises at least 15 contiguous nucleotides differing by no more than 3 nucleotides from the nucleotide sequence of SEQ ID NO:1, and said antisense strand comprises at least 15 contiguous nucleotides differing by no more than 3 nucleotides from the nucleotide sequence of SEQ ID NO:2,
wherein substantially all of the nucleotides of at least one strand comprise a nucleotide modification, and
wherein said sense strand is conjugated to a ligand attached at the 3′-terminus.

2. The double stranded RNAi agent of claim 1, wherein said antisense strand comprises at least 15 contiguous nucleotides differing by no more than 3 nucleotides from any one of the antisense strand nucleotide sequences listed in Tables 4A, 4B, 5, 8, 9, 10, 11A, 11B, and 12, 13.

3. The double stranded RNAi agent of claim 1, wherein at least one of said nucleotide is selected from the group consisting of a 3′-terminal deoxy-thymine (dT) nucleotide modification, a 2′-O-methyl modification, a 2′-fluoro modification, a 2′-deoxy-modification, a locked nucleotide modification, an unlocked nucleotide, a conformationally restricted nucleotide modification, a constrained ethyl nucleotide modification, an abasic nucleotide modification, a 2′-amino-modified nucleotide modification, a 2′-O-allyl-modification, 2′-C-alkyl-modification, 2′-hydroxly-modification, a 2′-methoxyethyl modification, a 2′-O-alkyl-modification, a morpholino nucleotide modification, a phosphoramidate, modification a non-natural base comprising nucleotide modification, a tetrahydropyran modification, a 1,5-anhydrohexitol modification, a cyclohexenyl modification, a nucleotide comprising a 5′-phosphorothioate group modification, a nucleotide comprising a 5′-methylphosphonate group modification, a nucleotide comprising a 5′ phosphate or 5′ phosphate mimic modification, a nucleotide comprising vinyl phosphate modification, a nucleotide comprising adenosine-glycol nucleic acid (GNA) modification, a nucleotide comprising thymidine-glycol nucleic acid (GNA) S-Isomer modification, a nucleotide comprising 2-hydroxymethyl-tetrahydrofurane-5-phosphate modification, a nucleotide comprising 2′-deoxythymidine-3′phosphate modification, a nucleotide comprising 2′-deoxyguanosine-3′-phosphate modification, and a terminal nucleotide linked to a cholesteryl derivative or a dodecanoic acid bisdecylamide group modification.

4. (canceled)

5. The double stranded RNAi agent of claim 1, wherein all of the nucleotides of said sense strand comprise a nucleotide modification; all of the nucleotides of said antisense strand comprise a nucleotide modification; or all of the nucleotides of said sense strand and all of the nucleotides of said antisense strand comprise a nucleotide modification.

6. The double stranded RNAi agent of claim 1, wherein at least one strand comprises a 3′ overhang of at least 1 nucleotide; or at least one strand comprises a 3′ overhang of at least 2 nucleotides.

7.-8. (canceled)

9. The double stranded RNAi agent of claim 1, wherein the double-stranded region is 15-30 nucleotide pairs in length; 17-23 nucleotide pairs in length; 17-25 nucleotide pairs in length; 23-27 nucleotide pairs in length; 19-21 nucleotide pairs in length; or 21-23 nucleotide pairs in length.

10. The double stranded RNAi agent of claim 1, wherein each strand is independently 15-30 nucleotides in length; or 19-30 nucleotides in length.

11. The double stranded RNAi agent of claim 1, wherein the nucleotide modifications are 2′-O-methyl modifications and 2′-fluoro modifications.

12. The double stranded RNAi agent of claim 1, wherein the ligand is one or more GalNAc derivatives attached through a bivalent or trivalent branched linker.

13. The double stranded RNAi agent of claim 1, wherein the ligand is

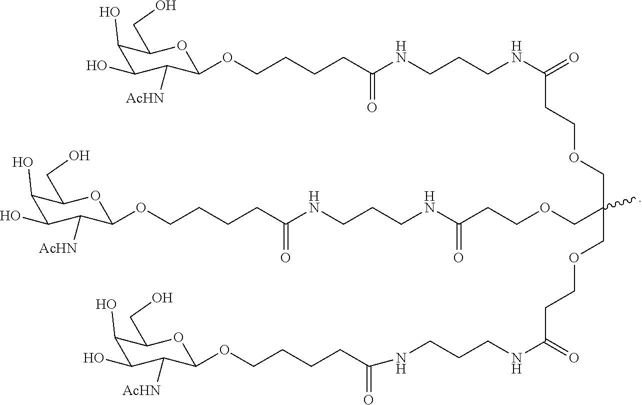 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

14. The double stranded RNAi agent of claim 1, wherein the ligand is attached to the 3′ end of the sense strand.

15. The double stranded RNAi agent of claim 14, wherein the double stranded RNAi agent is conjugated to the ligand as shown in the following schematic 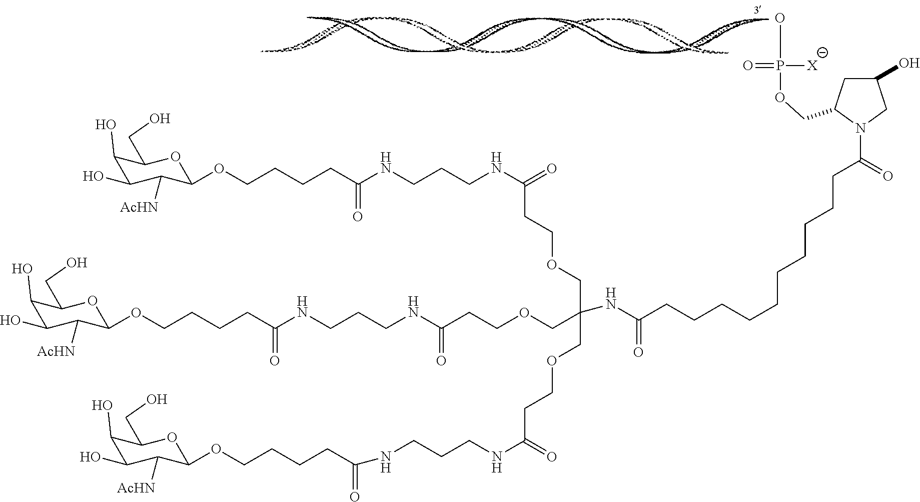
wherein X is O or S.

16. The double stranded RNAi agent of claim 1, wherein said RNAi agent further comprises at least one phosphorothioate or methylphosphonate internucleotide linkage.

17. A cell containing the double stranded RNAi agent of claim 1.

18. A pharmaceutical composition comprising the double stranded RNAi agent of claim 1.

19. (canceled)

20. The pharmaceutical composition of claim 18, wherein the double stranded RNAi agent is present in an unbuffered solution; or a buffered solution.

21. A method of inhibiting apolipoprotein C3 (APOC3) expression in a cell, the method comprising:
(a) contacting the cell with the double stranded RNAi agent of claim 1; and
(b) maintaining the cell produced in step (a) for a time sufficient to obtain degradation of the mRNA transcript of a APOC3 gene, thereby inhibiting expression of the APOC3 gene in the cell.

22. (canceled)

23. A method of treating a subject having an apolipoprotein C3 (APOC3) associated disease, comprising administering to the subject a therapeutically effective amount of the double stranded RNAi agent of claim 1, thereby treating said subject.

24. The method of claim 23, wherein the APOC3 associated disease is selected from the group consisting of hypertriglyceridemia, non-alcoholic fatty liver disease, non-alcoholic steatohepatitis, polycystic ovary syndrome, kidney disease, obesity, type 2 diabetes mellitus (insulin resistance), hypertension, atherosclerosis and pancreatitis.

25.-26. (canceled)
